(19) United States
(12) Patent Application Publication
(10) Pub. No.: US 2024/0300927 A1
(43) Pub. Date: Sep. 12, 2024

(54) AMIDES AS CBL-B INHIBITORS

(71) Applicant: Genentech, Inc., South San Francisco, CA (US)

(72) Inventors: Jun Liang, Los Altos Hills, CA (US); Araz Jakalian, Montreal (CA); Michael John Lambrecht, Burlingame, CA (US); Robin Larouche-Gauthier, Montreal (CA); Malcolm Huestis, San Francisco, CA (US); Man Un Ung, San Mateo, CA (US); Xiaojing Wang, Belmont, CA (US); Arun Yadav, Montreal (CA); Jason Robert Zbieg, Montara, CA (US); Fabio Broccatelli, San Diego, CA (US)

(73) Assignee: Genentech, Inc., South San Francisco, CA (US)

(21) Appl. No.: 18/275,655

(22) PCT Filed: Feb. 3, 2022
(86) PCT No.: PCT/US2022/015153

§ 371 Date: Aug. 3, 2023

Related U.S. Application Data
(60) Provisional application No. 63/145,401, filed on Feb. 3, 2021.

(51) Int. Cl. C07D 405/14; A61K 31/4439

(52) U.S. Cl. CPC C07D 405/14; A61K 31/4439

(57) ABSTRACT

Various amide compounds that bind Cbl-B, including those that are selective for C-Cbl, and methods of making and using the same. Representative amide compounds include molecules falling within the following formulae:

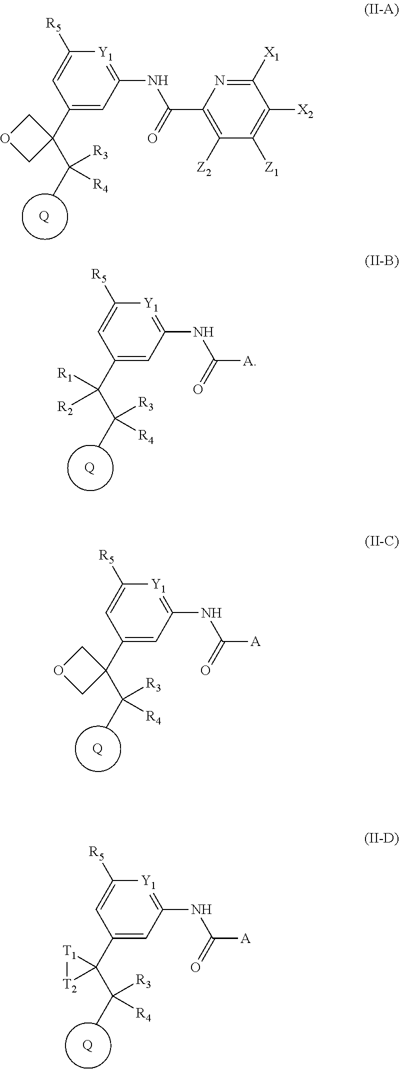

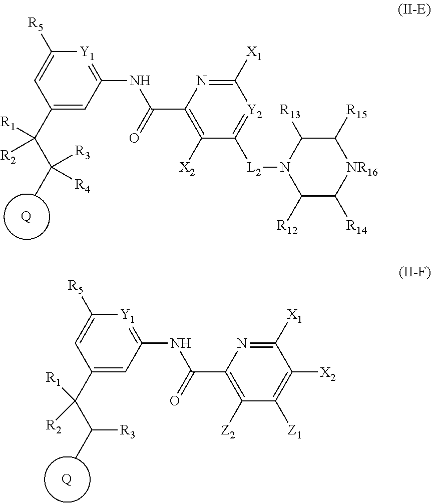

### CLAIM OF PRIORITY

This application claims benefit of priority to U.S. provisional patent application No. 63/145,401, filed Feb. 3, 2021, the contents of which are incorporated by reference herein.

### TECHNICAL FIELD

The technology described herein generally relates to inhibitors of Cbl-B and which also have activity against, or which can be selective over, C-Cbl, and additionally relates to methods of making and using the same.

### BACKGROUND

Casitas B-lineage lymphoma-b (Cbl-b) is a member of the Cbl family of RING E3 ubiquitin ligases. A common function of Cbl family proteins is the negative regulation of receptor tyrosine kinase signaling. Since Cbl-b inhibition leads to immune activation, it has been expected that Cbl-b inhibitors could be broadly active in multiple oncology indications.

Cbl proteins comprise three principal domains: a conserved N-terminal tyrosine kinase binding (TKB) domain, a short linker region, and a RING finger (RF) domain. The TKB domain is, in turn, composed of three subdomains: a 4-helix bundle (4H), a calcium-binding domain with an EF-hand fold, and a variant Src homology region 2 (SH2) domain, all three of which are involved in phosphotyrosine binding. The TKB domain binds substrates, such as ZAP70, that contain phosphotyrosine motifs.

The conserved RF domain, which has intrinsic E3 ligase activity, can recruit E2 ubiquitin-conjugating enzymes, and mediate the transfer of ubiquitin to substrates.

Phosphorylation of Cbl-b at Y363 within the linker domain regulates its E3 ubiquitin ligase activity by removing the masking of the RF domain by the TKB domain.

In T cells, Cbl-b is a key tolerogenic factor that directly regulates the cells' activation. Specifically, Cbl-b is highly expressed in murine and human CD4+ and CD8+ T cells, where it functions as a potent negative regulator of T cell activation by controlling activation thresholds and the requirement for co-stimulation. Mechanistically Cbl-b acts by ubiquitinating multiple substrates downstream of the T cell receptor (TCR), including ZAP70, resulting in TCR internalization and termination of signaling. Loss of Cbl-b in T cells leads to prolonged TCR surface expression, and in combination with TCR stimulation results in increased expression of activation markers, such as CD25, cytokine production and proliferation.

Mouse models have surprisingly demonstrated that the loss of Cbl-b leads to increased adaptive and innate anti-tumor immunity, mediated by enhanced T cell effector function as well as increased natural killer (NK) cell activity. Cbl-b deficient mice spontaneously reject a variety of cancer tumors, including spontaneous solid tumors and hematopoietic malignancies, in a CD8 T cell-dependent manner. Adoptive transfer of Cbl-b−/− CD8+ T cells is sufficient to reject tumors, demonstrating that Cbl-b has a non-redundant role in regulating T-cell-mediated anti-tumor activity.

Consequently, developing a small molecule approach to inhibit Cbl-b is a promising but challenging goal for cancer immunotherapy.

Nevertheless, c-Cbl, a closely related family member to Cbl-b, shares high sequence homology with Cbl-b at the N-terminus, including in the TKB and RING domains. c-Cbl negatively regulates signaling of a number of growth factor receptors, including Flt3 and c-Kit. Among other defects, c-Cbl deficient mice exhibit expansion of hematopoietic stem cells and multipotent progenitors in the bone marrow. In mice that are conditionally deficient in both c-Cbl and Cbl-b this defect is amplified, and the mice develop a rapidly-progressive and lethal myeloproliferative disease accompanied by splenomegaly by around 8 weeks of age. Given a broad spectrum of functions of c-Cbl in growth factor receptor regulation, and a strong amplification in the dysregulation of these pathways in the absence of both c-Cbl and Cbl-b, compounds with selectivity for Cbl-b over c-Cbl are likely to be highly desirable as cancer immunotherapy agents. Compounds having activity against both Cbl-b, and c-Cbl, which could be termed “pan-cbl”, may yet prove beneficial if their inhibitory effect against Cbl-b is potent enough.

Accordingly, there is a need for compounds that both inhibit Cbl-b and that either exhibit selectivity over binding to c-Cbl or have a known activity against c-Cbl.

The discussion of the background herein is included to explain the context of the technology. This is not to be taken as an admission that any of the material referred to was publicly available, known, or part of the common general knowledge as at the priority date of any of the claims found appended hereto.

Throughout the description and claims of the application the word “comprise” and variations thereof, such as “comprising” and “comprises”, is not intended to exclude other additives, components, integers or steps.

### SUMMARY

The instant disclosure addresses compounds for inhibiting the Cbl-B receptor that also exhibit inhibitory activity against, and in some cases are selective over, the C-Cbl receptor. In particular, the disclosure comprises a number of such compounds and methods for using the same.

The present disclosure further provides for compounds of formulae (II-A), (II-B), (II-C), (II-D), (II-E) and (II-F):

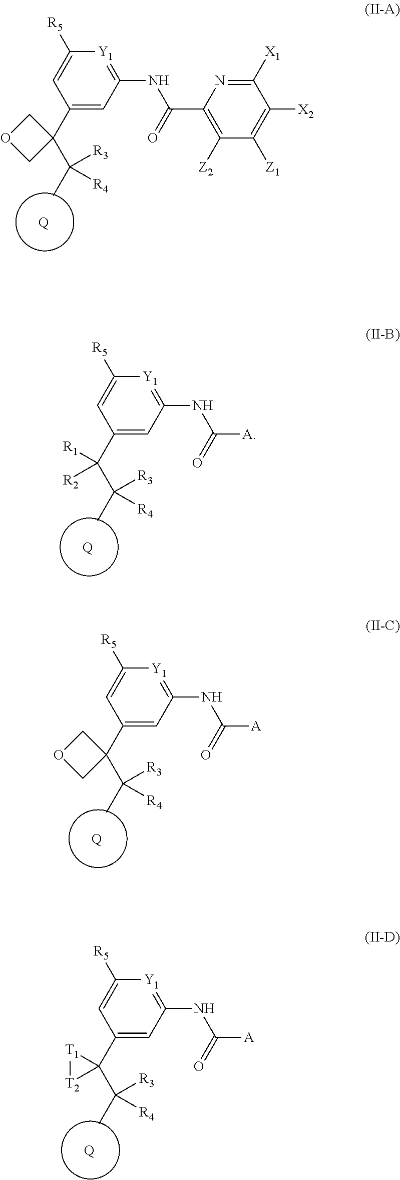

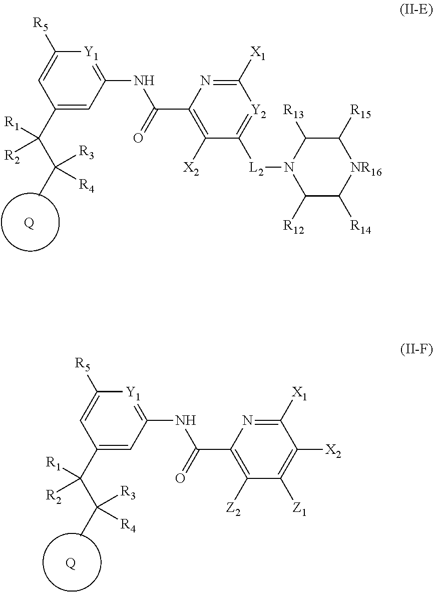

In formulae (II-A), (II-B), (II-C), (II-D), (II-E), and (II-F), Q is a 5-membered heteroaryl group, optionally substituted by one or more alkyl, cycloalkyl, or haloalkyl groups.

The present disclosure includes a process for making compounds of formula (II-A), (II-B), (II-C), (II-D), (II-E), and (II-F).

The present disclosure further includes a method of treatment comprising administering a compound of formula (II-A), (II-B), (II-C), (II-D), (II-E), and (II-F) optionally in combination with another agent, such as a checkpoint inhibitor, to a patient suffering from cancer.

### BRIEF DESCRIPTION OF THE DRAWINGS

FIG. 1 shows an exemplary synthetic scheme for compounds described herein;

FIG. 2 shows an exemplary synthetic scheme for compounds described herein;

FIG. 3 shows an exemplary synthetic scheme for compounds described herein;

FIG. 4 shows an exemplary synthetic scheme for compounds described herein;

FIGS. 5A-5H show exemplary synthetic schemes to intermediate compounds in synthesis of compounds disclosed herein;

FIGS. 6-20 show exemplary synthetic schemes to various exemplary compounds disclosed herein.

Like reference symbols in the various drawings indicate like elements.

### DETAILED DESCRIPTION

The instant disclosure is directed to compounds that bind to the Cbl-b inhibitor and that either exhibit selectivity over C-Cbl or have inhibitory activity against c-Cbl. Methods of making such compounds, as well as assays for assessing their potency and selectivity, as well as metabolic and permeability properties, are also described herein.

### Structures

A compound of formula (II-A),

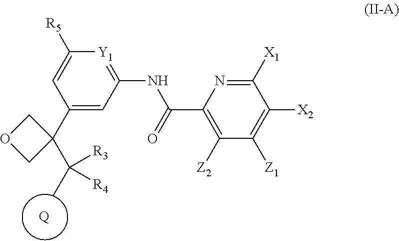

wherein:
Q is a 5-membered heteroaryl, optionally substituted by one or more alkyl, cycloalkyl, or haloalkyl groups;
Y₁ is CH, CF, or N;
R₃, R₄ are independently selected from: H, halogen, alkyl, CN, OH, alkoxy, and haloalkyl;
R₅ is selected from: H, halo, or L₁-R₁₀, wherein L₁ is —N(R₁₁)—, —C(═O)N(R₁₁)—, O, S, carbonyl, or a bond, wherein R₁₀ and R₁₁ are independently H, alkyl, cycloalkyl, alkenyl, alkynyl, aryl, heteroaryl, or heterocyclyl, and R₁₀ and R₁₁ are optionally substituted by one or more groups selected from: halo, CN, amino, carbonyl, OH, alkoxy, alkyl, or haloalkyl, heterocyclyl, aryl, and heteroaryl;
X₁ and X₂ are independently H, halo, haloalkyl, or cycloalkyl, wherein at least one of
X₁ and X₂ is not H;
and
one of Z₁ and Z₂ is H and the other is -L₂NR₇R₈,
wherein:
L₂ is —C(H)R₆—, or —C(═O)—, or a bond;
R₆=H, alkyl, cycloalkyl, or haloalkyl; and
R₇ and R₈ are independently selected from H, alkyl, cycloalkyl, hydroxyalkyl, heterocyclyl, and haloalkyl,
wherein, if any of R₇ or R₃ is cycloalkyl or heterocyclyl, said cycloalkyl or heterocyclyl group is optionally substituted with one or more groups selected from: sulfonyl, halo, hydroxyl, alkoxy, alkyl, cycloalkyl, alkoxyalkyl, alkenyl, hydroxyalkyl, cyano, carboxyalkyl, or haloalkyl;
or
R₇ and R₈ together with the nitrogen atom to which they are both bonded form a:
   3-8 membered saturated monocyclic ring,
   wherein the saturated monocyclic ring is optionally substituted with one or more groups selected from: sulfonyl, halo, hydroxyl, alkoxy, alkyl, cycloalkyl, alkoxyalkyl, alkenyl, hydroxyalkyl, oxo, carboxyalkyl, or haloalkyl;
   with the proviso that:
      when Y₁ is CH, R₃=R₄=R₅=H, L₂ is CH₂, X₁=CF₃, X₂=H, Z₂=H, and Q is 2-methyl triazol-1-yl, the substituted saturated monocyclic ring is not 3-fluoro-pyrrolidin-1-yl or 2-methyl morpholin-4-yl;
or
R₇ and R₈ together with the nitrogen atom to which they are both bonded form a:
   3-8 membered saturated spirocyclic ring,
   wherein the saturated spirocyclic ring is optionally substituted with one or more groups selected from: sulfonyl, halo, hydroxyl, cyano, alkyl, alkenyl, cycloalkyl, alkoxy, alkoxyalkyl, hydroxyalkyl, oxo, carboxyalkyl, or haloalkyl;
   with the proviso that:
   when Y₁ is CH, R₃=R₄=R₅=H, L₂ is CH₂, X₁=CF₃, X₂=H, Z₂=H, and Q is 2-methyl triazol-1-yl, the substituted saturated spirocyclic ring is not 5-azaspiro[2.4]hept-5-yl;
or
R₇ and R₈ together with the nitrogen atom to which they are both bonded form a:
   3-8 membered saturated fused bicyclic ring,
   wherein the saturated fused bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxyalkyl, or haloalkyl,
   with the proviso that:
   when Y₁ is CH, R₃=R₄=R₅=H, L₂ is CH₂, X₁=CF₃, X₂=H, and Q is 2-methyl triazol-1-yl, the fused bicyclic ring is not 2-azabicyclo[3.1.0]hex-2-yl, 6,6-difluoro-3-azabicyclo[3.1.0]hex-3-yl, or 6-oxa-3-azabicyclo[3.1.0]hex-3-yl;
or
R₇ and R₈ together with the nitrogen atom to which they are both bonded form a:
   3-8 membered saturated bridged bicyclic ring,
   wherein the saturated bridged bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxyalkyl, or haloalkyl;
or one of Z₁ and Z₂ is H and the other is

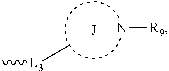

wherein:
   L₃ is —C(H)R₆—, —N(R₆)—, O, S, or a bond;
   J is a saturated 3-10 membered monocyclic ring, spirocyclic ring, bridged bicyclic ring, or fused bicyclic ring, a 5 or 6 member heteroaromatic ring, or a 3-10 member fused heteroaromatic ring system, and J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, or haloalkyl groups; and R₉=H, alkyl, cycloalkyl, aminoalkyl, haloalkyl, or carboxyalkyl;
else
Z₁=Z₂=H;
or an enantiomer, diastereomer or a pharmaceutically acceptable salt thereof.

A number of embodiments of formula (II-A) are also included, as follows, where it is to be understood that any specific embodiment can additionally comprise the feature(s) of any one or more of the other embodiments except where such feature(s) would be in conflict.

In some embodiments, the compound has formula (II-A), wherein Z₂=X₂=H.

In some embodiments, the compound has formula (II-A), wherein Q is 2-methyl triazol-1-yl or imidazolyl.

In some embodiments, the compound has formula (II-A), wherein R₅ is H.

In some embodiments, the compound has formula (II-A), wherein Y₁=CH.

In some embodiments, the compound has formula (II-A), wherein X₁=CF₃.

In some embodiments, the compound has formula (II-A), wherein Z₁ is -L₂NR₇R₈, and L₂ is CH₂ or C(H)Me.

In some embodiments, the compound has formula (II-A), wherein Z₁ is —C(H)R₆NR₇R₈ and R₇ and R₈ together with the nitrogen atom to which they are both bonded form a 3-8 membered saturated monocyclic ring that is optionally substituted with one or more groups selected from: methyl, fluoromethyl, hydroxyethyl, chloromethyl, hydroxyl, propyl, isopropyl, methoxy, methoxymethyl, difluoromethyl, methoxyethyl, vinyl, methylsulfonyl, 2-fluoroethyl, acetyl, and 1,1,1-trifluoroethyl.

In some embodiments, the compound has formula (II-A), wherein Z₁ is -L₂NR₇R₈, and L₂ is CH₂ or C(H)Me, and R₇ and R₈ and the nitrogen to which they are both bonded form a piperazin-1-yl ring substituted with one or more groups selected from: alkyl, sulfonyl, acetyl, haloalkyl, cycloalkyl, and oxetanyl.

A compound of formula (II-B),

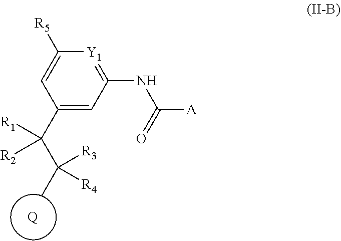

wherein:

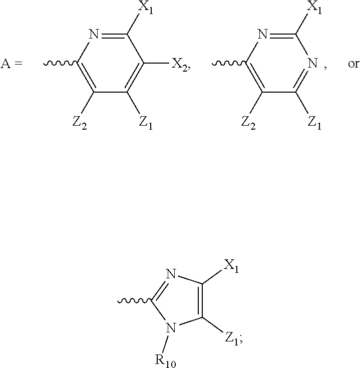

Y₁=CH, CF, or N;
R₁, R₂, R₃, R₄ are independently selected from: H, halo, alkyl, cycloalkyl, CN, OH, alkoxy, and haloalkyl; or
R₂ and R₃ together with the carbon atoms to which they are respectively bonded form a 3-6 membered cycloalkyl, heterocyclyl, or heteroaryl ring, optionally substituted with one or more groups selected from: halo, OH, alkoxy, alkyl, or fluoroalkyl;
R₅ is selected from: H, halo, or L₁-R₁₀, wherein L₁ is —N(R₁₁)—, —C(═O)N(R₁₁)—, O, S, carbonyl, or a bond, wherein R₁₀ and R₁₁ are independently H, alkyl, cycloalkyl, alkenyl, alkynyl, aryl, heteroaryl, or heterocyclyl, and R₁₀ and R₁₁ are optionally substituted by one or more groups selected from: halo, CN, amino, carbonyl, OH, alkoxy, alkyl, or haloalkyl, heterocyclyl, aryl, and heteroaryl;
Q is a 5-membered heteroaryl, optionally substituted by one or more alkyls or fluoro alkyls;
X₁ and X₂ are independently H, halo, haloalkyl, or cycloalkyl, wherein at least one of X₁ and X₂ is not H; and
one of Z₁ and Z₂ is H and the other is —C(H)(R₆)NR₇R₃, wherein:
R₆=H, alkyl, cycloalkyl, or haloalkyl; and
R₇ and R₈ are independently selected from H, alkyl, cycloalkyl, hydroxyalkyl, heterocyclyl, and haloalkyl,
or
R₇ and R₈ together with the nitrogen atom to which they are both bonded form a:
   3-8 membered saturated monocyclic ring,
   wherein the saturated monocyclic ring is optionally substituted with one or more groups selected from: sulfonyl, halo, hydroxyl, alkoxy, alkyl, cycloalkyl, alkoxyalkyl, alkenyl, hydroxyalkyl, oxo, carboxyalkyl, or haloalkyl;
   with the proviso that:
      when Y₁ is CH, R₃=R₄=R₅=H, L₂ is CH₂, X₁=CF₃, X₂=H, Z₂=H, A is a substituted pyrimidinyl ring, and Q is 2-methyl triazol-1-yl, the substituted saturated monocyclic ring is not 3-fluoro-pyrrolidin-1-yl;
or
R₇ and R₈ together with the nitrogen atom to which they are both bonded form a:
   3-8 membered saturated spirocyclic ring,
   wherein the saturated spirocyclic ring is optionally substituted with one or more groups selected from: sulfonyl, halo, hydroxyl, cyano, alkyl, alkenyl, cycloalkyl, alkoxy, alkoxyalkyl, hydroxyalkyl, oxo, carboxyalkyl, or haloalkyl;
or
R₇ and R₈ together with the nitrogen atom to which they are both bonded form a:
   3-8 membered saturated fused bicyclic ring,
   wherein the saturated fused bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxyalkyl, or haloalkyl;
or
R₇ and R₈ together with the nitrogen atom to which they are both bonded form a:
   3-8 membered saturated bridged bicyclic ring,
   wherein the saturated bridged bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxyalkyl, or haloalkyl;
or one of Z₁ and Z₂ is H and the other is

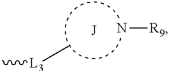

wherein:
   L₃ is —C(H)R₆—, —N(R₆)—, O, S, or a bond;
   J is a saturated 3-10 membered monocyclic ring, spirocyclic ring, bridged bicyclic ring, or fused bicyclic ring, a 5 or 6 member heteroaromatic ring, or a 3-10 member fused heteroaromatic ring system, and J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, or haloalkyl groups; and
   R₉=H, alkyl, or carboxyalkyl;
else Z₁=Z₂=H;
or an enantiomer, or diastereomer, or a pharmaceutically acceptable salt thereof.

A number of embodiments of formula (II-B) are also included, as follows, where it is to be understood that any specific embodiment can additionally comprise the feature(s) of any one or more of the other embodiments except where such feature(s) would be in conflict.

In some embodiments, the compound has formula (II-B), wherein X₂ and Z₂ are both H.

In some embodiments, the compound has formula (II-B), wherein Q is 2-methyl triazol-1-yl or imidazolyl.

In some embodiments, the compound has formula (II-B), wherein R₅ is H.

In some embodiments, the compound has formula (II-B), wherein R₅ is H, alkyl, or cycloalkyl.

In some embodiments, the compound has formula (II-B), wherein Y₁=CH.

In some embodiments, the compound has formula (II-B), wherein X₁=CF₃.

In some embodiments, the compound has formula (II-B), wherein Z₁ is -L₂NR₇R₈, and L₂ is CH₂ or C(H)Me.

In some embodiments, the compound has formula (II-B), wherein Z₁ is —C(H)R₆NR₇R₃ and R₇ and R₈ together with the nitrogen atom to which they are both bonded form a 3-8 membered saturated monocyclic ring that is optionally substituted with one or more groups selected from: methyl, fluoromethyl, hydroxyethyl, chloromethyl, hydroxyl, propyl, isopropyl, methoxy, methoxymethyl, difluoromethyl, methoxyethyl, vinyl, methylsulfonyl, 2-fluoroethyl, acetyl, and 1,1,1-trifluoroethyl.

In some embodiments, the compound has formula (II-B), wherein Z₁ is -L₂NR₇R₈, and L₂ is CH₂ or C(H)Me, and R₇ and R₈ and the nitrogen to which they are both bonded form a piperazin-1-yl ring substituted with one or more groups selected from: alkyl, sulfonyl, acetyl, haloalkyl, cycloalkyl, and oxetanyl.

In some embodiments, the compound has formula (II-B), wherein A=2-pyridyl.

In some embodiments, the compound has formula (II-B), wherein R₁ is methyl or fluoro and R₂ is H.

In some embodiments, the compound has formula (II-B), wherein R₁ is methyl, R₂ is fluoro, R₃ is fluoro, and R₄ is H.

In some embodiments, the compound has formula (II-B), wherein R₃ is fluoro and R₄ is H.

A compound of formula (II-C),

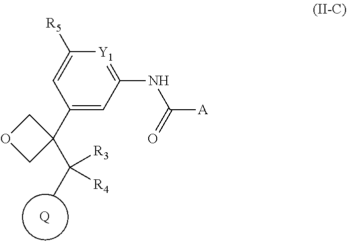

wherein:
Q is a 5-membered heteroaryl ring, optionally substituted by one or more alkyl, cycloalkyl, or haloalkyl groups;

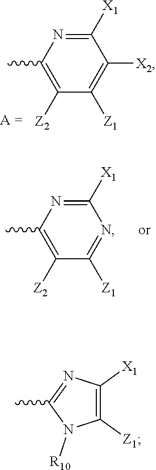

Y₁ is CH, CF, or N;
R₃, R₄ are independently selected from: H, halogen, alkyl, CN, OH, alkoxy, and haloalkyl, and at least one of R₃ and R₄ is halogen;
R₅ is selected from: H, halo, or L₁-R₁₀, wherein L₁ is —N(R₁₁)—, —C(═O)N(R₁₁)—, O, S, carbonyl, or a bond, wherein R₁₀ and R₁₁ are independently H, alkyl, cycloalkyl, alkenyl, alkynyl, aryl, heteroaryl, or heterocyclyl, and R₁₀ and R₁₁ are optionally substituted by one or more groups selected from: halo, CN, amino, carbonyl, OH, alkoxy, alkyl, or haloalkyl, heterocyclyl, aryl, and heteroaryl;
X₁ and X₂ are independently H, halo, haloalkyl, or cycloalkyl, wherein at least one of
X₁ and X₂ is not H;
and
one of Z₁ and Z₂ is H and the other is -L₂NR₇R₈, -L₂C(═O)NR₇R₃, or -L₂CR₇R₃(OH);
wherein:
L₂ is —C(H)R₆ₐ—, —C(H)CH₂(R_{6b}), or —C(═O)—, or a bond;
R₆=H, alkyl, cycloalkyl, or haloalkyl;
R_{6b}=cycloalkyl, or halo; and
R₇ and R₈ are independently selected from H, alkyl, cycloalkyl, hydroxyalkyl, heterocyclyl, and haloalkyl,
wherein, if any of R₇ or R₃ is cycloalkyl or heterocyclyl, said cycloalkyl or heterocyclyl group is optionally substituted with one or more groups selected from: sulfonyl, halo, hydroxyl, alkoxy, alkyl, cycloalkyl, alkoxyalkyl, alkenyl, hydroxyalkyl, cyano, carboxyalkyl, and haloalkyl;
or
R₇ and R₈ together with the nitrogen atom to which they are both bonded form a:
   3-8 membered saturated monocyclic ring,
   wherein the saturated monocyclic ring is optionally substituted with one or more groups selected from: sulfonyl, halo, hydroxyl, alkoxy, alkyl, cycloalkyl, alkoxyalkyl, alkenyl, hydroxyalkyl, oxo, carboxyalkyl, and haloalkyl;
or
R₇ and R₈ together with the nitrogen atom to which they are both bonded form a:
   3-8 membered saturated spirocyclic ring,
   wherein the saturated spirocyclic ring is optionally substituted with one or more groups selected from: sulfonyl, halo, hydroxyl, cyano, alkyl, alkenyl, cycloalkyl, alkoxy, alkoxyalkyl, hydroxyalkyl, oxo, carboxyalkyl, and haloalkyl;
or
R₇ and R₈ together with the nitrogen atom to which they are both bonded form a:
   3-8 membered saturated fused bicyclic ring,
   wherein the saturated fused bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxyalkyl, and haloalkyl;
or
R₇ and R₈ together with the nitrogen atom to which they are both bonded form a:
   3-8 membered saturated bridged bicyclic ring,
   wherein the saturated bridged bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxyalkyl, and haloalkyl;
or one of Z₁ and Z₂ is H and the other is

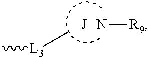

wherein:
   L₃ is —C(H)R₆—, —N(R₆)—, O, S, or a bond;
   J is a saturated 3-10 membered monocyclic ring, spirocyclic ring, bridged bicyclic ring, or fused bicyclic ring, a 5 or 6 member heteroaromatic ring, or a 3-10 member fused heteroaromatic ring system, and J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, or haloalkyl groups; and
   R₉=H, alkyl, cycloalkyl, haloalkyl, aminoalkyl, or carboxyalkyl;
else
Z₁=Z₂=H;
or an enantiomer, diastereomer or a pharmaceutically acceptable salt thereof.

A number of embodiments of formula (II-C) are also included, as follows, where it is to be understood that any specific embodiment can additionally comprise the feature(s) of any one or more of the other embodiments except where such feature(s) would be in conflict.

In some embodiments, the compound has formula (II-C), wherein Z₂=X₂=H.

In some embodiments, the compound has formula (II-C), wherein Q is 2-methyl triazol-1-yl or imidazolyl.

In some embodiments, the compound has formula (II-C), wherein R₅ is H.

In some embodiments, the compound has formula (II-C), wherein Y₁=CH.

In some embodiments, the compound has formula (II-C), wherein X₁=CF₃.

In some embodiments, the compound has formula (II-C), wherein Z₁ is -L₂NR₇R₈, and L₂ is CH₂ or C(H)Me.

In some embodiments, the compound has formula (II-C), wherein Z₁ is —C(H)R₆NR₇R₃ and R₇ and R₈ together with the nitrogen atom to which they are both bonded form a 3-8 membered saturated monocyclic ring that is optionally substituted with one or more groups selected from: methyl, fluoromethyl, hydroxyethyl, chloromethyl, hydroxyl, propyl, isopropyl, methoxy, methoxymethyl, difluoromethyl, methoxyethyl, vinyl, methylsulfonyl, 2-fluoroethyl, acetyl, and 1,1,1-trifluoroethyl.

In some embodiments, the compound has formula (II-C), wherein Z₁ is -L₂NR₇R₈, and L₂ is CH₂ or C(H)Me, and R₇ and R₈ and the nitrogen to which they are both bonded form a piperazin-1-yl ring substituted with one or more groups selected from: alkyl, sulfonyl, acetyl, haloalkyl, cycloalkyl, and oxetanyl.

In some embodiments, the compound has formula (II-C), wherein A=2-pyridyl.

A compound of formula (II-D),

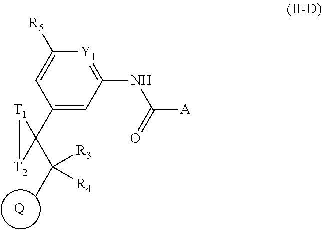

wherein:

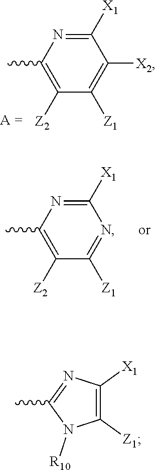

Q is a 5-membered heteroaryl, optionally substituted by one or more alkyl, cycloalkyl, or haloalkyl groups;
Y₁ is CH, CF, or N;
T₁ and T₂ are each independently a group selected from: [—C(R₁)(R₂)—]ₙ, —O—, —C(R₁)(R₂)—O—, >C═O, —C(R₁)(R₂)—C(═O)—, —C(R₁)(R₂)—S(═O)₂—, and >S(═O)₂, with the proviso that T₁ and T₂ together are not —CH₂—O—, —O—O—, or —O—C(═O)—O—; wherein n=0, 1 or 2, and each of R₁ and R₂ is independently selected from: H, halo, alkyl, alkenyl, cyano, hydroxyl, alkoxy, cycloalkyl, hydroxyalkyl, and haloalkyl, with the proviso that, when T₁ and T₂ together are —CH₂C(R₁)(R₂)CH₂—, neither of R₁ and R₂ is cyano;
or R₁ and R₂ together with the carbon atom to which they are bonded form a cycloalkyl or heterocyclyl ring;
R₃, and R₄ are independently selected from: H, and halo, alkyl, CN, OH, alkoxy, and haloalkyl;
R₅ is selected from: H, halo, or L₁-R₁₀, wherein L₁ is —N(R₁₁)—, —C(═O)N(R₁₁)—, O, S, carbonyl, or a bond, wherein R₁₀ and R₁₁ are independently H, alkyl, cycloalkyl, alkenyl, alkynyl, aryl, heteroaryl, or heterocyclyl, and R₁₀ and R₁₁ are optionally substituted by one or more groups selected from: halo, CN, amino, carbonyl, OH, alkoxy, alkyl, or haloalkyl, heterocyclyl, aryl, and heteroaryl;
X₁ and X₂ are independently H, halo, haloalkyl, or cycloalkyl, wherein at least one of
X₁ and X₂ is not H; and
one of Z₁ and Z₂ is H and the other is -L₂NR₇R₈,
wherein:
L₂ is —C(H)R₆—, —C(═O)—, or a bond;
R₆=H, alkyl, cycloalkyl, or haloalkyl; and
R₇ and R₈ are independently selected from H, alkyl, cycloalkyl, hydroxyalkyl, heterocyclyl, and haloalkyl,
wherein, if any of R₇ or R₃ is cycloalkyl or heterocyclyl, said cycloalkyl or heterocyclyl group is optionally substituted with one or more groups selected from: sulfonyl, halo, hydroxyl, alkoxy, alkyl, cycloalkyl, alkoxyalkyl, alkenyl, hydroxyalkyl, cyano, carboxyalkyl, and haloalkyl;
or
R₇ and R₈ together with the nitrogen atom to which they are both bonded form a:
   3-8 membered saturated monocyclic ring,
   wherein the saturated monocyclic ring is optionally substituted with one or more groups selected from: sulfonyl, halo, hydroxyl, alkoxy, alkyl, cycloalkyl, alkoxyalkyl, alkenyl, hydroxyalkyl, oxo, carboxyalkyl, and haloalkyl;
or
R₇ and R₈ together with the nitrogen atom to which they are both bonded form a:
   3-8 membered saturated spirocyclic ring,
   wherein the saturated spirocyclic ring is optionally substituted with one or more groups selected from: sulfonyl, halo, hydroxyl, cyano, alkyl, alkenyl, cycloalkyl, alkoxy, alkoxyalkyl, hydroxyalkyl, oxo, carboxyalkyl, and haloalkyl;
or
R₇ and R₈ together with the nitrogen atom to which they are both bonded form a:
   3-8 membered saturated fused bicyclic ring,
   wherein the saturated fused bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxyalkyl, and haloalkyl;
or
R₇ and R₈ together with the nitrogen atom to which they are both bonded form a:
   3-8 membered saturated bridged bicyclic ring,
   wherein the saturated bridged bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxyalkyl, and haloalkyl;
or one of Z₁ and Z₂ is H and the other is

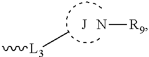

wherein:
   L₃ is —C(H)R₆—, —N(R₆)—, O, S, or a bond;
   J is a saturated 3-10 membered monocyclic ring, spirocyclic ring, bridged bicyclic ring, or fused bicyclic ring, a 5 or 6 member heteroaromatic ring, or a 3-10 member fused heteroaromatic ring system, and J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, and haloalkyl groups; and
   R₉=H, alkyl, cycloalkyl, aminoalkyl, haloalkyl, or carboxyalkyl;
else
Z₁=Z₂=H;
or an enantiomer, diastereomer or a pharmaceutically acceptable salt thereof.

A number of embodiments of formula (II-D) are also included, as follows, where it is to be understood that any specific embodiment can additionally comprise the feature(s) of any one or more of the other embodiments except where such feature(s) would be in conflict.

In some embodiments, the compound has formula (II-D), wherein X₂=Z₂=H.

In some embodiments, the compound has formula (II-D), wherein Q is 2-methyl triazol-1-yl or imidazolyl.

In some embodiments, the compound has formula (II-D), wherein R₅ is H.

In some embodiments, the compound has formula (II-D), wherein Y₁=CH.

In some embodiments, the compound has formula (II-D), wherein X₁=CF₃.

In some embodiments, the compound has formula (II-D), wherein Z₁ is -L₂NR₇R₈, and L₂ is CH₂ or C(H)Me.

In some embodiments, the compound has formula (II-D), wherein Z₁ is —C(H)R₆NR₇R₃ and R₇ and R₈ together with the nitrogen atom to which they are both bonded form a 3-8 membered saturated monocyclic ring that is optionally substituted with one or more groups selected from: methyl, fluoromethyl, hydroxyethyl, chloromethyl, hydroxyl, propyl, isopropyl, methoxy, methoxymethyl, difluoromethyl, methoxyethyl, vinyl, methylsulfonyl, 2-fluoroethyl, acetyl, and 1,1,1-trifluoroethyl.

In some embodiments, the compound has formula (II-D), wherein Z₁ is -L₂NR₇R₈, and L₂ is CH₂ or C(H)Me, and R₇ and R₈ and the nitrogen to which they are both bonded form a piperazin-1-yl ring substituted with one or more groups selected from: alkyl, sulfonyl, acetyl, haloalkyl, cycloalkyl, and oxetanyl.

In some embodiments, the compound has formula (II-D), wherein R₁₀ is H.

In some embodiments, the compound has formula (II-D), wherein A=2-pyridyl.

In some embodiments, the compound has formula (II-D), wherein T₁ and T₂, together with the carbon atom to which they are both bonded are selected from: 3,3-difluoro-1-cyclobutyl; and 3-fluoro-1-cyclobutyl.

A compound of formula (II-E),

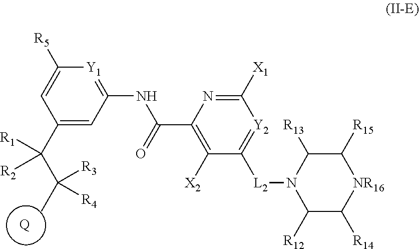

wherein:
Q is a 5-membered heteroaryl group, optionally substituted by one or more alkyl, cycloalkyl, or haloalkyl groups;
Y₁ and Y₂ are independently CH, CF, or N;
R₁, R₂, R₃, R₄ are independently selected from: H, halogen, alkyl, cycloalkyl, CN, OH, alkoxy, and haloalkyl; or
R₁ and R₂, together with the carbon atom to which they are both bonded form a 3-5 membered cycloalkyl or heterocyclyl, optionally substituted with one or more groups independently selected from: halogen, CN, OH, sulfonyl, alkoxy, alkyl, cycloalkyl, hydroxyalkyl, or haloalkyl; or
R₂ and R₃ together with the two carbon atoms to which they are respectively bonded form a 3-6 membered cycloalkyl, cycloalkenyl, heterocyclyl, heteroaryl ring, optionally substituted with one or more groups independently selected from: halogen, OH, alkoxy, cyano, sulfonyl, haloalkyl, hydroxyalkyl, alkyl, cycloalkyl, or alkoxyalkyl;
R₅ is selected from: H, halo, or L₁-R₁₀, wherein L₁ is —N(R₁₁)—, —C(═O)N(R₁₁)—, O, S, carbonyl, or a bond, wherein R₁₀ and R₁₁ are independently H, alkyl, cycloalkyl, alkenyl, alkynyl, aryl, heteroaryl, or heterocyclyl, and R₁₀ and R₁₁ are optionally substituted by one or more groups selected from: halo, CN, amino, carbonyl, OH, alkoxy, alkyl, or haloalkyl, heterocyclyl, aryl, and heteroaryl;
X₁ and X₂ are independently H, halo, haloalkyl, or cycloalkyl, but at least one of X₁ and X₂ is not H; and
L₂ is —C(H)R₁₇—, —C(═O)—, or a bond, wherein R₁₇=H, alkyl, cycloalkyl, or haloalkyl;
R₁₂, R₁₃, R₁₄, R₁₅, and R₁₆ are each independently selected from: H, sulfonyl, halo, hydroxyl, alkoxy, alkyl, cycloalkyl, heterocycloakyl, alkoxyalkyl, alkenyl, hydroxyalkyl, oxo, carboxylalkyl, and haloalkyl, wherein, if either of R₁₂, R₁₃, R₁₄, R₁₅, or R₁₆ is alkyl, alkenyl, or haloalkyl, then said alkyl, alkenyl, or haloalkyl may optionally be substituted by one or more cycloalkyl or heterocyclyl groups;
with the proviso that, when Y₁ and Y₂ are both CH, R₁=F, R₂=methyl, R₃=R₄=F, R₅=H, R₁₃=R₁₄=R₁₅=H, X₁=CF₃, X₂=H, Q is 2-methyl triazol-1-yl, and L₁ is a bond, then R₁₂ is not methyl, and R₁₆ is not H or methyl,
or an enantiomer, diastereomer or a pharmaceutically acceptable salt thereof.

A number of embodiments of formula (II-E) are also included, as follows, where it is to be understood that any specific embodiment can additionally comprise the feature(s) of any one or more of the other embodiments except where such feature(s) would be in conflict.

In some embodiments, the compound has formula (II-E), wherein X₂=H.

In some embodiments, the compound has formula (II-E), wherein Q is 2-methyl triazol-1-yl or imidazolyl.

In some embodiments, the compound has formula (II-E), wherein R₅ is H.

In some embodiments, the compound has formula (II-E), wherein Y₁=CH.

In some embodiments, the compound has formula (II-E), wherein X₁=CF₃.

In some embodiments, the compound has formula (II-E), wherein R₁₀ is H.

In some embodiments, the compound has formula (II-E), wherein R₁ and R₂, together with the carbon atom to which they are both bonded are oxetan-3-yl, and R₃ is fluoro.

A compound of formula (II-F),

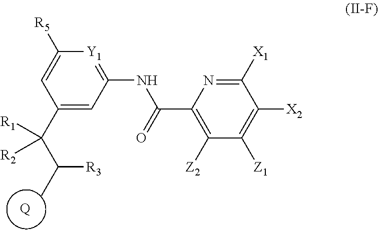

wherein:
Q is a 5-membered heteroaryl, optionally substituted by one or more alkyl, cycloalkyl, or haloalkyl groups;
Y₁ is CH, CF, or N;
R₁ is alkyl and R₂ is H, or R₁ and R₂ together are —CH₂OCH₂—;
R₃ is H or alkyl;
R₅ is selected from: H, halo, or L₁-R₁₀, wherein L₁ is —N(R₁₁)—, —C(═O)N(R₁₁)—, O, S, carbonyl, or a bond, wherein R₁₀ and R₁₁ are independently H, alkyl, cycloalkyl, alkenyl, alkynyl, aryl, heteroaryl, or heterocyclyl, and R₁₀ and R₁₁ are optionally substituted by one or more groups selected from: halo, CN, amino, carbonyl, OH, alkoxy, alkyl, or haloalkyl, heterocyclyl, aryl, and heteroaryl;
X₁ and X₂ are independently H, halo, haloalkyl, or cycloalkyl, wherein at least one of
X₁ and X₂ is not H;
and
one of Z₁ and Z₂ is H and the other is -L₂NR₇R₈,
wherein:
L₂ is —C(H)R₆—, —C(═O)—, or a bond;
R₆=H, alkyl, cycloalkyl, or haloalkyl; and
R₇ and R₈ are independently selected from H, alkyl, cycloalkyl, hydroxyalkyl, aminoalkyl, heterocyclyl, and haloalkyl,
wherein, if any of R₇ or R₃ is cycloalkyl or heterocyclyl, said cycloalkyl or heterocyclyl group is optionally substituted with one or more groups selected from: sulfonyl, halo, hydroxyl, alkoxy, alkyl, cycloalkyl, alkoxyalkyl, alkenyl, hydroxyalkyl, cyano, carboxylalkyl, and haloalkyl;
or
R₇ and R₈ together with the nitrogen atom to which they are both bonded form a 3-8-membered saturated monocyclic ring, optionally substituted with one or more groups selected from: sulfonyl, halo, alkoxy, alkyl, cycloalkyl, alkoxyalkyl, alkenyl, aminoalkyl, hydroxyalkyl, carboxylalkyl, and haloalkyl; or
or one of Z₁ and Z₂ is H and the other is

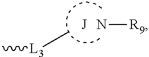

wherein:
   L₃ is —C(H)R₆—, —N(R₆)—, O, S, or a bond;
   J is a saturated 3-10 membered monocyclic ring, spirocyclic ring, bridged bicyclic ring, or fused bicyclic ring, a 5 or 6 member heteroaromatic ring, or a 3-10 member fused heteroaromatic ring system, and J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, or haloalkyl groups; and
   R₉=H, alkyl, aminoalkyl, haloalkyl, or carboxyalkyl;
or an enantiomer, diastereomer or a pharmaceutically acceptable salt or solvate thereof.

A number of embodiments of formula (II-F) are also included, as follows, where it is to be understood that any specific embodiment can additionally comprise the feature(s) of any one or more of the other embodiments except where such feature(s) would be in conflict.

In some embodiments, the compound has formula (II-F), wherein Z₂=X₂=H.

In some embodiments, the compound has formula (II-F), wherein Q is 2-methyl triazol-1-yl or imidazolyl.

In some embodiments, the compound has formula (II-F), wherein R₅ is H.

In some embodiments, the compound has formula (II-F), wherein Y₁=CH.

In some embodiments, the compound has formula (II-F), wherein X₁=CF₃.

In some embodiments, the compound has formula (II-F), wherein Z₁ is -L₂NR₇R₈, and L₂ is CH₂ or C(H)Me.

In some embodiments, the compound has formula (II-F), wherein Z₁ is —C(H)R₆NR₇R₈ and R₇ and R₈ together with the nitrogen atom to which they are both bonded form a 3-8 membered saturated monocyclic ring that is optionally substituted with one or more groups selected from: methyl, fluoromethyl, hydroxyethyl, chloromethyl, hydroxyl, propyl, isopropyl, methoxy, methoxymethyl, difluoromethyl, methoxyethyl, vinyl, methylsulfonyl, 2-fluoroethyl, acetyl, and 1,1,1-trifluoroethyl.

In some embodiments, the compound has formula (II-A), (II-B), (II-C), (II-D), (II-E), or (II-F), or any of the foregoing embodiments of those formulae herein, wherein Q is 4-methyl-4H-1,2,4-triazol-3-yl.

In some embodiments, the compound has formula (II-A), (II-B), (II-C), (II-D), or (II-F), or any of the foregoing embodiments of those formulae herein, wherein R₇ and R₈, together with the nitrogen atom to which they are both bonded, form a group selected from: azaspiro[2.4]heptan-5-yl, fluoroazetidin-1-yl, and 3-fluoro-3-methylazetidin-1-yl.

In some embodiments, the compound has formula (II-A), (II-B), (II-C), (II-D), or (II-E), or any of the foregoing embodiments of those formulae herein, wherein A=imidazolyl and R₁₀=methyl or H.

Any compound as explicitly identified herein is also to be considered within the scope of the present invention, including but not limited to:

| | |
|---|---|
| 1 | (S)-4-(1-(3-fluoroazetidin-1-yl)ethyl)-N-(3-(3-((4-methyl-4H-1,2,4-triazol- |
| | 3-yl)methyl)oxetan-3-yl)phenyl)-6-(trifluoromethyl)picolinamide |
| 2 | (R)-4-(1-(3-fluoroazetidin-1-yl)ethyl)-N-(3-(3-((4-methyl-4H-1,2,4-triazol- |
| | 3-yl)methyl)oxetan-3-yl)phenyl)-6-(trifluoromethyl)picolinamide |
| 3 | (S)-4-(1-(5-azaspiro[2.4]heptan-5-yl)ethyl)-N-(3-(3-((4-methyl-4H-1,2,4- |
| | triazol-3-yl)methyl)oxetan-3-yl)phenyl)-6-(trifluoromethyl)picolinamide |
| 4 | (R)-4-(1-(5-azaspiro[2.4]heptan-5-yl)ethyl)-N-(3-(3-((4-methyl-4H-1,2,4- |
| | triazol-3-yl)methyl)oxetan-3-yl)phenyl)-6-(trifluoromethyl)picolinamide |
| 5 | (R)-4-(5-azaspiro[2.4]heptan-5-ylmethyl)-6-cyclopropyl-N-(3-(3-(fluoro(4- |
| | methyl-4H-1,2,4-triazol-3-yl)methyl)oxetan-3-yl)phenyl)picolinamide |
| 6 | (S)-4-(5-azaspiro[2.4]heptan-5-ylmethyl)-6-cyclopropyl-N-(3-(3-(fluoro(4- |
| | methyl-4H-1,2,4-triazol-3-yl)methyl)oxetan-3-yl)phenyl)picolinamide |
| 7 | (R)-6-cyclopropyl-N-(3-(3-(fluoro(4-methyl-4H-1,2,4-triazol-3- |
| | yl)methyl)oxetan-3-yl)phenyl)-4-((3-fluoro-3-methylazetidin-1- |
| | yl)methyl)picolinamide |
| 8 | (S)-6-cyclopropyl-N-(3-(3-(fluoro(4-methyl-4H-1,2,4-triazol-3- |
| | yl)methyl)oxetan-3-yl)phenyl)-4-((3-fluoro-3-methylazetidin-1- |
| | yl)methyl)picolinamide |
| 16 | N-(3-(3,3-difluoro-1-((4-methyl-4H-1,2,4-triazol-3- |
| | yl)methyl)cyclobutyl)phenyl)-6-(trifluoromethyl)picolinamide |
| 18 | N-(3-((1R,3S)-3-fluoro-1-((4-methyl-4H-1,2,4-triazol-3- |
| | yl)methyl)cyclobutyl)phenyl)-6-(trifluoromethyl)picolinamide |
| 19 | N-(3-((1S,3R)-3-fluoro-1-((4-methyl-4H-1,2,4-triazol-3- |
| | yl)methyl)cyclobutyl)phenyl)-6-(trifluoromethyl)picolinamide |
| 20 | (R)-2-cyclopropyl-N-(3-(3-(fluoro(4-methyl-4H-1,2,4-triazol-3- |
| | yl)methyl)oxetan-3-yl)phenyl)-6-((3-fluoro-3-methylazetidin-1- |
| | yl)methyl)pyrimidine-4-carboxamide |
| 21 | 2-cyclopropyl-6-[(3-fluoro-3-methyl-azetidin-1-yl)methyl]-N-[3-[3-[(S)- |
| | fluoro-(4-methyl-1,2,4-triazol-3-yl)methyl]oxetan-3-yl]phenyl]pyrimidine- |
| | 4-carboxamide |
| 22 | 4-((S)-1-(5-azaspiro[2.4]heptan-5-yl)ethyl)-6-cyclopropyl-N-(3-(3-((R)- |
| | fluoro(4-methyl-4H-1,2,4-triazol-3-yl)methyl)oxetan-3-yl)phenyl)- |
| | picolinamide |
| 23 | 4-((R)-1-(5-azaspiro[2.4]heptan-5-yl)ethyl)-6-cyclopropyl-N-(3-(3-((R)- |
| | fluoro(4-methyl-4H-1,2,4-triazol-3-yl)methyl)oxetan-3-yl)phenyl)- |
| | picolinamide |
| 24 | 4-((S)-1-(5-azaspiro[2.4]heptan-5-yl)ethyl)-6-cyclopropyl-N-(3-(3-((S)- |
| | fluoro(4-methyl-4H-1,2,4-triazol-3-yl)methyl)oxetan-3-yl)phenyl)- |
| | picolinamide |
| 25 | 4-((R)-1-(5-azaspiro[2.4]heptan-5-yl)ethyl)-6-cyclopropyl-N-(3-(3-((S)- |
| | fluoro(4-methyl-4H-1,2,4-triazol-3-yl)methyl)oxetan-3- |
| | yl)phenyl)picolinamide |
| 26 | (R)-5-(5-azaspiro[2.4]heptan-5-ylmethyl)-N-(3-(1-(4-methyl-4H-1,2,4- |
| | triazol-3-yl)propan-2-yl)phenyl)-4-(trifluoromethyl)-1H-imidazole-2- |
| | carboxamide |
| 35 | 4-(5-azaspiro[2.4]heptan-5-ylmethyl)-6-cyclopropyl-N-(3-((1S,2R)-1,2- |
| | difluoro-1-(4-methyl-4H-1,2,4-triazol-3-yl)propan-2- |
| | yl)phenyl)picolinamide |
| 36 | 4-(5-azaspiro[2.4]heptan-5-ylmethyl)-6-cyclopropyl-N-(3-((1S,2S)-1,2- |
| | difluoro-1-(4-methyl-4H-1,2,4-triazol-3-yl)propan-2- |
| | yl)phenyl)picolinamide |
| 37 | 4-(5-azaspiro[2.4]heptan-5-ylmethyl)-6-cyclopropyl-N-(3-((1R,2S)-1,2- |
| | difluoro-1-(4-methyl-4H-1,2,4-triazol-3-yl)propan-2- |
| | yl)phenyl)picolinamide |
| 38 | 4-(5-azaspiro[2.4]heptan-5-ylmethyl)-6-cyclopropyl-N-(3-((1R,2R)-1,2- |
| | difluoro-1-(4-methyl-4H-1,2,4-triazol-3-yl)propan-2- |
| | yl)phenyl)picolinamide |
| 39 | 6-(5-azaspiro[2.4]heptan-5-ylmethyl)-2-cyclopropyl-N-(3-((1S,2R)-1,2- |
| | difluoro-1-(4-methyl-4H-1,2,4-triazol-3-yl)propan-2-yl)phenyl)pyrimidine- |
| | 4-carboxamide |
| 40 | 6-(5-azaspiro[2.4]heptan-5-ylmethyl)-2-cyclopropyl-N-(3-((1S,2S)-1,2- |
| | difluoro-1-(4-methyl-4H-1,2,4-triazol-3-yl)propan-2-yl)phenyl)pyrimidine- |
| | 4-carboxamide |
| 41 | 6-(5-azaspiro[2.4]heptan-5-ylmethyl)-2-cyclopropyl-N-(3-((1R,2S)-1,2- |
| | difluoro-1-(4-methyl-4H-1,2,4-triazol-3-yl)propan-2-yl)phenyl)pyrimidine- |
| | 4-carboxamide |
| 42 | 6-(5-azaspiro[2.4]heptan-5-ylmethyl)-2-cyclopropyl-N-(3-((1R,2R)-1,2- |
| | difluoro-1-(4-methyl-4H-1,2,4-triazol-3-yl)propan-2-yl)phenyl)pyrimidine- |
| | 4-carboxamide |
| 43 | 2-cyclopropyl-N-(3-((1S,2R)-1,2-difluoro-1-(4-methyl-4H-1,2,4-triazol-3- |
| | yl)propan-2-yl)phenyl)-6-((3-fluoro-3-methylazetidin-1- |
| | yl)methyl)pyrimidine-4-carboxamide |
| 44 | 2-cyclopropyl-N-(3-((1S,2S)-1,2-difluoro-1-(4-methyl-4H-1,2,4-triazol-3- |
| | yl)propan-2-yl)phenyl)-6-((3-fluoro-3-methylazetidin-1- |
| | yl)methyl)pyrimidine-4-carboxamid |
| 45 | 2-cyclopropyl-N-(3-((1R,2S)-1,2-difluoro-1-(4-methyl-4H-1,2,4-triazol-3- |
| | yl)propan-2-yl)phenyl)-6-((3-fluoro-3-methylazetidin-1- |
| | yl)methyl)pyrimidine-4-carboxamide |
| 46 | 2-cyclopropyl-N-(3-((1R,2R)-1,2-difluoro-1-(4-methyl-4H-1,2,4-triazol-3- |
| | yl)propan-2-yl)phenyl)-6-((3-fluoro-3-methylazetidin-1- |
| | yl)methyl)pyrimidine-4-carboxamide |
| 60 | (R)-N-(3-(3-(fluoro(4-methyl-4H-1,2,4-triazol-3-yl)methyl)oxetan-3- |
| | yl)phenyl)-4-((3-fluoro-3-methylazetidin-1-yl)methyl)-6- |
| | (trifluoromethyl)picolinamide |
| 61 | (S)-N-(3-(3-(fluoro(4-methyl-4H-1,2,4-triazol-3-yl)methyl)oxetan-3- |
| | yl)phenyl)-4-((3-fluoro-3-methylazetidin-1-yl)methyl)-6- |
| | (trifluoromethyl)picolinamide |
| 62 | (R)-6-((5-azaspiro[2.4]heptan-5-yl)methyl)-2-cyclopropyl-N-(3-(3- |
| | (fluoro(4-methyl-4H-1,2,4-triazol-3-yl)methyl)oxetan-3- |
| | yl)phenyl)pyrimidine-4-carboxamide |
| 63 | (S)-6-((5-azaspiro[2.4]heptan-5-yl)methyl)-2-cyclopropyl-N-(3-(3- |
| | (fluoro(4-methyl-4H-1,2,4-triazol-3-yl)methyl)oxetan-3- |
| | yl)phenyl)pyrimidine-4-carboxamide |
| 64 | (R)-4-(aminomethyl)-N-(3-(3-(fluoro(4-methyl-4H-1,2,4-triazol-3- |
| | yl)methyl)oxetan-3-yl)phenyl)-6-(trifluoromethyl)picolinamide formate |
| 65 | (S)-4-(aminomethyl)-N-(3-(3-(fluoro(4-methyl-4H-1,2,4-triazol-3- |
| | yl)methyl)oxetan-3-yl)phenyl)-6-(trifluoromethyl)picolinamide formate |
| 67 | 4-((dimethylamino)methyl)-N-(3-(3-((4-methyl-4H-1,2,4-triazol-3- |
| | yl)methyl)oxetan-3-yl)phenyl)-6-(trifluoromethyl)picolinamide |
| 98 | 4-((5-azaspiro[2.4]heptan-5-yl)methyl)-N-(3-(3-((4-methyl-4H-1,2,4- |
| | triazol-3-yl)methyl)oxetan-3-yl)phenyl)-6-(trifluoromethyl)picolinamide |
| 112 | 6-cyclopropyl-N-(3-(3-((R)-fluoro(4-methyl-4H-1,2,4-triazol-3- |
| | yl)methyl)oxetan-3-yl)phenyl)-4-((S)-1-(3-fluoro-3-methylazetidin-1- |
| | yl)ethyl)picolinamide |
| 113 | 6-cyclopropyl-N-(3-(3-((R)-fluoro(4-methyl-4H-1,2,4-triazol-3- |
| | yl)methyl)oxetan-3-yl)phenyl)-4-((R)-1-(3-fluoro-3-methylazetidin-1- |
| | yl)ethyl)picolinamide |
| 114 | 6-cyclopropyl-N-(3-(3-((S)-fluoro(4-methyl-4H-1,2,4-triazol-3- |
| | yl)methyl)oxetan-3-yl)phenyl)-4-((S)-1-(3-fluoro-3-methylazetidin-1- |
| | yl)ethyl)picolinamide |
| 115 | 6-cyclopropyl-N-(3-(3-((S)-fluoro(4-methyl-4H-1,2,4-triazol-3- |
| | yl)methyl)oxetan-3-yl)phenyl)-4-((R)-1-(3-fluoro-3-methylazetidin-1- |
| | yl)ethyl)picolinamide |
| 121 | (R)-1-methyl-N-(3-(1-(4-methyl-4H-1,2,4-triazol-3-yl)propan-2- |
| | yl)phenyl)-4-(trifluoromethyl)-1H-imidazole-2-carboxamide |
| 122 | (R)-4-cyclopropyl-N-(3-(1-(4-methyl-4H-1,2,4-triazol-3-yl)propan-2- |
| | yl)phenyl)-1H-imidazole-2-carboxamide |
| 123 | N-(3-(3-((4-methyl-4H-1,2,4-triazol-3-yl)methyl)oxetan-3-yl)phenyl)-4- |
| | (trifluoromethyl)-1H-imidazole-2-carboxamide |
| 124 | N-(3-(3-(fluoro(4-methyl-4H-1,2,4-triazol-3-yl)methyl)oxetan-3- |
| | yl)phenyl)-4-(trifluoromethyl)-1H-imidazole-2-carboxamide |
| 223 | 6-cyclopropyl-N-(3-(3,3-difluoro-1-((S)-fluoro(4-methyl-4H-1,2,4-triazol- |
| | 3-yl)methyl)cyclobutyl)phenyl)-4-(((S)-2-isopropyl-4-methylpiperazin-1- |
| | yl)methyl)picolinamide |
| 224 | (S)-6-cyclopropyl-N-(3-(3,3-difluoro-1-((4-methyl-4H-1,2,4-triazol-3- |
| | yl)methyl)cyclobutyl)phenyl)-4-((2-isopropyl-4-methylpiperazin-1- |
| | yl)methyl)picolinamide |
| 225 | 6-cyclopropyl-N-(3-(3,3-difluoro-1-((R)-fluoro(4-methyl-4H-1,2,4-triazol- |
| | 3-yl)methyl)cyclobutyl)phenyl)-4-(((S)-2-isopropyl-4-methylpiperazin-1- |
| | yl)methyl)picolinamide |
| 495 | 6-cyclopropyl-N-(3-(3,3-difluoro-1-((4-methyl-4H-1,2,4-triazol-3- |
| | yl)methyl)cyclobutyl)phenyl)-4-((3-hydroxy-3-methylazetidin-1- |
| | yl)methyl)picolinamide |
| 496 | 6-cyclopropyl-N-(3-(3,3-difluoro-1-((4-methyl-4H-1,2,4-triazol-3- |
| | yl)methyl)cyclobutyl)phenyl)-4-((3-(hydroxymethyl)-3-methylazetidin-1- |
| | yl)methyl)picolinamide |
| 497 | (R)-6-cyclopropyl-N-(3-(3,3-difluoro-1-(fluoro(4-methyl-4H-1,2,4-triazol- |
| | 3-yl)methyl)cyclobutyl)phenyl)-4-((3-(hydroxymethyl)-3-methylazetidin-1- |
| | yl)methyl)picolinamide |
| 498 | (S)-6-cyclopropyl-4-((2-isopropyl-4-methylpiperazin-1-yl)methyl)-N-(3-(3- |
| | ((4-methyl-4H-1,2,4-triazol-3-yl)methyl)oxetan-3-yl)phenyl)picolinamide |
| 499 | 6-cyclopropyl-N-(3-(3-((R)-fluoro(4-methyl-4H-1,2,4-triazol-3- |
| | yl)methyl)oxetan-3-yl)phenyl)-4-(((S)-2-isopropyl-4-methylpiperazin-1- |
| | yl)methyl)picolinamide |
| 500 | 6-cyclopropyl-N-(3-(3-((S)-fluoro(4-methyl-4H-1,2,4-triazol-3- |
| | yl)methyl)oxetan-3-yl)phenyl)-4-(((S)-2-isopropyl-4-methylpiperazin-1- |
| | yl)methyl)picolinamide |
| 501 | N-(3-((S)-2-((S)-fluoro(4-methyl-4H-1,2,4-triazol-3-yl)methyl)oxetan-2- |
| | yl)phenyl)-6-(trifluoromethyl)picolinamide |
| 502 | N-(3-((S)-2-((R)-fluoro(4-methyl-4H-1,2,4-triazol-3-yl)methyl)oxetan-2- |
| | yl)phenyl)-6-(trifluoromethyl)picolinamide |
| 503 | N-(3-((R)-2-((S)-fluoro(4-methyl-4H-1,2,4-triazol-3-yl)methyl)oxetan-2- |
| | yl)phenyl)-6-(trifluoromethyl)picolinamide |
| 504 | N-(3-((R)-2-((R)-fluoro(4-methyl-4H-1,2,4-triazol-3-yl)methyl)oxetan-2- |
| | yl)phenyl)-6-(trifluoromethyl)picolinamide |
| 505 | 4-(2-cyclopropyl-1-(5-azaspiro[2.4]heptan-5-yl)ethyl)-N-(3-(3-((4-methyl- |
| | 4H-1,2,4-triazol-3-yl)methyl)oxetan-3-yl)phenyl)-6- |
| | (trifluoromethyl)picolinamide |
| 506 | 4-(2-hydroxyethyl)-N-(3-(3-((4-methyl-4H-1,2,4-triazol-3- |
| | yl)methyl)oxetan-3-yl)phenyl)-6-(trifluoromethyl)-picolinamide |
| 507 | 4-(2-amino-2-oxoethyl)-N-(3-(3-((4-methyl-4H-1,2,4-triazol-3- |
| | yl)methyl)oxetan-3-yl)phenyl)-6-(trifluoromethyl)picolinamide |

or a pharmaceutically acceptable salt or solvate thereof.

The disclosure further comprises a method of treating a cancer, comprising administering to a subject in need thereof, a therapeutically effective amount of compound of formula (II-A), (II-B), (II-C), (II-D), (II-E), or (II-F), or a pharmaceutically acceptable salt or solvate thereof.

### Definitions

It is to be noted that the term “a” or “an” object may refer to one or more of that object. As such, the terms “a” (or “an”), “one or more,” and “at least one” can be used interchangeably herein.

All technical and scientific terms used herein have the same meaning as those understood respectively to those skilled in the art, unless otherwise specifically defined herein. Efforts have been made to ensure accuracy with respect to numbers used (e.g. amounts, temperature, etc.) but some experimental errors and deviations should be accounted for when reading this disclosure.

Throughout the specification and the claims, the words “comprise,” “comprises,” “comprising” are used in a non-exclusive sense, except where the context requires otherwise. It is to be understood that embodiments described herein include “consisting of” and/or “consisting essentially of” embodiments refer to some aspect as being exclusively defined.

As used herein, the term “about,” when referring to a value is meant to encompass variations of, in some embodiments ±50%, in some embodiments ±20%, in some embodiments ±10%, in some embodiments ±5%, in some embodiments 1%, in some embodiments ±0.5%, and in some embodiments ±0.1% from the specified amount, as such variations are appropriate to perform the disclosed methods or employ the disclosed compositions.

Where a range of values is provided and unless the context clearly dictates otherwise, it is understood that each intervening value, to the tenth of the unit of the lower limit, between the upper and lower limit of the range, is also disclosed, and that any other stated or intervening value in that stated range, is encompassed within the invention. The upper and lower limits of these small ranges which may independently be included in the smaller rangers is also encompassed within the invention, subject to any specifically excluded limit in the stated range. Where the stated range includes one or both of the limits, ranges excluding either or both of those included limits are also included in the invention.

Chemical structures are depicted herein according to customary display principles of organic chemistry: specifically, carbon atoms are shown only with bonds to non-hydrogen atoms; hydrogen atoms bonded to carbon atoms are not shown (except where necessary to specify stereochemistry). Correspondingly, when considering alternative substituents or groups occupying specific positions in a formula, it is to be assumed that a sufficient number hydrogen atoms (if otherwise unspecified) are present so that ordinary valences are fulfilled. An ordinary valence of carbon is 4.

“Geminal” refers to the relationship between two moieties that are attached to the same atom. For example, in the moiety —CH₂—CR^{x}R^{y}—, R^{x} and R^{y} are geminal to one another, and R^{x} may be referred to as a geminal R group to R^{y}.

“Vicinal” refers to the relationship between two moieties that are attached to adjacent atoms. For example, in the residue —CHR^{x}—CHR^{y}—, R^{x} and R^{y} are vicinal and R^{x} may be referred to as a vicinal R group to R^{y}.

“Optionally substituted” unless otherwise specified means that a group may be unsubstituted, or it may be substituted by one or more (e.g., 1, 2, 3, 4 or 5) non-hydrogen atoms or monovalent groups, such that the substituents may be the same or different from one another. In one embodiment, a group that is optionally substituted has one substituent. In another embodiment, a group that is optionally substituted has two substituents. In another embodiment, a group that is optionally substituted group has three substituents. In another embodiment, a group that is optionally substituted group has four substituents. In some embodiments, a group that is optionally substituted group has 1 to 2, 1 to 3, 1 to 4 or 1 to 5 substituents. When a group includes an atom (e.g., a ring carbon atom, or a terminal methyl group) that can itself accept more than one substituent, then “optionally substituted” as it applies to that group includes groups in which one atom is substituted with two or more substituents as applicable.

Heteroatom refers to any atom other than carbon or hydrogen. Typical heteroatoms found in small organic molecules are selected from: nitrogen, oxygen, fluorine, phosphorous, sulfur, chlorine, and bromine. It is to be understood that, where a heteroatom is specified as a possible member of a ring (or in another bivalent context), then monovalent atoms such as the halogens are excluded in that instance.

“Alkyl” as used herein refers to a saturated linear (i.e., unbranched) or branched univalent hydrocarbon functional group derived by the removal of one hydrogen atom from one carbon atom of a parent alkane. An alkyl group having n carbon atoms, as a radical, has formula CₙH₂ₙ₊₁. Alkyl groups having a given number of carbon atoms can be designated as follows: Cₙ-alkyl to denote any alkyl radical having n carbon atoms, or Cₙ₁₋ₙ₂-alkyl to denote any alkyl radical having from n1 to n2 carbon atoms. Thus C₁₋₁₀ means any alkyl radical having from one to ten carbon atoms. Particular alkyl groups of interest herein are those having 1 to 20 carbon atoms (a “C₁₋₂₀-alkyl”), those having 1 to 12 carbon atoms (a “C₁₋₁₂-alkyl”), those having 1 to 6 carbon atoms (a “C₁₋₆-alkyl”), having 2 to 6 carbon atoms (a “C₂₋₆-alkyl”), or having 1 to 4 carbon atoms (a “C₁₋₄-alkyl”). Examples of alkyl groups include, but are not limited to: methyl, ethyl, n-propyl, isopropyl, n-butyl, t-butyl, isobutyl, sec-butyl. It is to be understood that an alkyl group can bond to another group or moiety at any carbon atom in its structure: thus, for example, butan-1-yl (n-butyl) and butan-2-yl (sec-butyl) are contemplated by the definition herein.

Alkylamino refers to an amino group that has at least one alkyl substituent. Dialkylamino is a special case of alkylamino.

“Alkenyl” as used herein refers to an unsaturated linear (i.e., unbranched) or branched univalent hydrocarbon functional group having at least one site of olefinic unsaturation, i.e., having at least one instance of a carbon-carbon double bond (represented by the formula C═C), and having the number of carbon atoms designated. An alkenyl group having n carbon atoms and a single double-bond, as a radical, has formula CₙH₂ₙ₋₁ and is derived by the removal of one hydrogen atom from one carbon atom of a parent alkene. Alkenyl groups having a given number of carbon atoms can be designated as follows: Cₙ-alkenyl to denote any alkenyl radical having n carbon atoms, or Cₙ₁₋ₙ₂-alkenyl to denote any alkenyl radical having from n1 to n2 carbon atoms. Thus, C₂₋₁₀-alkenyl means an alkenyl group having from two to ten carbon atoms. An alkenyl group may contain constituent carbon atoms that are in “cis” or “trans” configurations, or “E” or “Z” configurations, with respect to a given double bond. Particular alkenyl groups are those having 2 to 20 carbon atoms (a “C₂₋₂₀-alkenyl”), having 2 to 8 carbon atoms (a “C₂₋₈-alkenyl”), having 2 to 6 carbon atoms (a “C₂₋₆-alkenyl”), or having 2 to 4 carbon atoms (a “C₂₋₄-alkenyl”). Preferred alkenyl groups have one double bond. Other alkenyl groups may have two double bonds (and may be referred to as dienyl). In alkenyl groups having more than one double bond, a pair of double bonds may be separated by one carbon-carbon single bond, in which case the arrangement is referred to as “conjugated”, or they may be separated by more than one carbon-carbon single bond. Examples of alkenyl group include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkynyl” as used herein refers to an unsaturated linear (i.e., unbranched) or branched univalent hydrocarbon having at least one site of acetylenic unsaturation, i.e., having at least one instance of a carbon-carbon triple bond (represented by the formula formula C≡C) and having the number of carbon atoms designated. An alkynyl group having n carbon atoms and a single triple-bond, as a radical, has formula CₙH₂ₙ₋₃, and is derived by the removal of one hydrogen atom from one carbon atom of a parent alkyne. Alkynyl groups having a given number of carbon atoms can be designated as follows: Cₙ-alkynyl to denote any alkynyl radical having n carbon atoms, or Cₙ₁₋ₙ₂-alkynyl to denote any alkynyl radical having from n1 to n2 carbon atoms. Particular alkynyl groups are those having 2 to 20 carbon atoms (a “C₂₋₂₀-alkynyl”), having 2 to 8 carbon atoms (a “C₂₋₈-alkynyl”), having 2 to 6 carbon atoms (a “C₂₋₆-alkynyl”), or having 2 to 4 carbon atoms (a “C₂₋₄-alkynyl”). Examples of alkynyl groups include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Alkylenyl” as used herein refers to a saturated linear (i.e., unbranched) or branched bivalent hydrocarbon group having the number of carbon atoms designated. An alkylene group having n carbon atoms, as a radical, has formula —CₙH₂ₙ—. Particular alkylene groups are those having 1 to 6 carbon atoms (a “C₁₋₆-alkylene”), 1 to 5 carbon atoms (a “C₁₋₅-alkylene”), having 1 to 4 carbon atoms (a “C₁₋₄-alkylene”), or 2 to 3 carbon atoms (a “C₂₋₃-alkylene”). Examples of alkylene radicals include, but are not limited to, methylene (—CH₂—), ethylene (—CH₂—CH₂—), propylene (—CH₂—CH₂—CH₂—), butylene (—CH₂—CH₂—CH₂—CH₂—), sec-butylene (—CH(CH₃)—CH₂—CH₂—) and the like.

Cyclic (ring-containing) moieties comprise atoms bonded together in a ring, and have one or more substituents other than hydrogen atoms bonded to one or more ring atoms. Each atom in the ring defines a vertex of a polygon. A cyclic radical, denoted cyclyl, is derived by the removal of one hydrogen atom from one ring atom.

Cyclic moieties may be carbocyclic or heterocyclic. Cyclic moieties include monocyclic, fused ring systems, spiro-ring systems, and bridged ring systems.

Two ring atoms are adjacent to one another in a ring if they are bonded to one another in that same ring. In rings having 4 or more ring atoms, adjacent atoms are bonded to one another but to no other atom in the same ring. In a three-membered ring, each atom is necessarily bonded to each other atom in the ring. Two adjacent ring atoms define one “edge” of the ring.

Two or more cyclic moieties may join to one another in one of several ways to form ring systems that comprise more than one ring. Bicyclic ring systems are those that contain two or more rings that are joined together.

Two rings are fused to one another if two ring atoms are adjacent to one another in both rings and are common to both rings. Such rings are said to share an “edge”.

Spirocyclic ring systems comprise a pair of rings that share a single vertex. Such systems contain a ring junction at which the two rings share a single ring atom. Spirocyclic ring systems may contain one or more heteroatoms as ring atoms.

Bridged ring systems contain at least a pair of rings in which two or more non-adjacent ring atoms are shared by two or more rings. The two non-adjacent ring atoms in question are referred to as “bridgehead” atoms and the pair of bridgehead atoms are members of three different rings, even though the simplest such ring systems are typically referred to as “bridged bicyclic rings”. Examples of carbocyclic radicals containing bridged bicyclic rings are norbornyl and adamantyl. Bridged bicyclic ring systems may contain one or more heteroatoms as ring atoms.

Chained ring systems contain two rings that are joined to one another but do not share any ring atom in common: one ring is a substituent of the other, and vice versa. Biphenyl is an example of a chained ring system.

Ring systems may contain pairs of rings that are fused or chained to one another, spiro-joined, or bridged, or in the case of three or more rings, joined in combinations of ways thereof.

“Carbocycle” as used herein refers to aromatic, saturated or unsaturated cyclic univalent hydrocarbon groups having the number of annular (i.e., ring) carbon atoms designated (i.e., C₃₋₁₀ means three to ten annular carbon atoms). Carbocyclic groups have a single ring (“monocycles”) or more than one ring (“bicycles”, “tricycles”, or polycycles, more generally). Two or more carbocyclic rings may be joined to one another by fused, spiro, bridged, or chained connections as further described elsewhere herein.

It is intended herein that the term carbocycle encompasses radicals having one or more adjacent pairs of ring atoms between which are double bonds, and that, where more than one such double bond is present, the double bonds may or may not form a conjugated system within the ring. Thus carbocycles may be more specifically designated according to whether they are fully saturated (“cycloalkyl”), unsaturated at least in part (“cycloalkenyl”), or fully conjugated, (“aromatic” or “aryl”). Cycloalkyl groups are fully saturated radicals and are derived by the removal of one hydrogen atom from one carbon atom of a parent cycloalkane. Particular cycloalkyl groups are those having from 3 to 12 annular carbon atoms (C₃₋₁₂-cycloalkyl). A preferred cycloalkyl is a monocyclic hydrocarbon having from 3 to 8 annular carbon atoms (a “C₃₋₈-cycloalkyl”), or having 3 to 6 carbon atoms (a “C₃₋₆-cycloalkyl”). Single ring cycloalkyl radicals have formula CₙH₂ₙ₋₁. Examples of cycloalkyl groups include, but are not limited to, cyclopropyl, cyclobutyl, cyclopentyl, cyclohexyl, and cycloheptyl. Cycloalkenyl groups have one or more double bonds between adjacent ring carbon atoms. Examples of cycloalkenyl groups include 1-cyclohex-1-enyl, and 1-cyclohex-3-enyl.

“Aryl” as used herein refers to a carbocyclic group having a aromatic single ring (e.g., phenyl) or multiple aromatic rings fused to one another (e.g., naphthyl). Preferably, an aryl group comprises from 6 to 20 carbon atoms, more preferably between 6 to 12 carbon atoms. Particularly preferred aryl groups are those having from 6 to 14 annular carbon atoms (a “C₆₋₁₄-aryl”). The term aromatic is used herein as it is typically used in organic chemistry, meaning, with a few understood exceptions, rings and ring systems in which the annular atoms contribute a total of (4n+2) pi electrons to a set of delocalized molecular orbitals, where n is a non-zero positive integer.

Typical aryl groups include, but are not limited to, groups derived from fused ring systems that comprise one or more aromatic rings, or conjugated ring systems, such as but not limited to aceanthrylene, acenaphthylene, acephenanthrylene, anthracene, azulene, benzene, chrysene, coronene, fluoranthene, heptaphene, hexacene, hexaphene, as-indacene, s-indacene, indene, naphthalene (hexalene), octacene, octaphene, octalene, ovalene, pentacene, pentalene, pentaphene, perylene, phenalene, phenanthrene, picene, pleiadene, pyrene, pyranthrene, rubicene, tetraphenylene, triphenylene, and trinaphthalene.

A “heterocyclic”, or “heterocyclyl”, group as used herein refers to a saturated or an unsaturated but non-aromatic, cyclic group having one or more rings that comprises at least one carbon atom and one or more heteroatoms. Typically such a ring has from 1 to 14 ring carbon atoms and from 1 to 6 ring heteroatoms that can be same or different from each other. Such a group is typically derived by the removal of one hydrogen atom from one ring atom of a parent heterocycle. Therefore a heterocyclyl group can bond to a position on a scaffold through either a ring carbon atom or a ring heteroatom such as a nitrogen atom. It is intended herein that the term heterocyclyl encompasses radicals having one or more double bonds between adjacent ring atoms, and that where more than one such double bond is present, the double bonds do not form a conjugated system within the ring.

Particularly preferred heterocyclyl groups are: 3- to 14-membered rings having 1 to 13 annular carbon atoms and 1 to 6 annular heteroatoms independently selected from nitrogen, phosphorus, oxygen and sulfur; 3- to 12-membered rings having 1 to 11 annular carbon atoms and 1 to 6 annular heteroatoms independently selected from nitrogen, phosphorus, oxygen and sulfur; 3- to 10-membered rings having 1 to 9 annular carbon atoms and 1 to 4 annular heteroatoms independently selected from nitrogen, phosphorus, oxygen and sulfur; 3- to 8-membered rings having 1 to 7 annular carbon atoms and 1 to 4 annular heteroatoms independently selected from nitrogen, phosphorus, oxygen and sulfur; and 3- to 6-membered rings having 1 to 5 annular carbon atoms and 1 to 4 annular heteroatoms independently selected from nitrogen, phosphorus, oxygen and sulfur. In one variation, heterocyclyl include monocyclic 3-, 4-, 5-, 6- or 7-membered rings having from 1 to 2, 1 to 3, 1 to 4, 1 to 5 or 1 to 6 annular carbon atoms and 1 to 2, 1 to 3 or 1 to 4 annular heteroatoms independently selected from from nitrogen, phosphorus, oxygen and sulfur. In another variation, heterocyclyl includes polycyclic non-aromatic rings having from 1 to 12 annular carbon atoms and 1 to 6 annular heteroatoms independently selected from nitrogen, phosphorus, oxygen and sulfur. Exemplary heterocyclic rings include: aziridine, azetidine, pyrrolidine, piperazine, piperidine, oxetane, tetrahydrofuran, and morpholine.

A heterocyclic ring may make fused, spiro, or bridged, connections or make any combination of such connections to one or more other rings.

“Heteroaryl” or “heteroaromatic”, as used herein, refers to an aromatic cyclic group having from 1 to 14 ring carbon atoms and at least one ring heteroatom, including but not limited to heteroatoms such as nitrogen, phosphorus, oxygen and sulfur. The term refers to a monovalent heteroaromatic radical derived by the removal of one hydrogen atom from a single ring atom of a parent heteroaromatic ring system. A heteroaryl group may have a single ring (e.g., pyridyl, furyl) or multiple fused rings (e.g., indolizinyl, benzothienyl). Particular heteroaryl groups are 5- to 14-membered rings having 1 to 12 annular (i.e., ring) carbon atoms and 1 to 6 annular (i.e., ring) heteroatoms independently selected from nitrogen, phosphorus, oxygen and sulfur; 5- to 10-membered rings having 1 to 8 annular carbon atoms and 1 to 4 annular heteroatoms independently selected from nitrogen, phosphorus, oxygen and sulfur; and 5-, 6- or 7-membered rings having 1 to 5 annular carbon atoms and 1 to 4 annular heteroatoms independently selected from nitrogen, oxygen and sulfur In one variation, heteroaryl include monocyclic aromatic 5-, 6- or 7-membered rings having from 1 to 6 annular carbon atoms and 1 to 4 annular heteroatoms independently selected from nitrogen, oxygen and sulfur. In another variation, heteroaryl includes polycyclic aromatic rings having from 1 to 12 annular carbon atoms and 1 to 6 annular heteroatoms independently selected from nitrogen, phosphorus, oxygen and sulfur.

Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferred heteroaryl groups are those derived from thiophene, pyrrole, benzothiophene, benzofuran, indole, pyridine, quinoline, imidazole, oxazole and pyrazine.

Rings of different categories may be connected to one another, such as by fused, spiro, or bridged, connections, or by combinations thereof. Such a ring system can be referred to as a “mixed” ring system.

For example, at least one ring of a multiple ring system can be aromatic on its own, though one or more of the remaining fused rings may be not aromatic. Examples of fused ring systems that contain at least one aromatic ring and at least one partially saturated ring include fluorene, indane, and biphenylene.

A mixed ring system having more than one ring where at least one ring is aromatic and at least one ring is non-aromatic may be connected to another structure by bonding to either an aromatic ring atom or a non-aromatic ring atom.

A heteroaryl group having more than one ring where at least one ring is non-aromatic may be connected to another structure at either an aromatic ring position or at a non-aromatic ring position.

Similarly, carbocyclic and heterocyclic groups may join to one another in one of several ways to form ring systems that comprise more than one ring.

“Halogen” refers to an atom selected from fluorine, chlorine, bromine and iodine. The terms “halide” or “halo” refer to halogens as substituents, in which each is individually referred to as fluoro, chloro, bromo, and/or iodo, or as “fluoride”, “chloride”, “bromide”, or “iodide”. An alkyl group in which one or more hydrogen atoms is each replaced by a halogen atom is referred to as a “haloalkyl”. For example, “C₁₋₆-haloalkyl” refers to an alkyl group having from 1-6 carbon atoms in which at least one hydrogen atom is replaced by a halogen atom. Where a moiety is substituted with more than one instance of a given halogen atom, it may be referred to by using a prefix corresponding to the number of halogen moieties attached, e.g., dihaloaryl, trihaloaryl, refer to aryl groups substituted with two (“di”) or three (“tri”) halo groups respectively. It is to be understood that, where more than one halo groups are present they are not necessarily the same as one another. Thus 4-chloro-3-fluorophenyl is within the scope of dihaloaryl. An alkyl group in which every hydrogen is replaced with a halogen atom is referred to as a “perhaloalkyl.” A preferred perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbonyl” refers to the group C═O.

“Thiocarbonyl” refers to the group C═S.

“Oxo” refers to the moiety ═O, i.e., an oxygen atom double-bonded to a second atom other than oxygen. A carbon atom in a chain or a ring that is bonded to an oxo moiety is also referred to as a carbonyl group. A sulfur atom in a chain or a ring can accept two oxo substituents.

“Prodrug” refers to a pharmacologically inactive derivative of a drug molecule that requires a transformation within the body, usually a metabolic transformation, to release the active drug.

“Promoiety” refers to a form of protecting group that, when used to mask a functional group within a drug molecule converts the drug into a prodrug. Typically, the promoiety will be attached to the drug via bond(s) that are cleaved by enzymatic or non-enzymatic means in vivo. Ideally, the promoiety is rapidly cleared from the body upon cleavage from the prodrug.

“Protecting group” refers to a grouping of atoms that when attached to a reactive group in a molecule masks, reduces or prevents that reactivity. Examples of protecting groups can be found in Green et al., “Protective Groups in Organic Chemistry”, (Wiley, 2nd ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

Compounds of Formula (II-A), (II-B), (II-C), (II-D), (II-E), and (II-F), collectively “Formulae (II-A)-(II-F)”, described herein, or a salt or solvate thereof may exist in stereoisomeric forms (e.g., such a compound contains one or more asymmetric carbon atoms). The individual stereoisomers (such as purified enantiomers and diastereomers) and mixtures of these or enantiomerically/diastereomerically enriched mixtures are included within the scope of the subject matter disclosed herein.

It is further understood that a compound or salt of Formulae (II-A)-(II-F) may exist in tautomeric forms other than that shown in the formula and these are also included within the scope of the subject matter disclosed herein.

The subject matter disclosed herein also includes isotopically-labelled forms of the compounds described herein, i.e., compounds that have the formulae shown herein but for the fact that one or more constituent atoms are replaced by an atom having an atomic mass or mass number different from the atomic mass or mass number usually found in nature and/or at an abundance not normally found in nature. Examples of isotopes that can be incorporated into compounds described herein and pharmaceutically acceptable salts thereof, at levels that differ from the natural distribution of such isotopes, include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶C, ¹²³I and ¹²⁵I.

The subject matter disclosed herein further includes prodrugs, metabolites, and pharmaceutically acceptable salts of compounds of Formulae (II-A)-(II-F). Metabolites of the compounds of Formulae (II-A)-(II-F) include compounds produced by a process comprising contacting a compound of Formulae (II-A)-(II-F) with a mammal for a period of time sufficient to yield a metabolic product thereof.

In some embodiments, the salts of the compounds of the invention are pharmaceutically acceptable salts. “Pharmaceutically acceptable salts” are those salts that retain at least some of the biological activity of the free (non-salt) compound and that can be administered as drugs or pharmaceuticals to a subject. Such salts, for example, include: (1) acid addition salts; (2) salts formed when an acidic proton is replaced by a metal ion; or (3) an acidic proton coordinates with an organic base. Pharmaceutically acceptable salts can be prepared in situ in the manufacturing process, or by separately reacting a purified compound of the invention in its free acid or base form with a suitable organic or inorganic base or acid respectively, and isolating the salt thus formed during subsequent purification.

If the compound of Formulae (II-A)-(II-F) is a base, the desired pharmaceutically acceptable salt may be prepared as an acid addition salt by any suitable method available in the art, for example, treatment of the free base with an inorganic acid, such as hydrochloric acid, hydrobromic acid, sulfuric acid, nitric acid, methanesulfonic acid, phosphoric acid and others of like property, or with an organic acid, such as acetic acid, maleic acid, succinic acid, mandelic acid, fumaric acid, propionic acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, and others of like property.

If the compound of Formulae (II-A)-(II-F) is an acid, one or more acidic protons present may be replaced by a metal ion, e.g., an alkali metal ion, an alkaline earth ion, or an aluminum ion. The desired pharmaceutically acceptable salt may then be prepared by any suitable method, for example, treatment of the free acid with an inorganic or organic base, such as an amine (primary, secondary or tertiary), an alkali metal hydroxide or alkaline earth metal hydroxide, or the like. Examples of suitable salts include, but are not limited to, organic salts derived from amino acids, such as glycine and arginine, ammonia, and cyclic amines, such as piperidine, morpholine and piperazine, alcoholamines such as ethanolamine, diethanolamine, and triethanolamine, and inorganic salts derived from sodium, calcium, potassium, magnesium, manganese, iron, copper, zinc, aluminum and lithium. Acceptable inorganic bases include aluminum hydroxide, calcium hydroxide, potassium hydroxide, sodium carbonate, sodium hydroxide, and the like.

The compounds herein may also be present as solvates, such as crystallized with a corresponding quantity of a solvent molecule, in a ratio that may or may not be stoichiometric. In preferred embodiments the solvent is water, in which case the solvate is a hydrate. In preferred embodiments, one or more solvent molecules are present in stoichiometric ratios relative to molecules of the compound.

A compound of Formulae (II-A)-(II-F) can also be in the form of a “prodrug,” which includes compounds with moieties that can be metabolized in vivo. Generally, prodrugs are metabolized in vivo by esterases or by other mechanisms to form active drugs inside the patient's body. Examples of prodrugs and their uses are well known in the art (see, e.g., Berge et al. (1977) “Pharmaceutical Salts”, J. Pharm. Sci., 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs which are converted to active forms through other mechanisms in vivo are also included. In aspects, the compounds of the invention are prodrugs of any of the formulae herein.

It is also to be understood that the subject matter disclosed herein includes combinations and subsets of the particular categories (e.g., salt forms, tautomers, stereoisomeric forms) described herein.

The terms “treat” and “treatment” refer to therapeutic treatment, wherein an object is to slow down, diminish, or attenuate, an undesired physiological change or disorder, such as the development or spread of arthritis or cancer. For purposes of this disclosure, beneficial or desired clinical results include, but are not limited to: alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to a patient's expected survival if not receiving treatment. Those in need of treatment include those with the condition or disorder, and further include those who are only experiencing an early stage of the disorder or disease, in which one or more typical symptoms may yet to manifest.

The phrase “therapeutically effective amount” means an amount of a compound of the present invention, or a salt thereof, that is sufficient to produce a desired therapeutic outcome. Such an amount is sufficient to (i) treat the particular disease, condition, or disorder, (ii) attenuate, ameliorate, or eliminate one or more symptoms (such as biochemical, histologic and/or behavioral) of the particular disease, condition, or disorder, (iii) prevent or delay the onset of one or more symptoms of the particular disease, condition, or disorder described herein, or (iv) reduce the severity or duration of, stabilize the severity of, or eliminate one or more symptoms of the disease, condition or disorder. Other beneficial or desired results of a therapeutic use include, e.g., decreasing one or more symptoms resulting from the disease, including its complications and intermediate pathological phenotypes presenting during development of the disease, increasing the quality of life of those suffering from the disease, decreasing the dose of other medications required to treat the disease, enhancing effect of another medication, delaying the progression of the disease, and/or prolonging survival of patients. In the case of a cancer, the therapeutically effective amount of the drug may reduce the number of cancer cells; reduce a tumor size or check its rate of growth; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR). In various embodiments, the amount is sufficient to ameliorate, palliate, lessen, and/or delay one or more of symptoms of cancer.

The term “cancer” refers to or describe the physiological condition in mammals that is typically characterized by unregulated cell growth and an invasive nature, wherein the cancerous cells are capable of local invasion and/or metastasis to noncontiguous sites. As used herein, “cancer cells,” “cancerous cells,” or “tumor cells” refer to the cells that are characterized by this unregulated cell growth and invasive property. A “tumor” comprises more than one cancerous cells. Cancers can further be divided into liquid or solid types. The term “cancer” as used herein generally encompasses all types of cancers, subject to specific context. Examples of cancers include, but are not limited to, carcinoma, melanoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, esophageal cancer, endometrial or uterine carcinoma, salivary gland carcinoma, kidney or renal cancer, prostate cancer, vulval cancer, thyroid cancer, hepatic carcinoma, anal carcinoma, penile carcinoma, Ewing's sarcoma, medulloblastomer, as well as head and neck cancer.

A “chemotherapeutic agent” is a chemical compound or biologic useful in the treatment of cancer. A chemotherapeutic agent can be an immunotherapeutic agent. As used herein, an “immunotherapeutic agent” is a compound that enhances the immune system to help fight cancer, specifically or non-specifically. Immunotherapeutics include monoclonal antibodies and non-specific immunotherapies that boost the immune system,

As used herein, a “combination therapy” is a therapy that includes two or more different compounds, administered simultaneously or contemporaneously. Typically, each of the two or more different compounds has a different mechanism of action. Thus, in one aspect, a combination therapy comprising a compound detailed herein and another compound is provided. In some variations, the combination therapy optionally includes one or more pharmaceutically acceptable carriers or excipients, non-pharmaceutically active compounds, and/or inert substances. Combination therapies can comprise two more compounds in a single delivery vehicle such as a tablet, or can comprise doses in separate formulations, such as different tablets, or a tablet and an injectable solution.

As used herein, the term “effective amount” means such an amount of a compound of the invention that, in combination with its parameters of efficacy and toxicity, should be effective in a given administered form. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial results may be or is achieved. Suitable doses of any of the co-administered compounds may optionally be lowered due to the combined action (e.g., additive or synergistic effects) of the compounds.

A “prophylactically effective amount” refers to an amount of a compound, or pharmaceutically acceptable salt thereof, sufficient to prevent or reduce the severity of one or more future symptoms of a disease or disorder when administered to a subject who is susceptible and/or who may develop the disease or disorder. For prophylactic use, beneficial or desired results include, e.g., results such as eliminating or reducing the risk, lessening the severity of future disease, or delaying the onset of the disease (e.g., delaying biochemical, histologic and/or behavioral symptoms of the disease, its complications, and intermediate pathological phenotype presenting during future development of the disease).

It is understood that an effective amount of a compound as disclosed herein, or pharmaceutically acceptable salt thereof, including a prophylactically effective amount, may be given to a subject in the adjuvant setting, which refers to a clinical setting in which a subject has had a history of the disease or disorder, and generally (but not necessarily) has been responsive to therapy, which includes, but is not limited to, surgery (e.g., surgical resection), radiotherapy, and chemotherapy. However, because of their or their family's history of the disease or disorder, these subjects are considered at risk of developing it. Treatment or administration in the “adjuvant setting” refers to a subsequent mode of treatment.

As used herein, “unit dosage form” refers to physically discrete units, suitable as unit dosages, each unit containing a predetermined quantity of active ingredient calculated to produce a desired therapeutic effect, in association with the required pharmaceutical carrier or excipient. Unit dosage forms may contain a single compound or a combination therapy.

As used herein, the term “controlled release” refers to a formulation or fraction thereof containing an active pharmaceutical ingredient in which release of the pharmaceutical is not immediate. Thus, with a “controlled release” formulation, administration to a subject does not result in immediate release of the drug into the subject's circulation. The term encompasses depot formulations designed to gradually release the drug compound over an extended period of time. Controlled release formulations can include a wide variety of drug delivery systems, generally involving mixing the drug compound with carriers, polymers or other compounds having the desired release characteristics (e.g., pH-dependent or non-pH-dependent solubility, different degrees of water solubility, and the like), and formulating the mixture according to the desired route of delivery, (e.g., coated capsules, implantable reservoirs, injectable solutions containing biodegradable capsules, and the like).

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared and updated by the U.S. Food and Drug Administration.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, or xanthan gum. Coatings include, e.g., cellulose acetate phthalate, ethylcellulose, gellan gum, maltodextrin, enteric coatings. Compression/encapsulation aids include, e.g., calcium carbonate, dextrose, fructose dc (dc means “directly compressible”), honey dc, lactose (anhydrate or monohydrate; optionally in combination with aspartame, cellulose, or microcrystalline cellulose), starch dc, sucrose. Disintegrants include, e.g., croscarmellose sodium, gellan gum, sodium starch glycolate. Creams or lotions include, e.g., maltodextrin, carrageenans. Lubricants include, e.g., magnesium stearate, stearic acid, sodium stearyl fumarate. Materials for chewable tablets include, e.g. dextrose, fructose dc, lactose (monohydrate, optionally in combination with aspartame or cellulose). Suspending/gelling agents include, e.g., carrageenan, sodium starch glycolate, xanthan gum. Sweeteners include, e.g., aspartame, dextrose, fructose dc, sorbitol, sucrose dc, etc.; and wet granulation agents include, e.g., calcium carbonate, maltodextrin, microcrystalline cellulose, etc. In some cases, the terms “excipient” and “carrier” are used interchangeably.

The term “subject” or “patient” refers humans, whether adult, juvenile or infant, but may also encompass other higher animals such as mammals, in which case the term may include, but is not limited to, primates, cows, sheep, goats, horses, dogs, cats, rabbits, rats, mice.

Representative amide compounds according to formulae (II-A), (II-B), (II-C), (II-D), (II-E) and (II-F) are shown in Table 1. Additional compounds falling within Formulae I-A-I-G are provided in the Examples. It is understood that individual enantiomers and diastereomers are included in the table below, as applicable. It is further to be understood that inclusion of a single particular enantiomer or diastereomer of a particular molecule does not preclude its partner enantiomer, or another stereoisomer, from being encompassed by the current disclosure. The numbering of compounds herein (such as in the first column of Table 1), including both the use or omission of particular numbers or sequences of numbers, is arbitrary.

TABLE 1
Amides Comprising Picolinamide and Other Scaffolds

| Compound # | Structure | Name |
|---|---|---|
| 1 | 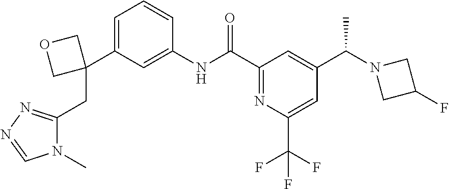 [SMILES: C[C@@H](c1cc(C(=O)Nc2cccc(C3(Cc4nncn4C)COC3)c2)nc(C(F)(F)F)c1)N1CC(F)C1] | (S)-4-(1-(3-fluoroazetidin-1-yl)ethyl)-N- (3-(3-((4-methyl-4H-1,2,4-triazol-3- yl)methyl)oxetan-3-yl)phenyl)-6- (trifluoromethyl)picolinamide |
| 2 | 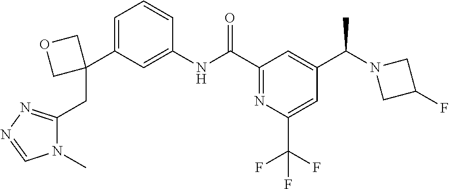 [SMILES: C[C@H](c1cc(C(=O)Nc2cccc(C3(Cc4nncn4C)COC3)c2)nc(C(F)(F)F)c1)N1CC(F)C1] | (R)-4-(1-(3-fluoroazetidin-1-yl)ethyl)-N- (3-(3-((4-methyl-4H-1,2,4-triazol-3- yl)methyl)oxetan-3-yl)phenyl)-6- (trifluoromethyl)picolinamide |
| 3 | 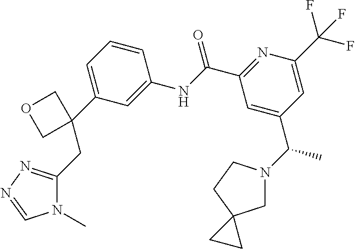 [SMILES: C[C@@H](c1cc(C(=O)Nc2cccc(C3(Cc4nncn4C)COC3)c2)nc(C(F)(F)F)c1)N1CCC2(CC2)C1] | (S)-4-(1-(5-azaspiro[2.4]heptan-5- yl)ethyl)-N-(3-(3-((4-methyl-4H-1,2,4- triazol-3-yl)methyl)oxetan-3-yl)phenyl)- 6-(trifluoromethyl)picolinamide |
| 4 | 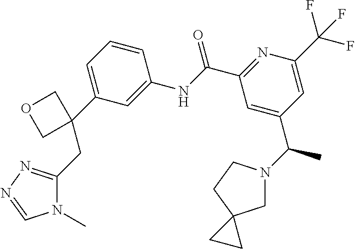 [SMILES: C[C@H](c1cc(C(=O)Nc2cccc(C3(Cc4nncn4C)COC3)c2)nc(C(F)(F)F)c1)N1CCC2(CC2)C1] | (R)-4-(1-(5-azaspiro[2.4]heptan-5- yl)ethyl)-N-(3-(3-((4-methyl-4H-1,2,4- triazol-3-yl)methyl)oxetan-3-yl)phenyl)- 6-(trifluoromethyl)picolinamide |
| 5 | 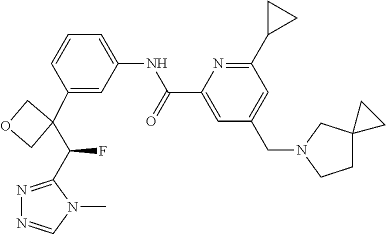 [SMILES: Cn1cnnc1[C@H](F)C1(c2cccc(NC(=O)c3cc(CN4CCC5(CC5)C4)cc(C4CC4)n3)c2)COC1] | (R)-4-(5-azaspiro[2.4]heptan-5- ylmethyl)-6-cyclopropyl-N-(3-(3- (fluoro(4-methyl-4H-1,2,4-triazol-3- yl)methyl)oxetan-3- yl)phenyl)picolinamide |
| 6 | 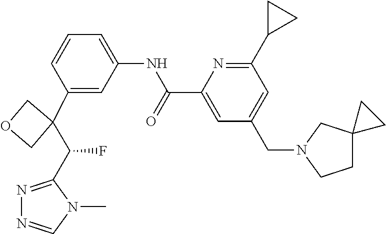 [SMILES: Cn1cnnc1[C@@H](F)C1(c2cccc(NC(=O)c3cc(CN4CCC5(CC5)C4)cc(C4CC4)n3)c2)COC1] | (S)-4-(5-azaspiro[2.4]heptan-5- ylmethyl)-6-cyclopropyl-N-(3-(3- (fluoro(4-methyl-4H-1,2,4-triazol-3- yl)methyl)oxetan-3- yl)phenyl)picolinamide |
| 7 | 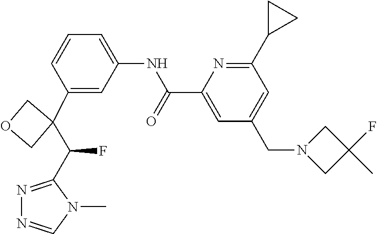 [SMILES: Cn1cnnc1[C@H](F)C1(c2cccc(NC(=O)c3cc(CN4CC(C)(F)C4)cc(C4CC4)n3)c2)COC1] | (R)-6-cyclopropyl-N-(3-(3-(fluoro(4- methyl-4H-1,2,4-triazol-3- yl)methyl)oxetan-3-yl)phenyl)-4-((3- fluoro-3-methylazetidin-1- yl)methyl)picolinamide |
| 8 | 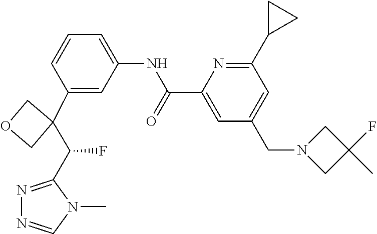 [SMILES: Cn1cnnc1[C@@H](F)C1(c2cccc(NC(=O)c3cc(CN4CC(C)(F)C4)cc(C4CC4)n3)c2)COC1] | (S)-6-cyclopropyl-N-(3-(3-(fluoro(4- methyl-4H-1,2,4-triazol-3- yl)methyl)oxetan-3-yl)phenyl)-4-((3- fluoro-3-methylazetidin-1- yl)methyl)picolinamide |
| 16 | 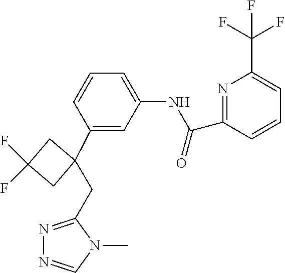 [SMILES: Cn1cnnc1CC1(c2cccc(NC(=O)c3cccc(C(F)(F)F)n3)c2)CC(F)(F)C1] | N-(3-(3,3-difluoro-1-((4-methyl-4H- 1,2,4-triazol-3- yl)methyl)cyclobutyl)phenyl)-6- (trifluoromethyl)picolinamide |
| 18 | 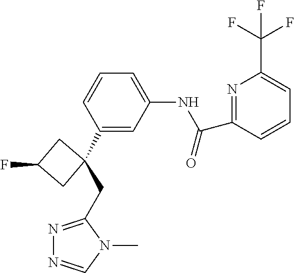 [SMILES: Cn1cnnc1C[C@]1(c2cccc(NC(=O)c3cccc(C(F)(F)F)n3)c2)C[C@H](F)C1] | N-(3-((1R,3S)-3-fluoro-1-((4-methyl-4H- 1,2,4-triazol-3- yl)methyl)cyclobutyl)phenyl)-6- (trifluoromethyl)picolinamide |
| 19 | 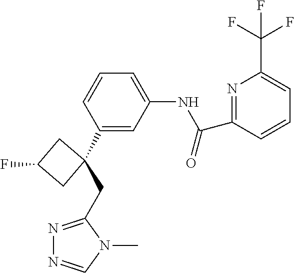 [SMILES: Cn1cnnc1C[C@]1(c2cccc(NC(=O)c3cccc(C(F)(F)F)n3)c2)C[C@@H](F)C1] | N-(3-((1S,3R)-3-fluoro-1-((4-methyl-4H- 1,2,4-triazol-3- yl)methyl)cyclobutyl)phenyl)-6- (trifluoromethyl)picolinamide |
| 20 | 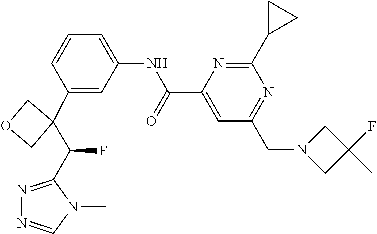 [SMILES: Cn1cnnc1[C@H](F)C1(c2cccc(NC(=O)c3cc(CN4CC(C)(F)C4)nc(C4CC4)n3)c2)COC1] | (R)-2-cyclopropyl-N-(3-(3-(fluoro(4- methyl-4H-1,2,4-triazol-3- yl)methyl)oxetan-3-yl)phenyl)-6-((3- fluoro-3-methylazetidin-1- yl)methyl)pyrimidine-4-carboxamide |
| 21 | 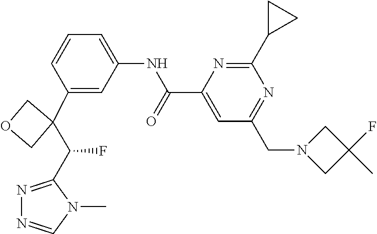 [SMILES: Cn1cnnc1[C@@H](F)C1(c2cccc(NC(=O)c3cc(CN4CC(C)(F)C4)nc(C4CC4)n3)c2)COC1] | 2-cyclopropyl-6-[(3-fluoro-3-methyl- azetidin-1-yl)methyl]-N-[3-[3-[(S)-fluoro- (4-methyl-1,2,4-triazol-3- yl)methyl]oxetan-3- yl]phenyl]pyrimidine-4-carboxamide |
| 22 | 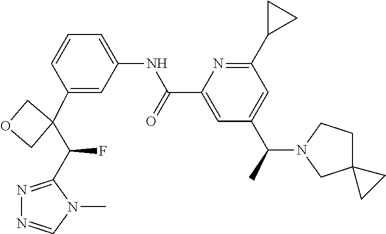 [SMILES: C[C@@H](c1cc(C(=O)Nc2cccc(C3([C@@H](F)c4nncn4C)COC3)c2)nc(C2CC2)c1)N1CCC2(CC2)C1] | 4-((S)-1-(5-azaspiro[2.4]heptan-5- yl)ethyl)-6-cyclopropyl-N-(3-(3-((R)- fluoro(4-methyl-4H-1,2,4-triazol-3- yl)methyl)oxetan-3- yl)phenyl)picolinamide |
| 23 | 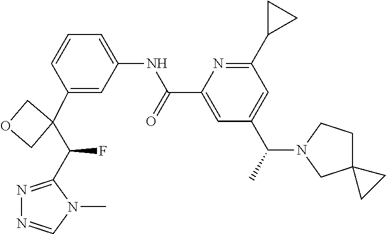 [SMILES: C[C@H](c1cc(C(=O)Nc2cccc(C3([C@@H](F)c4nncn4C)COC3)c2)nc(C2CC2)c1)N1CCC2(CC2)C1] | 4-((R)-1-(5-azaspiro[2.4]heptan-5- yl)ethyl)-6-cyclopropyl-N-(3-(3-((R)- fluoro(4-methyl-4H-1,2,4-triazol-3- yl)methyl)oxetan-3- yl)phenyl)picolinamide |
| 24 | 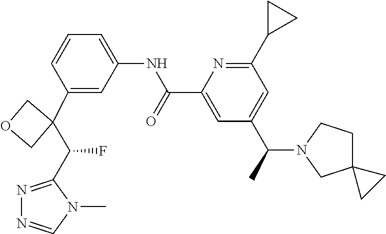 [SMILES: C[C@@H](c1cc(C(=O)Nc2cccc(C3([C@H](F)c4nncn4C)COC3)c2)nc(C2CC2)c1)N1CCC2(CC2)C1] | 4-((S)-1-(5-azaspiro[2.4]heptan-5- yl)ethyl)-6-cyclopropyl-N-(3-(3-((S)- fluoro(4-methyl-4H-1,2,4-triazol-3- yl)methyl)oxetan-3- yl)phenyl)picolinamide |
| 25 | 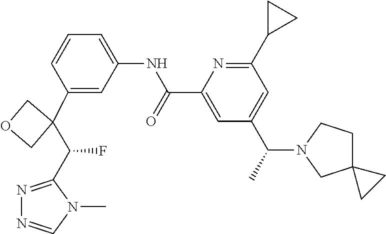 [SMILES: C[C@H](c1cc(C(=O)Nc2cccc(C3([C@H](F)c4nncn4C)COC3)c2)nc(C2CC2)c1)N1CCC2(CC2)C1] | 4-((R)-1-(5-azaspiro[2.4]heptan-5- yl)ethyl)-6-cyclopropyl-N-(3-(3-((S)- fluoro(4-methyl-4H-1,2,4-triazol-3- yl)methyl)oxetan-3- yl)phenyl)picolinamide |
| 26 | 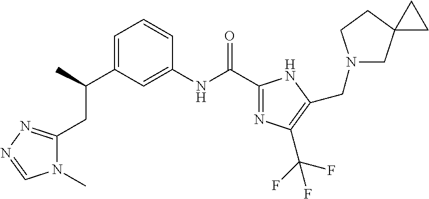 [SMILES: C[C@H](Cc1nncn1C)c1cccc(NC(=O)c2nc(C(F)(F)F)c(CN3CCC4(CC4)C3)[nH]2)c1] | (R)-5-(5-azaspiro[2.4]heptan-5- ylmethyl)-N-(3-(1-(4-methyl-4H-1,2,4- triazol-3-yl)propan-2-yl)phenyl)-4- (trifluoromethyl)-1H-imidazole-2- carboxamide |
| 35 | 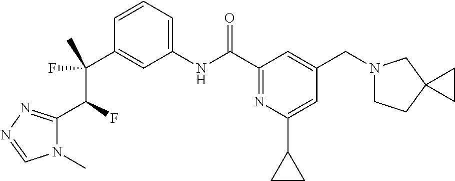 [SMILES: Cn1cnnc1[C@H](F)[C@](C)(F)c1cccc(NC(=O)c2cc(CN3CCC4(CC4)C3)cc(C3CC3)n2)c1] | 4-(5-azaspiro[2.4]heptan-5-ylmethyl)-6- cyclopropyl-N-(3-((1S,2R)-1,2-difluoro- 1-(4-methyl-4H-1,2,4-triazol-3- yl)propan-2-yl)phenyl)picolinamide |
| 36 | 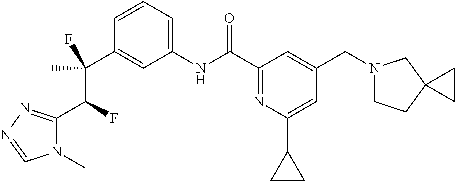 [SMILES: Cn1cnnc1[C@H](F)[C@@](C)(F)c1cccc(NC(=O)c2cc(CN3CCC4(CC4)C3)cc(C3CC3)n2)c1] | 4-(5-azaspiro[2.4]heptan-5-ylmethyl)-6- cyclopropyl-N-(3-((1S,2S)-1,2-difluoro- 1-(4-methyl-4H-1,2,4-triazol-3- yl)propan-2-yl)phenyl)picolinamide |
| 37 | 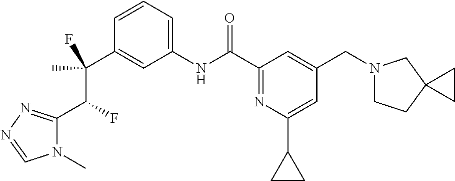 [SMILES: Cn1cnnc1[C@@H](F)[C@@](C)(F)c1cccc(NC(=O)c2cc(CN3CCC4(CC4)C3)cc(C3CC3)n2)c1] | 4-(5-azaspiro[2.4]heptan-5-ylmethyl)-6- cyclopropyl-N-(3-((1R,2S)-1,2-difluoro- 1-(4-methyl-4H-1,2,4-triazol-3- yl)propan-2-yl)phenyl)picolinamide |
| 38 | 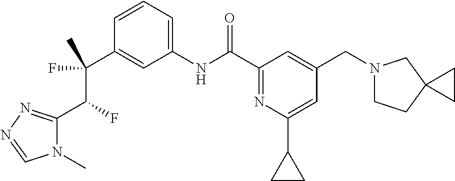 [SMILES: Cn1cnnc1[C@@H](F)[C@](C)(F)c1cccc(NC(=O)c2cc(CN3CCC4(CC4)C3)cc(C3CC3)n2)c1] | 4-(5-azaspiro[2.4]heptan-5-ylmethyl)-6- cyclopropyl-N-(3-((1R,2R)-1,2-difluoro- 1-(4-methyl-4H-1,2,4-triazol-3- yl)propan-2-yl)phenyl)picolinamide |
| 39 | 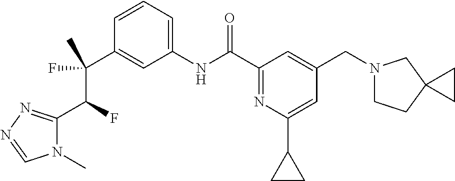 [SMILES: Cn1cnnc1[C@H](F)[C@](C)(F)c1cccc(NC(=O)c2cc(CN3CCC4(CC4)C3)cc(C3CC3)n2)c1] | 6-(5-azaspiro[2.4]heptan-5-ylmethyl)-2- cyclopropyl-N-(3-((1S,2R)-1,2-difluoro- 1-(4-methyl-4H-1,2,4-triazol-3- yl)propan-2-yl)phenyl)pyrimidine-4- carboxamide |
| 40 | 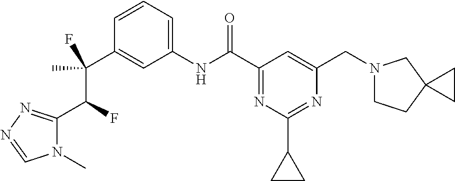 [SMILES: Cn1cnnc1[C@H](F)[C@@](C)(F)c1cccc(NC(=O)c2cc(CN3CCC4(CC4)C3)nc(C3CC3)n2)c1] | 6-(5-azaspiro[2.4]heptan-5-ylmethyl)-2- cyclopropyl-N-(3-((1S,2S)-1,2-difluoro- 1-(4-methyl-4H-1,2,4-triazol-3- yl)propan-2-yl)phenyl)pyrimidine-4- carboxamide |
| 41 | 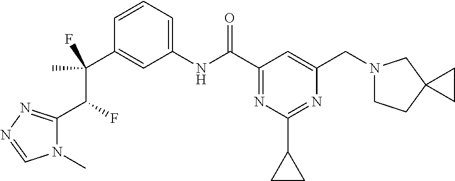 [SMILES: Cn1cnnc1[C@@H](F)[C@@](C)(F)c1cccc(NC(=O)c2cc(CN3CCC4(CC4)C3)nc(C3CC3)n2)c1] | 6-(5-azaspiro[2.4]heptan-5-ylmethyl)-2- cyclopropyl-N-(3-((1R,2S)-1,2-difluoro- 1-(4-methyl-4H-1,2,4-triazol-3- yl)propan-2-yl)phenyl)pyrimidine-4- carboxamide |
| 42 | 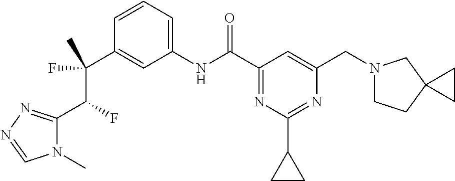 [SMILES: Cn1cnnc1[C@@H](F)[C@](C)(F)c1cccc(NC(=O)c2cc(CN3CCC4(CC4)C3)nc(C3CC3)n2)c1] | 6-(5-azaspiro[2.4]heptan-5-ylmethyl)-2- cyclopropyl-N-(3-((1R,2R)-1,2-difluoro- 1-(4-methyl-4H-1,2,4-triazol-3- yl)propan-2-yl)phenyl)pyrimidine-4- carboxamide |
| 43 | 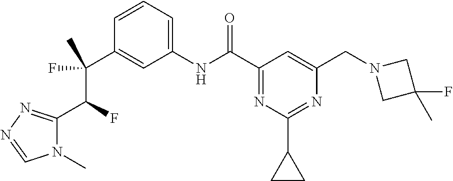 [SMILES: Cn1cnnc1[C@H](F)[C@](C)(F)c1cccc(NC(=O)c2cc(CN3CC(C)(F)C3)nc(C3CC3)n2)c1] | 2-cyclopropyl-N-(3-((1S,2R)-1,2- difluoro-1-(4-methyl-4H-1,2,4-triazol-3- yl)propan-2-yl)phenyl)-6-((3-fluoro-3- methylazetidin-1-yl)methyl)pyrimidine- 4-carboxamide |
| 44 | 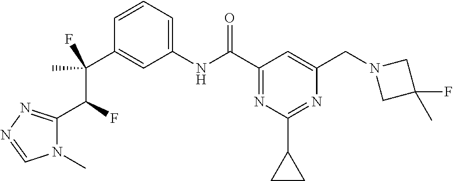 [SMILES: Cn1cnnc1[C@H](F)[C@@](C)(F)c1cccc(NC(=O)c2cc(CN3CC(C)(F)C3)nc(C3CC3)n2)c1] | 2-cyclopropyl-N-(3-((1S,2S)-1,2- difluoro-1-(4-methyl-4H-1,2,4-triazol-3- yl)propan-2-yl)phenyl)-6-((3-fluoro-3- methylazetidin-1-yl)methyl)pyrimidine- 4-carboxamid |
| 45 | 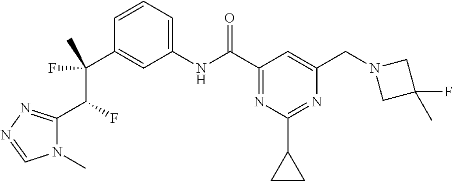 [SMILES: Cn1cnnc1[C@@H](F)[C@](C)(F)c1cccc(NC(=O)c2cc(CN3CC(C)(F)C3)nc(C3CC3)n2)c1] | 2-cyclopropyl-N-(3-((1R,2S)-1,2- difluoro-1-(4-methyl-4H-1,2,4-triazol-3- yl)propan-2-yl)phenyl)-6-((3-fluoro-3- methylazetidin-1-yl)methyl)pyrimidine- 4-carboxamide |
| 46 | 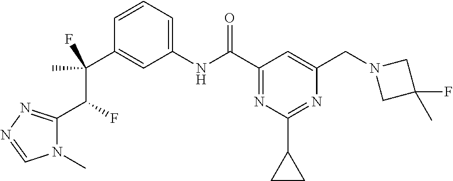 [SMILES: Cn1cnnc1[C@@H](F)[C@@](C)(F)c1cccc(NC(=O)c2cc(CN3CC(C)(F)C3)nc(C3CC3)n2)c1] | 2-cyclopropyl-N-(3-((1R,2R)-1,2- difluoro-1-(4-methyl-4H-1,2,4-triazol-3- yl)propan-2-yl)phenyl)-6-((3-fluoro-3- methylazetidin-1-yl)methyl)pyrimidine- 4-carboxamide |
| 60 | 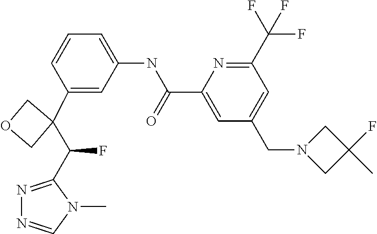 [SMILES: Cn1cnnc1[C@H](F)C1(c2cccc(NC(=O)c3cc(CN4CC(C)(F)C4)cc(C(F)(F)F)n3)c2)COC1] | (R)-N-(3-(3-(fluoro(4-methyl-4H-1,2,4- triazol-3-yl)methyl)oxetan-3-yl)phenyl)- 4-((3-fluoro-3-methylazetidin-1- yl)methyl)-6- (trifluoromethyl)picolinamide |
| 61 | 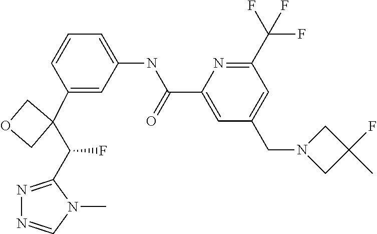 [SMILES: Cn1cnnc1[C@@H](F)C1(c2cccc(NC(=O)c3cc(CN4CC(C)(F)C4)cc(C(F)(F)F)n3)c2)COC1] | (S)-N-(3-(3-(fluoro(4-methyl-4H-1,2,4- triazol-3-yl)methyl)oxetan-3-yl)phenyl)- 4-((3-fluoro-3-methylazetidin-1- yl)methyl)-6- (trifluoromethyl)picolinamide |
| 62 | 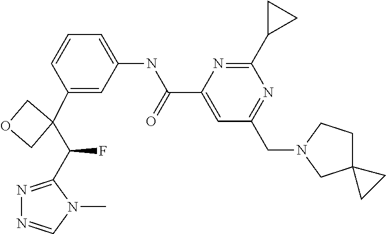 [SMILES: Cn1cnnc1[C@H](F)C1(c2cccc(NC(=O)c3cc(CN4CCC5(CC5)C4)nc(C4CC4)n3)c2)COC1] | (R)-6-((5-azaspiro[2.4]heptan-5- yl)methyl)-2-cyclopropyl-N-(3-(3- (fluoro(4-methyl-4H-1,2,4-triazol-3- yl)methyl)oxetan-3- yl)phenyl)pyrimidine-4-carboxamide |
| 63 | 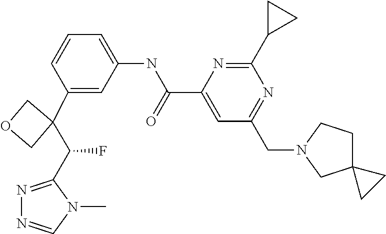 [SMILES: Cn1cnnc1[C@@H](F)C1(c2cccc(NC(=O)c3cc(CN4CCC5(CC5)C4)nc(C4CC4)n3)c2)COC1] | (S)-6-((5-azaspiro[2.4]heptan-5- yl)methyl)-2-cyclopropyl-N-(3-(3- (fluoro(4-methyl-4H-1,2,4-triazol-3- yl)methyl)oxetan-3- yl)phenyl)pyrimidine-4-carboxamide |
| 64 | 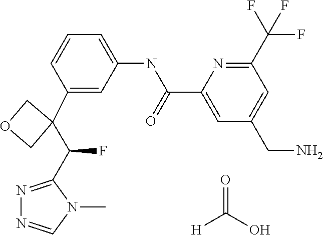 [SMILES: Cn1cnnc1[C@H](F)C1(c2cccc(NC(=O)c3cc(CN)cc(C(F)(F)F)n3)c2)COC1 ; O=CO] | (R)-4-(aminomethyl)-N-(3-(3-(fluoro(4- methyl-4H-1,2,4-triazol-3- yl)methyl)oxetan-3-yl)phenyl)-6- (trifluoromethyl)picolinamide formate |
| 65 | 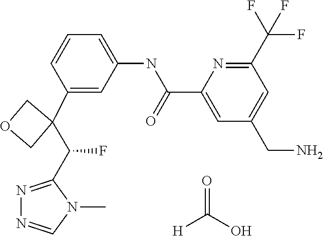 [SMILES: Cn1cnnc1[C@@H](F)C1(c2cccc(NC(=O)c3cc(CN)cc(C(F)(F)F)n3)c2)COC1 ; O=CO] | (S)-4-(aminomethyl)-N-(3-(3-(fluoro(4- methyl-4H-1,2,4-triazol-3- yl)methyl)oxetan-3-yl)phenyl)-6- (trifluoromethyl)picolinamide formate |
| 67 | 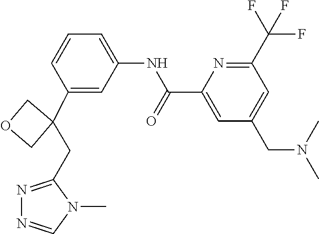 [SMILES: CN(C)Cc1cc(C(=O)Nc2cccc(C3(Cc4nncn4C)COC3)c2)nc(C(F)(F)F)c1] | 4-((dimethylamino)methyl)-N-(3-(3-((4- methyl-4H-1,2,4-triazol-3- yl)methyl)oxetan-3-yl)phenyl)-6- (trifluoromethyl)picolinamide |
| 98 | 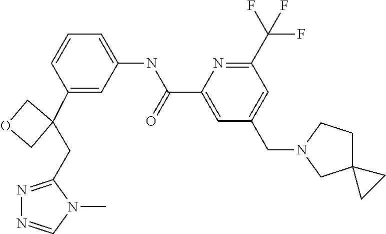 [SMILES: Cn1cnnc1CC1(c2cccc(NC(=O)c3cc(CN4CCC5(CC5)C4)cc(C(F)(F)F)n3)c2)COC1] | 4-((5-azaspiro[2.4]heptan-5-yl)methyl)- N-(3-(3-((4-methyl-4H-1,2,4-triazol-3- yl)methyl)oxetan-3-yl)phenyl)-6- (trifluoromethyl)picolinamide |
| 112 | 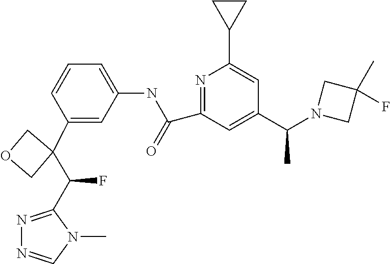 [SMILES: C[C@@H](c1cc(C(=O)Nc2cccc(C3([C@@H](F)c4nncn4C)COC3)c2)nc(C2CC2)c1)N1CC(C)(F)C1] | 6-cyclopropyl-N-(3-(3-((R)-fluoro(4- methyl-4H-1,2,4-triazol-3- yl)methyl)oxetan-3-yl)phenyl)-4-((S)-1- (3-fluoro-3-methylazetidin-1- yl)ethyl)picolinamide |
| 113 | 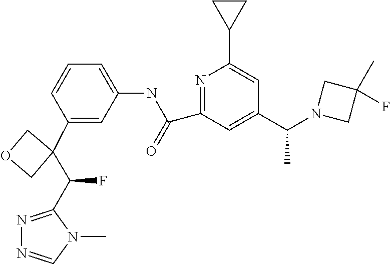 [SMILES: C[C@H](c1cc(C(=O)Nc2cccc(C3([C@@H](F)c4nncn4C)COC3)c2)nc(C2CC2)c1)N1CC(C)(F)C1] | 6-cyclopropyl-N-(3-(3-((R)-fluoro(4- methyl-4H-1,2,4-triazol-3- yl)methyl)oxetan-3-yl)phenyl)-4-((R)-1- (3-fluoro-3-methylazetidin-1- yl)ethyl)picolinamide |
| 114 | 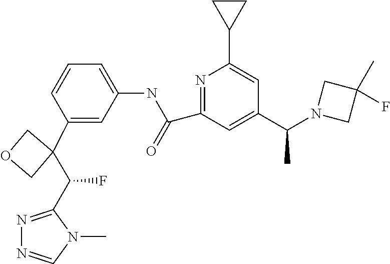 [SMILES: C[C@@H](c1cc(C(=O)Nc2cccc(C3([C@H](F)c4nncn4C)COC3)c2)nc(C2CC2)c1)N1CC(C)(F)C1] | 6-cyclopropyl-N-(3-(3-((S)-fluoro(4- methyl-4H-1,2,4-triazol-3- yl)methyl)oxetan-3-yl)phenyl)-4-((S)-1- (3-fluoro-3-methylazetidin-1- yl)ethyl)picolinamide |
| 115 | 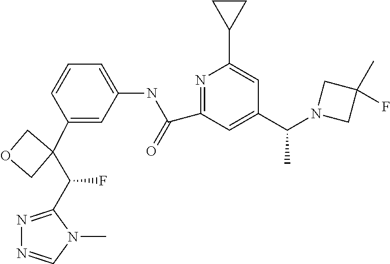 [SMILES: C[C@H](c1cc(C(=O)Nc2cccc(C3([C@H](F)c4nncn4C)COC3)c2)nc(C2CC2)c1)N1CC(C)(F)C1] | 6-cyclopropyl-N-(3-(3-((S)-fluoro(4- methyl-4H-1,2,4-triazol-3- yl)methyl)oxetan-3-yl)phenyl)-4-((R)-1- (3-fluoro-3-methylazetidin-1- yl)ethyl)picolinamide |
| 121 | 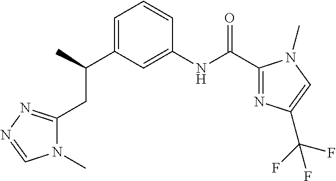 [SMILES: C[C@H](Cc1nncn1C)c1cccc(NC(=O)c2nc(C(F)(F)F)cn2C)c1] | (R)-1-methyl-N-(3-(1-(4-methyl-4H- 1,2,4-triazol-3-yl)propan-2-yl)phenyl)-4- (trifluoromethyl)-1H-imidazole-2- carboxamide |
| 122 | 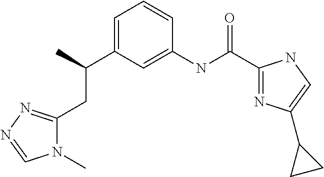 [SMILES: C[C@H](Cc1nncn1C)c1cccc(NC(=O)c2nc(C3CC3)c[nH]2)c1] | (R)-4-cyclopropyl-N-(3-(1-(4-methyl- 4H-1,2,4-triazol-3-yl)propan-2- yl)phenyl)-1H-imidazole-2-carboxamide |
| 123 | 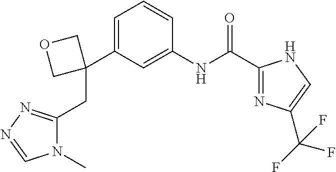 [SMILES: Cn1cnnc1CC1(c2cccc(NC(=O)c3nc(C(F)(F)F)c[nH]3)c2)COC1] | N-(3-(3-((4-methyl-4H-1,2,4-triazol-3- yl)methyl)oxetan-3-yl)phenyl)-4- (trifluoromethyl)-1H-imidazole-2- carboxamide |
| 124 | 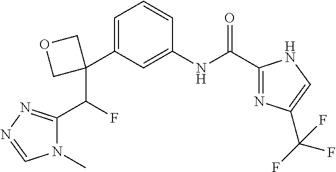 [SMILES: Cn1cnnc1C(F)C1(c2cccc(NC(=O)c3nc(C(F)(F)F)c[nH]3)c2)COC1] | N-(3-(3-(fluoro(4-methyl-4H-1,2,4- triazol-3-yl)methyl)oxetan-3-yl)phenyl)- 4-(trifluoromethyl)-1H-imidazole-2- carboxamide |
| 223 | 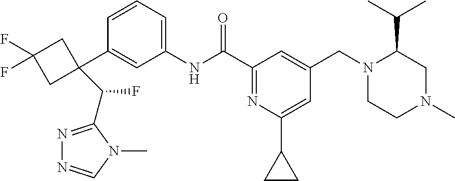 [SMILES: CC(C)[C@H]1CN(C)CCN1Cc1cc(C(=O)Nc2cccc(C3([C@H](F)c4nncn4C)CC(F)(F)C3)c2)nc(C2CC2)c1] | 6-cyclopropyl-N-(3-(3,3-difluoro-1-((S)- fluoro(4-methyl-4H-1,2,4-triazol-3- yl)methyl)cyclobutyl)phenyl)-4-(((S)-2- isopropyl-4-methylpiperazin-1- yl)methyl)picolinamide |
| 224 | 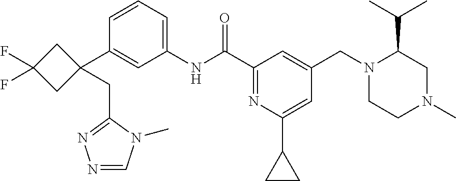 [SMILES: CC(C)[C@H]1CN(C)CCN1Cc1cc(C(=O)Nc2cccc(C3(Cc4nncn4C)CC(F)(F)C3)c2)nc(C2CC2)c1] | (S)-6-cyclopropyl-N-(3-(3,3-difluoro-1- ((4-methyl-4H-1,2,4-triazol-3- yl)methyl)cyclobutyl)phenyl)-4-((2- isopropyl-4-methylpiperazin-1- yl)methyl)picolinamide |
| 225 | 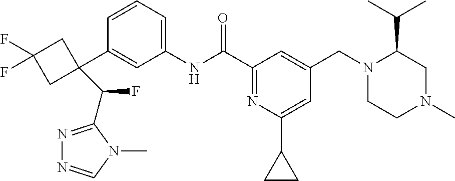 [SMILES: CC(C)[C@H]1CN(C)CCN1Cc1cc(C(=O)Nc2cccc(C3([C@@H](F)c4nncn4C)CC(F)(F)C3)c2)nc(C2CC2)c1] | 6-cyclopropyl-N-(3-(3,3-difluoro-1-((R)- fluoro(4-methyl-4H-1,2,4-triazol-3- yl)methyl)cyclobutyl)phenyl)-4-(((S)-2- isopropyl-4-methylpiperazin-1- yl)methyl)picolinamide |
| 495 | 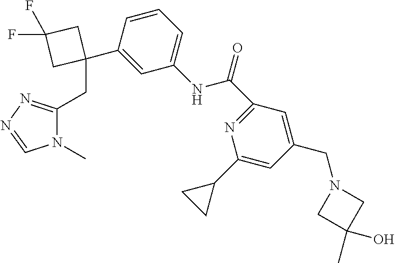 [SMILES: Cn1cnnc1CC1(c2cccc(NC(=O)c3cc(CN4CC(C)(O)C4)cc(C4CC4)n3)c2)CC(F)(F)C1] | 6-cyclopropyl-N-(3-(3,3-difluoro-1-((4- methyl-4H-1,2,4-triazol-3- yl)methyl)cyclobutyl)phenyl)-4-((3- hydroxy-3-methylazetidin-1- yl)methyl)picolinamide |
| 496 | 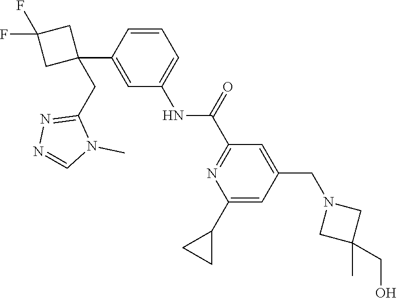 [SMILES: Cn1cnnc1CC1(c2cccc(NC(=O)c3cc(CN4CC(C)(CO)C4)cc(C4CC4)n3)c2)CC(F)(F)C1] | 6-cyclopropyl-N-(3-(3,3-difluoro-1-((4- methyl-4H-1,2,4-triazol-3- yl)methyl)cyclobutyl)phenyl)-4-((3- (hydroxymethyl)-3-methylazetidin-1- yl)methyl)picolinamide |
| 497 | 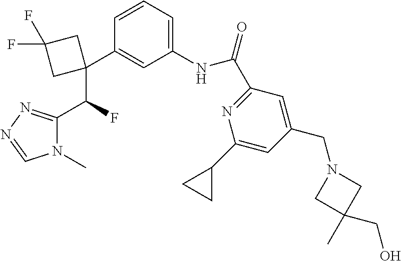 [SMILES: Cn1cnnc1[C@H](F)C1(c2cccc(NC(=O)c3cc(CN4CC(C)(CO)C4)cc(C4CC4)n3)c2)CC(F)(F)C1] | (R)-6-cyclopropyl-N-(3-(3,3-difluoro-1- (fluoro(4-methyl-4H-1,2,4-triazol-3- yl)methyl)cyclobutyl)phenyl)-4-((3- (hydroxymethyl)-3-methylazetidin-1- yl)methyl)picolinamide |
| 498 | 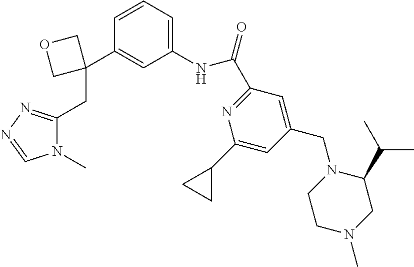 [SMILES: CC(C)[C@H]1CN(C)CCN1Cc1cc(C(=O)Nc2cccc(C3(Cc4nncn4C)COC3)c2)nc(C2CC2)c1] | (S)-6-cyclopropyl-4-((2-isopropyl-4- methylpiperazin-1-yl)methyl)-N-(3-(3- ((4-methyl-4H-1,2,4-triazol-3- yl)methyl)oxetan-3- yl)phenyl)picolinamide |
| 499 | 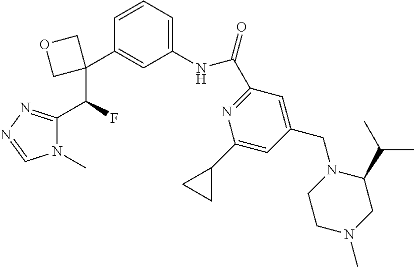 [SMILES: CC(C)[C@H]1CN(C)CCN1Cc1cc(C(=O)Nc2cccc(C3([C@@H](F)c4nncn4C)COC3)c2)nc(C2CC2)c1] | 6-cyclopropyl-N-(3-(3-((R)-fluoro(4- methyl-4H-1,2,4-triazol-3- yl)methyl)oxetan-3-yl)phenyl)-4-(((S)-2- isopropyl-4-methylpiperazin-1- yl)methyl)picolinamide |
| 500 | 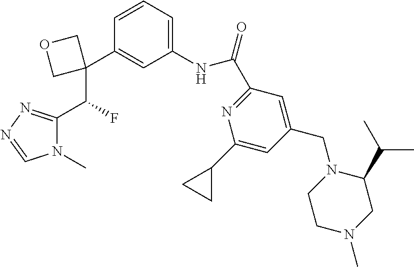 [SMILES: CC(C)[C@H]1CN(C)CCN1Cc1cc(C(=O)Nc2cccc(C3([C@H](F)c4nncn4C)COC3)c2)nc(C2CC2)c1] | 6-cyclopropyl-N-(3-(3-((S)-fluoro(4- methyl-4H-1,2,4-triazol-3- yl)methyl)oxetan-3-yl)phenyl)-4-(((S)-2- isopropyl-4-methylpiperazin-1- yl)methyl)picolinamide |
| 501 | 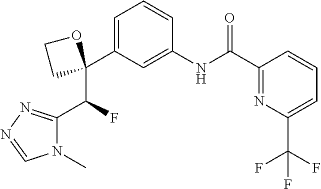 [SMILES: Cn1cnnc1[C@H](F)[C@@]1(c2cccc(NC(=O)c3cccc(C(F)(F)F)n3)c2)CCO1] | N-(3-((S)-2-((S)-fluoro(4-methyl-4H- 1,2,4-triazol-3-yl)methyl)oxetan-2- yl)phenyl)-6- (trifluoromethyl)picolinamide |
| 502 | 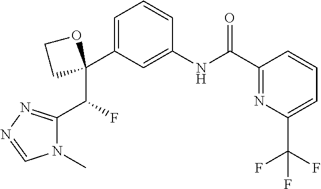 [SMILES: Cn1cnnc1[C@@H](F)[C@@]1(c2cccc(NC(=O)c3cccc(C(F)(F)F)n3)c2)CCO1] | N-(3-((S)-2-((R)-fluoro(4-methyl-4H- 1,2,4-triazol-3-yl)methyl)oxetan-2- yl)phenyl)-6- (trifluoromethyl)picolinamide |
| 503 | 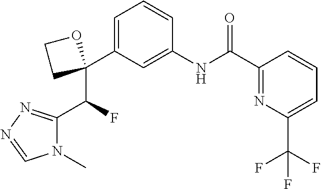 [SMILES: Cn1cnnc1[C@H](F)[C@]1(c2cccc(NC(=O)c3cccc(C(F)(F)F)n3)c2)CCO1] | N-(3-((R)-2-((S)-fluoro(4-methyl-4H- 1,2,4-triazol-3-yl)methyl)oxetan-2- yl)phenyl)-6- (trifluoromethyl)picolinamide |
| 504 | 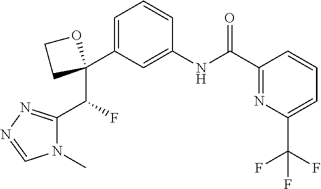 [SMILES: Cn1cnnc1[C@@H](F)[C@]1(c2cccc(NC(=O)c3cccc(C(F)(F)F)n3)c2)CCO1] | N-(3-((R)-2-((R)-fluoro(4-methyl-4H- 1,2,4-triazol-3-yl)methyl)oxetan-2- yl)phenyl)-6- (trifluoromethyl)picolinamide |
| 505 | 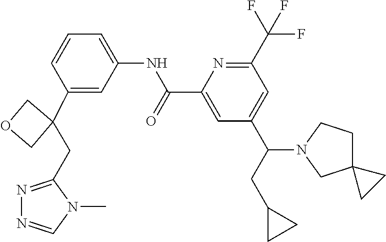 [SMILES: Cn1cnnc1CC1(c2cccc(NC(=O)c3cc(C(CC4CC4)N4CCC5(CC5)C4)cc(C(F)(F)F)n3)c2)COC1] | 4-(2-cyclopropyl-1-(5- azaspiro[2.4]heptan-5-yl)ethyl)-N-(3-(3- ((4-methyl-4H-1,2,4-triazol-3- yl)methyl)oxetan-3-yl)phenyl)-6- (trifluoromethyl)picolinamide |
| 506 | 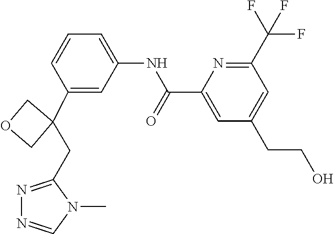 [SMILES: Cn1cnnc1CC1(c2cccc(NC(=O)c3cc(CCO)cc(C(F)(F)F)n3)c2)COC1] | 4-(2-hydroxyethyl)-N-(3-(3-((4-methyl- 4H-1,2,4-triazol-3-yl)methyl)oxetan-3- yl)phenyl)-6-(trifluoromethyl)- picolinamide |
| 507 | 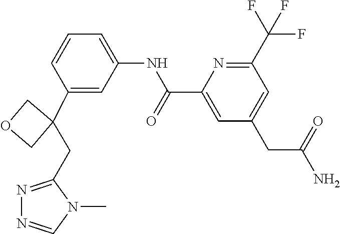 [SMILES: Cn1cnnc1CC1(c2cccc(NC(=O)c3cc(CC(N)=O)cc(C(F)(F)F)n3)c2)COC1] | 4-(2-amino-2-oxoethyl)-N-(3-(3-((4- methyl-4H-1,2,4-triazol-3- yl)methyl)oxetan-3-yl)phenyl)-6- (trifluoromethyl)picolinamide |

### Pharmaceutical Compositions and Formulations

The presently disclosed compounds can be formulated into pharmaceutical compositions along with a pharmaceutically acceptable carrier or excipient. According to this aspect, there is provided a pharmaceutical composition comprising a compound of Formulae (II-A)-(II-F) in association with a pharmaceutically acceptable excipient, diluent or carrier.

The formulations of Compounds of Formulae (II-A)-(II-F) include those suitable for the administration routes detailed herein. They may conveniently be presented in unit dosage form and can be formulated in accordance with standard pharmaceutical practice as a pharmaceutical composition. Techniques and formulations generally and suitable for use herein are found in Remington's Pharmaceutical Sciences (16^{th} edition, Osol, A. Ed. (1980); Mack Publishing Co., Easton, PA). Such methods include the step of bringing into association the active ingredient with the excipient or carrier which constitutes one or more accessory ingredients. In general the formulations are prepared by uniformly and intimately bringing into association the active ingredient with liquid excipients or carriers or finely divided solid excipients or carriers or both, and then, if necessary, shaping the product.

A typical formulation is prepared by mixing a compound of Formulae (II-A)-(II-F), and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of Formulae (II-A)-(II-F), is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of Formulae (II-A)-(II-F), or pharmaceutical composition thereof) or aid in the manufacturing of the pharmaceutical product (i.e., medicament).

The formulations may be prepared using conventional dissolution and mixing procedures. For example, the bulk drug substance (i.e., compound of Formulae (II-A)-(II-F), or stabilized form of the Compound of Formulae (II-A)-(II-F), (e.g., complex with a cyclodextrin derivative or other known complexation agent) is dissolved in a suitable solvent in the presence of one or more of the excipients described above. The compound of Formulae (II-A)-(II-F) is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations may be prepared for various routes and types of administration. For example, a compound of Formulae (II-A)-(II-F) having the desired degree of purity may optionally be mixed with pharmaceutically acceptable diluents, carriers, excipients or stabilizers (Remington's Pharmaceutical Sciences (1980) 16^{th} edition, Osol, A. Ed.), in the form of a lyophilized formulation, milled powder, or an aqueous solution. Formulation may be conducted by mixing at ambient temperature at the appropriate pH, and at the desired degree of purity, with physiologically acceptable excipients or carriers, i.e., excipients or carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The compounds of Formulae (II-A)-(II-F) can be sterile. In particular, formulations to be used for in vivo administration should be sterile. Such sterilization is readily accomplished by filtration through sterile filtration membranes.

The compound of Formulae (II-A)-(II-F) ordinarily can be stored as a solid composition, a lyophilized formulation or as an aqueous solution.

The pharmaceutical compositions comprising a compound of Formulae (II-A)-(II-F) can be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. In some embodiments, the amount is below the amount that is toxic to the host or renders the host more susceptible to bleeding.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16^{th} edition, Osol, A. Ed. (1980).

Sustained-release preparations of Formulae (II-A)-(II-F) compounds may be prepared. Suitable examples of sustained-release preparations include semipermeable matrices of solid hydrophobic polymers containing a compound of Formulae (II-A)-(II-F), which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

Formulations of a compound of Formulae (II-A)-(II-F) suitable for oral administration may be prepared as discrete units such as pills, capsules, cachets or tablets each containing a predetermined amount of a compound of Formulae (II-A)-(II-F).

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g., gelatin capsules, syrups or elixirs may be prepared for oral use. Formulations of compounds of Formulae (II-A)-(II-F) intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400), and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions may be constituted from known ingredients in a known manner. While the phase may comprise solely an emulsifier, it may also comprise a mixture of at least one emulsifier and a fat or oil, or both a fat and an oil. A hydrophilic emulsifier included together with a lipophilic emulsifier may act as a stabilizer. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation include Tween® 60, Span® 80, cetostearyl alcohol, benzyl alcohol, myristyl alcohol, glyceryl mono-stearate and sodium lauryl sulfate.

Aqueous suspensions of Formulae (II-A)-(II-F) compounds contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

The pharmaceutical compositions of compounds of of Formulae (II-A)-(II-F), may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such 1,3-butanediol. The sterile injectable preparation may also be prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the excipient or carrier material to produce a single dosage form will vary depending upon the host treated and the particular mode of administration. For example, a time-release formulation intended for oral administration to humans may contain approximately 1 to 1000 mg of active material compounded with an appropriate and convenient amount of excipient or carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops in which the active ingredient is dissolved or suspended in a suitable excipient or carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of disorders as described below.

Formulations suitable for vaginal administration may be presented as pessaries, tampons, creams, gels, pastes, foams or spray formulations containing in addition to the active ingredient such excipients or carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid excipient or carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The subject matter further provides veterinary compositions comprising at least one active ingredient as above defined together with a veterinary excipient or carrier therefore. Veterinary excipients or carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

In particular embodiments the pharmaceutical composition comprising the presently disclosed compounds further comprise a chemotherapeutic agent. In some of these embodiments, the chemotherapeutic agent is an immunotherapeutic agent.

### Kits

Further provided are kits for carrying out the methods detailed herein, which kits comprise one or more compounds described herein or a pharmaceutical composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for use in the treatment of a disorder such as cancer. In some embodiments, the kit contains instructions for use in the treatment of a cancer.

Kits generally comprise suitable packaging. The kits may comprise one or more containers comprising any compound described herein. Each component (if there is more than one component) can be packaged in separate containers or some components can be combined in one container where cross-reactivity and shelf life permit. One or more components of a kit may be sterile and/or may be contained within sterile packaging.

The kits may be in unit dosage forms, bulk packages (e.g., multi-dose packages) or sub-unit doses. For example, kits may be provided that contain sufficient dosages of a compound as disclosed herein (e.g., a therapeutically effective amount) and/or a second pharmaceutically active compound useful for a disorder (e.g., cancer) to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and may be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to a subject.

### Methods of Use

The presently disclosed compounds find use in inhibiting the activity of Cbl-B. Many of the compounds additionally do so with an inhibitory effect that is greater than that for C-cbl.

In one embodiment, the subject matter disclosed herein is directed to a method of inhibiting Cbl-B, the method comprising contacting one or more cells containing active Cbl-B proteins with an effective amount of a compound of Formulae (II-A)-(II-F), or a pharmaceutical composition described herein. By “contacting” is meant, bringing the compound within close enough proximity to an isolated Cbl-B enzyme or a cell expressing Cbl-B (e.g., T cell, B cell, dendritic cell) such that the compound is able to bind to and inhibit the activity of Cbl-B. The compound can be contacted with Cbl-B in vitro or in vivo via administration of the compound to a subject.

In an embodiment, the subject matter disclosed herein is directed to a method for enhancing an immune response in a subject in need thereof, wherein the method comprises administering to said subject an effective amount of a compound of Formulae (II-A)-(II-F)), or a pharmaceutical composition described herein. In certain aspects of this embodiment, the T cells in the subject have at least one of enhanced priming, enhanced activation, enhanced migration, enhanced proliferation, enhanced survival, and enhanced cytolytic activity relative to prior to the administration of the compound or pharmaceutical composition. In certain aspects of this embodiment, the T cell activation is characterized by an elevated frequency of y-IFN+ CDS T cells, an elevated frequency of y-IFN+ CD4 T cells, or enhanced levels of IL-2 or granzyme B production by T cells, relative to prior to administration of the compound or pharmaceutical composition. In certain aspects of this embodiment, the number of T cells is elevated relative to prior to administration of the compound or pharmaceutical composition. In certain aspects of this embodiment, the T cell is an antigen-specific CDS T cell. In certain aspects of this embodiment, the T cell is an antigen specific CD4 T cell. In certain aspects of this embodiment, the antigen presenting cells in the subject have enhanced maturation and activation relative prior to the administration of the compound or pharmaceutical composition. In certain aspects of this embodiment, the antigen presenting cells are dendritic cells. In certain aspects of this embodiment, the maturation of the antigen presenting cells is characterized by increased frequency of CD83+ dendritic cells. In certain aspects of this embodiment, the activation of the antigen presenting cells is characterized by elevated expression of CD80 and CD86 on dendritic cells. In some aspects, compounds of Formulae (II-A)-(II-F), or variations thereof, or a pharmaceutical composition thereof provides general priming of the immune response (i.e., vaccines) to tumors or viruses for boosting/generating anti-viral/tumor immunity.

In another embodiment, the subject matter disclosed herein is directed to a method for treating a cancer, the method comprising administering to a subject in need thereof an effective amount of a compound of Formulae (II-A)-(II-F), or a pharmaceutical composition thereof as further described herein. It is understood that the compound functions by inhibiting Cbl-B in a manner that leads to activated T cells that are able to kill cancer cells, regardless of their origin in the body. In certain aspects of this embodiment, the cancer comprises at least one cancer selected from the group consisting of colorectal cancer, melanoma, non-small cell lung cancer, ovarian cancer, breast cancer, pancreatic cancer, a hematological malignancy, and a renal cell carcinoma. In certain aspects of this embodiment, the cancer has elevated levels of T-cell infiltration. In certain aspects of this embodiment, the cancer cells in the subject selectively have elevated expression of MHC class I antigen expression relative to prior to the administration of the compound or composition.

In the methods described herein, the method can further comprise administering a therapeutic, or chemotherapeutic agent to said subject. For example, such an agent may be an inhibitor of PD-L1/PD-1. In certain aspects of this embodiment, the therapeutic or chemotherapeutic agent is administered to the subject simultaneously with the compound or the composition. In certain aspects of this embodiment, the therapeutic or chemotherapeutic agent is administered to the subject prior to administration of the compound or the composition. In certain aspects of this embodiment, the therapeutic or chemotherapeutic agent is administered to the subject after administration of the compound or said composition.

As used herein, “enhancing an immune response” refers to an improvement in any immunogenic response to an antigen. Non-limiting examples of improvements in an immunogenic response to an antigen include enhanced maturation or migration of dendritic cells, enhanced activation of T cells (e.g., CD4 T cells, CDS T cells), enhanced T cell (e.g., CD4 T cell, CDS T cell) proliferation, enhanced B cell proliferation, increased survival of T cells and/or B cells, improved antigen presentation by antigen presenting cells (e.g., dendritic cells), improved antigen clearance, increase in production of cytokines by T cells (e.g., interleukin-2), increased resistance to prostaglandin E2-induced immune suppression, and enhanced priming and/or cytolytic activity of CDS T cells.

In some embodiments, the CDS T cells in the subject have enhanced priming, activation, proliferation and/or cytolytic activity relative to prior to the administration of the compound of Formula (I), or variations thereof such as Formula (IA), (IB) and (IC), or a pharmaceutically acceptable salt, prodrug, metabolite, or derivative thereof. In some embodiments, the CDS T cell priming is characterized by elevated CD44 expression and/or enhanced cytolytic activity in CDS T cells. In some embodiments, the CDS T cell activation is characterized by an elevated frequency of y-IFN+ CDS T cells. In some embodiments, the CDS T cell is an antigen-specific T-cell.

In some embodiments, the CD4 T cells in the subject have enhanced priming, activation, proliferation and/or cytolytic activity relative to prior to the administration of the compound of Formulae (II-A)-(II-F), or a pharmaceutically acceptable salt, prodrug, metabolite, or derivative thereof. In some embodiments, the CD4 T cell priming is characterized by elevated CD44 expression and/or enhanced cytolytic activity in CD4 T cells. In some embodiments, the CD4 T cell activation is characterized by an elevated frequency of y-IFN+ CD4 T cells. In some embodiments, the CD4 T cell is an antigen-specific T-cell.

Accordingly, the presently disclosed compounds of Formulae (II-A)-(II-F), or pharmaceutically acceptable salts, prodrugs, metabolites, or derivatives thereof are useful in treating T cell dysfunctional disorders. A “T cell dysfunctional disorder” is a disorder or condition of T cells characterized by decreased responsiveness to antigenic stimulation.

Thus, the presently disclosed compounds can be used in treating conditions where enhanced immunogenicity is desired, such as increasing tumor immunogenicity for the treatment of cancer.

“Immunogenicity” refers to the ability of a particular substance to provoke an immune response. Tumors are immunogenic and enhancing tumor immunogenicity aids in the clearance of the tumor cells by the immune response. Viruses may also be immunogenic and enhancing/activating immunogenicity may aid in clearance of viral particles by the immune response.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

The compounds herein may be used in conjunction with one or more chemotherapeutic agents, in the course of treating a patient. A “chemotherapeutic agent” is a chemical compound or biologic useful in the treatment of cancer. Examples of chemotherapeutic agents include alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CBI-TMI); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII (see, e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, and floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Additional examples of chemotherapeutic agents that can be deployed in treatment protocols that involve the Cbl-B inhibitor compounds herein, include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LYII 7018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); an antiestrogen such as fulvestrant; EGFR inhibitor such as erlotinib or cetuximab; an anti-VEGF inhibitor such as bevacizumab; arinotecan; rmRH (e.g., ABARELIX®); 17AAG (geldanamycin derivative that is a heat shock protein (Hsp) 90 poison), and pharmaceutically acceptable salts, acids or derivatives of any of the above.

Also included in the definition of “chemotherapeutic agent” are: anti-hormonal agents that act to regulate or inhibit hormone action on tumors such as anti-estrogens and selective estrogen receptor modulators (SERMs), including, for example, tamoxifen (including NOL V ADEX®; tamoxifen citrate), raloxifene, droloxifene, 4-hydroxytamoxifen, trioxifene, keoxifene, LYII 7018, onapristone, and FARESTON® (toremifine citrate); aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); antiandrogens such as flutamide, nilutamide, bicalutamide, leuprolide, and goserelin; as well as troxacitabine (a 1,3-dioxolane nucleoside cytosine analog); protein kinase inhibitors; lipid kinase inhibitors; antisense oligonucleotides, particularly those which inhibit expression of genes in signaling pathways implicated in aberrant cell proliferation, such as, for example, PKC-alpha, Ralf and H-Ras; ribozymes such as VEGF expression inhibitors (e.g., ANGIOZYME®) and HER2 expression inhibitors; vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; antiangiogenic agents such as bevacizumab (AV ASTIN®, Genentech); and pharmaceutically acceptable salts, acids and derivatives of any of the above.

In some embodiments, the chemotherapeutic agent is an immunotherapeutic agent. As used herein, an “immunotherapeutic agent” is a compound that enhances the immune system to help fight cancer, specifically or non-specifically. Immunotherapeutics include monoclonal antibodies and non-specific immunotherapies that boost the immune system, such as cytokines, interleukins (e.g., IL-2, IL-7, IL-12, IL-15, IL-21), interferons (e.g., IFN-α, IFN-˜, IFN-γ), GMCSF, thalidomide, (THALOMID®, Celgene), lenalidomide (REVLIMID®, Celgene), pomalidomide (POMALYST®, Celgene), imiquimod (ZYCLARA®, Valeant). Non-limiting examples of monoclonal antibodies that are useful as a chemotherapeutic agent include trastuzumab (HERCEPTIN®, Genentech), bevacizumab (AV ASTIN®, Genentech), cetuximab (ERBITUX®, Bristol-Myers Squibb), panitumumab (VECTIBIX®, Amgen), ipilimumab (YERVOY®, Bristol-Myers Squibb), rituximab (RITUXAN®, Genentech), alemtuzumab (CAMPATH®, Genzyme), ofatumumab (ARZERRA®, Genmab), gemtuzumab ozogamicin (MYLOTARG®, Wyeth), brentuximab vedotin (ADCETRIS®, Seattle Genetics), 90Y-labelled ibritumomab tiuxetan (ZEVALIN®, Biogen Idec), 1311-labelled tositumomab (BEXXAR®, GlaxoSmithKline), ado-trastuzumab emtansine (KADCYLA®, Genentech) blinatumomab (BLINCYTO®, Amgen), pertuzumab (PERJETA®, Genentech), obinutuzumab (GAZYVA®, Genentech), nivolumab (OPDIVO®), Bristol-Myers Squibb), pembrolizumab (KEYTRUDA®, Merck), pidilizumab (CureTech), Tiragolumab (Roche/Genentech, described in WHO Drug Information (International Nonproprietary Names for Pharmaceutical Substances), Proposed INN: List 117, Vol. 31, No. 2, Jun. 9, 2017 (page 343)), MPDL3280A (Atezolizumab, Roche/Genentech), MDX-1105 (described in WO2007/005874), and MEDI4736 (IMFINZI®, Durvalumab, Medarex). Another useful immunotherapeutic agent is AMP-224 (described in WO2010/027827 and WO2011/066342).

In some embodiments, the compound is administered to the subject at a dose of between about 0.001 μg/kg and about 1000 mg/kg, including but not limited to about 0.001 μg/kg, about 0.01 μg/kg, about 0.05 μg/kg, about 0.1 μg/kg, about 0.5 μg/kg, about 1 μg/kg, about 10 μg/kg, about 25 μg/kg, about 50 μg/kg, about 100 μg/kg, about 250 μg/kg, about 500 μg/kg, about 1 mg/kg, about 5 mg/kg, about 10 mg/kg, about 25 mg/kg, about 50 mg/kg, about 100 mg/kg, and about 200 mg/kg.

### Exemplary Synthetic Methods

Compounds of Formulae (II-A)-(II-F) can individually be synthesized via one or more of the general synthetic Schemes A-D. It would be understood by a skilled organic chemist that other synthetic pathways could also be devised and that the synthetic methods described herein are neither exclusive nor limiting.

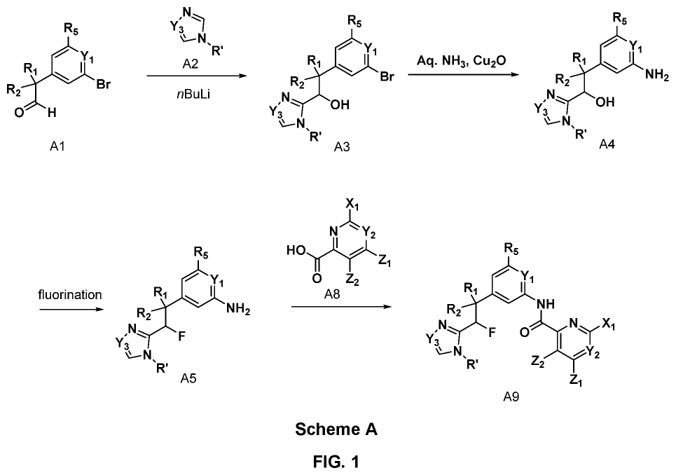

Scheme A (FIG. 1) shows a first method of synthesizing compounds of formulae II-A, II-B, II-C, and II-D. In Scheme A, Y₁ and Y₂ are as elsewhere described herein; X, X₁, Z, Z₁, and Z₂ are as described elsewhere herein; R₁, R₂ and R₅ are as described elsewhere herein. R′ is H or methyl, and Y₃ is N or CH.

Reaction of aldehyde A1 with an aryl lithium, generated when A2 is treated with nBuLi, gives intermediate A3. Subsequently, A3 is converted to aniline A4. This is followed by fluorination reaction of A4, which yields intermediate A5. Intermediate A5 can be coupled with an acid (A8) to give compound A9.

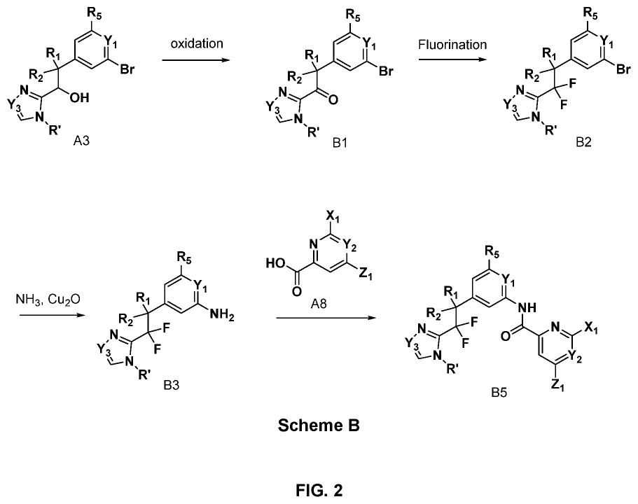

Scheme B (FIG. 2) shows a second method of synthesizing compounds falling within Formulae II-A, II-B, II-C, and II-D. In Scheme B, Y₁ and Y₂ are as elsewhere described herein; X, X₁, Z, Z₁, and Z₂ are as described elsewhere herein; R₁, R₂, R₃, R₄, and R₅ are as described elsewhere herein. R′ is H or methyl, and Y₃ is N or CH.

In Scheme B, intermediate A3 can be oxidized to give ketone B1. Treatment of B1 with a fluorinating reagent gives di-fluoro intermediate B2, which is subsequently transformed to aniline B3. Intermediate B3 can be coupled with an acid (A8), to give amide B5.

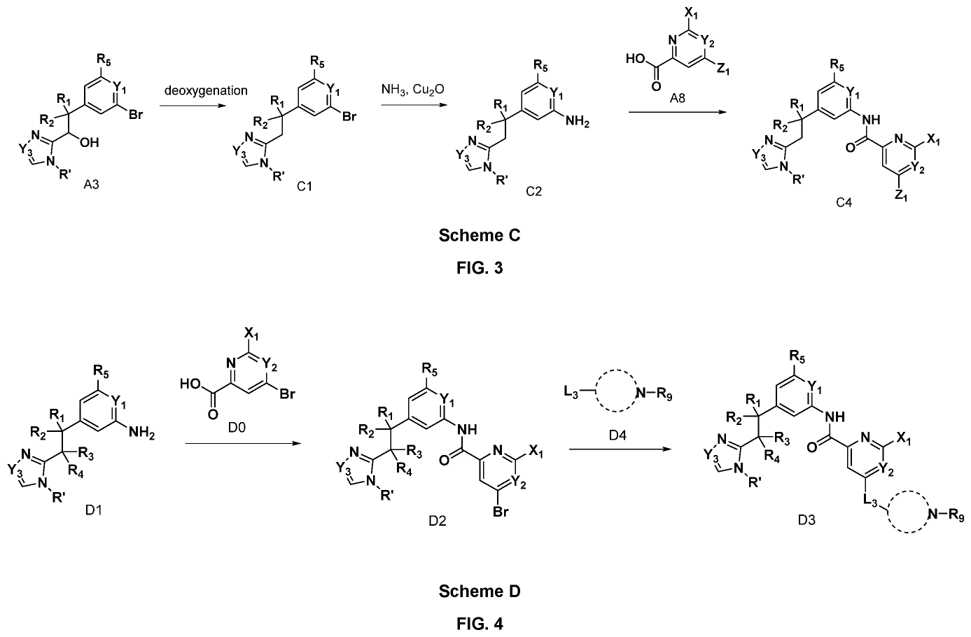

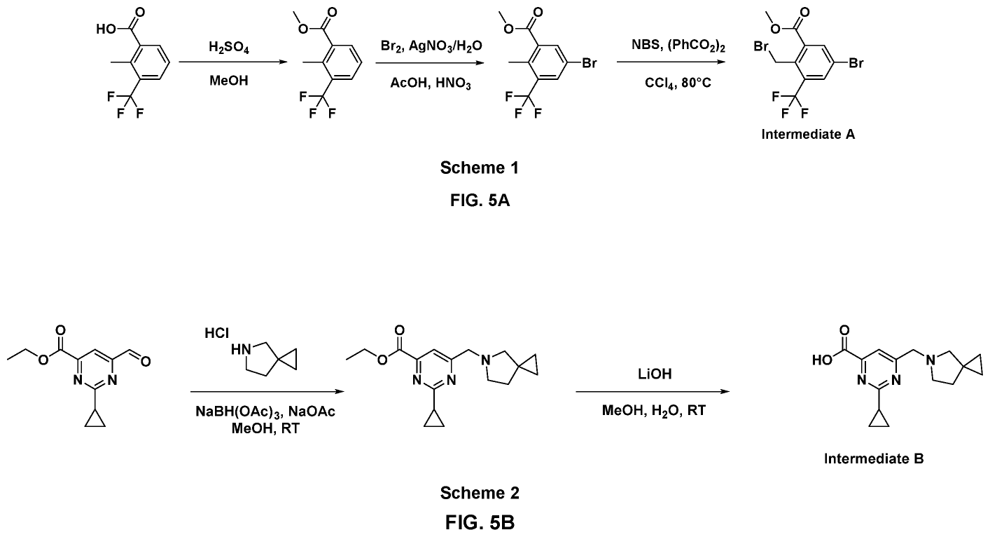

Scheme C (FIG. 3) shows a third method of synthesizing compounds of formulae II-A, II-B, II-C, and II-D. In Scheme C, Y₁ and Y₂ are as elsewhere described herein; X, X₁, Z, Z₁, and Z₂ are as described elsewhere herein; R₁, R₂ and R₅ are as described elsewhere herein. R′ is H or methyl, and Y₃ is N or CH.

In Scheme C, intermediate A3 can be deoxygenated to give intermediate C1, which can be transformed to aniline C2. An amide coupling reaction of C2 with acid (A8) leads to compound C4.

Scheme D shows a fourth method of synthesizing compounds of formulae II-A, II-B, II-C, II-D, and II-E. In Scheme D, Y₁ and Y₂ are as elsewhere described herein; X, X₁, Z, Z₁, and Z₂ are as described elsewhere herein; R₁, R₂, R₃, R₄, and R₅ are as described elsewhere herein. R′ is H or methyl, and Y₃ is N or CH.

In Scheme D, intermediate D1 can be coupled with an acid (DO), to give intermediate D2. The Br intermediate D2 can subsequently be transformed to compound D3 via a transition metal-catalyzed coupling reaction (with D4) or a photoredox reaction.

The following examples are offered by way of illustration and not by way of limitation.

### EXAMPLES

Synthesis of various compounds of the invention can be accomplished by utilizing various intermediates, as disclosed in Examples 1-15.

### Example 1: Intermediate A (methyl 5-bromo-2-(bromomethyl)-3-(trifluoromethyl)-benzoate)

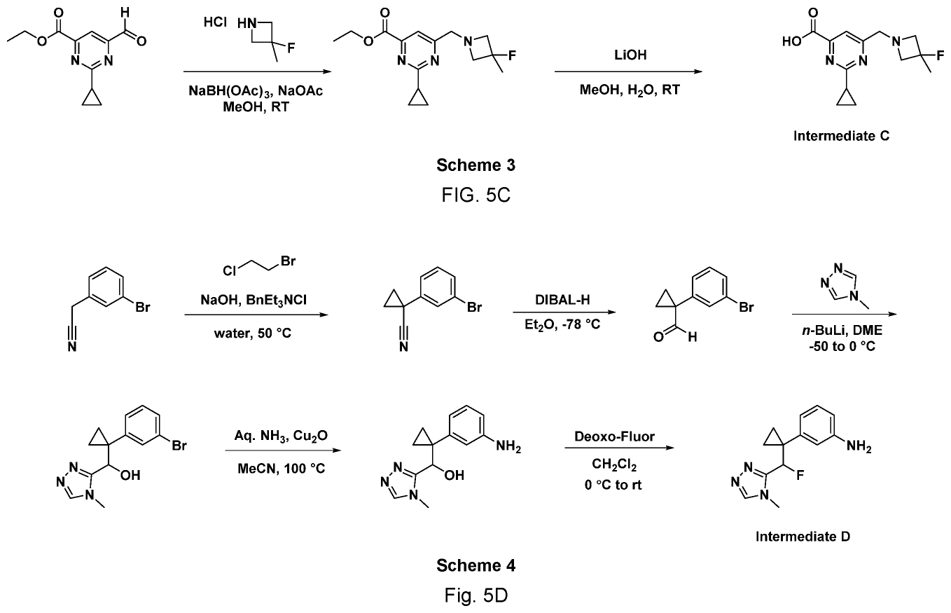

Intermediate A can be synthesized according to Scheme 1, FIG. 5A.

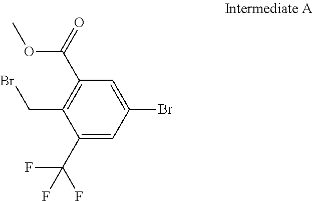 [SMILES: COC(=O)c1cc(Br)cc(C(F)(F)F)c1CBr]

Step A-1 involves synthesis of methyl 2-methyl-3-(trifluoromethyl)benzoate. To a solution of 2-methyl-3-(trifluoromethyl)benzoic acid (10.0 g, 49.0 mmol) in methanol (196 mL) was added sulfuric acid (5.0 mL, 93.1 mmol) and the resulting mixture was heated to 65° C. for 23 h. The reaction was cooled to RT, concentrated, diluted with water (250 mL)/sat. aqueous sodium bicarbonate (250 mL) and extracted with EtOAc (3×200 mL). The organics were combined, dried over sodium sulfate, filtered and evaporated to afford methyl 2-methyl-3-(trifluoromethyl)benzoate (10.3 g, 97% yield). ¹H NMR (400 MHz, CDCl₃) δ 7.91 (d, J=7.8 Hz, 1H), 7.76 (d, J=7.8 Hz, 1H), 7.33 (t, J=7.9 Hz, 1H), 3.92 (s, 3H), 2.64 (q, J=1.4 Hz, 3H).

Step A-2 involves synthesis of methyl 5-bromo-2-methyl-3-(trifluoromethyl)-benzoate. To a solution of methyl 2-methyl-3-(trifluoromethyl)benzoate (10.3 g, 47.3 mmol) in acetic acid (65 mL) were added HNO₃, 70% in water (21.1 mL, 473 mmol) and bromine (2.67 mL, 52.0 mmol) followed by dropwise addition of silver nitrate, 2.5 M in water (24.6 mL, 61.5 mmol) using an addition funnel. The mixture was then stirred at RT for 17 h. The reaction mixture was then poured on ice, diluted with 1N NaOH (200 mL) and extracted with EtOAc (3×100 mL). The organics were combined, washed with water (3×100 mL), dried over sodium sulfate, filtered and evaporated. The crude mixture was diluted with EtOAc (200 mL) and washed with saturated NaHCO₃ (5×100 mL) and saturated Na₂CO₃ (2×100 mL) to get rid of AcOH. The organic phase was dried over sodium sulfate, filtered and evaporated to afford methyl 5-bromo-2-methyl-3-(trifluoromethyl)benzoate (14.4 g, 100% yield). The product was used without any more purification in next step. ¹H NMR (400 MHz, CDCl₃) δ 8.06 (d, J=2.0 Hz, 1H), 7.88 (d, J=1.9 Hz, 1H), 3.93 (s, 3H), 2.58 (q, J=1.5 Hz, 3H).

Step A-3 involves making methyl 5-bromo-2-(bromomethyl)-3-(trifluoromethyl)-benzoate. A mixture of methyl 5-bromo-2-methyl-3-(trifluoromethyl)benzoate (9.87 g, 33.2 mmol), N-bromosuccinimide (17.7 g, 99.7 mmol) and benzoyl peroxide (3.22 g, 13.3 mmol) in carbon tetrachloride (111 mL) was heated to 75° C. and stirred for 20 h. The mixture was cooled to RT, filtered and concentrated. The residue was purified by chromatography on silica gel (100% heptanes) to afford methyl 5-bromo-2-(bromomethyl)-3-(trifluoromethyl)benzoate (11.29 g, 90% yield). ¹H NMR (400 MHz, CDCl₃) δ 8.18 (d, J=2.1 Hz, 1H), 7.94 (d, J=2.1 Hz, 1H), 3.99 (s, 3H).

### Example 2: Intermediate B (6-(5-Azaspiro[2.4]heptan-5-ylmethyl)-2-cyclopropylpyrimidine-4-carboxylic acid)

Intermediate B can be synthesized according to Scheme 2, FIG. 5B.

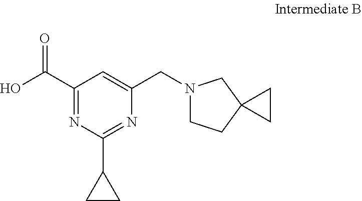 [SMILES: O=C(O)c1cc(CN2CCC3(CC3)C2)nc(C2CC2)n1]

In step B-1, ethyl 6-(5-azaspiro[2.4]heptan-5-ylmethyl)-2-cyclopropylpyrimidine-4-carboxylate is made as follows. To ethyl 2-cyclopropyl-6-formylpyrimidine-4-carboxylate (300 mg, 1.36 mmol) in methanol (6.8 mL) were added 5-azaspiro[2.4]heptane hydrochloride (310 mg, 2.32 mmol) and sodium acetate (340 mg, 4.09 mmol). The reaction mixture was stirred at RT for 15 min and then sodium triacetoxyborohydride (858 mg, 4.09 mmol) was added. The reaction was stirred at RT for 16 h. The reaction was diluted with sat. aqueous sodium bicarbonate (25 mL) and extracted with DCM (3×25 mL). The organic phases were combined, dried over sodium sulfate, filtered and concentrated under reduced pressure to afford ethyl 6-(5-azaspiro[2.4]heptan-5-ylmethyl)-2-cyclopropylpyrimidine-4-carboxylate which was used without further purification.

In step B-2, 6-(5-azaspiro[2.4]heptan-5-ylmethyl)-2-cyclopropylpyrimidine-4-carboxylic acid is made as follows. To ethyl 6-(5-azaspiro[2.4]heptan-5-ylmethyl)-2-cyclopropylpyrimidine-4-carboxylate previously obtained in methanol (10 mL) was added 1 M lithium hydroxide (10 mL, 10.0 mmol). The reaction mixture was stirred at RT for 50 min. The reaction was diluted with 1 M HCl until pH 7 was reached and the mixture was concentrated to about 2 mL total volume. The residue was purified by chromatography on C18 silica gel (0-60% acetonitrile in ammonium bicarbonate, pH=10) to afford 6-(5-azaspiro[2.4]heptan-5-ylmethyl)-2-cyclopropylpyrimidine-4-carboxylic acid (246 mg, 66%) as a white solid. LCMS (ESI) m/z: 274.2 [M+H]+. 1H NMR (400 MHz, CD3OD) 7.66 (s, 1H), 3.75 (s, 2H), 2.85 (t, J=6.9 Hz, 2H), 2.61 (s, 2H), 2.37-2.18 (m, 1H), 1.85 (t, J=6.9 Hz, 2H), 1.27-1.13 (m, 2H), 1.12-0.96 (m, 2H), 0.65-0.45 (m, 4H).

### Example 3: Intermediate C (2-Cyclopropyl-6-((3-fluoro-3-methylazetidin-1-yl)methyl)pyrimidine-4-carboxylic acid)

Intermediate C can be synthesized according to Scheme 3, FIG. 5C.

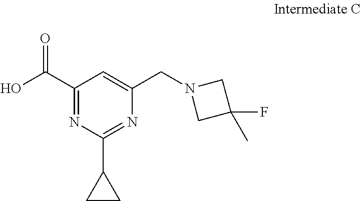 [SMILES: CC1(F)CN(Cc2cc(C(=O)O)nc(C3CC3)n2)C1]

In step C-1, ethyl 2-cyclopropyl-6-((3-fluoro-3-methylazetidin-1-yl)methyl)pyrimidine-4-carboxylate is made as follows. To ethyl 2-cyclopropyl-6-formylpyrimidine-4-carboxylate (300 mg, 1.36 mmol) in methanol (6.8 mL) were added 3-fluoro-3-methyl-azetidine hydrochloride (288 mg, 2.32 mmol) and triethylamine (0.32 mL, 2.32 mmol). The reaction mixture was stirred at 100° C. for 1 min in the microwave and was cooled to RT. Sodium cyanoborohydride (171 mg, 2.72 mmol) was added and the reaction was stirred at 100° C. for 2 h in the microwave. The reaction mixture was taken directly to the next step.

In step C-2, 2-cyclopropyl-6-((3-fluoro-3-methylazetidin-1-yl)methyl)pyrimidine-4-carboxylic acid is made as follows. The mixture containing ethyl 2-cyclopropyl-6-((3-fluoro-3-methylazetidin-1-yl)methyl)pyrimidine-4-carboxylate previously obtained was diluted with methanol (4 mL) and 1 M lithium hydroxide (12 mL, 12.0 mmol) was added. The reaction mixture was stirred at RT for 50 min. The reaction was diluted with 1 M HCl until pH 7 was concentrated to about 2 mL total volume. The residue was purified by chromatography on C18 silica gel (0-70% acetonitrile in ammonium bicarbonate, pH=10) to afford 2-cyclopropyl-6-((3-fluoro-3-methylazetidin-1-yl)methyl)pyrimidine-4-carboxylic acid (90 mg, 25%) as a yellow solid. LCMS (ESI) m/z: 266.1 [M+H]+. ¹H NMR (400 MHz, CD₃OD) 7.57 (s, 1H), 3.79 (s, 2H), 3.59-3.37 (m, 4H), 2.33-2.17 (m, 1H), 1.62 (t, J=26.3 Hz, 3H), 1.23-1.13 (m, 2H), 1.08-1.00 (m, 2H).

### Example 4: Intermediate D (3-(1-(fluoro(4-methyl-4H-1,2,4-triazol-3-yl)methyl)cyclopropyl)aniline)

Intermediate D can be synthesized according to Scheme 4, FIG. 5D.

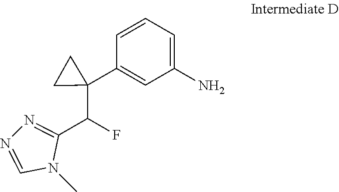 [SMILES: Cn1cnnc1C(F)C1(c2cccc(N)c2)CC1]

In step D-1, 1-(3-bromophenyl)cyclopropanecarbonitrile is made as follows. A mixture of 2-(3-bromophenyl)acetonitrile (6.00 g, 30.6 mmol), 1-bromo-2-chloroethane (3.82 mL, 45.9 mmol) and benzyltriethylammonium chloride (139 mg, 0.61 mmol) in 6 N aqueous sodium hydroxide (38.3 mL, 223 mmol) was stirred for 18 h at 50° C. The reaction mixture was diluted with EtOAc (200 mL) and water (200 mL). The layers were separated, the aqueous phase was extracted with EtOAc (200 mL), the combined organic phases were washed with 1 N HCl (100 mL), washed with 1 N aqueous potassium carbonate (100 mL), washed with brine (100 mL), dried over magnesium sulfate, filtered and concentrated under reduced pressure to afford 1-(3-bromophenyl)cyclopropanecarbonitrile (6.57 g, 97% yield) as a dark brown solid. ¹H NMR (400 MHz, CDCl₃) 7.44-7.40 (m, 2H), 7.27-7.20 (m, 2H), 1.77-1.73 (m, 2H), 1.43-1.39 (m, 2H).

In step D-2, 1-(3-bromophenyl)cyclopropanecarbaldehyde is made as follows. DIBAL-H, 1.0 M in hexanes (51.6 mL, 51.6 mmol) was added slowly to a solution of 1-(3-bromophenyl)cyclopropanecarbonitrile (7.64 g, 34.4 mmol) in diethyl ether (115 mL) at −78° C. The resulting mixture was stirred for 2 h at the same temperature. The reaction was carefully quenched with 50 mL of 10% aqueous HCl and allowed to warm to RT. The reaction mixture was diluted with EtOAc (200 mL) and water (200 mL). The layers were separated, the aqueous phase was extracted with EtOAc (200 mL), the combined organic phases were washed with saturated sodium bicarbonate (100 mL), washed with water (100 mL), washed with brine (100 mL), dried over magnesium sulfate, filtered and concentrated under reduced pressure to afford 1-(3-bromophenyl)cyclopropanecarbaldehyde (7.62 g, 98% yield) as an orange oil. ¹H NMR (400 MHz, DMSO-d6) 13.48 (s, 1H), 9.91 (s, 1H), 8.77-8.75 (m, 1H), 8.09-8.05 (m, 2H), 7.63 (t, J=8.0 Hz, 1H), 6.54 (s, 1H).

In step D-3, (1-(3-bromophenyl)cyclopropyl)(4-methyl-4H-1,2,4-triazol-3-yl)methanol is made as follows. A solution of n-butyllithium, 2.64 M in hexanes (12.7 mL, 33.6 mmol) was added drop wise to a solution of 4-methyl-1,2,4-triazole (2.79 g, 33.6 mmol) in anhydrous DME (600 mL) at −50° C. The resulting mixture was stirred at −50° C. for 1 h before a solution of 1-(3-bromophenyl)cyclopropanecarbaldehyde (6.88 g, 30.6 mmol) in DME (25 mL) was added dropwise. The reaction was gradually allowed to warm to 0° C. over 1 h. The reaction was quenched with water (100 mL) and DME was evaporated under reduced pressure. The residue was diluted with 4:1 CHCl₃/IPA (200 mL). The layers were separated, the aqueous phase was extracted with 4:1 CHCl₃/IPA (4×200 mL), the combined organic phases were dried over magnesium sulfate, filtered and concentrated under reduced pressure. The residue was purified by chromatography on silica gel (2-12% MeOH in DCM) to afford (1-(3-bromophenyl)cyclopropyl)(4-methyl-4H-1,2,4-triazol-3-yl)methanol (4.82 g, 51% yield) as a white solid. LCMS (ESI) m/z: 308.2/310.2 [M+H]⁺ (Br pattern). ¹H NMR (400 MHz, CDCl₃) 7.81 (s, 1H), 7.37-7.33 (m, 2H), 7.11-7.04 (m, 2H), 5.83 (s, 1H), 4.70 (s, 1H), 3.07 (s, 3H), 1.22-1.16 (m, 1H), 1.15-1.10 (m, 1H), 0.93-0.87 (m, 1H), 0.84-0.78 (m, 1H).

In step D-4, (1-(3-Aminophenyl)cyclopropyl)(4-methyl-4H-1,2,4-triazol-3-yl)methanol is made as follows. Copper(I) oxide (278 mg, 1.95 mmol) was added to a mixture of (1-(3-bromophenyl)cyclopropyl)(4-methyl-4H-1,2,4-triazol-3-yl)methanol (1.00 g, 3.25 mmol) and conc. aqueous ammonia (6.0 mL) in acetonitrile (6.5 mL) under nitrogen in a microwave vial. The vial was sealed and the reaction was stirred for 18 h at 100° C. The reaction was diluted with 4:1 CHCl₃/IPA (50 mL), water (25 mL) and conc. aqueous ammonia (25 mL). The layers were separated, the aqueous phase was extracted with 4:1 CHCl3/IPA (10×50 mL), the combined organic phases were dried over magnesium sulfate, filtered and concentrated under reduced pressure. The residue was purified by chromatography on C18 silica gel (0-50% acetonitrile in ammonium bicarbonate, pH=10) to afford (1-(3-aminophenyl)cyclopropyl)(4-methyl-4H-1,2,4-triazol-3-yl)methanol (472 mg, 60% yield) as a white solid. LCMS (ESI) m/z: 245.2 [M+H]⁺.

In step D-5, 3-(1-(fluoro(4-methyl-4H-1,2,4-triazol-3-yl)methyl)cyclopropyl)aniline is made as follows. To a solution of (1-(3-aminophenyl)cyclopropyl)(4-methyl-4H-1,2,4-triazol-3-yl)methanol (652 mg, 2.67 mmol) in DCM (27 mL) was added Deoxo-Fluor (50% w/w in toluene) (3.91 mL, 8.84 mmol) dropwise at 0° C. (monitoring internal temperature <5° C.). The reaction was allowed to warm to RT and stirred for 1 h. The reaction was cooled to 0° C. and was quenched with slow addition of water (25 mL). The reaction mixture was extracted with 4:1 CHCl₃/IPA (6×50 mL) and the combined organic layers were dried over sodium sulfate. The residue was purified by chromatography on C18 silica gel (0-50% acetonitrile in ammonium formate, pH=4) to afford 3-(1-(fluoro(4-methyl-4H-1,2,4-triazol-3-yl)methyl)cyclopropyl)aniline (393 mg, 60% yield) as a yellow solid. LCMS (ESI) m/z: 247.3 [M+H]⁺.

### Example 5: Intermediate E (3-(1,1-Difluoro-1-(4-methyl-4H-1,2,4-triazol-3-yl)propan-2-yl)aniline)

Intermediate E can be synthesized according to Scheme 5, FIG. 5E.

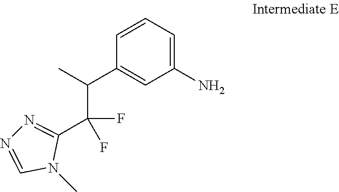 [SMILES: CC(c1cccc(N)c1)C(F)(F)c1nncn1C]

In step E-1, 1-bromo-3-(1-methoxyprop-1-en-2-yl)benzene is made as follows. To a stirred suspension of methoxymethyl triphenylphosphonium chloride (111 g, 324 mmol) in diethyl ether (1.1 L) at 0° C. was added potassium tert-butoxide (38.8 g, 346 mmol) in portions. After 30 min, a solution of 3′-bromoacetophenone (28.6 mL, 216 mmol) in diethyl ether (150 mL) was added dropwise to the reaction mixture, which was then allowed to warm up to RT and stirred for 17 h. The reaction mixture was concentrated under reduced pressure to around 600 mL and washed with sat. aqueous ammonium chloride (200 mL). The organic layer was separated and concentrated under reduced pressure to give and solid suspension. This solid suspension was diluted with heptanes (300 mL) and stirred for 30 min. The precipitate was filtered through sand and washed with heptanes. The filtrate was concentrated under reduced pressure and passed through a silica gel pad eluting with 5% ethyl acetate in heptanes to afford 1-bromo-3-(1-methoxyprop-1-en-2-yl)benzene (47.0 g, 96% yield).

In step E-2, 2-(3-bromophenyl)propanal is made as follows. HBr, 48% in water (25.8 mL, 228 mmol) was added dropwise to a solution of 1-bromo-3-(1-methoxyprop-1-en-2-yl)benzene (47.0 g, 207 mmol) in acetone (200 mL) and water (51 mL) cooled to 0° C. The reaction was then allowed to warm to RT and stirred for 3 days. The reaction was then quenched with sat. aqueous sodium bicarbonate and acetone was evaporated. The resulting aqueous mixture was extracted with DCM (3×200 mL). The organic phases were combined, dried over sodium sulfate, filtered and concentrated under reduced pressure. The residue was purified by chromatography on silica gel (0-30% DCM in heptanes) to afford 2-(3-bromophenyl)propanal (32.0 g, 73% yield). ¹H NMR (400 MHz, CDCl₃) 9.67 (d, J=1.4 Hz, 1H), 7.44 (ddd, J=8.0, 1.9, 1.0 Hz, 1H), 7.37 (t, J=1.8 Hz, 1H), 7.25 (t, J=8.0 Hz, 1H), 7.16-7.12 (m, 1H), 3.61 (qd, J=7.1, 1.1 Hz, 1H), 1.45 (t, J=8.0 Hz, 1H).

In step E-3, 2-(3-bromophenyl)-1-(4-methyl-4H-1,2,4-triazol-3-yl)propan-1-ol is made as follows. A solution of n-butyllithium, 2.5 M in hexanes (16.5 mL, 41.3 mmol) was added drop wise to a solution of 4-methyl-1,2,4-triazole (3.43 g, 41.3 mmol) in anhydrous DME (375 mL) at −50° C. The resulting mixture was stirred at −50° C. for 1 h before a solution of 2-(3-bromophenyl)propanal (8.0 g, 37.6 mmol) in DME (30 mL) was added dropwise. The reaction was gradually allowed to warm to 0° C. over 1 h. The reaction was quenched with water (100 mL) and DME was evaporated under reduced pressure. The residue was diluted with 4:1 CHCl₃/IPA (200 mL). The layers were separated, the aqueous phase was extracted with 4:1 CHCl₃/IPA (4×200 mL), the combined organic phases were dried over magnesium sulfate, filtered and concentrated under reduced pressure. The residue was purified by chromatography on silica gel (0-15% MeOH in DCM) to afford 2-(3-bromophenyl)-1-(4-methyl-4H-1,2,4-triazol-3-yl)propan-1-ol (6.20 g, 56% yield). LCMS (ESI) m/z: 296.1/298.2 [M+H]⁺ (Br pattern).

In step E-4, 2-(3-bromophenyl)-1-(4-methyl-4H-1,2,4-triazol-3-yl)propan-1-one is made as follows. DMP (17.8 g, 42 mmol) was added in one portion to a solution of 2-(3-bromophenyl)-1-(4-methyl-4H-1,2,4-triazol-3-yl)propan-1-ol (6.22 g, 21.0 mmol) in DCM (100 mL) at RT. The resulting mixture was stirred for 20 h at the same temperature. The reaction was quenched with water (100 mL) and diluted 4:1 CHCl₃/IPA (100 mL). The layers were separated, the aqueous phase was extracted with 4:1 CHCl₃/IPA (3×100 mL), the combined organic phases were dried over sodium sulfate, filtered and concentrated under reduced pressure. The residue was purified by chromatography on silica gel (0-100% EtOAc in DCM) to afford 2-(3-bromophenyl)-1-(4-methyl-4H-1,2,4-triazol-3-yl)propan-1-one (5.20 g, 84% yield). LCMS (ESI) m/z: 294.2/296.1 [M+H]⁺ (Br pattern).

In step E-5, 3-(2-(3-bromophenyl)-1,1-difluoropropyl)-4-methyl-4H-1,2,4-triazole is made as follows. A mixture of DAST (25 mL, 189 mmol) and 2-(3-bromophenyl)-1-(4-methyl-4H-1,2,4-triazol-3-yl)propan-1-one (2.70 g, 9.18 mmol) as stirred at 60° C. for 60 h. The reaction was cooled to 0° C., carefully quenched with sat. aqueous sodium bicarbonate until pH 8 was reached and diluted with DCM (100 mL). The layers were separated, the aqueous phase was extracted with DCM (2×100 mL), the combined organic phases were washed with brine (100 mL), dried over sodium sulfate, filtered and concentrated under reduced pressure. The residue was purified by chromatography on silica gel (0-100% EtOAc in DCM) to afford 3-(2-(3-bromophenyl)-1,1-difluoropropyl)-4-methyl-4H-1,2,4-triazole (900 mg, 31% yield). LCMS (ESI) m/z: 316.1/318.12 [M+H]⁺ (Br pattern).

In step E-6, 3-(1,1-Difluoro-1-(4-methyl-4H-1,2,4-triazol-3-yl)propan-2-yl)aniline is made as follows. Copper(I) oxide (462 mg, 3.23 mmol) was added to a mixture of 3-(2-(3-bromophenyl)-1,1-difluoropropyl)-4-methyl-4H-1,2,4-triazole (1.70 g, 5.38 mmol) and conc. aqueous ammonia (35 mL) in acetonitrile (15 mL) under nitrogen in a sealed tube. The tube was sealed and the reaction was stirred for 18 h at 100° C. The reaction was diluted with 4:1 CHCl₃/IPA (100 mL), water (50 mL) and conc. aqueous ammonia (50 mL). The layers were separated, the aqueous phase was extracted with 4:1 CHCl3/IPA (4×100 mL), the combined organic phases were dried over magnesium sulfate, filtered and concentrated under reduced pressure to afford 3-(1,1-difluoro-1-(4-methyl-4H-1,2,4-triazol-3-yl)propan-2-yl)aniline (1.20 g, 88% yield). LCMS (ESI) m/z: 253.3 [M+H]⁺.

### Example 6: Intermediate F (3-(1-fluoro-1-(4-methyl-4H-1,2,4-triazol-3-yl)propan-2-yl)aniline)

Intermediate F can be synthesized according to Scheme 6, FIG. 5F.

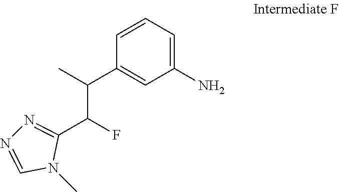 [SMILES: CC(c1cccc(N)c1)C(F)c1nncn1C]

In step F-1, 3-(2-(3-bromophenyl)-1-fluoropropyl)-4-methyl-4H-1,2,4-triazole is made as follows. To a solution of 2-(3-bromophenyl)-1-(4-methyl-4H-1,2,4-triazol-3-yl)propan-1-ol (3.13 g, 10.6 mmol) (previously prepared in synthesis of Intermediate E) in DCM (151 mL) was added Deoxo-Fluor (50% w/w in toluene) (10.2 mL, 23.3 mmol) dropwise at 0° C. (monitoring internal temperature <5° C.). The reaction was allowed to warm to RT and stirred for 1 h. The reaction was cooled to 0° C. and was quenched with slow addition of water (100 mL). The reaction mixture was extracted with DCM (3×100 mL) and the combined organic layers were dried over sodium sulfate. The residue was purified by chromatography on silica gel (0-5% MeOH in DCM) to afford 3-(2-(3-bromophenyl)-1-fluoropropyl)-4-methyl-4H-1,2,4-triazole (2.09 g, 66% yield). LCMS (ESI) m/z: 298.2/300.1 [M+H]⁺ (Br pattern).

In step F-2, 3-(1-fluoro-1-(4-methyl-4H-1,2,4-triazol-3-yl)propan-2-yl)aniline, is made as follows. Copper(I) oxide (602 mg, 4.21 mmol) was added to a mixture of 3-(2-(3-bromophenyl)-1-fluoropropyl)-4-methyl-4H-1,2,4-triazole (2.09 g, 7.00 mmol) and conc. aqueous ammonia (35 mL) in acetonitrile (14 mL) under nitrogen in a sealed tube. The tube was sealed and the reaction was stirred for 18 h at 100° C. The reaction was diluted with 4:1 CHCl₃/IPA (100 mL), water (50 mL) and conc. aqueous ammonia (50 mL). The layers were separated, the aqueous phase was extracted with 4:1 CHCl3/IPA (5×100 mL), the combined organic phases were dried over sodium sulfate, filtered and concentrated under reduced pressure to afford 3-(1-fluoro-1-(4-methyl-4H-1,2,4-triazol-3-yl)propan-2-yl)aniline (1.82 g, 100% yield). LCMS (ESI) m/z: 235.3 [M+H]⁺.

### Example 7: Intermediate G ((S)-3-(2-fluoro-1-(4-methyl-4H-1,2,4-triazol-3-yl)propan-2-yl)aniline)

Intermediate G can be synthesized according to Scheme 7, FIG. 5G.

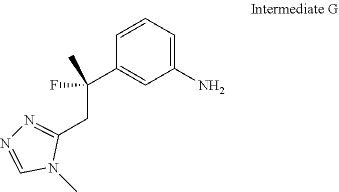 [SMILES: Cn1cnnc1C[C@](C)(F)c1cccc(N)c1]

In step G-1, (R)-2-(3-bromophenyl)-2-fluoropropan-1-ol is made as follows. Sodium bicarbonate (840 mg, 10.0 mmol) and N-fluorobenzenesulfonimide (3.15 g, 10.0 mmol) were added sequentially to a mixture of 2-(3-bromophenyl)propanal (2.13 g, 10.0 mmol), N-[(1R,2R)-2-aminocyclohexyl]-2,6-diphenyl-benzamide (741 mg, 2.00 mmol) and trifluoroacetic acid (154 uL, 2.00 mmol) in THF (30 mL) at RT. The resulting mixture was stirred at RT for 4 h, diluted with MeOH (100 mL) and cooled to 0° C. Sodium borohydride (3.78 g, 100 mmol) was added in 3 portions over the course of 10 min, the reaction was allowed to warm to RT and stirred for 90 min. The reaction was quenched with sat. aqueous ammonium chloride (50 mL), diluted with ethyl acetate (250 mL) and the resulting mixture was stirred for 30 min. The layers were separated and the aqueous phase was extracted with EtOAc (4×100 mL). The combined organic layers were dried over sodium sulfate, filtered, and concentrated under reduced pressure. The residue was purified by chromatography on silica gel (0-60% EtOAc in heptanes) to afford (R)-2-(3-bromophenyl)-2-fluoropropan-1-ol (1.89 g, 81% yield) as a colorless oil. ¹H NMR (400 MHz, CDCl₃) 7.58-7.52 (m, 1H), 7.48-7.42 (m, 1H), 7.31-7.23 (m, 2H), 3.86-3.68 (m, 2H), 1.67 (d, J=22.6 Hz, 3H). % ee was determined to be 80% (Analytical Column: ChiralPak IA, 250 mm×4.6 mm ID, 5 μm; Mobile Phase: 5:95 EtOH:Hexane (0.1% DEA); Isocratic Flow: 1 mL/min, (pressure was 54.4 bars); Column Temp.: ˜ 26° C.; Run Time: 18 min.; Wavelength: 220 nm; RT peak #1=RT1=8.3 min. (width at mid height=W1=0.1469 min.); RT peak #2=RT2=14.6 min. (width at mid height=W2=0.2974 min.)).

In step G-2, (R)-2-(3-bromophenyl)-2-fluoropropanal is made as follows. DMP (3.78 g, 8.92 mmol) was added in one portion to a solution of (R)-2-(3-bromophenyl)-2-fluoropropan-1-ol (1.89 g, 8.11 mmol) in DCM (25 mL) at RT. The resulting mixture was stirred for 1 h at the same temperature. The reaction was filtered through a pad of silica gel (eluting with 100 mL of DCM) and the filtrate was concentrated under reduced pressure to afford (R)-2-(3-bromophenyl)-2-fluoropropanal (1.72 g, 92% yield). ¹H NMR (400 MHz, CDCl₃) 9.70 (d, J=4.7 Hz, 1H), 7.58 (t, J=1.8 Hz, 1H), 7.50 (dt, J=7.3, 1.5 Hz, 1H), 7.31 (ddd, J=14.2, 9.6, 4.8 Hz, 2H), 1.77 (d, J=22.7 Hz, 3H).

In step G-3, (2R)-2-(3-bromophenyl)-2-fluoro-1-(4-methyl-4H-1,2,4-triazol-3-yl)propan-1-ol is made as follows. A solution of n-butyl-lithium, 2.5 M in hexanes (1.90 mL, 4.76 mmol) was added drop wise to a solution of 4-methyl-1,2,4-triazole (396 mg, 4.76 mmol) in anhydrous DME (60 mL) at −50° C. The resulting mixture was stirred at −50° C. for 1 h before a solution of (R)-2-(3-bromophenyl)-2-fluoropropanal (1.00 g, 4.33 mmol) in DME (5 mL) was added dropwise. The reaction was gradually allowed to warm to 0° C. over 1 h. The reaction was quenched with water (25 mL) and DME was evaporated under reduced pressure. The residue was diluted with 4:1 CHCl₃/IPA (50 mL). The layers were separated, the aqueous phase was extracted with 4:1 CHCl₃/IPA (4×50 mL), the combined organic phases were dried over magnesium sulfate, filtered and concentrated under reduced pressure. The residue was purified by chromatography on silica gel (0-15% MeOH in DCM) to afford (2R)-2-(3-bromophenyl)-2-fluoro-1-(4-methyl-4H-1,2,4-triazol-3-yl)propan-1-ol (675 mg, 50% yield). LCMS (ESI) m/z: 314.0/316.0 [M+H]⁺ (Br pattern).

In step G-4, O-((2R)-2-(3-bromophenyl)-2-fluoro-1-(4-methyl-4H-1,2,4-triazol-3-yl)propyl) 1H-imidazole-1-carbothioate is made as follows. 1,1′-Thiocarbonyldiimidazole (82 uL, 0.61 mmol) and was added slowly to a solution of (2R)-2-(3-bromophenyl)-2-fluoro-1-(4-methyl-4H-1,2,4-triazol-3-yl)propan-1-ol (160 mg, 0.51 mmol) and DMAP (3.1 mg, 0.030 mmol) in anhydrous DCM (3 mL) at 20° C. The resulting mixture was stirred at 20° C. for 1 h. The reaction was directly purified by chromatography on silica gel (0-10% MeOH in DCM) to afford O-((2R)-2-(3-bromophenyl)-2-fluoro-1-(4-methyl-4H-1,2,4-triazol-3-yl)propyl) 1H-imidazole-1-carbothioate (200 mg, 93% yield). LCMS (ESI) m/z: 424.0/426.0 (Br pattern).

In step G-5, (S)-3-(2-(3-bromophenyl)-2-fluoropropyl)-4-methyl-4H-1,2,4-triazole is made as follows. A mixture of O-((2R)-2-(3-bromophenyl)-2-fluoro-1-(4-methyl-4H-1,2,4-triazol-3-yl)propyl) 1H-imidazole-1-carbothioate (260 mg, 0.61 mmol), tributyltin hydride (1.65 mL, 6.13 mmol and AIBN (15.1 mg, 0.090 mmol) in anhydrous toluene (10 mL) was stirred at 80° C. for 1 h. To the reaction mixture was added potassium fluoride (2 g) and silica gel (20 g) and the resulting mixture was concentrated under reduced pressure. The residue was directly purified by chromatography on silica gel (0-20% MeOH in DCM) to afford (S)-3-(2-(3-bromophenyl)-2-fluoropropyl)-4-methyl-4H-1,2,4-triazole (98 mg, 54% yield). LCMS (ESI) m/z: 298.0/300.0 (Br pattern).

In step G-6, (S)-3-(2-fluoro-1-(4-methyl-4H-1,2,4-triazol-3-yl)propan-2-yl)aniline is made as follows. Copper(I) oxide (28.22 mg, 0.2000 mmol) was added to a solution of 4-methyl-3-[(2S)-2-(3-bromophenyl)-2-fluoro-propyl]-1,2,4-triazole (98 mg, 0.3300 mmol) in conc. aqueous NH₃ (5 mL)/MeCN (2 mL) under nitrogen in a pressure flask. The flask was sealed and the reaction was stirred for 5 h at 110° C. LCMS showed complete conversion with the presence of elimination byproduct. The reaction was diluted with EtOAc/water (20 mL/5 mL). The organic layer were separated, the aqueous phase was extracted with EtOAc (4×20 mL). The combined organic phases were dried with Na₂SO₄, filtered and concentrated under reduced pressure to give the crude product. The crude was purified by flash column chromatography (silica, pretreated with TEA, from 0% MeOH in DCM (0.5% TEA) to 15% MeOH in DCM (0.5% TEA)) to give 4-methyl-3-[(2S)-2-(3-bromophenyl)-2-fluoro-propyl]-1,2,4-triazole (98 mg, 99% yield). LCMS (ESI) [M+H]⁺=235.2.

### Example 8: Intermediate N (6-Cyclopropyl-N-(3-(1,2-difluoro-1-(4-methyl-4H-1,2,4-triazol-3-yl)propan-2-yl)phenyl)-4-formylpicolinamide)

Intermediate N can be synthesized according to Scheme 8, FIG. 5H.

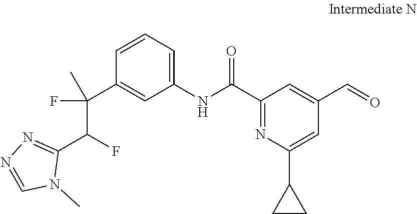 [SMILES: Cn1cnnc1C(F)C(C)(F)c1cccc(NC(=O)c2cc(C=O)cc(C3CC3)n2)c1]

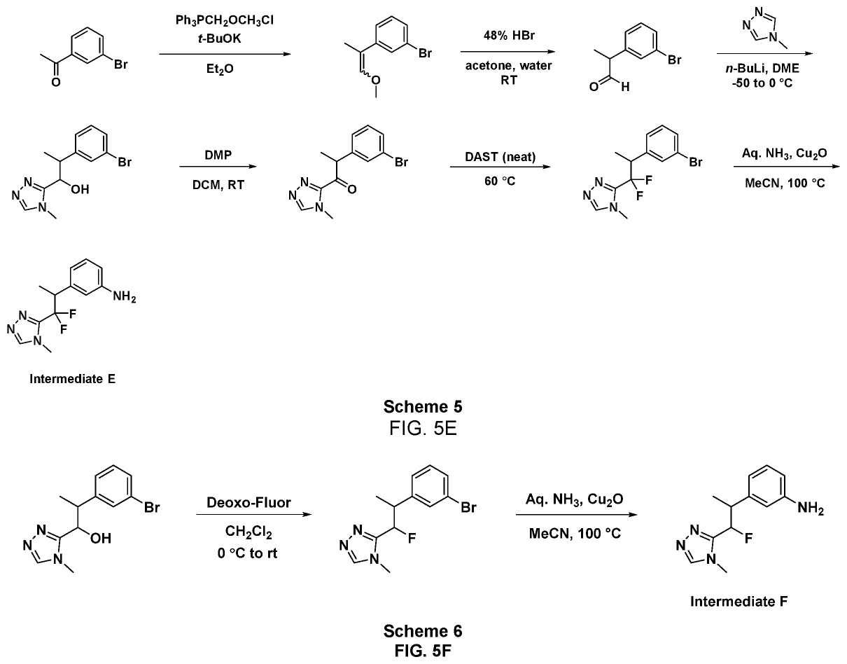

In a single step 1, Intermediate N is made as follows. 3-[1,2-difluoro-1-methyl-2-(4-methyl-1,2,4-triazol-3-yl)ethyl]aniline, prepared similarly to chemistry outlined in Scheme 4 (FIG. 6D) (220 mg, 0.87 mmol), 6-cyclopropyl-4-formyl-pyridine-2-carboxylic acid (167 mg, 0.87 mmol) and propylphosphonic anhydride solution (50% in EtOAc) (1.23 mL, 1.74 mmol) were dissolved in acetonitrile (4.4 mL) before pyridine (0.14 mL, 1.74 mmol) was added. The resulting reaction mixture was stirred at RT for 18 h. The reaction was diluted with water (15 mL) and extracted with CH₂Cl₂ (3×15 mL). The combined organic layers were washed with water (30 mL), brine (30 mL), dried over sodium sulfate and concentrated under reduced pressure. The residue was purified by chromatography on silica gel (0-10% methanol in CH₂Cl₂) afford 6-cyclopropyl-N-[3-[1,2-difluoro-1-methyl-2-(4-methyl-1,2,4-triazol-3-yl)ethyl]phenyl]-4-formyl-pyridine-2-carboxamide (64 mg, 17% yield) as a pink solid. LCMS (ESI) m/z: 465.0 [M+H]⁺.

Examples 9 to 28 describe synthesis of various exemplary picolinamide compounds according to the invention.

### Example 9: Compounds 1 and 2

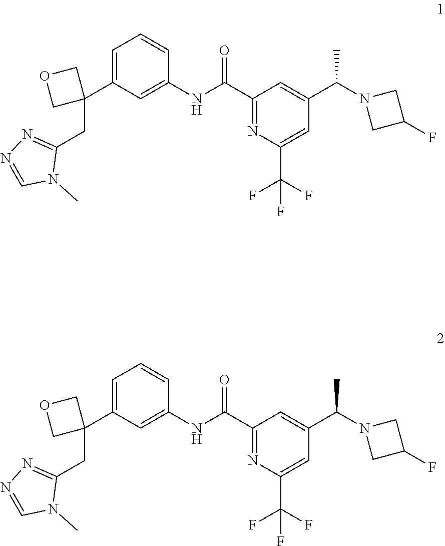 [SMILES: C[C@@H](c1cc(C(=O)Nc2cccc(C3(Cc4nncn4C)COC3)c2)nc(C(F)(F)F)c1)N1CC(F)C1 ; C[C@H](c1cc(C(=O)Nc2cccc(C3(Cc4nncn4C)COC3)c2)nc(C(F)(F)F)c1)N1CC(F)C1]

Compounds 1 ((S)-4-(1-(3-fluoroazetidin-1-yl)ethyl)-N-(3-(3-((4-methyl-4H-1,2,4-triazol-3-yl)methyl)oxetan-3-yl)phenyl)-6-(trifluoromethyl)picolinamide) and 2 ((R)-4-(1-(3-fluoroazetidin-1-yl)ethyl)-N-(3-(3-((4-methyl-4H-1,2,4-triazol-3-yl)methyl)oxetan-3-yl)phenyl)-6-(trifluoromethyl)picolinamide) can be synthesized according to Scheme 9 (FIG. 6), in which various intermediates are synthesized as follows.

A first intermediate (1-(2-chloro-6-(trifluoromethyl)pyridin-4-yl)ethan-1-one) is made as follows. To a mixture of 2-chloro-4-iodo-6-(trifluoromethyl)pyridine (5.00 g, 16.26 mmol; CAS No.: 205444-22-0) and bis(triphenylphosphine) palladium(II)dichloride (570.8 mg, 0.81 mmol) in toluene (90 mL) was added tributyl(1-ethoxyvinyl)stannane (6.6 mL, 19.38 mmol). The reaction mixture was stirred for 16 h at 110° C. under nitrogen protection and then cooled to room temperature. The mixture was added HCl solution (4.0 M aqueous, 20.33 mL, 81.32 mmol) and stirred at room temperature for 3 h. The reaction mixture was diluted with ethyl acetate (200 mL), which was washed with water (2×100 mL). The organic layer was separated, dried over anhydrous sodium sulfate and concentrated under reduced pressure. The residue was purified by silica gel chromatography (mobile phase: ethyl acetate/petroleum ether, gradient 0% to 40%) to afford 1-(2-chloro-6-(trifluoromethyl)pyridin-4-yl)ethan-1-one (3.10 g, 85% yield) as a yellow oil. LCMS [M+H]⁺=223.7.

A second intermediate, methyl 4-acetyl-6-(trifluoromethyl)picolinate is made as follows. To a solution of 1-(2-chloro-6-(trifluoromethyl)pyridin-4-yl)ethan-1-one (2.70 g, 12.08 mmol) in methanol (20 mL) and N,N-dimethylformamide (20 mL) was added 1,1′-Bis(diphenylphosphino)ferrocene]dichloropalladium(II) (441.8 mg, 0.60 mmol) and triethylamine (5.0 mL, 36.23 mmol). The reaction mixture was stirred at 80° C. for 16 h under CO (2.0 MPa) atmosphere. The reaction mixture was filtered through a pad of Celite and diluted with water (100 mL). The mixture was extracted with ethyl acetate (2×100 mL). The combined organic layers were washed with brine (50 mL), dried over anhydrous sodium sulfate and concentrated under reduced pressure. The residue was purified by silica gel chromatography (mobile phase: ethyl acetate/petroleum ether, gradient 0% to 10%) to afford methyl 4-acetyl-6-(trifluoromethyl)picolinate (1.25 g, 42% yield) as a white solid. ¹H NMR (400 MHz, methanol-d₄): δ 8.71 (s, 1H), 8.40 (d, J=1.2 Hz, 1H), 4.04 (s, 3H), 3.31 (s, 3H).

A further intermediate, 4-acetyl-6-(trifluoromethyl)picolinic acid, is made as follows. To a solution of methyl 4-acetyl-6-(trifluoromethyl)pyridine-2-carboxylate (1.25 g, 5.06 mmol) in tetrahydrofuran (20 mL) and water (20 mL) was added lithium hydroxide hydrate (193.8 mg, 8.09 mmol). The reaction mixture was stirred for 16 h at room temperature and then diluted with water (20 mL). The mixture was adjusted to pH=6 by carefully addition of 1 M HCl solution. The residue was extracted with ethyl acetate (2×20 mL). The organic layers were combined and concentrated under reduced pressure to afford crude 4-acetyl-6-(trifluoromethyl)picolinic acid (1.03 g, 88% yield) as a white solid.

A final intermediate, 4-acetyl-N-(3-(3-((4-methyl-4H-1,2,4-triazol-3-yl)methyl)oxetan-3-yl)phenyl)-6-(trifluoromethyl)picolinamide, is made as follows. To a solution of 4-acetyl-6-(trifluoromethyl)picolinic acid (1.03 g, 4.42 mmol) and 3-(3-((4-methyl-4H-1,2,4-triazol-3-yl)methyl)oxetan-3-yl)aniline (synthesized as shown in WO2019148005, 1.18 g, 4.86 mmol) in acetonitrile (40 mL) was added 2,4,6-tripropyl-1,3,5,2,4,6-trioxa-triphosphorinane-2,4,6-trioxide (4.21 g, 6.63 mmol, 50% in ethyl acetate) and pyridine (2.23 mL, 22.09 mmol). The reaction mixture was stirred at 25° C. for 16 h and concentrated under reduced pressure. The residue was purified by silica gel chromatography (mobile phase: methanol/dichloromethane, gradient 0% to 20%) to afford 4-acetyl-N-(3-(3-((4-methyl-4H-1,2,4-triazol-3-yl)methyl)oxetan-3-yl)phenyl)-6-(trifluoromethyl)picolinamide (1.30 g, 63% yield) as a white solid. ¹H NMR (400 MHz, DMSO-d₆): δ 10.48 (s, 1H), 8.68 (s, 1H), 8.44 (d, J=1.2 Hz, 1H), 8.20 (s, 1H), 7.81 (d, J=8.0 Hz, 1H), 7.49 (s, 1H), 7.30 (t, J=8.0 Hz, 1H), 6.70 (d, J=7.6 Hz, 1H), 4.94 (d, J=6.0 Hz, 2H), 4.87 (d, J=6.0 Hz, 2H), 3.50 (s, 2H), 2.93 (s, 3H), 2.77 (s, 3H). LCMS [M+H]⁺=459.9.

To a solution of 4-acetyl-N-(3-(3-((4-methyl-4H-1,2,4-triazol-3-yl)methyl)oxetan-3-yl)phenyl)-6-(trifluoromethyl)picolinamide (250.0 mg, 0.54 mmol) in methanol (5 mL) was added 3-fluoroazetidinehydrochloride (309.6 mg, 2.78 mmol) and triethylamine (0.37 mL, 2.67 mmol). The reaction mixture was heated under microwave irradiation at 100° C. for 1 minute and cooled down to room temperature. The mixture was then added sodium cyanoborohydride (61.5 mg, 0.98 mmol) and heated at 80° C. under microwave irradiation for another 30 minutes. The reaction was quenched with 1 M HCl solution and adjusted to pH=7 by addition of saturated NaHCO₃ solution. The resulting solution was extracted with dichloromethane (3×20 mL). The combined organic phases were dried over anhydrous sodium sulphate and concentrated under reduced pressure. The residue was purified by silica gel chromatography (mobile phase: methanol/dichloromethane, gradient 0% to 10%) to afford 4-(1-(3-fluoroazetidin-1-yl)ethyl)-N-(3-(3-((4-methyl-4H-1,2,4-triazol-3-yl)methyl)oxetan-3-yl)phenyl)-6-(trifluoromethyl)picolinamide (150.0 mg, 53% yield) as a colourless oil. LCMS [M+H]⁺=519.3.

The above racemate was further purified by chiral SFC (Column=Phenomenex-Cellulose-2; Column dimensions=250 mm×30 mm×5 μm; Detection wavelength=220 nm; Flow rate=55 mL/min; Run time=10 min; Column temperature=25° C.) with 0.1% ammonium hydroxide-35% methanol-carbon dioxide) to afford 1 and 2.

(S)-4-(1-(3-fluoroazetidin-1-yl)ethyl)-N-(3-(3-((4-methyl-4H-1,2,4-triazol-3-yl)methyl)oxetan-3-yl)phenyl)-6-(trifluoromethyl) picolinamide (Peak 2, retention time=7.657 min) (48.5 mg, 32% yield) as a white solid (1). ¹H NMR (400 MHz, methanol-d₄): δ 8.41 (s, 1H), 8.19 (s, 1H), 8.02 (s, 1H), 7.72 (d, J=8.0 Hz, 1H), 7.46-7.12 (m, 2H), 6.75-6.51 (m, 1H), 5.26-4.97 (m, 5H), 3.80-3.67 (m, 2H), 3.64 (s, 2H), 3.58-3.47 (m, 1H), 3.30-3.11 (m, 2H), 2.87 (s, 3H), 1.30 (d, J=6.8 Hz, 3H). LCMS [M+H]⁺ or [M−H]⁻: 519.1.

(R)-4-(1-(3-fluoroazetidin-1-yl)ethyl)-N-(3-(3-((4-methyl-4H-1,2,4-triazol-3-yl)methyl)oxetan-3-yl)phenyl)-6-(trifluoromethyl)picolinamide (Peak 1, retention time=7.267 min) (50.4 mg, 32.2% yield) as a white solid (2). ¹H NMR (400 MHz, methanol-d₄): δ 8.40 (s, 1H), 8.18 (s, 1H), 8.00 (s, 1H), 7.73-7.69 (m, 1H), 7.35-7.30 (m, 2H), 6.64 (d, J=7.6 Hz, 1H), 5.23-5.02 (m, 5H), 3.76-3.70 (m, 2H), 3.63 (s, 2H), 3.57-3.46 (m, 1H), 3.30-3.25 (m, 2H), 2.86 (s, 3H), 1.29 (d, J=6.4 Hz, 3H). LCMS [M+H]⁺ or [M−H]⁻: 519.1.

### Example 10 Compounds 3 and 4

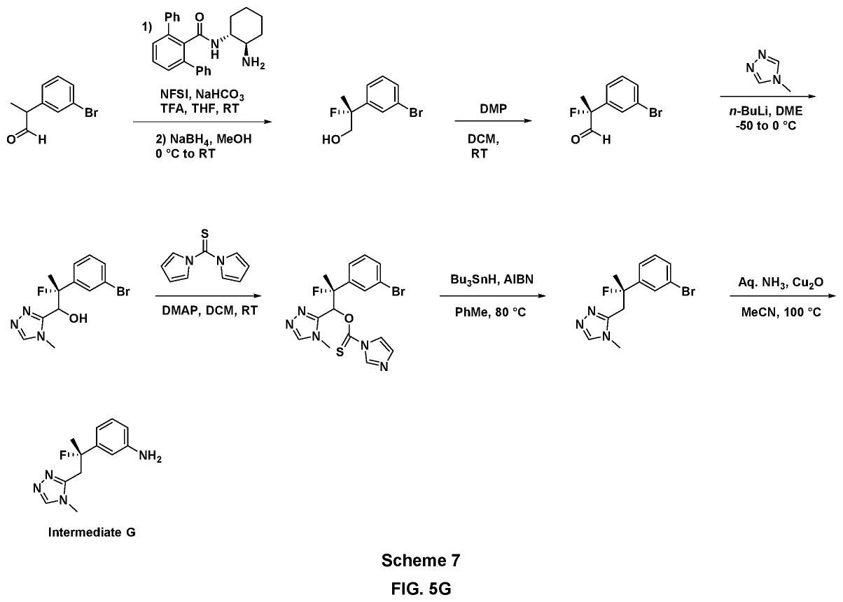

Compounds 3 ((S)-4-(1-(5-azaspiro[2.4]heptan-5-yl)ethyl)-N-(3-(3-((4-methyl-4H-1,2,4-triazol-3-yl)methyl)oxetan-3-yl)phenyl)-6-(trifluoromethyl)picolinamide), and 4 ((R)-4-(1-(5-azaspiro[2.4]heptan-5-yl)ethyl)-N-(3-(3-((4-methyl-4H-1,2,4-triazol-3-yl)methyl)oxetan-3-yl)phenyl)-6-(trifluoromethyl)picolinamide) can be synthesized according to Scheme 10, in FIG. 7.

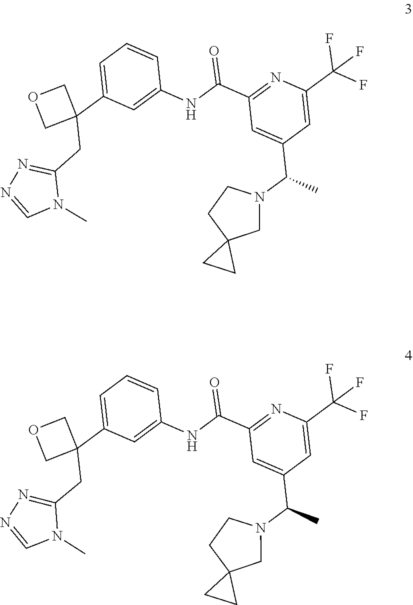 [SMILES: C[C@@H](c1cc(C(=O)Nc2cccc(C3(Cc4nncn4C)COC3)c2)nc(C(F)(F)F)c1)N1CCC2(CC2)C1 ; C[C@H](c1cc(C(=O)Nc2cccc(C3(Cc4nncn4C)COC3)c2)nc(C(F)(F)F)c1)N1CCC2(CC2)C1]

To a solution of 4-acetyl-N-(3-(3-((4-methyl-4H-1,2,4-triazol-3-yl)methyl)oxetan-3-yl)phenyl)-6-(trifluoromethyl)picolinamide, which can be prepared as in Example 16, (300.0 mg, 0.65 mmol) in methanol (4 mL) was added 5-azaspiro[2.4] heptane hydrochloride (445.0 mg, 3.33 mmol) and triethylamine (0.44 mL, 3.20 mmol). The mixture was heated under microwave irradiation at 100° C. for 1 minute and cooled down to room temperature. The reaction mixture was then added sodium cyanoborohydride (73.9 mg, 1.18 mmol) and heated at 80° C. under microwave irradiation for another 30 minutes. The reaction was quenched with 1 M HCl solution and adjusted to pH=7 by addition of saturated NaHCO₃ solution. The resulting solution was extracted with dichloromethane (3×20 mL). The combined organic phases were dried over anhydrous sodium sulphate and concentrated under reduced pressure. The residue was purified by silica gel chromatography (mobile phase: methanol/dichloromethane, gradient 0% to 10%) to afford 4-(1-(5-azaspiro[2.4]heptan-5-yl)ethyl)-N-(3-(3-((4-methyl-4H-1,2,4-triazol-3-yl)methyl)oxetan-3-yl)phenyl)-6-(trifluoromethyl)picolinamide (130.0 mg, 37% yield) as a colourless oil. LCMS [M+H]⁺=541.3.

The above racemate (150.0 mg, 0.28 mmol) was further purified by chiral SFC (Column=Daicel Chiralpak IC; Column dimensions=250 mm×30 mm×10 μm; Detection wavelength=220 nm; Flow rate=80 mL/min; Run time=14 min; Column temperature=25° C.) with 0.1% ammonium hydroxide-55% methanol-carbon dioxide) to afford:

(S)-4-(1-(5-azaspiro[2.4]heptan-5-yl)ethyl)-N-(3-(3-((4-methyl-4H-1,2,4-triazol-3-yl)methyl)oxetan-3-yl)phenyl)-6-(trifluoromethyl)picolinamide (Peak 2, retention time=9.089 min) (68.9 mg, 45% yield) as a white solid (3). ¹H NMR (400 MHz, methanol-d₄): δ 8.46 (s, 1H), 8.19 (s, 1H), 8.08 (s, 1H), 7.72 (d, J=8.0 Hz, 1H), 7.44-7.22 (m, 2H), 6.65 (d, J=7.6 Hz, 1H), 5.12-4.97 (m, 4H), 3.71-3.54 (m, 3H), 2.93-2.77 (m, 4H), 2.71-2.55 (m, 2H), 2.44 (d, J=9.2 Hz, 1H), 1.97-1.79 (m, 2H), 1.45-1.43 (m, 3H), 0.64-0.49 (m, 4H). LCMS [M+H]⁺ or [M−H]⁻: 541.1.

(R)-4-(1-(5-azaspiro[2.4]heptan-5-yl)ethyl)-N-(3-(3-((4-methyl-4H-1,2,4-triazol-3-yl)methyl)oxetan-3-yl)phenyl)-6-(trifluoromethyl)picolinamide (Peak 1, retention time=7.828 min) (56.9 mg, 36.8% yield) as a white solid (4). ¹H NMR (400 MHz, methanol-d₄): δ 8.46 (s, 1H), 8.19 (s, 1H), 8.08 (s, 1H), 7.72 (d, J=8.0 Hz, 1H), 7.36-7.31 (m, 2H), 6.65 (d, J=7.6 Hz, 1H), 5.08-5.03 (m, 4H), 3.64-3.57 (m, 3H), 2.88-2.82 (m, 4H), 2.65-2.60 (m, 2H), 2.44 (d, J=9.2 Hz, 1H), 1.89-1.83 (m, 2H), 1.44 (d, J=6.8 Hz, 3H), 0.62-0.54 (m, 4H). LCMS [M+H]⁺ or [M−H]⁻: 541.2.

### Example 11: Compounds 5 and 6

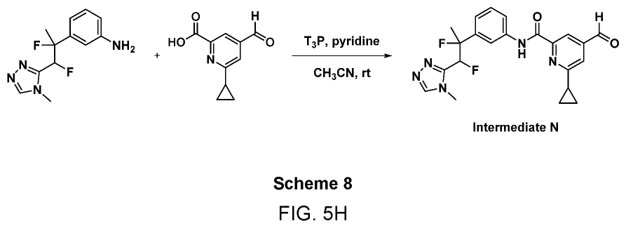

Compounds 5 ((R)-4-(5-azaspiro[2.4]heptan-5-ylmethyl)-6-cyclopropyl-N-(3-(3-(fluoro(4-methyl-4H-1,2,4-triazol-3-yl)methyl)oxetan-3-yl)phenyl)picolinamide) and 6 ((S)-4-(5-azaspiro[2.4]heptan-5-ylmethyl)-6-cyclopropyl-N-(3-(3-(fluoro(4-methyl-4H-1,2,4-triazol-3-yl)methyl)oxetan-3-yl)phenyl)picolinamide) can be synthesized as in Scheme 11, in FIG. 8.

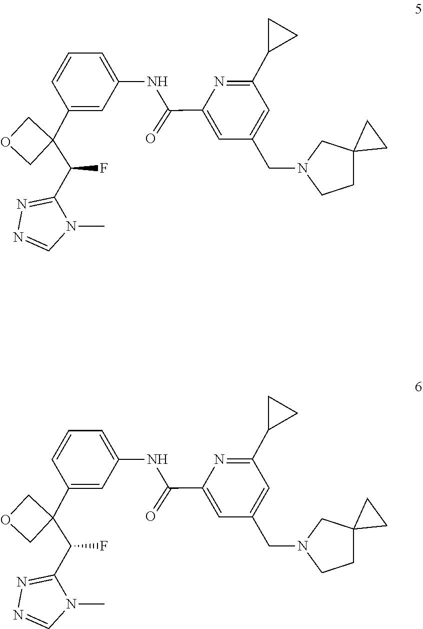 [SMILES: Cn1cnnc1[C@H](F)C1(c2cccc(NC(=O)c3cc(CN4CCC5(CC5)C4)cc(C4CC4)n3)c2)COC1 ; Cn1cnnc1[C@@H](F)C1(c2cccc(NC(=O)c3cc(CN4CCC5(CC5)C4)cc(C4CC4)n3)c2)COC1]

An intermediate (6-cyclopropyl-N-(3-(3-(fluoro(4-methyl-4H-1,2,4-triazol-3-yl)methyl)oxetan-3-yl)phenyl)-4-formylpicolinamide) can be synthesized as follows. To a mixture of 3-[3-[fluoro-(4-methyl-1,2,4-triazol-3-yl)methyl]oxetan-3-yl]aniline (synthesized as shown in WO2019148005; 500 mg, 1.91 mmol) and 6-cyclopropyl-4-formyl-pyridine-2-carboxylic acid (380 mg, 2.00 mmol) in acetonitrile (18 mL) were added 2,4,6-tripropyl-1,3,5,2,4,6-trioxatriphosphorinane-2,4,6-trioxide (1.82 g, 2.86 mmol, 50% in ethyl acetate) and pyridine (0.46 mL, 5.72 mmol). The reaction mixture was stirred at 25° C. for 12 h then filtered. The collected solid was washed with petroleum ether (15 mL), and dried to give 6-cyclopropyl-N-[3-[3-[fluoro-(4-methyl-1,2,4-triazol-3-yl)methyl]oxetan-3-yl]phenyl]-4-formyl-pyridine-2-carboxamide (470.0 mg, 57% yield) as a white solid. LCMS [M+H]⁺=436.2.

A mixture of 6-cyclopropyl-N-[3-[3-[fluoro-(4-methyl-1,2,4-triazol-3-yl)methyl]oxetan-3-yl]phenyl]-4-formyl-pyridine-2-carboxamide (180 mg, 0.41 mmol), sodium triacetoxyborohydride (175 mg, 0.83 mmol), triethylamine (0.09 mL, 0.62 mmol) and 5-azaspiro[2.4]heptane hydrochloride (66.3 mg, 0.50 mmol) in dichloromethane (5.5 mL) was stirred at 25° C. for 2 h. The reaction mixture was diluted with water (25 mL) and extracted with ethyl acetate (3×25 mL). The combined organics were washed with brine (2×25 mL), dried over sodium sulfate and concentrated under reduced pressure. The residue was purified by silica gel chromatography (mobile phase: methanol/dichloromethane, gradient 0% to 10%) to afford 4-(5-azaspiro[2.4]heptan-5-ylmethyl)-6-cyclopropyl-N-(3-(3-(fluoro(4-methyl-4H-1,2,4-triazol-3-yl)methyl)oxetan-3-yl)phenyl)picolinamide (130 mg, 61% yield) as a white solid. LCMS [M+H]⁺=517.2.

The above racemate (130 mg, 0.25 mmol) was further purified by chiral SFC (Column=Daicel Chiralpak OD-H; Column dimensions=250 mm×30 mm×5 μm; Detection wavelength=220 nm; Flow rate=60 mL/min; Run time=4 min; Column temperature=25° C.) with 0.1% ammonium hydroxide-40% methanol-carbon dioxide) to give compounds 5 and 6.

(R)-4-(5-azaspiro[2.4]heptan-5-ylmethyl)-6-cyclopropyl-N-(3-(3-(fluoro(4-methyl-4H-1,2,4-triazol-3-yl)methyl)oxetan-3-yl)phenyl) picolinamide (Peak 1, retention time=1.719 min) (25.5 mg, 20% yield) as a white solid (5). ¹H NMR (400 MHz, methanol-d₄): δ 8.28 (s, 1H), 7.95 (d, J=1.2 Hz, 1H), 7.70 (dd, J=1.2, 8.0 Hz, 1H), 7.44 (d, J=1.2 Hz, 1H), 7.43 (t, J=1.6 Hz, 1H), 7.35 (t, J=8.0 Hz, 1H), 6.78 (d, J=8.0 Hz, 1H), 6.29 (d, J=45.2 Hz, 1H), 5.47 (d, J=6.4 Hz, 1H), 5.30 (d, J=6.4 Hz, 1H), 5.19 (dd, J=2.0, 6.8 Hz, 1H), 5.01 (dd, J=4.0, 6.4 Hz, 1H), 3.74 (s, 2H), 3.07 (s, 3H), 2.81 (t, J=6.8 Hz, 2H), 2.55 (s, 2H), 2.27-2.18 (m, 1H), 1.87 (t, J=6.8 Hz, 2H), 1.18-1.06 (m, 4H), 0.58 (d, J=6.4 Hz, 4H). LCMS [M+H]⁺ or [M−H]⁻: 517.2.

(S)-4-(5-azaspiro[2.4]heptan-5-ylmethyl)-6-cyclopropyl-N-(3-(3-(fluoro(4-methyl-4H-1,2,4-triazol-3-yl)methyl)oxetan-3-yl)phenyl)picolinamide (Peak 2, retention time=1.918 min) (30.7 mg, 24% yield) as a white solid (6). ¹H NMR (400 MHz, methanol-d₄): δ 8.28 (s, 1H), 7.95 (d, J=1.2 Hz, 1H), 7.70 (dd, J=1.2, 8.4 Hz, 1H), 7.45 (d, J=1.2 Hz, 1H), 7.43 (t, J=2.0 Hz, 1H), 7.35 (t, J=8.0 Hz, 1H), 6.78 (d, J=8.0 Hz, 1H), 6.29 (d, J=45.2 Hz, 1H), 5.47 (d, J=6.4 Hz, 1H), 5.30 (d, J=6.4 Hz, 1H), 5.19 (dd, J=1.6, 6.4 Hz, 1H), 5.01 (dd, J=4.0, 6.0 Hz, 1H), 3.76 (s, 2H), 3.07 (s, 3H), 2.83 (t, J=6.8 Hz, 2H), 2.57 (s, 2H), 2.27-2.18 (m, 1H), 1.88 (t, J=6.8 Hz, 2H), 1.18-1.06 (m, 4H), 0.58 (d, J=6.4 Hz, 4H). LCMS [M+H]⁺ or [M−H]⁻: 517.2.

### Example 12: Compounds 7 and 8

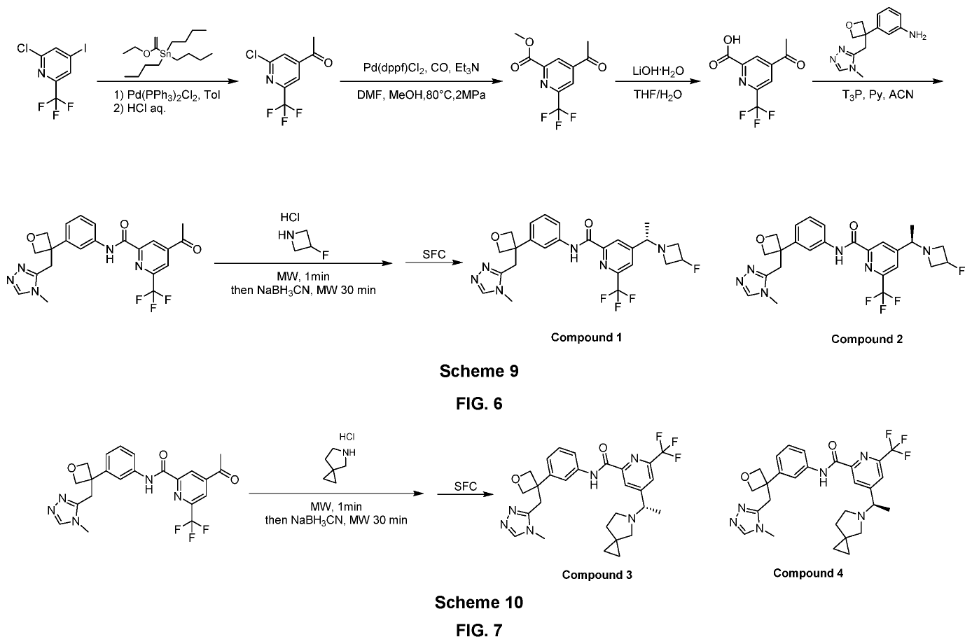

Compounds 7 ((R)-6-cyclopropyl-N-(3-(3-(fluoro(4-methyl-4H-1,2,4-triazol-3-yl)methyl)oxetan-3-yl)phenyl)-4-((3-fluoro-3-methylazetidin-1-yl)methyl)picolinamide), and 8 ((S)-6-cyclopropyl-N-(3-(3-(fluoro(4-methyl-4H-1,2,4-triazol-3-yl)methyl)oxetan-3-yl)phenyl)-4-((3-fluoro-3-methylazetidin-1-yl)methyl)picolinamide) can be made according to Scheme 12 (FIG. 9).

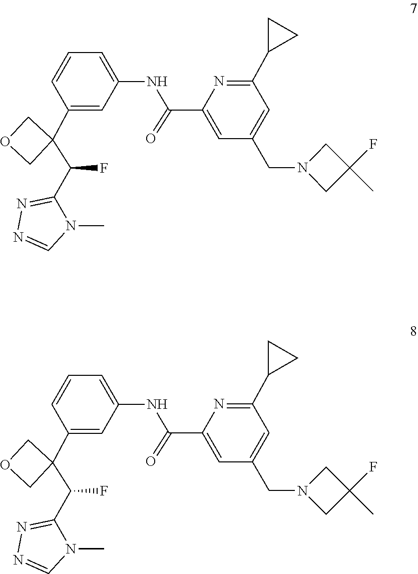 [SMILES: Cn1cnnc1[C@H](F)C1(c2cccc(NC(=O)c3cc(CN4CC(C)(F)C4)cc(C4CC4)n3)c2)COC1 ; Cn1cnnc1[C@@H](F)C1(c2cccc(NC(=O)c3cc(CN4CC(C)(F)C4)cc(C4CC4)n3)c2)COC1]

A mixture of 6-cyclopropyl-N-[3-[3-[fluoro-(4-methyl-1,2,4-triazol-3-yl)methyl]-oxetan-3-yl]phenyl]-4-formyl-pyridine-2-carboxamide, which can be synthesized as in Example 18, (180.0 mg, 0.41 mmol), sodiumtriacetoxyborohydride (175.2 mg, 0.83 mmol), triethylamine (0.09 mL, 0.62 mmol) and 3-fluoro-3-methyl-azetidine hydrochloride (62.3 mg, 0.50 mmol) in dichloromethane (5.0 mL) was stirred at 25° C. for 12 h. The reaction was diluted with water (25 mL) and extracted with ethyl acetate (3×25 mL). The organics were washed with brine (2×25 mL), dried over sodium sulfate, filtered and concentrated. The residue was purified by chromatography on silica (solvent gradient: 0% to 6% methanol in dichloromethane) to afford 6-cyclopropyl-N-(3-(3-(fluoro(4-methyl-4H-1,2,4-triazol-3-yl)methyl)oxetan-3-yl)phenyl)-4-formylpicolinamide (120.0 mg, 57.1% yield) as colorless oil.

The above racemate (120 mg, 0.28 mmol) was purified by SFC (Column=Phenomenex-Cellulose-2; Column dimensions=250 mm×30 mm×5 μm; Detection wavelength=220 nm; Flow rate=50 mL/min; Run time=7 min; Column temperature=25° C.) with 0.1% ammonium hydroxide-40% ethanol-carbon dioxide) to give:

(R)-6-cyclopropyl-N-(3-(3-(fluoro(4-methyl-4H-1,2,4-triazol-3-yl)methyl)oxetan-3-yl)phenyl)-4-((3-fluoro-3-methylazetidin-1-yl)methyl)picolinamide (Peak 1, retention time=3.095 min) (28.27 mg, 20% yield) as a white solid (7). ¹H NMR (400 MHz, methanol-d₄): δ 8.28 (s, 1H), 7.89 (d, J=1.2 Hz, 1H), 7.70 (dd, J=1.2 Hz, 8.4 Hz, 1H), 7.43 (t, J=2.0 Hz, 1H), 7.38 (br. s, 1H), 7.35 (t, J=8.0 Hz, 1H), 6.79 (d, J=7.6 Hz, 1H), 6.30 (d, J=45.2 Hz, 1H), 5.47 (d, J=6.4 Hz, 1H), 5.30 (d, J=6.4 Hz, 1H), 5.19 (dd, J=1.6, 6.4 Hz, 1H), 5.01 (dd, J=3.6, 6.4 Hz, 1H), 3.81 (s, 2H), 3.48-3.36 (m, 4H), 3.07 (s, 3H), 2.26-2.18 (m, 1H), 1.59 (d, J=22.0 Hz, 3H), 1.18-1.12 (m, 2H), 1.12-1.06 (m, 2H). LCMS [M+H]⁺ or [M−H]⁻: 509.2.

(S)-6-cyclopropyl-N-(3-(3-(fluoro(4-methyl-4H-1,2,4-triazol-3-yl)methyl)oxetan-3-yl)phenyl)-4-((3-fluoro-3-methylazetidin-1-yl)methyl)picolinamide (Peak 2, retention time=4.599 min) (39.6 mg, 28% yield) as a white solid (8). ¹H NMR (400 MHz, methanol-d₄): δ 8.28 (s, 1H), 7.89 (d, J=1.2 Hz, 1H), 7.70 (dd, J=1.2, 8.0 Hz, 1H), 7.43 (t, J=2.0 Hz, 1H), 7.38 (br. s, 1H), 7.35 (t, J=7.6 Hz, 1H), 6.78 (d, J=7.6 Hz, 1H), 6.29 (d, J=45.2 Hz, 1H), 5.47 (d, J=6.4 Hz, 1H), 5.30 (d, J=6.4 Hz, 1H), 5.19 (dd, J=2.0, 6.8 Hz, 1H), 5.01 (dd, J=4.0, 6.0 Hz, 1H), 3.80 (s, 2H), 3.48-3.36 (m, 4H), 3.07 (s, 3H), 2.26-2.17 (m, 1H), 1.59 (d, J=22.4 Hz, 3H), 1.18-1.12 (m, 2H), 1.12-1.06 (m, 2H). LCMS [M+H]⁺ or [M−H]⁻: 509.2.

### Example 13: Compound 16

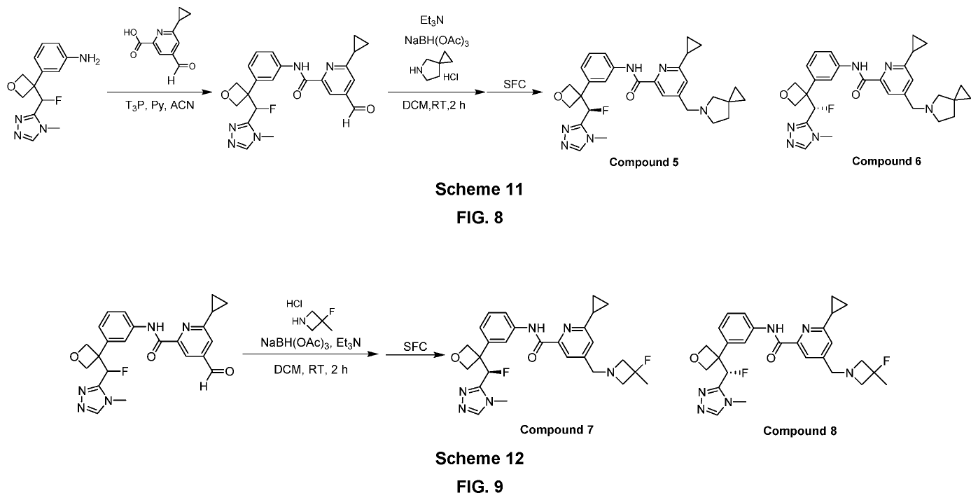

Compound 16 (N-(3-(3,3-difluoro-1-((4-methyl-4H-1,2,4-triazol-3-yl)methyl)-cyclobutyl)phenyl)-6-(trifluoromethyl)picolinamide) has been synthesized according to Scheme 13, FIG. 10, in which various intermediates are prepared as follows.

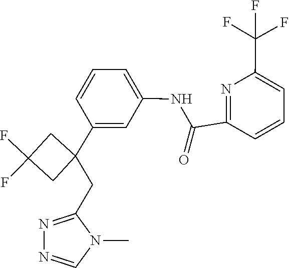 [SMILES: Cn1cnnc1CC1(c2cccc(NC(=O)c3cccc(C(F)(F)F)n3)c2)CC(F)(F)C1]

A first intermediate, ethyl 2-(3-(benzyloxy)cyclobutylidene)acetate, can be made as follows. To a solution of 3-(benzyloxy)cyclobutan-1-one (10.0 g, 56.75 mmol) in dichloromethane (300 mL) was added ethyl 2-(triphenyl-phosphaneylidene)acetate (19.8 g, 56.75 mmol) at 0° C. The resulted mixture was stirred at 25° C. for 16 h and washed with water (2×100 mL). The separated organic layer was dried over anhydrous sodium sulfate and concentrated under reduced pressure. The residue was purified by silica gel chromatography (mobile phase: ethyl acetate/petroleum ether, gradient 0% to 5%) to afford ethyl 2-(3-benzyloxycyclobutylidene)acetate (10.0 g, 72% yield) as colorless oil.

A second intermediate, ethyl 2-(3-(benzyloxy)-1-(3-nitrophenyl)cyclobutyl)acetate, is made as follows. To a solution of chloro(1,5-cyclooctadiene)rhodium(I) dimer (900.9 mg, 1.83 mmol) in 1,4-dioxane (36 mL) was added aqueous potassium hydroxide (1.5M, 3.7 mL, 5.48 mmol). The mixture was stirred at 20° C. for 30 minutes, and a solution of (3-nitrophenyl)boronic acid (9.39 g, 56.23 mmol) in 1,4-dioxane (90 mL) and water (18 mL) was added. The resulting mixture was stirred at 20° C. for 30 minutes, then a solution of Ethyl 2-(3-benzyloxycyclobutylidene)acetate (9.0 g, 36.54 mmol) in 1,4 dioxane (30 mL) was added dropwise. After addition, the mixture was stirred at 25° C. for 1 h, and another portion of (3-nitrophenyl)boronic acid (9.39 g, 56.23 mmol) was added. The resulting mixture was stirred at 20° C. for 16 h and diluted with water (200 mL). The mixture was extracted with ethyl acetate (2×200 mL). The combined organic layers were washed with brine (100 mL), dried over anhydrous sodium sulfate and concentrated under reduced pressure. The residue was purified by silica gel chromatography (mobile phase: ethyl acetate/petroleum ether, gradient 0% to 10%) to afford ethyl 2-[3-benzyloxy-1-(3-nitrophenyl)cyclobutyl]acetate (1.8 g, 13% yield) as a yellow oil. ¹H NMR (400 MHz, CDCl₃): δ 8.09-8.03 (m, 2H), 7.51-7.47 (m, 2H), 7.35-7.31 (m, 5H), 4.45 (s, 2H), 3.97-3.93 (m, 2H), 2.93-2.88 (m, 2H), 2.74 (s, 2H), 2.44-2.41 (m, 2H), 1.11-1.06 (m, 3H).

A third intermediate, ethyl 2-(3-hydroxy-1-(3-nitrophenyl)cyclobutyl)acetate, can be prepared in the following step. To a mixture of ethyl 2-[3-benzyloxy-1-(3-nitrophenyl)cyclobutyl]acetate (1.5 g, 4.06 mmol) in dichloromethane (60 mL) was added tribromoborane (1.5 mL, 15.57 mmol) at −40° C. The mixture was stirred for 3 h and slowly quenched by addition of methanol (10 mL). The solution was concentrated under reduced pressure and the residue was purified by preparative TLC (mobile phase: 20% ethyl acetate in petroleum ether) to afford ethyl 2-[3-hydroxy-1-(3-nitrophenyl)cyclobutyl]acetate (550 mg, 49% yield) as a yellow oil. ¹H NMR (400 MHz, CDCl₃): δ 8.08-8.01 (m, 2H), 7.52-7.47 (m, 2H), 4.56-4.50 (m, 1H), 3.97-3.92 (m, 2H), 2.98-2.93 (m, 2H), 2.73 (s, 2H), 2.34-2.29 (m, 2H), 1.07 (t, J=7.2 Hz, 3H).

A fourth intermediate, ethyl 2-(1-(3-nitrophenyl)-3-oxocyclobutyl)acetate, can be made in the following step. To a mixture of ethyl 2-[3-hydroxy-1-(3-nitrophenyl)cyclobutyl]acetate (550.0 mg, 1.97 mmol) in dichloromethane (50 mL) was added Dess-Martin periodinane (1.67 g, 3.94 mmol) at 0° C. The mixture was stirred for 2 h at 0° C. and quenched by addition of saturated aqueous Na₂SO₃ (5 mL). The solution was extracted with dichloromethane (3×10 mL). The combined the organic layers was dried over anhydrous sodium sulfate and concentrated under reduced pressure. The residue was purified by silica gel chromatography (mobile phase: ethyl acetate/petroleum ether, gradient 0% to 20%) to afford ethyl 2-[1-(3-nitrophenyl)-3-oxo-cyclobutyl]acetate (500.0 mg, 92% yield) as a yellow oil.

A fifth intermediate, ethyl 2-(3,3-difluoro-1-(3-nitrophenyl)cyclobutyl)acetate, can be made in the following step. To a mixture of ethyl 2-[1-(3-nitrophenyl)-3-oxo-cyclobutyl]acetate (400.0 mg, 1.44 mmol) in dichloromethane (15 mL) was added DAST (697.6 mg, 4.33 mmol) at 18° C. The mixture was stirred for 16 h and quenched by addition of saturated aqueous NaHCO₃ (15 mL). The solution was extracted with dichloromethane (3×15 mL). The combined organic layers were dried over sodium sulfate and concentrated under reduced pressure. The residue was purified by preparative TLC (mobile phase: 20% ethyl acetate in petroleum ether) to afford ethyl 2-[3,3-difluoro-1-(3-nitrophenyl)cyclobutyl]acetate (300 mg, 70% yield) as a white solid. ¹H NMR (400 MHz, CDCl₃): δ 8.14-8.09 (m, 2H), 7.64-7.48 (m, 2H), 4.02-3.96 (m, 2H), 3.16-3.02 (m, 4H), 2.91 (s, 2H), 1.12 (t, J=7.2 Hz, 3H).

A sixth intermediate, (2-(3,3-difluoro-1-(3-nitrophenyl)cyclobutyl)acetohydrazide) is prepared as follows. To a solution of ethyl 2-[3,3-difluoro-1-(3-nitrophenyl)cyclobutyl]acetate (300.0 mg, 1.0 mmol) in ethanol (5 mL) was added hydrazine monohydrate (3.0 mL, 52.0 mmol). The mixture was heated at 80° C. for 16 h and then concentrated under reduced pressure to afford crude 2-[3,3-difluoro-1-(3-nitrophenyl)cyclobutyl]acetohydrazide (285.0 mg, 99% yield) as a yellow oil. LCMS [M+H]+=286.1.

A seventh intermediate (2-(2-(3,3-difluoro-1-(3-nitrophenyl)cyclobutyl)acetyl)-N-methylhydrazinecarbothioamide) can be prepared as follows. To a mixture of 2-[3,3-difluoro-1-(3-nitrophenyl)cyclobutyl]acetohydrazide (285.0 mg, 1.0 mmol) in tetrahydrofuran (20 mL) was added methyl isothiocyanate (146.1 mg, 2 mmol) at 25° C. The mixture was stirred at 25° C. for 4 h and concentrated under reduced pressure to afford crude 2-(2-(3,3-difluoro-1-(3-nitrophenyl)cyclobutyl)acetyl)-N-methylhydrazinecarbothioamide (356 mg, 99% yield) as a yellow solid. LCMS [M+H]⁺=359.1.

An eighth intermediate, 5-((3,3-difluoro-1-(3-nitrophenyl)cyclobutyl)methyl)-4-methyl-4H-1,2,4-triazole-3-thiol, can be prepared as follows. A mixture of 1-[[2-[3,3-difluoro-1-(3-nitrophenyl)cyclobutyl]acetyl]amino]-3-methyl-thiourea (358.0 mg, 1.0 mmol) in aqueous sodium hydroxide (1 M, 20.0 mL, 20.0 mmol) was stirred at 25° C. for 1 h. The reaction mixture was adjusted to pH=5 by addition of 1 M HCl and concentrated under reduced pressure to afford crude 5-[[3,3-difluoro-1-(3-nitrophenyl)cyclobutyl]methyl]-4-methyl-4H-1,2,4-triazole-3-thiol (335 mg, 99% yield) as a yellow solid. LCMS [M+H]⁺=341.1.

A ninth intermediate, 3-((3,3-difluoro-1-(3-nitrophenyl)cyclobutyl)methyl)-4-methyl-4H-1,2,4-triazole, is prepared as follows. To a solution of 5-((3,3-difluoro-1-(3-nitrophenyl)cyclobutyl)methyl)-4-methyl-4H-1,2,4-triazole-3-thiol (340.0 mg, 1.00 mmol) in water (10 mL) and acetonitrile (9 mL) was added sodium nitrite (689.3 mg, 9.99 mmol), followed by addition of 1 M nitric acid (10.0 mL, 10.0 mmol) dropwise at 0° C. After addition, the mixture was stirred for 1 h at 20° C. and quenched by addition of saturated aqueous NaHCO₃ (10 mL). The resulting mixture was extracted with dichloromethane (3×20 mL). The combined organic layers were washed with brine (30 mL), dried and concentrated under reduced pressure. The residue was purified by silica gel chromatography (mobile phase: methanol/dichloromethane, gradient 0% to 10%) to afford 3-((3,3-difluoro-1-(3-nitrophenyl)cyclobutyl)methyl)-4-methyl-4H-1,2,4-triazole (250.0 mg, 81% yield) as brown oil. ¹H NMR (400 MHz, methanol-d₄): δ 8.20 (s, 1H), 8.15-8.10 (m, 1H), 7.90 (s, 1H), 7.57-7.53 (m, 1H), 7.43-7.42 (m, 1H), 3.40 (s, 2H), 3.36-3.33 (m, 2H), 3.12-2.88 (m, 2H), 2.87 (s, 3H). LCMS [M+H]⁺=309.1.

A final intermediate, 3-(3,3-difluoro-1-((4-methyl-4H-1,2,4-triazol-3-yl)methyl)cyclobutyl)aniline, is prepared as follows. To a mixture of 3-[[3,3-difluoro-1-(3-nitrophenyl)cyclobutyl]methyl]-4-methyl-1,2,4-triazole (60.0 mg, 0.19 mmol) in ethanol (3 mL) and water (3 mL) was added iron powder (195.7 mg, 3.50 mmol) and ammonium chloride (155 mg, 2.92 mmol). The mixture was stirred at 80° C. for 2 h under nitrogen atmosphere. After cooling, the mixture was filtered and the filtrate was concentrated under reduced pressure. The residue was dissolved in dichloromethane/methanol (10/1, 30 mL) and filtered. The cake was washed with dichloromethane (2×10 mL). The combined organic layers were concentrated under reduced pressure and the residue was purified by preparative TLC (mobile phase: 10% methanol in dichloromethane) to afford crude 3-(3,3-difluoro-1-((4-methyl-4H-1,2,4-triazol-3-yl)methyl)cyclobutyl)aniline (50 mg, 92% yield) as yellow oil. LCMS [M+H]⁺=279.1.

Compound 16 is prepared in a single step from the final intermediate, as follows. To a solution of 3-(3,3-difluoro-1-((4-methyl-4H-1,2,4-triazol-3-yl)methyl)cyclobutyl)aniline (50.0 mg, 0.18 mmol) and 2-trifluoromethyl-6-pyridinecarboxylic acid (68.7 mg, 0.36 mmol) in acetonitrile (3 mL) was added 2,4,6-tripropyl-1,3,5,2,4,6-trioxa-triphosphorinane-2,4,6-trioxide (50 wt % in ethyl acetate, 0.25 mL, 0.36 mmol) and pyridine (0.07 mL, 0.90 mmol). The reaction was stirred at 20° C. for 3 h and concentrated under reduced pressure. The residue was purified by RP-HPLC (water 0.05% NH₃H₂O+10 mM NH₄HCO₃)-ACN 35% to 65%) to afford N-(3-(3,3-difluoro-1-((4-methyl-4H-1,2,4-triazol-3-yl)methyl)cyclobutyl)-phenyl)-6-(trifluoromethyl)picolinamide (19.6 mg, 24% yield) as a white solid. ¹H NMR (400 MHz, methanol-d₄): δ 8.45 (d, J=8.0 Hz, 1H), 8.32-8.27 (m, 1H), 8.17 (s, 1H), 8.05 (d, J=7.6 Hz, 1H), 7.73-7.70 (m, 1H), 7.48 (s, 1H), 7.35-7.31 (m, 1H), 6.74 (d, J=8.0 Hz, 1H), 3.35 (s, 2H), 3.28-3.24 (m, 2H), 3.08-3.00 (m, 2H), 2.76 (s, 3H). LCMS [M+H]⁺ or [M−H]⁻: 452.2.

### Example 14: Compounds 18 and 19

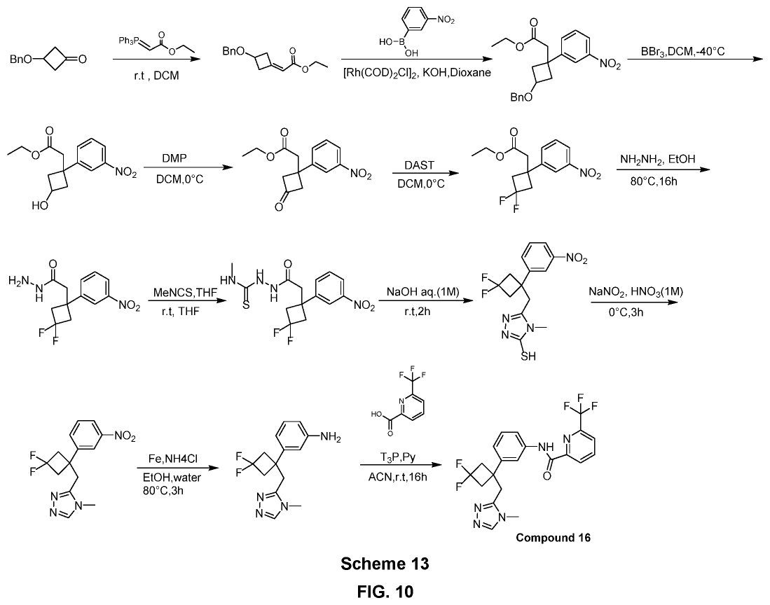

Compounds 18 (N-(3-((1R,3S)-3-fluoro-1-((4-methyl-4H-1,2,4-triazol-3-yl)methyl)-cyclobutyl)phenyl)-6-(trifluoromethyl)picolinamide) and 19 (N-(3-((1S,3R)-3-fluoro-1-((4-methyl-4H-1,2,4-triazol-3-yl)methyl)cyclobutyl)phenyl)-6-(trifluoromethyl)picolinamide) are synthesized according to Scheme 14, FIG. 11.

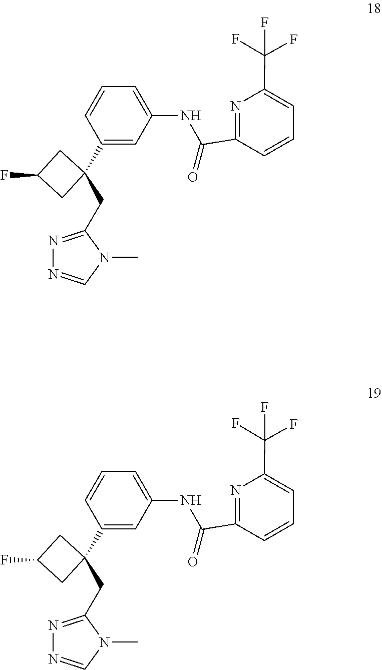 [SMILES: Cn1cnnc1C[C@]1(c2cccc(NC(=O)c3cccc(C(F)(F)F)n3)c2)C[C@H](F)C1 ; Cn1cnnc1C[C@]1(c2cccc(NC(=O)c3cccc(C(F)(F)F)n3)c2)C[C@@H](F)C1]

A first intermediate, ethyl 2-((1S,3S)-3-hydroxy-1-(3-nitrophenyl)cyclobutyl)-acetate, can be made as follows. To a mixture of ethyl 2-[3-benzyloxy-1-(3-nitrophenyl)-cyclobutyl]acetate (3.00 g, 8.12 mmol) in dichloromethane (115 mL) was added tribromoborane (3.0 mL, 31.14 mmol) at −40° C. The mixture was stirred at −40° C. for 3 h and quenched by slowly adding methanol (10 mL). The resulting mixture was concentrated under reduced pressure. The residue was purified by silica gel chromatography (mobile phase: ethyl acetate/petroleum ether, gradient 0% to 20%) to afford ethyl 2-(3-hydroxy-1-(3-nitrophenyl)cyclobutyl)acetate (1.80 g, 79% yield) as a yellow solid. The above racemate (1.80 g, 6.45 mmol) was further purified by chiral SFC (Column=Daicel Chiralcel OJ-H; Column dimensions=250 mm×30 mm×5 μm; Detection wavelength=250 nM; Flow rate=60 mL/min; Run time=4 min; Column temperature=25° C.) with 0.1% ammonium hydroxide-30% ethanol-carbon dioxide) to afford ethyl 2-((1S,3S)-3-hydroxy-1-(3-nitrophenyl)cyclobutyl)acetate (peak 1, retention time=2.122 min) (900.0 mg, 50% yield) as a white solid. ¹H NMR (400 MHz, CDCl₃): δ 8.06-8.02 (m, 1H), 7.99-7.98 (m, 1H), 7.50-7.43 (m, 2H), 4.54-4.45 (m, 1H), 3.93 (q, J=7.2 Hz, 2H), 2.95-2.89 (m, 2H), 2.71 (s, 2H), 2.32-2.26 (m, 2H), 1.06 (t, J=7.2 Hz, 3H).

A second intermediate, ethyl 2-((1R,3S)-3-fluoro-1-(3-nitrophenyl)cyclobutyl)-acetate, can be made as follows. To a solution of ethyl 2-((1 S, 3S)-3-hydroxy-1-(3-nitrophenyl)cyclobutyl)acetate (300.0 mg, 1.07 mmol) and perfluorobutanesulfonyl fluoride (486.7 mg, 1.61 mmol) in dichloromethane (10 mL) was added 2-tert-butyl-1,1,3,3-tetramethylguanidine (331.2 mg, 1.93 mmol) at 25° C. The mixture was stirred for 16 h and then concentrated under reduced pressure. The residue was purified by silica gel chromatography (mobile phase: ethyl acetate/petroleum ether, gradient 0% to 20%) to afford ethyl 2-((1R,3S)-3-fluoro-1-(3-nitrophenyl)cyclobutyl)acetate (200 mg, 66% yield) as a colorless oil. ¹H NMR (400 MHz, CDCl₃): δ 8.15 (s, 1H), 8.10 (d, J=8.4 Hz, 1H), 7.64 (d, J=7.6 Hz, 1H), 7.55-7.45 (m, 1H), 5.11-4.85 (m, 1H), 3.98 (q, J=7.2 Hz, 2H), 3.06-2.94 (m, 2H), 2.92 (s, 2H), 2.81-2.58 (m, 2H), 1.12 (t, J=7.2 Hz, 3H). Structure was assigned by NOE.

A third intermediate, 2-((1R,3S)-3-fluoro-1-(3-nitrophenyl)cyclobutyl)acetohydrazide, is made as follows. To a solution of ethyl 2-((1R,3S)-3-fluoro-1-(3-nitrophenyl)-cyclobutyl)acetate (490.0 mg, 1.74 mmol) in methanol (5 mL) was added hydrazine monohydrate (0.64 mL, 17.42 mmol). The mixture was heated at 80° C. for 12 h and concentrated under reduced pressure to afford crude 2-((1R,3S)-3-fluoro-1-(3-nitrophenyl)cyclobutyl)acetohydrazide (465.0 mg, 99% yield) as yellow oil. LCMS [M+H]⁺=268.0.

A fourth intermediate, 2-(2-((1R,3S)-3-fluoro-1-(3-nitrophenyl)cyclobutyl)acetyl)-N-methylhydrazinecarbothioamide, can be made from the third intermediate in the following step. To a mixture of ethyl 2-((1R,3S)-3-fluoro-1-(3-nitrophenyl)cyclobutyl)acetohydrazide (465.0 mg, 1.74 mmol) in tetrahydrofuran (10 mL) was added methyl isothiocyanate (152.7 mg, 2.09 mmol) at 25° C. The mixture was stirred at 25° C. for 16 h and concentrated under reduced pressure to afford crude 2-(2-((1R,3S)-3-fluoro-1-(3-nitrophenyl)cyclobutyl)acetyl)-N-methylhydrazinecarbothioamide (590.0 mg, 99% yield) as a yellow solid. LCMS [M+H]⁺=341.1.

A fifth intermediate, 5-(((1R,3S)-3-fluoro-1-(3-nitrophenyl)cyclobutyl)methyl)-4-methyl-4H-1,2,4-triazole-3-thiol, can be made from the fourth intermediate in the following way. A mixture of 2-(2-((1R,3S)-3-fluoro-1-(3-nitrophenyl)cyclobutyl)acetyl)-N-methylhydrazinecarbothioamide (590.0 mg, 1.73 mmol) in aqueous sodium hydroxide (1 M, 5.0 mL, 5.0 mmol) was stirred at 25° C. for 1 h and then adjusted to pH=5 by addition of 1 M HCl solution. The formed solid was collected by filtration and dried to afford crude 5-(((1R,3S)-3-fluoro-1-(3-nitrophenyl)cyclobutyl)methyl)-4-methyl-4H-1,2,4-triazole-3-thiol (555 mg, 99% yield) as a yellow solid. LCMS [M+H]⁺=323.0.

A sixth intermediate, 3-((3-fluoro-1-(3-nitrophenyl)cyclobutyl)methyl)-4-methyl-4H-1,2,4-triazole, can be made from the fifth intermediate in the following way. To a solution of 5-(((1R,3S)-3-fluoro-1-(3-nitrophenyl)cyclobutyl)methyl)-4-methyl-4H-1,2,4-triazole-3-thiol (555.0 mg, 1.72 mmol) in water (5 mL) and acetonitrile (2 mL) was added sodium nitrite (590 mg, 8.61 mmol), followed by addition of 1 M nitric acid (8.6 mL, 8.6 mmol) dropwise at 0° C. After addition, the mixture was stirred for another 1 h at 20° C. and quenched by addition of saturated aqueous NaHCO₃ (13 mL). The resulting mixture was extracted with dichloromethane (3×30 mL). The combined organic layers were washed with brine (20 mL), dried and concentrated under reduced pressure. The residue was purified by silica gel chromatography (mobile phase: methanol/dichloromethane, gradient 0% to 10%) to afford 3-((3-fluoro-1-(3-nitrophenyl)cyclobutyl)methyl)-4-methyl-4H-1,2,4-triazole (400 mg, 80% yield) as a yellow solid. ¹H NMR indicated the product was a 2/3 mixture of cis/trans mixture at this stage. ¹H NMR (400 MHz, CDCl₃): δ 8.10-7.88 (m, 3H), 7.50-7.39 (m, 1H), 7.37-7.35 (m, 0.6H), 7.16-7.14 (m, 0.4H), 5.47-5.26 (m, 0.4H), 5.10-4.90 (m, 0.6H), 3.34-3.09 (m, 3H), 3.09-2.78 (m, 6H), 2.68-2.57 (m, 1H).

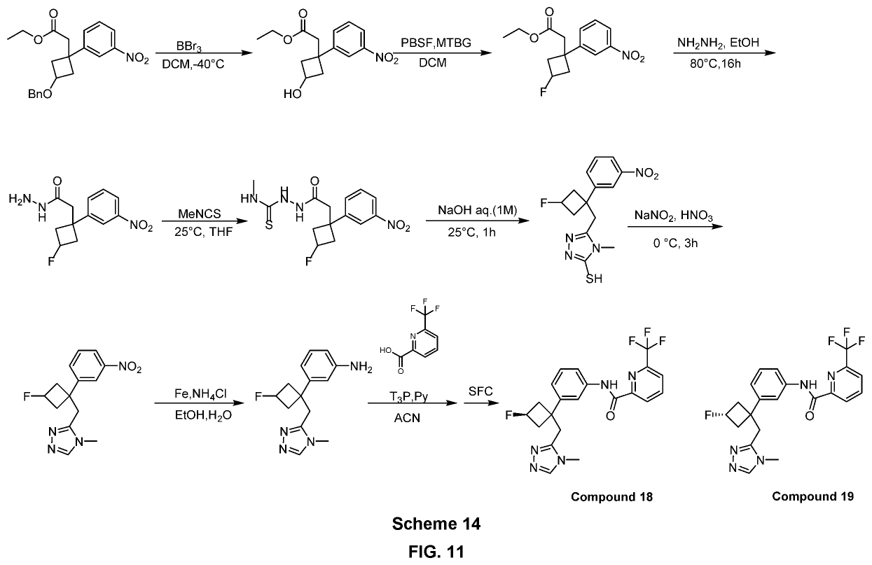

A final intermediate on the route of FIG. 12 to compounds 18 and 19, 3-(3-fluoro-1-((4-methyl-4H-1,2,4-triazol-3-yl)methyl)cyclobutyl)aniline, can be made as follows. To a mixture of 3-((3-fluoro-1-(3-nitrophenyl)cyclobutyl)methyl)-4-methyl-4H-1,2,4-triazole (200.0 mg, 0.69 mmol) in ethanol (10 mL) and water (10 mL) was added iron powder (384.8 mg, 6.89 mmol) and ammonium chloride (368.5 mg, 6.89 mmol). The mixture was stirred at 80° C. for 2 h under nitrogen atmosphere. After cooling, the mixture was filtered and the filtrate was concentrated under reduced pressure. The residue was dissolved in dichloromethane/methanol (v/v=10/1, 30 mL) and filtered, the filtrate cake was washed with dichloromethane (2×10 mL). The combined the organic phases were concentrated under reduced pressure. The residue was purified by preparative TLC (mobile phase: 10% methanol in dichloromethane) to afford 3-(3-fluoro-1-((4-methyl-4H-1,2,4-triazol-3-yl)methyl)cyclobutyl)aniline (140 mg, 78% yield) as yellow oil. ¹H NMR (400 MHz, CDCl₃): δ 7.85 (s, 1H), 7.04-6.99 (m, 1H), 6.54-6.49 (m, 1H), 6.38-6.24 (m, 1H), 6.18-6.04 (m, 1H), 5.45-5.24 (m, 0.5H), 5.03-4.85 (m, 0.5H), 3.21 (s, 1H), 3.16-3.10 (m, 1H), 3.01 (s, 1H), 2.88-2.77 (m, 2H), 2.69-2.61 (m, 3H), 2.58-2.43 (m, 1H). LCMS [M+H]⁺=261.1.

Compounds 18 and 19 can be made from the final intermediate in the following way. To a mixture of 3-(3-fluoro-1-((4-methyl-4H-1,2,4-triazol-3-yl)methyl)cyclobutyl)aniline (70.0 mg, 0.27 mmol) and 2-trifluoromethyl-6-pyridinecarboxylic acid (102.8 mg, 0.54 mmol) in acetonitrile (10 mL) was added 2,4,6-tripropyl-1,3,5,2,4,6-trioxatriphosphorinane-2,4,6-trioxide (50 wt % in ethyl acetate, 0.38 mL, 0.54 mmol) and pyridine (0.1 mL, 1.34 mmol). The mixture was stirred at 20° C. for 16 h and concentrated under reduced pressure. The residue was purified by preparative TLC (mobile phase: 10% methanol in dichloromethane) to afford N-(3-(3-fluoro-1-((4-methyl-4H-1,2,4-triazol-3-yl)methyl)cyclobutyl)phenyl)-6-(trifluoromethyl)picolinamide (90 mg, 77% yield) as a yellow solid.

The above racemate was further purified by chiral SFC (Column=Daicel Chiralpak OJ-H; Column dimensions=250 mm×30 mm×10 μm; Detection wavelength=220 nm; Flow rate=70 mL/min; Run time=7 min; Column temperature=25° C.) with 0.1% ammonium hydroxide-40% ethanol-carbon dioxide) to give compounds 18 and 19 as follows.

N-(3-((1S,3R)-3-fluoro-1-((4-methyl-4H-1,2,4-triazol-3-yl)methyl)cyclobutyl)-phenyl)-6-(trifluoromethyl)picolinamide (Peak 1, retention time=3.518 min) (28 mg, 24% yield) as yellow solid (18). ¹H NMR (400 MHz, methanol-d₄): δ 8.47-8.45 (m, 1H), 8.31-8.30 (m, 1H), 8.20 (d, J=4.0 Hz, 1H), 8.07 (d, J=7.2 Hz, 1H), 7.70-7.68 (m, 1H), 7.43 (d, J=2.0 Hz, 1H), 7.34-7.30 (m, 1H), 6.71-6.69 (m, 1H), 5.48-5.27 (m, 1H), 3.22 (s, 2H), 3.19-3.14 (m, 2H), 2.76 (s, 3H), 2.74-2.54 (m, 2H). LCMS [M+H]⁺ or [M−H]⁻: 434.1.

N-(3-((1R,3S)-3-fluoro-1-((4-methyl-4H-1,2,4-triazol-3-yl)methyl)cyclobutyl)-phenyl)-6-(trifluoromethyl)picolinamide (Peak 2, retention time=3.773 min) (38.0 mg, 33% yield) as yellow solid (19). ¹H NMR (400 MHz, methanol-d₄): δ 8.45 (d, J=7.9 Hz, 1H), 8.29 (t, J=7.9 Hz, 1H), 8.17 (s, 1H), 8.06 (d, J=7.6 Hz, 1H), 7.68 (d, J=8 Hz, 1H), 7.57 (s, 1H), 7.34-7.30 (m, 1H), 6.85 (d, J=7.6 Hz, 1H), 5.03-4.96 (m, 1H), 3.35 (s, 2H), 3.03-2.92 (m, 2H), 2.83 (s, 3H), 2.82-2.70 (m, 2H). LCMS [M+H]⁺ or [M−H]⁻: 434.1.

### Example 15: Compounds 20 and 21

Compounds 20 ((R)-2-cyclopropyl-N-(3-(3-(fluoro(4-methyl-4H-1,2,4-triazol-3-yl)methyl)oxetan-3-yl)phenyl)-6-((3-fluoro-3-methylazetidin-1-yl)methyl)pyrimidine-4-carboxamide) and Compound 21 (2-cyclopropyl-6-[(3-fluoro-3-methyl-azetidin-1-yl)methyl]-N-[3-[3-[(S)-fluoro-(4-methyl-1,2,4-triazol-3-yl)methyl]oxetan-3-yl]phenyl]pyrimidine-4-carboxamide) can be made according to Scheme 15, shown in FIG. 12.

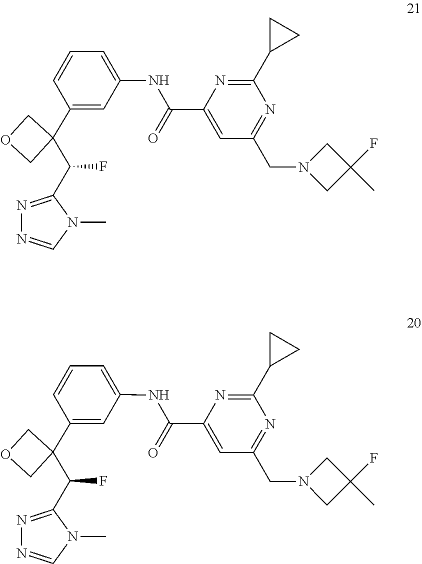 [SMILES: Cn1cnnc1[C@@H](F)C1(c2cccc(NC(=O)c3cc(CN4CC(C)(F)C4)nc(C4CC4)n3)c2)COC1 ; Cn1cnnc1[C@H](F)C1(c2cccc(NC(=O)c3cc(CN4CC(C)(F)C4)nc(C4CC4)n3)c2)COC1]

A first intermediate, methyl 2-cyclopropyl-6-((3-fluoro-3-methylazetidin-1-yl)-methyl)pyrimidine-4-carboxylate, can be made as follows. To a solution of ethyl 2-cyclopropyl-6-formylpyrimidine-4-carboxylate (150.0 mg, 0.68 mmol) in methanol (5 mL) was added 3-fluoro-3-methyl-azetidine hydrochloride (128.3 mg, 1.02 mmol) and triethylamine (142.0 μL, 1.02 mmol). The mixture was heated under microwave irradiation at 100° C. for 1 minute and cooled down to room temperature. The mixture was then added sodium cyanoborohydride (64.2 mg, 1.02 mmol) and heated at 80° C. under microwave irradiation for another 45 minutes. The reaction was quenched with 1 M HCl solution and adjusted to pH=7 by addition of saturated NaHCO₃ solution. The resulting solution was extracted with dichloromethane (3×20 mL). The combined organic phases were dried over anhydrous sodium sulphate and concentrated under reduced pressure. The residue was purified by preparative TLC (mobile phase: 10% methanol in dichloromethane) to afford methyl 2-cyclopropyl-6-((3-fluoro-3-methylazetidin-1-yl)methyl)pyrimidine-4-carboxylate (170 mg, 89.4% yield) as a yellow oil. LCMS [M+H]⁺=280.1.

A second intermediate, 2-cyclopropyl-6-((3-fluoro-3-methylazetidin-1-yl)methyl)-pyrimidine-4-carboxylic acid, can be prepared as follows. To a solution of methyl 2-cyclo-propyl-6-[(3-fluoro-3-methyl-azetidin-1-yl)methyl]pyrimidine-4-carboxylate (170.0 mg, 0.61 mmol) in methanol (3 mL) and water (3 mL) was added lithium hydroxide hydrate (38.3 mg, 0.91 mmol). The reaction mixture was stirred for 16 h at 25° C. and then adjusted to pH=6 by addition of saturated KHSO₄ solution. The mixture was concentrated under reduced pressure to afford crude 2-cyclopropyl-6-((3-fluoro-3-methylazetidin-1-yl)methyl)pyrimidine-4-carboxylic acid (160 mg, 99.1% yield) as a light-yellow solid. LCMS [M+H]⁺=266.1.

Compounds 20 and 21 can be made as follows, starting from the second intermediate.

To a mixture of (R)-3-(3-(fluoro(4-methyl-4H-1,2,4-triazol-3-yl)methyl)oxetan-3-yl)aniline (synthesized as shown in WO2019148005) (50.0 mg, 0.19 mmol) and 2-cyclopropyl-6-((3-fluoro-3-methylazetidin-1-yl)methyl)pyrimidine-4-carboxylic acid (55.63 mg, 0.21 mmol) in acetonitrile (3 mL) were added 2,4,6-tripropyl-1,3,5,2,4,6-trioxatriphosphorinane-2,4,6-trioxide (50 wt % in ethyl acetate, 0.27 mL, 0.38 mmol) and pyridine (0.05 mL, 0.57 mmol). The reaction mixture was stirred for 2 h at 25° C. and concentrated under reduced pressure. The residue was purified by RP-HPLC (water (0.2% formic acid)-ACN 12% to 42%) to afford (R)-2-cyclopropyl-N-(3-(3-(fluoro(4-methyl-4H-1,2,4-triazol-3-yl)methyl)oxetan-3-yl)phenyl)-6-((3-fluoro-3-methylazetidin-1-yl)methyl)pyrimidine-4-carboxamide (5.6 mg, 5.7% yield) as a white solid (20). ¹H NMR (400 MHz, CDCl₃): δ 9.80 (s, 1H), 7.91 (s, 2H), 7.77-7.69 (m, 1H), 7.35-7.28 (m, 2H), 6.71-6.68 (m, 1H), 6.47 (d, J=45.6 Hz, 1H), 5.30-5.20 (m, 3H), 5.00-4.96 (m, 1H), 4.18-3.84 (m, 4H), 3.76-3.59 (m, 2H), 3.00 (s, 3H), 2.40-2.35 (m, 1H), 1.70 (d, J=22.0 Hz, 3H), 1.23-1.15 (m, 4H). LCMS [M+H]⁺ or [M−H]⁻=510.1.

To a mixture of 3-[3-[(S)-fluoro-(4-methyl-1,2,4-triazol-3-yl)methyl]oxetan-3-yl]aniline (synthesized as shown in WO2019148005) (50.0 mg, 0.19 mmol) and 2-cyclopropyl-6-((3-fluoro-3-methylazetidin-1-yl)methyl)pyrimidine-4-carboxylic acid (55.63 mg, 0.21 mmol) in acetonitrile (3 mL) was added 2,4,6-tripropyl-1,3,5,2,4,6-trioxatriphosphorinane-2,4,6-trioxide (50 wt % in ethyl acetate, 0.27 mL, 0.38 mmol) and pyridine (0.05 mL, 0.57 mmol). The reaction mixture was stirred for 2 h at 25° C. then concentrated under reduced pressure. The residue was purified by RP-HPLC (water (0.2% formic acid)-ACN 12% to 42%) to afford 2-cyclopropyl-6-[(3-fluoro-3-methyl-azetidin-1-yl)methyl]-N-[3-[3-[(S)-fluoro-(4-methyl-1,2,4-triazol-3-yl)methyl]oxetan-3-yl]phenyl]-pyrimidine-4-carboxamide (21), (4.93 mg, 5% yield) as a white solid. ¹H NMR (400 MHz, CDCl₃): δ 9.81 (s, 1H), 7.92 (s, 2H), 7.74-7.71 (m, 1H), 7.37-7.29 (m, 2H), 6.72-6.70 (m, 1H), 6.48 (d, J=46 Hz, 1H), 5.32-5.22 (m, 3H), 5.02-4.98 (m, 1H), 4.19-3.90 (m, 4H), 3.81-3.63 (m, 2H), 3.00 (s, 3H), 2.42-2.36 (m, 1H), 1.72 (d, J=22.0 Hz, 3H), 1.27-1.18 (m, 4H). LCMS [M+H]⁺ or [M−H]⁻=510.1.

### Example 16: Compounds 22 and 23

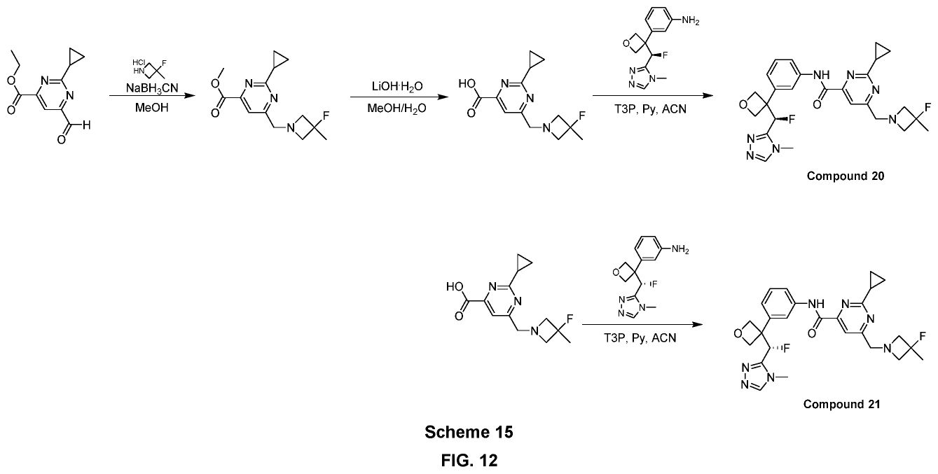

Compound 22, 4-((S)-1-(5-azaspiro[2.4]heptan-5-yl)ethyl)-6-cyclopropyl-N-(3-(3-((R)-fluoro(4-methyl-4H-1,2,4-triazol-3-yl)methyl)oxetan-3-yl)phenyl)picolinamide, and 23, 4-((R)-1-(5-azaspiro[2.4]heptan-5-yl)ethyl)-6-cyclopropyl-N-(3-(3-((R)-fluoro(4-methyl-4H-1,2,4-triazol-3-yl)methyl)oxetan-3-yl)phenyl)picolinamide can be synthesized according to Scheme 16, in FIG. 13.

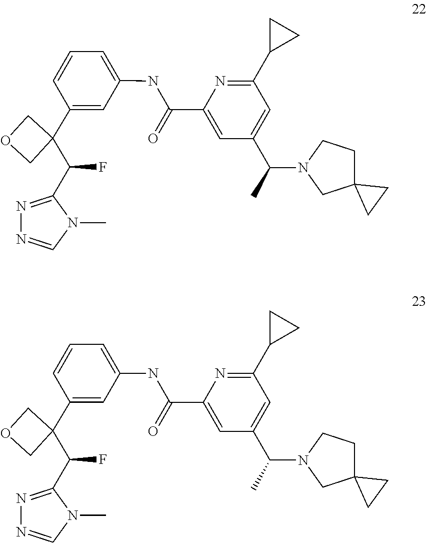 [SMILES: C[C@@H](c1cc(C(=O)Nc2cccc(C3([C@@H](F)c4nncn4C)COC3)c2)nc(C2CC2)c1)N1CCC2(CC2)C1 ; C[C@H](c1cc(C(=O)Nc2cccc(C3([C@@H](F)c4nncn4C)COC3)c2)nc(C2CC2)c1)N1CCC2(CC2)C1]

A first Intermediate, 2,6-dichloro-N-methoxy-N-methylisonicotinamide, can be formed as follows. To a solution of 2,6-dichloroisonicotinic acid (5.0 g, 26.04 mmol) and 1-[bis(dimethylamino)methylene]-1H-1,2,3-triazolo[4,5-b]pyridinium-3-oxide hexafluoro-phosphate (12.4 g, 32.55 mmol) in dichloromethane (100 mL) was added diisopropylethylamine (12.9 mL, 78.13 mmol) and N,O-dimethylhydroxylamine hydrochloride (3.2 g, 32.55 mmol). The mixture was stirred at 25° C. for 1 h under nitrogen and concentrated. The residue was purified by silica gel chromatography (mobile phase: ethyl acetate/petroleum ether, gradient 0% to 2%) to afford 2,6-dichloro-N-methoxy-N-methylisonicotinamide (5.5 g, 89.8% yield) as a colorless oil.

A second intermediate, 1-(2,6-dichloropyridin-4-yl)ethanone, can be made from the first intermediate, as follows. To a solution of 2,6-dichloro-N-methoxy-N-methylisonicotinamide (3.3 g, 14.04 mmol) in tetrahydrofuran (60.0 mL) was added methyl magnesium bromide (3.0 M in tetrahydrofuran, 6.3 mL, 18.95 mmol) at −78° C. The resulting mixture was stirred at −78° C. for 1 h and quenched by addition of water (100 mL). The solution was extracted with ethyl acetate (3×100 mL). The combined organic phases were washed with brine (100 mL), dried over sodium sulfate and concentrated. The residue was purified by silica gel chromatography (mobile phase: ethyl acetate/petroleum ether, gradient 0% to 8%) to afford 1-(2,6-dichloropyridin-4-yl)ethanone (2.5 g, 93.7% yield) as a colorless oil.

A fourth intermediate, 1-(2-chloro-6-cyclopropylpyridin-4-yl)ethanone, is made as follows. To a mixture of the second intermediate, (1-(2,6-dichloro-4-pyridyl)ethanone), (500.0 mg, 2.63 mmol) and cyclopropylboronic acid (248.6 mg, 2.89 mmol) in toluene (30 mL) and water (3 mL) was added palladium (II) acetate (59.1 mg, 0.26 mmol), tricyclohexylphosphane (147.6 mg, 0.53 mmol) and potassium pyrophosphate (2.0 g, 9.47 mmol). The mixture was stirred at 100° C. for 8 h and filtered. The filtrate was concentrated under reduced pressure and the residue was purified by silica gel chromatography (mobile phase: ethyl acetate/petroleum ether, gradient 0% to 5%) to afford 1-(2-chloro-6-cyclopropylpyridin-4-yl)ethanone (250.0 mg, 48.6% yield) as colorless oil. LCMS [M+H]⁺=196.1.

A fourth intermediate, methyl 4-acetyl-6-cyclopropylpicolinate, can be made as follows. To a solution of 1-(2-chloro-6-cyclopropyl-4-pyridyl)ethanone (1.5 g, 7.67 mmol) in methanol (20.0 mL) and N,N-dimethylformamide (5.0 mL) was added 1,1-bis(diphenylphosphino)ferrocene-palladium(II) (561.0 mg, 0.77 mmol) and trimethylamine (3.2 mL, 230 mmol). The mixture was stirred at 80° C. for 16 h under CO (45 psi) and cooled. The mixture was diluted with water (100 mL) and extracted with ethyl acetate (3×100 mL). The combined the organic layers were washed with brine (50 mL), dried over anhydrous sodium sulphate and concentrated. The residue was purified by silica gel chromatography (mobile phase: ethyl acetate/petroleum ether, gradient 0% to 10%) to afford methyl 4-acetyl-6-cyclopropylpicolinate (1.1 g, 65.4% yield) as a yellow solid. LCMS [M+H]⁺=220.1.

A fifth intermediate, 4-acetyl-6-cyclopropylpicolinic acid, can be made as follows, from the fourth intermediate. A mixture of methyl 4-acetyl-6-cyclopropyl-pyridine-2-carboxylate (290.0 mg, 1.32 mmol) and lithium hydroxide hydrate (126.7 mg, 5.29 mmol) in methanol (10 mL) and water (1 mL) was stirred at 25° C. for 12 h and concentrated under reduced pressure. The residue was diluted with water (15 mL) and adjusted to pH=5 by addition of 1 M HCl. The result solution was extracted with ethyl acetate (3×15 mL). The combined organic phases were washed with brine (2×15 mL), dried over sodium sulfate and concentrated to give 4-acetyl-6-cyclopropylpicolinic acid (160.0 mg, 58.9% yield) as light yellow solid. LCMS [M+H]⁺=206.1.

A sixth intermediate, (R)-4-acetyl-6-cyclopropyl-N-(3-(3-(fluoro(4-methyl-4H-1,2,4-triazol-3-yl)methyl)oxetan-3-yl)phenyl)picolinamide, can be synthesized in the following way. To a solution of 4-acetyl-6-cyclopropyl-pyridine-2-carboxylic acid (150.0 mg, 0.73 mmol) and 3-[3-[(R)-fluoro-(4-methyl-1,2,4-triazol-3-yl)methyl]oxetan-3-yl]aniline (synthesized as described in WO2019148005, 200.0 mg, 0.76 mmol) in acetonitrile (40 mL) was added 2,4,6-tripropyl-1,3,5,2,4,6-trioxa-triphosphorinane-2,4,6-trioxide (50 wt % in ethyl acetate, 606.6 mg, 0.95 mmol) and pyridine (153.4 uL, 1.91 mmol). The reaction was stirred at 25° C. for 16 h and concentrated under reduced pressure. The residue was purified by silica gel chromatography (mobile phase: methanol/dichloromethane, gradient 0% to 5%) to afford (R)-4-acetyl-6-cyclopropyl-N-(3-(3-(fluoro(4-methyl-4H-1,2,4-triazol-3-yl)methyl)oxetan-3-yl)phenyl)picolinamide (220.0 mg, 64.2% yield) as yellow solid. LCMS [M+H]⁺=450.1.

Finally, a racemic mixture of Compounds 22 and 23 can be made as follows. To a solution of the sixth intermediate, 4-acetyl-6-cyclopropyl-N-[3-[3-[rac-(R)-fluoro-(4-methyl-1,2,4-triazol-3-yl)methyl]oxetan-3-yl]phenyl]pyridine-2-carboxamide, (100.0 mg, 0.22 mmol) in methanol (5 mL) was added 5-azaspiro[2.4]heptane hydrochloride (53.5 mg, 0.40 mmol) and triethylamine (0.12 mL, 0.89 mmol). The mixture was heated under microwave irradiation at 100° C. for 1 minute and cooled down to room temperature. The mixture was then added sodium cyanoborohydride (55.9 mg, 0.89 mmol) and heated at 80° C. under microwave irradiation for another 30 minutes. The reaction was quenched with 1 M HCl solution and the result solution was adjusted to pH=7 by addition of saturated NaHCO₃ solution. The resulting solution was extracted with dichloromethane (3×20 mL). The combined organic phases were dried over anhydrous sodium sulphate and concentrated under reduced pressure. The residue was purified by preparative TLC (mobile phase: 10% methanol in dichloromethane) to give 4-(1-(5-azaspiro[2.4]heptan-5-yl)ethyl)-6-cyclopropyl-N-(3-(3-((R)-fluoro(4-methyl-4H-1,2,4-triazol-3-yl)methyl)oxetan-3-yl)phenyl)picolinamide (100.0 mg, 84.7% yield) as yellow solid. LCMS [M+H]⁺=531.2.

The above racemate (130 mg, 0.25 mmol) was further purified by chiral SFC [(Column=Daicel Chiralpak IC; Column dimensions=250 mm×30 mm×5 μm; Detection wavelength=250 nM; Flow rate=60 mL/min; Run time=12 min; Column temperature=25° C.) with 0.1% ammonium hydroxide-45% EtOH-carbon dioxide] to give compounds 22 and 23, as follows.

6-cyclopropyl-4-[(1 S)-1-(5-azaspiro[2.4]heptan-5-yl)ethyl]-N-[3-[3-[(R)-fluoro-(4-methyl-1,2,4-triazol-3-yl)methyl]oxetan-3-yl]phenyl]pyridine-2-carboxamide (Peak 1, retention time=8.39 min) (11.27 mg, 11% yield) as a white solid (22). ¹H NMR (400 MHz, methanol-d₄): δ 8.28 (s, 1H), 7.97 (s, 1H), 7.70 (d, J=8.0 Hz, 1H), 7.46-7.43 (m, 2H), 7.37-7.33 (m, 1H), 6.80-6.78 (m, 1H), 6.30 (d, J=40.0 Hz, 1H), 5.48-5.00 (m, 4H), 3.42-3.40 (m, 1H), 3.07 (s, 3H), 2.66-2.62 (m, 1H), 2.60-2.59 (m, 2H), 2.43-2.40 (m, 1H), 2.23-2.22 (m, 1H), 1.87-1.83 (m, 2H), 1.41 (d, J=8.0 Hz, 3H), 1.15-1.09 (m, 4H), 0.58-0.54 (m, 4H). LCMS [M+H]⁺ or [M−H]⁻=531.2.

6-cyclopropyl-4-[(1R)-1-(5-azaspiro[2.4]heptan-5-yl)ethyl]-N-[3-[3-[(R)-fluoro-(4-methyl-1,2,4-triazol-3-yl)methyl]oxetan-3-yl]phenyl]pyridine-2-carboxamide (Peak 2, retention time=9.678 min) (11.93 mg, 11.6% yield) as a white solid (23). ¹H NMR (400 MHz, methanol-d₄): δ 8.28 (s, 1H), 7.97 (s, 1H), 7.70 (d, J=8.0 Hz, 1H), 7.46-7.43 (m, 2H), 7.37-7.33 (m, 1H), 6.80-6.78 (m, 1H), 6.30 (d, J=40 Hz, 1H), 5.48-5.00 (m, 4H), 3.42-3.40 (m, 1H), 3.07 (s, 3H), 2.66-2.62 (m, 1H), 2.60-2.59 (m, 2H), 2.43-2.40 (m, 1H), 2.23-2.22 (m, 1H), 1.87-1.83 (m, 2H), 1.41 (d, J=8.0 Hz, 3H), 1.15-1.09 (m, 4H), 0.60-0.57 (m, 4H). LCMS [M+H]⁺ or [M−H]⁻=531.2.

### Example 17: Compounds 24 and 25

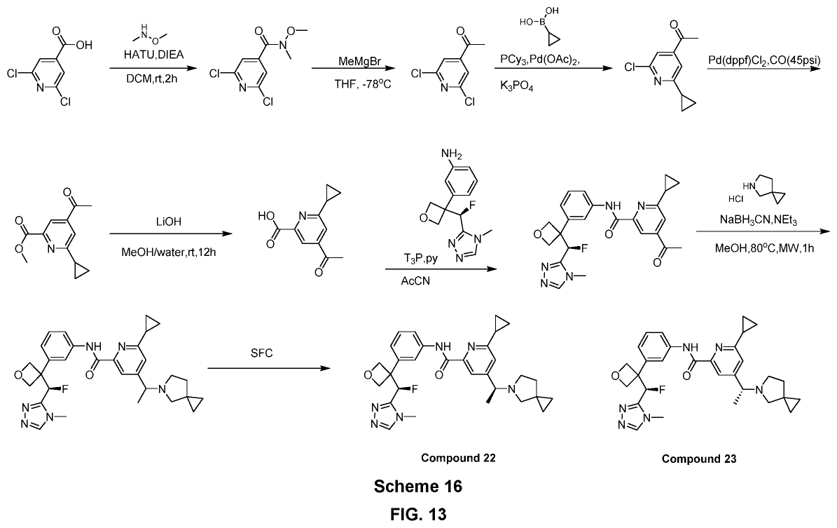

Compounds 24, 4-((S)-1-(5-azaspiro[2.4]heptan-5-yl)ethyl)-6-cyclopropyl-N-(3-(3-((S)-fluoro(4-methyl-4H-1,2,4-triazol-3-yl)methyl)oxetan-3-yl)phenyl)picolinamide, and 25, 4-((R)-1-(5-azaspiro[2.4]heptan-5-yl)ethyl)-6-cyclopropyl-N-(3-(3-((S)-fluoro(4-methyl-4H-1,2,4-triazol-3-yl)methyl)oxetan-3-yl)phenyl)picolinamide, can be made according to Scheme 17 in FIG. 14.

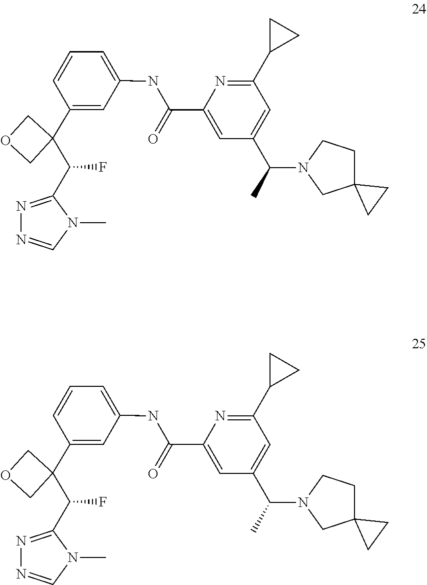 [SMILES: C[C@@H](c1cc(C(=O)Nc2cccc(C3([C@H](F)c4nncn4C)COC3)c2)nc(C2CC2)c1)N1CCC2(CC2)C1 ; C[C@H](c1cc(C(=O)Nc2cccc(C3([C@H](F)c4nncn4C)COC3)c2)nc(C2CC2)c1)N1CCC2(CC2)C1]

The (S) isomer of the sixth intermediate of Scheme 23 (as defined by the chirality at the fluorinated carbon), (S)-4-acetyl-6-cyclopropyl-N-(3-(3-(fluoro(4-methyl-4H-1,2,4-triazol-3-yl)methyl)oxetan-3-yl)phenyl)picolinamide, can be made as follows. To a solution of 4-acetyl-6-cyclopropyl-pyridine-2-carboxylic acid (300.0 mg, 1.46 mmol) and 3-[3-[(S)-fluoro-(4-methyl-1,2,4-triazol-3-yl)methyl]oxetan-3-yl]aniline (WO2019148005, 383.4 mg, 1.46 mmol) in acetonitrile (10 mL) was added 2,4,6-tripropyl-1,3,5,2,4,6-trioxa-triphosphorinane-2,4,6-trioxide (50 wt % in ethyl acetate, 1.4 g, 2.19 mmol) and pyridine (443.06 uL, 4.39 mmol). The reaction was stirred at 25° C. for 16 h and concentrated under reduced pressure. The residue was purified by silica gel chromatography (mobile phase: methanol/dichloromethane, gradient 0% to 8%) to afford (S)-4-acetyl-6-cyclopropyl-N-(3-(3-(fluoro(4-methyl-4H-1,2,4-triazol-3-yl)methyl)oxetan-3-yl)phenyl)picolinamide (600 mg, 91.3% yield) as white solid. LCMS [M+H]⁺=450.1.

A racemate of compounds 24 and 25 can be made as follows. To a solution of 4-acetyl-6-cyclopropyl-N-[3-[3-[rac-(S)-fluoro-(4-methyl-1,2,4-triazol-3-yl)methyl]oxetan-3-yl]phenyl]pyridine-2-carboxamide (180.0 mg, 0.40 mmol) in methanol (4 mL) was added 5-azaspiro[2.4]heptane;hydrochloride (160.5 mg, 1.2 mmol) and triethylamine (101.3 mg, 1.0 mmol). The mixture was heated under microwave irradiation at 100° C. for 1 minute and cooled down to room temperature. The mixture was then added sodium cyanoborohydride (45.3 mg, 0.72 mmol) and heated at 80° C. under microwave irradiation for another 30 minutes. The reaction was quenched with 1 M HCl solution and adjusted to pH=7 by addition of saturated NaHCO₃ solution. The resulting solution was extracted with dichloromethane (3×20 mL). The combined organic phases were dried over anhydrous sodium sulphate and concentrated under reduced pressure. The residue was purified by silica gel chromatography (mobile phase: methanol/dichloromethane, gradient 0% to 8%) to give 4-(1-(5-azaspiro[2.4]heptan-5-yl)ethyl)-6-cyclopropyl-N-(3-(3-((S)-fluoro(4-methyl-4H-1,2,4-triazol-3-yl)methyl)oxetan-3-yl)phenyl)picolinamide (160.0 mg, 75.3% yield) as yellow solid. LCMS [M+H]⁺=531.2.

The above racemate (130 mg, 0.25 mmol) was further purified by chiral SFC [(Column=Daicel Chiralcel OD; Column dimensions=250 mm×30 mm×5 μm; Detection wavelength=220 nm; Flow rate=60 mL/min; Run time=8 min; Column temperature=25° C.) with 0.1% ammonium hydroxide-40% EtOH-carbon dioxide] to give compounds 24 and 25 as follows.

4-((S)-1-(5-azaspiro[2.4]heptan-5-yl)ethyl)-6-cyclopropyl-N-(3-(3-((S)-fluoro(4-methyl-4H-1,2,4-triazol-3-yl)methyl)oxetan-3-yl)phenyl)picolinamide (Peak 1, retention time=3.986 min) (35.0 mg, 21.9% yield) as a white solid (24). ¹H NMR (400 MHz, CDCl₃): δ 9.89 (s, 1H), 7.97 (s, 1H), 7.89 (s, 1H), 7.61 (br d, J=8.0 Hz, 1H), 7.43 (br s, 1H), 7.33-7.28 (m, 2H), 6.59 (d, J=7.6 Hz, 1H), 6.48 (d, J=45.6 Hz, 1H), 5.30-5.21 (m, 3H), 4.98 (t, J=6.0 Hz, 1H), 3.35 (br s, 1H), 2.94 (s, 3H), 2.79 (br s, 1H), 2.70-2.50 (m, 2H), 2.43 (br s, 1H), 2.15 (br d, J=6.8 Hz, 1H), 1.86-1.83 (m, 2H), 1.40 (br d, J=6.0 Hz, 3H), 1.12-1.10 (m, 4H), 0.55 (s, 4H). LCMS [M+H]⁺ or [M−H]⁻=531.2.

4-((R)-1-(5-azaspiro[2.4]heptan-5-yl)ethyl)-6-cyclopropyl-N-(3-(3-((S)-fluoro(4-methyl-4H-1,2,4-triazol-3-yl)methyl)oxetan-3-yl)phenyl)picolinamide (Peak 2, retention time=4.140 min) (30.0 mg, 18.8% yield) as a white solid (25). ¹H NMR (400 MHz, CDCl₃): δ 9.90 (s, 1H), 7.97 (s, 1H), 7.88 (s, 1H), 7.62 (br d, J=8.0 Hz, 1H), 7.42 (s, 1H), 7.34-7.28 (m, 2H), 6.59 (d, J=7.6 Hz, 1H), 6.48 (d, J=45.6 Hz, 1H), 5.30-5.21 (m, 3H), 4.98 (t, J=6.0 Hz, 1H), 3.35 (br d, J=6.0 Hz, 1H), 2.94 (s, 3H), 2.81 (br d, J=7.6 Hz, 1H), 2.63 (br d, J=7.2 Hz, 1H), 2.55 (br d, J=8.8 Hz, 1H), 2.42 (m, 1H), 2.22-2.11 (m, 1H), 1.83 (br d, J=6.0 Hz, 2H), 1.40 (br d, J=6.4 Hz, 3H), 1.12-1.09 (m, 4H), 0.55 (s, 4H). LCMS [M+H]⁺ or [M−H]⁻=531.2.

### Example 18: Compound 26

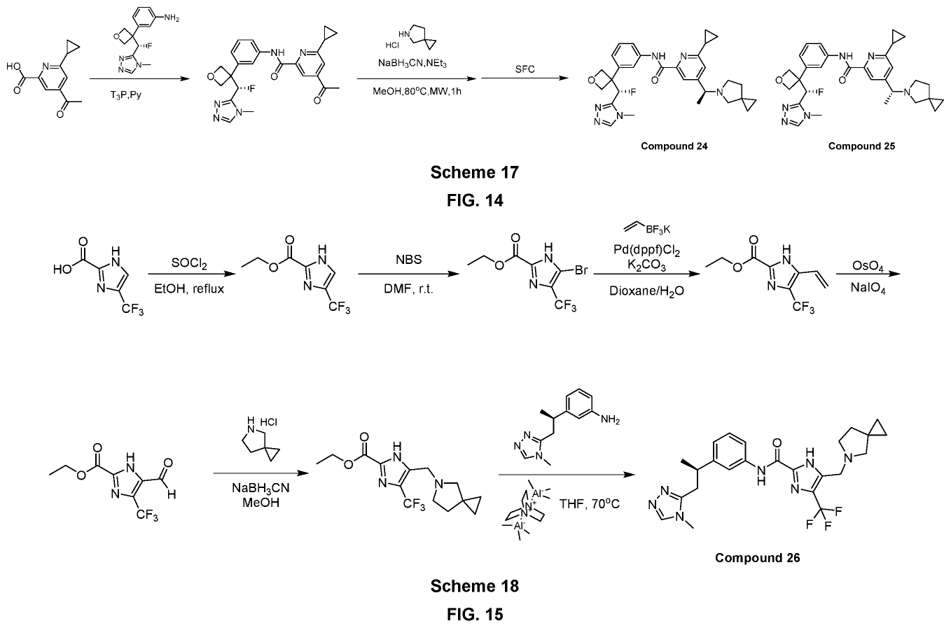

Compound 26, (R)-5-(5-azaspiro[2.4]heptan-5-ylmethyl)-N-(3-(1-(4-methyl-4H-1,2,4-triazol-3-yl)propan-2-yl)phenyl)-4-(trifluoromethyl)-1H-imidazole-2-carboxamide, can be synthesized according to Scheme 18, FIG. 15.

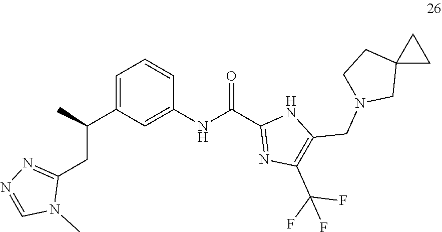 [SMILES: C[C@H](Cc1nncn1C)c1cccc(NC(=O)c2nc(C(F)(F)F)c(CN3CCC4(CC4)C3)[nH]2)c1]

A first intermediate, ethyl 4-(trifluoromethyl)-1H-imidazole-2-carboxylate, was synthesized from 4-(trifluoromethyl)-1H-imidazole-2-carboxylic acid (5.50 g, 30.54 mmol), suspended in ethanol (150 mL), to which thionyl chloride (22.16 mL, 305.42 mmol) was carefully added dropwise under a water bath. The resulting mixture was heated at 90° C. for 16 h and concentrated under reduced pressure. The residue was diluted with ethyl acetate (200 mL), washed with water (3×100 mL), brine (100 mL), dried and concentrated under reduced pressure to afford crude ethyl 4-(trifluoromethyl)-1H-imidazole-2-carboxylate (6.30 g, 99.1% yield) as a white solid.

A second intermediate, ethyl 5-bromo-4-(trifluoromethyl)-1H-imidazole-2-carboxylate, was made as follows. To a solution of ethyl 4-(trifluoromethyl)-1H-imidazole-2-carboxylate (4.00 g, 19.22 mmol) in N,N-dimethylformamide (130 mL) was added 1-bromo-2,5-pyrrolidinedione (6.84 g, 38.44 mmol) at 25° C. The reaction was stirred at 25° C. for 16 h and diluted with ethyl acetate (100 mL). The solution was washed with saturated aqueous sodium bicarbonate (150 mL), brine (150 mL), dried and concentrated to afford crude ethyl 5-bromo-4-(trifluoromethyl)-1H-imidazole-2-carboxylate (5.5 g, 99.7% yield) as a light yellow oil.

A third intermediate, ethyl 4-(trifluoromethyl)-5-vinyl-1H-imidazole-2-carboxylate, was made from the second intermediate, as follows. To a mixture of ethyl 5-bromo-4-(trifluoromethyl)-1H-imidazole-2-carboxylate (1.60 g, 5.57 mmol) in 1,4-dioxane (32 mL) and water (8 mL) was added 1,1′-bis(diphenylphosphino)ferrocene palladium dichloride (407.9 mg, 0.56 mmol), potassium vinyltrifluoroborate (1.34 g, 10.03 mmol) and potassium carbonate (1.54 g, 11.15 mmol). The reaction mixture was stirred at 90° C. for 16 h under nitrogen protection and filtered through a Celite pad. The filtrate was concentrated under reduced pressure and the residue was purified by silica gel chromatography (mobile phase: ethyl acetate/petroleum ether, gradient 0% to 8%) to afford ethyl 4-(trifluoromethyl)-5-vinyl-1H-imidazole-2-carboxylate (330.0 mg, 25.3% yield) as a dark yellow oil.

A fourth intermediate, ethyl 5-formyl-4-(trifluoromethyl)-1H-imidazole-2-carboxylate, was made as follows. To a solution of ethyl 4-(trifluoromethyl)-5-vinyl-1H-imidazole-2-carboxylate (400.0 mg, 1.71 mmol) in water (6 mL) and tetrahydrofuran (12 mL) was added osmium tetroxide (173.7 mg, 0.68 mmol). The reaction mixture was stirred at 25° C. for 16 h and diluted with ethyl acetate (25 mL). The solution was filtered through a Celite pad and the filtrate was concentrated under reduced pressure. The residue was purified by silica gel chromatography (mobile phase: ethyl acetate/petroleum ether, gradient 0% to 30%) to afford ethyl 5-formyl-4-(trifluoromethyl)-1H-imidazole-2-carboxylate (110 mg, 27.3% yield) as a light yellow oil.

A fifth intermediate, ethyl 5-(5-azaspiro[2.4]heptan-5-ylmethyl)-4-(trifluoromethyl)-1H-imidazole-2-carboxylate, was made from the fourth intermediate, as follows. To a solution of ethyl 5-formyl-4-(trifluoromethyl)-1H-imidazole-2-carboxylate (110.0 mg, 0.47 mmol) in methanol (4 mL) was added triethylamine (188.3 uL, 1.35 mmol) and 5-azaspiro[2.4]heptane hydrochloride (193.0 mg, 1.44 mmol). The reaction mixture was heated under microwave irritation for 1 minute at 100° C. and then sodium cyanoborohydride (32.2 mg, 0.51 mmol) was added. The reaction mixture was heated under microwave irritation at 80° C. for 45 minutes and cooled. The mixture was quenched by addition of 1 M HCl and adjusted to pH=7 by addition of saturated sodium bicarbonate solution. The resulting solution was extracted with ethyl acetate (3×15 mL). The combined organic layers were dried over anhydrous sodium sulfate and concentrated under reduced pressure. The residue was purified by preparative TLC (mobile phase: 70% ethyl acetate in petroleum ether) to afford ethyl 5-(5-azaspiro[2.4]heptan-5-ylmethyl)-4-(trifluoromethyl)-1H-imidazole-2-carboxylate (30.0 mg, 21.2% yield) and methyl 5-(5-azaspiro[2.4]heptan-5-ylmethyl)-4-(trifluoromethyl)-1H-imidazole-2-carboxylate (20.0 mg, 13.5% yield) both as colorless oil.

To a vial containing bis(trimethylaluminum)-1,4-diazabicyclo[2.2.2]octane adduct (50.7 mg, 0.20 mmol) was added 3-[(1R)-1-methyl-2-(4-methyl-1,2,4-triazol-3-yl)ethyl]aniline (42.8 mg, 0.20 mmol) in tetrahydrofuran (0.3 mL). The reaction mixture was stirred at 40° C. for 1 h, then a solution of the fifth intermediate, ethyl 5-(5-azaspiro[2.4]heptan-5-ylmethyl)-4-(trifluoromethyl)-1H-imidazole-2-carboxylate (20.0 mg, 0.06 mmol) and methyl 5-(5-azaspiro[2.4]heptan-5-ylmethyl)-4-(trifluoromethyl)-1H-imidazole-2-carboxylate (20.0 mg, 0.07 mmol), in tetrahydrofuran (0.3 mL) was added. The reaction mixture was stirred at 70° C. for 16 h and diluted with methanol (10 mL). The solution was filtered through a pad of Celite and rinsed with methanol (20 mL). The combined filtrate was concentrated under reduced pressure. The residue was purified by RP-HPLC (water (0.05% NH₃H₂O+10 mM NH₄HCO₃)-ACN 26% to 49%) to afford (R)-5-(5-azaspiro[2.4]heptan-5-ylmethyl)-N-(3-(1-(4-methyl-4H-1,2,4-triazol-3-yl)propan-2-yl)phenyl)-4-(trifluoromethyl)-1H-imidazole-2-carboxamide, Compound 26, (3.7 mg, 11.4% yield) as a white solid. ¹H NMR (400 MHz, CDCl₃): δ 8.92 (br s, 1H), 7.95 (s, 1H), 7.56 (s, 1H), 7.53 (d, J=8.0 Hz, 1H), 7.31-7.24 (m, 1H), 6.94 (d, J=7.2 Hz, 1H), 3.88 (s, 2H), 3.47-3.33 (m, 1H), 3.24 (s, 3H), 3.10 (dd, J=6.8 Hz, 14.8 Hz, 1H), 2.93 (dd, J=6.8 Hz, 14.8 Hz, 1H), 2.84 (t, J=7.2 Hz, 2H), 2.59 (s, 2H), 1.87 (t, J=6.8 Hz, 2H), 1.44 (d, J=6.8 Hz, 3H), 0.59 (s, 4H). LCMS [M+H]⁺ or [M−H]⁻=488.2.

### Example 19: Compound 35, Compound 36, Compound 37 and Compound 38

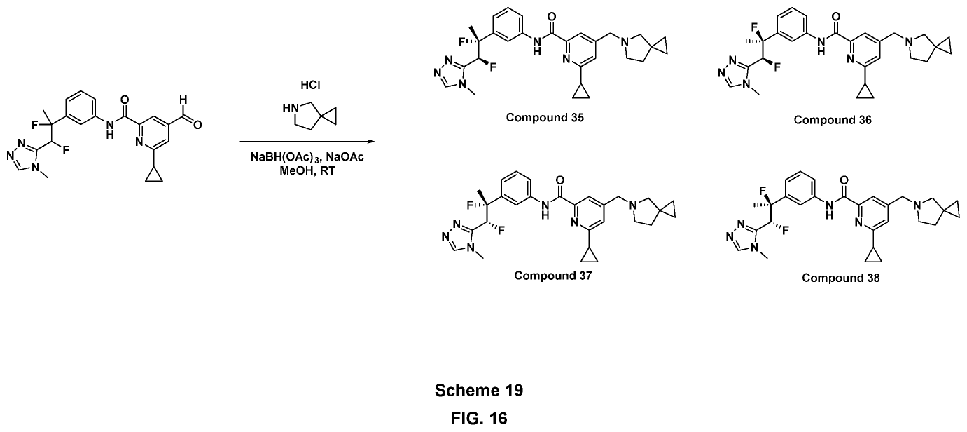

Compounds 35, 36, 37, and 38, stereoisomers of one another, can be synthesized according to Scheme 19, FIG. 16.

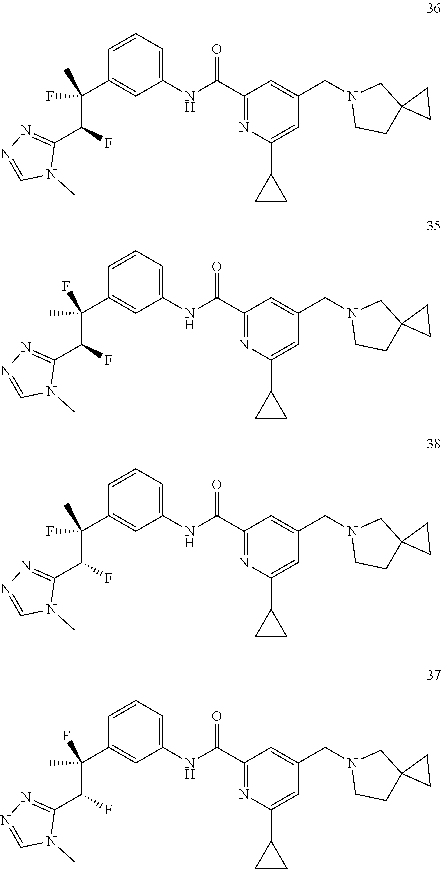 [SMILES: Cn1cnnc1[C@H](F)[C@](C)(F)c1cccc(NC(=O)c2cc(CN3CCC4(CC4)C3)cc(C3CC3)n2)c1 ; Cn1cnnc1[C@H](F)[C@@](C)(F)c1cccc(NC(=O)c2cc(CN3CCC4(CC4)C3)cc(C3CC3)n2)c1 ; Cn1cnnc1[C@@H](F)[C@](C)(F)c1cccc(NC(=O)c2cc(CN3CCC4(CC4)C3)cc(C3CC3)n2)c1 ; Cn1cnnc1[C@@H](F)[C@@](C)(F)c1cccc(NC(=O)c2cc(CN3CCC4(CC4)C3)cc(C3CC3)n2)c1]

In a first step, sodium acetate (37 mg, 0.451 mmol) was added to a mixture of 6-cyclopropyl-N-(3-(1,2-difluoro-1-(4-methyl-4H-1,2,4-triazol-3-yl)propan-2-yl)phenyl)-4-formylpicolinamide (Intermediate N, from Scheme 14, FIG. 6N; 64 mg, 0.150 mmol) and 5-azaspiro[2.4]heptane hydrochloride (40 mg, 0.301 mmol) in methanol (1.5 mL) at RT. The reaction was stirred for 15 min before sodium triacetoxyborohydride (95 mg, 0.451 mmol) was added and the resulting mixture was stirred at RT for 16 h. Three drops of sat. aqueous sodium bicarbonate were added and the reaction was concentrated under reduced pressure. The residue was purified by chromatography on C18 silica gel (0-100% acetonitrile in ammonium bicarbonate, pH=10. After chiral separation [First pass—isolation of the 2 major peaks: Anal. Column: ChiralPak IA, 250 mm×4.6 mm ID, 5 μm. Mobile Phase: 5:20:75 MeOH:DCM:Hexane (0.1% DEA). Isocratic Flow: 1 mL/min, (pressure was 54.5 bars). Column Temp.: ˜ 26° C. Run Time: 14 min.] [Second pass—isolation of the 2 minor peaks): Anal. Column: ChiralPak IA, 250 mm×4.6 mm ID, 5 μm. Mobile Phase: 15:5:80 MeOH:DCM:Hexane (0.1% DEA). Isocratic Flow: 0.8 mL/min, (pressure was 45.5 bars). Column Temp.: ˜ 26° C. Run Time: 18 min.], the desired products were obtained, and characterized as follows.

4-(5-azaspiro[2.4]heptan-5-ylmethyl)-6-cyclopropyl-N-(3-((1S,2R)-1,2-difluoro-1-(4-methyl-4H-1,2,4-triazol-3-yl)propan-2-yl)phenyl)picolinamide (5.2 mg, 6.8% yield), Compound 35. ¹H NMR (400 MHz, DMSO-d6) δ 10.24 (s, 1H), 8.50 (s, 1H), 7.97-7.77 (m, 3H), 7.61-7.36 (m, 2H), 7.19 (d, J=8.0 Hz, 1H), 6.32 (dd, J=43.6, 21.1 Hz, 1H), 3.68 (s, 2H), 3.49 (s, 3H), 2.74-2.64 (m, 2H), 2.30-2.21 (m, 1H), 1.87-1.71 (m, 5H), 1.22-1.13 (m, 5H), 1.08-1.00 (m, 2H), 0.52 (d, J=6.7 Hz, 4H). LCMS [M+H]⁺ or [M−H]⁻=507.3.

4-(5-azaspiro[2.4]heptan-5-ylmethyl)-6-cyclopropyl-N-(3-((1S,2S)-1,2-difluoro-1-(4-methyl-4H-1,2,4-triazol-3-yl)propan-2-yl)phenyl)picolinamide (4.4 mg, 5.8% yield). (36) ¹H NMR (400 MHz, DMSO-d6) δ 10.24 (s, 1H), 8.50 (s, 1H), 8.01-7.73 (m, 3H), 7.55-7.34 (m, 2H), 7.19 (d, J=7.3 Hz, 1H), 6.32 (dd, J=43.6, 21.1 Hz, 1H), 3.68 (s, 2H), 3.49 (s, 3H), 2.74-2.63 (m, 2H), 2.32-2.21 (m, 1H), 1.87-1.69 (m, 5H), 1.32-1.15 (m, 5H), 1.10-1.00 (m, 2H), 0.52 (d, J=6.8 Hz, 4H). LCMS [M+H]⁺ or [M−H]⁻=507.3.

4-(5-azaspiro[2.4]heptan-5-ylmethyl)-6-cyclopropyl-N-(3-((1R,2S)-1,2-difluoro-1-(4-methyl-4H-1,2,4-triazol-3-yl)propan-2-yl)phenyl)picolinamide (8.5 mg, 11% yield). (37) ¹H NMR (400 MHz, DMSO-d6) δ 10.18 (s, 1H), 8.41 (s, 1H), 7.98-7.65 (m, 2H), 7.62-7.30 (m, 2H), 7.07 (d, J=7.9 Hz, 1H), 6.28 (dd, J=42.5, 22.3 Hz, 1H), 3.66 (s, 2H), 3.51 (s, 3H), 2.95-2.82 (m, 1H), 2.78-2.55 (m, 1H), 2.31-2.17 (m, 1H), 1.90 (dd, J=23.4, 1.7 Hz, 3H), 1.83-1.65 (m, 1H), 1.40-0.92 (m, 7H), 0.61-0.43 (m, 4H). LCMS [M+H]⁺ or [M−H]⁻=507.3.

4-(5-azaspiro[2.4]heptan-5-ylmethyl)-6-cyclopropyl-N-(3-((1R,2R)-1,2-difluoro-1-(4-methyl-4H-1,2,4-triazol-3-yl)propan-2-yl)phenyl)picolinamide (6.5 mg, 8.5% yield). (38) ¹H NMR (400 MHz, DMSO-d6) δ 10.19 (s, 1H), 8.43 (s, 1H), 7.97-7.65 (m, 3H), 7.36 (t, J=8.0 Hz, 1H), 7.09 (d, J=7.6 Hz, 1H), 6.29 (dd, J=42.5, 22.3 Hz, 1H), 3.67 (s, 2H), 3.53 (s, 3H), 2.96-2.89 (m, 1H), 2.81-2.61 (m, 2H), 2.30-2.18 (m, 1H), 1.92 (d, J=23.4 Hz, 3H), 1.87-1.74 (m, 2H), 1.25-1.09 (m, 5H), 1.09-0.95 (m, 2H), 0.65-0.44 (m, 4H). LCMS [M+H]⁺ or [M−H]⁻=507.3.

### Example 20: Compound 39, Compound 40, Compound 41 and Compound 42

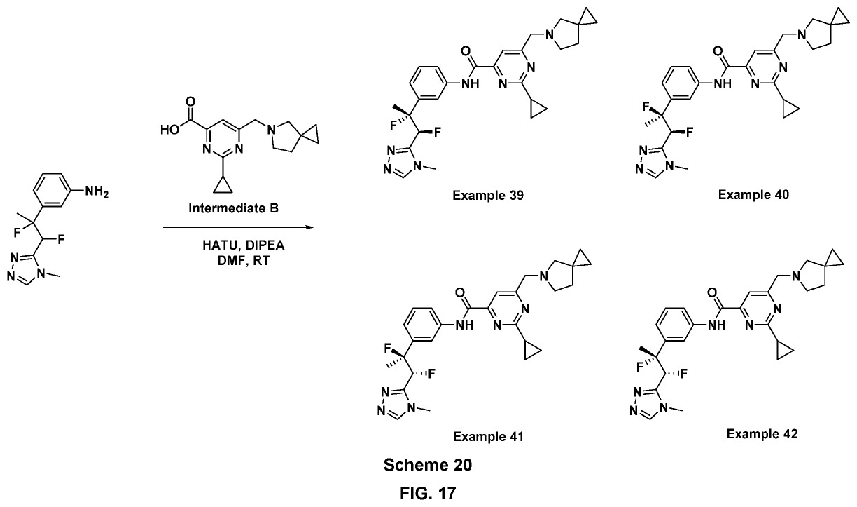

Compounds 39, 40, 41, and 42, stereoisomers, can be synthesized according to Scheme 20, FIG. 17.

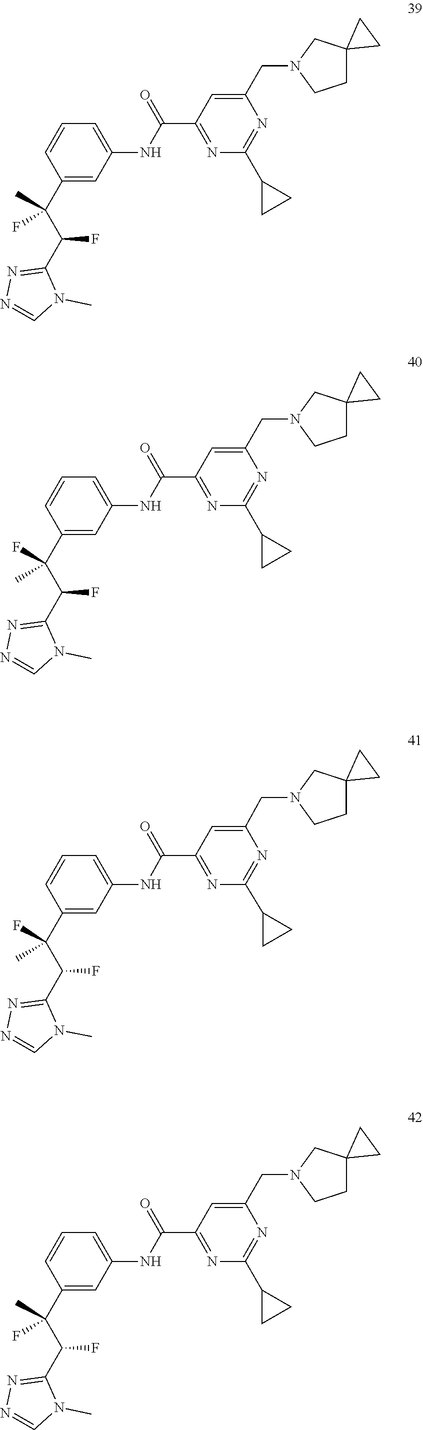 [SMILES: Cn1cnnc1[C@H](F)[C@](C)(F)c1cccc(NC(=O)c2cc(CN3CCC4(CC4)C3)nc(C3CC3)n2)c1 ; Cn1cnnc1[C@H](F)[C@@](C)(F)c1cccc(NC(=O)c2cc(CN3CCC4(CC4)C3)nc(C3CC3)n2)c1 ; Cn1cnnc1[C@@H](F)[C@@](C)(F)c1cccc(NC(=O)c2cc(CN3CCC4(CC4)C3)nc(C3CC3)n2)c1 ; Cn1cnnc1[C@@H](F)[C@](C)(F)c1cccc(NC(=O)c2cc(CN3CCC4(CC4)C3)nc(C3CC3)n2)c1]

In a first step, N,N-diisopropylethylamine (0.22 mL, 1.24 mmol) was added to a solution of 3-(1,2-difluoro-1-(4-methyl-4H-1,2,4-triazol-3-yl)propan-2-yl)aniline (110 mg, 0.434 mmol), 6-(5-azaspiro[2.4]heptan-5-ylmethyl)-2-cyclopropylpyrimidine-4-carboxylic acid (Intermediate B, Scheme 2, FIG. 6B; 113 mg, 0.413 mmol) and HATU (314 mg, 0.827 mmol) in DMF (2.1 mL) at RT and the resulting mixture was stirred at RT for 16 h. The reaction was diluted with water (10 mL) and DCM (10 mL). The layers were separated, the aqueous phase was extracted with DCM (2×10 mL). The organic layers were combined, dried over sodium sulfate, and concentrated under reduced pressure. The residue was purified by chromatography on C18 silica gel (0-100% acetonitrile in ammonium formate, pH=4. After chiral separation [Anal. Column: ChiralPak IA, 250 mm×4.6 mm ID, 5 μm. Mobile Phase: 5:15:80 MeOH:DCM:Hexane (0.1% DEA). Isocratic Flow: 1 mL/min, (pressure was 40.5 bars). Column Temp.: ˜26° C. Run Time: 25 min], desired products were obtained, characterized as follows.

6-(5-azaspiro[2.4]heptan-5-ylmethyl)-2-cyclopropyl-N-(3-((1S,2R)-1,2-difluoro-1-(4-methyl-4H-1,2,4-triazol-3-yl)propan-2-yl)phenyl)pyrimidine-4-carboxamide (15 mg, 7.0% yield), (39). ¹H NMR (400 MHz, DMSO-d6) δ 10.54 (s, 1H), 8.51 (s, 1H), 7.98-7.83 (m, 3H), 7.42 (t, J=8.0 Hz, 1H), 7.23 (d, J=7.8 Hz, 1H), 6.31 (dd, J=43.7, 21.0 Hz, 1H), 3.78 (ap s, 2H), 3.51-3.45 (m, 3H), 2.77 (ap s, 2H), 2.54 (ap s, 2H), 2.40-2.30 (m, 1H), 1.86-1.69 (m, 5H), 1.28-1.17 (m, 2H), 1.17-1.07 (m, 2H), 0.53 (d, J=5.9 Hz, 4H). LCMS [M+H]⁺ or [M−H]⁻=508.3.

6-(5-azaspiro[2.4]heptan-5-ylmethyl)-2-cyclopropyl-N-(3-((1S,2S)-1,2-difluoro-1-(4-methyl-4H-1,2,4-triazol-3-yl)propan-2-yl)phenyl)pyrimidine-4-carboxamide (12 mg, 5.7% yield), (40). ¹H NMR (400 MHz, CD3OD) δ 8.46 (s, 1H), 7.99 (s, 1H), 7.93-7.75 (m, 2H), 7.45 (t, J=8.0 Hz, 1H), 7.24 (d, J=8.3 Hz, 1H), 6.16 (dd, J=43.2, 20.5 Hz, 1H), 3.89 (s, 2H), 3.58 (s, 3H), 2.93 (t, J=6.9 Hz, 2H), 2.68 (s, 2H), 2.48-2.35 (m, 1H), 1.90-1.84 (m, 2H), 1.84-1.70 (m, 3H), 1.32-1.22 (m, 3H), 1.22-1.05 (m, 2H), 0.63-0.53 (m, 4H). LCMS [M+H]⁺ or [M−H]⁻=508.3.

6-(5-azaspiro[2.4]heptan-5-ylmethyl)-2-cyclopropyl-N-(3-((1R,2S)-1,2-difluoro-1-(4-methyl-4H-1,2,4-triazol-3-yl)propan-2-yl)phenyl)pyrimidine-4-carboxamide (16 mg, 7.4% yield), (41). ¹H NMR (400 MHz, DMSO-d6) δ 10.49 (s, 1H), 8.43 (s, 1H), 7.93-7.79 (m, 3H), 7.37 (t, J=7.9 Hz, 1H), 7.13 (d, J=7.9 Hz, 1H), 6.29 (dd, J=42.5, 22.1 Hz, 1H), 3.76 (ap s, 2H), 3.53 (s, 3H), 2.75 (ap s, 2H), 2.39-2.30 (m, 1H), 1.92 (dd, J=23.4, 1.7 Hz, 3H), 1.78 (t, J=6.8 Hz, 2H), 1.28-1.04 (m, 5H), 0.53 (d, J=6.0 Hz, 4H). LCMS [M+H]⁺ or [M−H]⁻=508.3.

6-(5-azaspiro[2.4]heptan-5-ylmethyl)-2-cyclopropyl-N-(3-((1R,2R)-1,2-difluoro-1-(4-methyl-4H-1,2,4-triazol-3-yl)propan-2-yl)phenyl)pyrimidine-4-carboxamide (12 mg, 5.7% yield), (42). ¹H NMR (400 MHz, DMSO-d6) δ 10.48 (s, 1H), 8.43 (s, 1H), 7.94-7.71 (m, 3H), 7.37 (t, J=8.0 Hz, 1H), 7.12 (d, J=8.0 Hz, 1H), 6.29 (dd, J=42.5, 22.1 Hz, 1H), 3.76 (s, 2H), 3.53 (s, 3H), 2.75 (t, J=6.8 Hz, 2H), 2.52 (s, 2H), 2.41-2.25 (m, 1H), 1.92 (dd, J=23.4, 1.9 Hz, 3H), 1.78 (t, J=6.8 Hz, 2H), 1.26-1.03 (m, 5H), 0.56-0.48 (m, 4H). LCMS [M+H]⁺ or [M−H]⁻=508.3.

### Example 21: Compound 43, Compound 44, Compound 45 and Compound 46

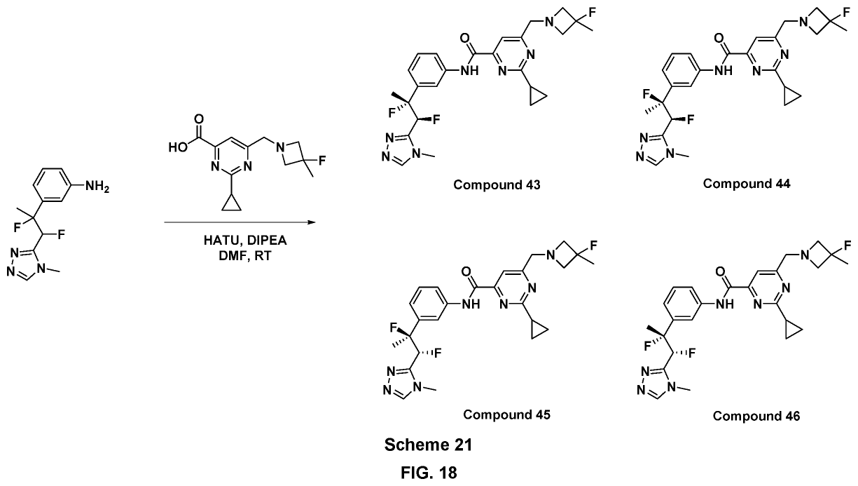

Compounds 43, 44, 45, and 46, stereoisomers, can be synthesized according to Scheme 21 in FIG. 18.

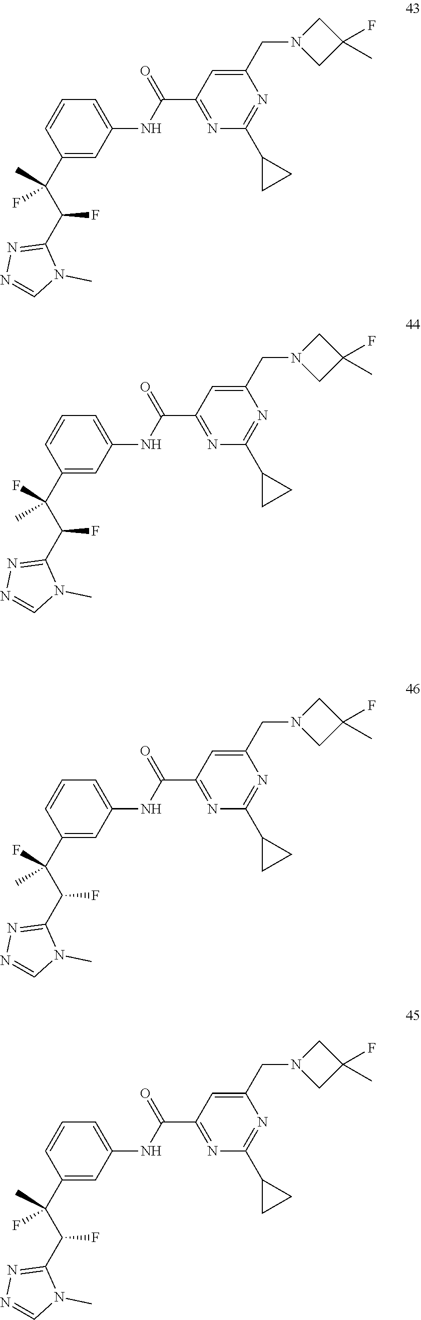 [SMILES: Cn1cnnc1[C@H](F)[C@](C)(F)c1cccc(NC(=O)c2cc(CN3CC(C)(F)C3)nc(C3CC3)n2)c1 ; Cn1cnnc1[C@H](F)[C@@](C)(F)c1cccc(NC(=O)c2cc(CN3CC(C)(F)C3)nc(C3CC3)n2)c1 ; Cn1cnnc1[C@@H](F)[C@@](C)(F)c1cccc(NC(=O)c2cc(CN3CC(C)(F)C3)nc(C3CC3)n2)c1 ; Cn1cnnc1[C@@H](F)[C@](C)(F)c1cccc(NC(=O)c2cc(CN3CC(C)(F)C3)nc(C3CC3)n2)c1]

In a single step, N,N-Diisopropylethylamine (0.18 mL, 1.01 mmol) was added to a solution of 3-(1,2-difluoro-1-(4-methyl-4H-1,2,4-triazol-3-yl)propan-2-yl)aniline (90.0 mg, 0.357 mmol), 2-cyclopropyl-6-((3-fluoro-3-methylazetidin-1-yl)methyl)pyrimidine-4-carboxylic acid (Intermediate C, FIG. 6C, Scheme 3; 89.7 mg, 0.340 mmol) and HATU (257 mg, 0.680 mmol) in DMF (1.8 mL) at RT and the resulting mixture was stirred at RT for 16 h. The reaction was diluted with water (10 mL) and DCM (10 mL). The layers were separated, the aqueous phase was extracted with 4:1 CHCl₃/IPA (3×10 mL), the organic layers were combined, dried over sodium sulfate, and concentrated under reduced pressure. The residue was purified by chromatography on C18 silica gel (0-100% acetonitrile in ammonium formate, pH=4. After chiral separation [Anal. Column: ChiralPak IA, 250 mm×4.6 mm ID, 5 μm. Mobile Phase: 10:15:75 MeOH:DCM:Hexane (0.1% DEA). Isocratic Flow: 0.8 mL/min, (pressure was 48.5 bars). Column Temp.: ˜ 26° C. Run Time: 18 min], desired products were obtained and characterized as follows.

2-cyclopropyl-N-(3-((1S,2R)-1,2-difluoro-1-(4-methyl-4H-1,2,4-triazol-3-yl)propan-2-yl)phenyl)-6-((3-fluoro-3-methylazetidin-1-yl)methyl)pyrimidine-4-carboxamide (5.4 mg, 3.0% yield), (43). ¹H NMR (400 MHz, DMSO-d6) δ 10.54 (s, 1H), 8.50 (s, 1H), 7.97 (s, 1H), 7.91 (d, J=7.8 Hz, 1H), 7.76 (s, 1H), 7.42 (t, J=7.9 Hz, 1H), 7.22 (d, J=8.1 Hz, 1H), 6.31 (dd, J=43.7, 21.0 Hz, 1H), 3.82 (s, 2H), 3.54-3.38 (m, 6H), 2.39-2.29 (m, 1H), 1.81 (d, J=23.6 Hz, 3H), 1.56 (d, J=22.3 Hz, 3H), 1.31-0.89 (m, 5H). LCMS [M+H]⁺ or [M−H]⁻=500.3.

2-cyclopropyl-N-(3-((1S,2S)-1,2-difluoro-1-(4-methyl-4H-1,2,4-triazol-3-yl)propan-2-yl)phenyl)-6-((3-fluoro-3-methylazetidin-1-yl)methyl)pyrimidine-4-carboxamide (4.5 mg, 2.5% yield), (44). ¹H NMR (400 MHz, DMSO-d6) δ 10.52 (s, 1H), 8.48 (s, 1H), 7.95 (t, J=1.9 Hz, 1H), 7.88 (dd, J=8.1, 1.5 Hz, 1H), 7.74 (s, 1H), 7.40 (t, J=7.8 Hz, 1H), 7.20 (d, J=7.9 Hz, 1H), 6.29 (dd, J=43.6, 20.9 Hz, 1H), 3.80 (s, 2H), 3.52-3.33 (m, 6H), 2.37-2.29 (m, 1H), 1.79 (d, J=23.4 Hz, 3H), 1.54 (d, J=22.4 Hz, 3H), 1.25-0.99 (m, 5H). LCMS [M+H]⁺ or [M−H]⁻=500.3.

2-cyclopropyl-N-(3-((1R,2S)-1,2-difluoro-1-(4-methyl-4H-1,2,4-triazol-3-yl)propan-2-yl)phenyl)-6-((3-fluoro-3-methylazetidin-1-yl)methyl)pyrimidine-4-carboxamide (5.3 mg, 3.0% yield), (45). ¹H NMR (400 MHz, DMSO-d6) δ 10.49 (s, 1H), 8.43 (s, 1H), 7.98-7.79 (m, 1H), 7.74 (s, 1H), 7.37 (t, J=8.0 Hz, 1H), 7.12 (d, J=8.5 Hz, 1H), 6.29 (dd, J=42.4, 22.0 Hz, 1H), 3.82 (s, 2H), 3.53-3.36 (m, 6H), 2.45-2.29 (m, 1H), 1.91 (dd, J=23.4, 1.9 Hz, 3H), 1.56 (d, J=22.4 Hz, 3H), 1.26-0.87 (m, 5H). LCMS [M+H]⁺ or [M−H]⁻=500.2.

2-cyclopropyl-N-(3-((1R,2R)-1,2-difluoro-1-(4-methyl-4H-1,2,4-triazol-3-yl)propan-2-yl)phenyl)-6-((3-fluoro-3-methylazetidin-1-yl)methyl)pyrimidine-4-carboxamide (4.5 mg, 2.5% yield), (46). ¹H NMR (400 MHz, DMSO-d6) δ 10.47 (s, 1H), 8.41 (s, 1H), 7.87-7.76 (m, 2H), 7.72 (s, 1H), 7.35 (t, J=7.8 Hz, 1H), 7.10 (d, J=7.7 Hz, 1H), 6.26 (dd, J=42.4, 22.0 Hz, 1H), 3.79 (s, 2H), 3.53-3.34 (m, 6H), 2.35-2.27 (m, 1H), 1.89 (dd, J=23.5, 1.9 Hz, 3H), 1.54 (d, J=22.4 Hz, 3H), 1.18-0.92 (m, 5H). LCMS [M+H]⁺ or [M−H]⁻=500.3.

### Example 22: Compound 506

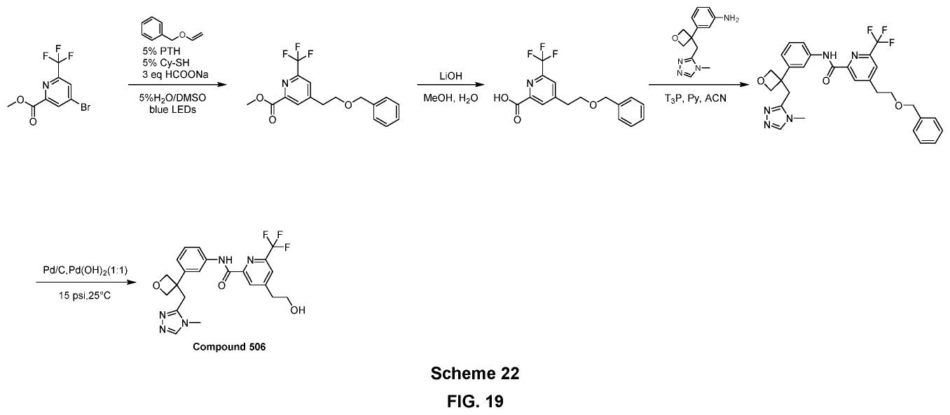

Compound 506, 4-(2-hydroxyethyl)-N-(3-(3-((4-methyl-4H-1,2,4-triazol-3-yl)-methyl)oxetan-3-yl)phenyl)-6-(trifluoromethyl)picolinamide, can be synthesized according to Scheme 22, FIG. 19.

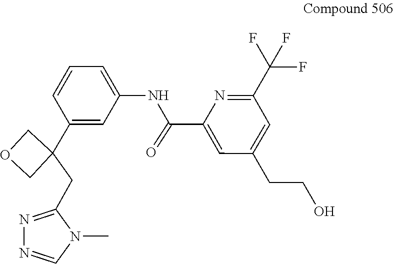 [SMILES: Cn1cnnc1CC1(c2cccc(NC(=O)c3cc(CCO)cc(C(F)(F)F)n3)c2)COC1]

A first intermediate, methyl 4-(2-(benzyloxy)ethyl)-6-(trifluoromethyl)picolinate, can be synthesized as follows. A mixture of ((vinyloxy)methyl)benzene (708.6 mg, 5.28 mmol), 10-phenylphenothiazine (29.1 mg, 0.11 mmol), sodium bicarbonate (532.4 mg, 6.34 mmol), methyl 4-bromo-6-(trifluoromethyl)picolinate (600.0 mg, 2.11 mmol) and cyclohexyl mercaptan (0.01 mL, 0.11 mmol) in dimethyl sulfoxide (6 mL) and water (0.3 mL) in a sealed tube was irradiated with 34 W Blue LED lamp (Kessil KSH150B LED Grow Light) for 16 h under nitrogen protection with cooling from a fan (vial temperature reached 37° C.). The reaction mixture was diluted with water (20 mL) then extracted with ethyl acetate (3×20 mL). The combined organic layers were dried over anhydrous sodium sulfate and concentrated under reduced pressure. The residue was purified by silica gel chromatography (mobile phase: ethyl acetate/petroleum ether, gradient 0% to 15%) to afford methyl 4-(2-(benzyloxy)ethyl)-6-(trifluoromethyl)picolinate (300.0 mg, 42% yield) as yellow oil. LCMS [M+H]⁺=340.1.

A second intermediate, 4-(2-(benzyloxy)ethyl)-6-(trifluoromethyl) picolinic acid, can be synthesized as follows. To a mixture of methyl 4-(2-(benzyloxy)ethyl)-6-(trifluoro-methyl)picolinate (300.0 mg, 0.88 mmol) and lithium hydroxide (111.3 mg, 2.65 mmol) in water (1 mL) and methanol (2 mL). The mixture was stirred 25° C. for 4 h and adjusted to pH=6 with hydrochloric acid (1 M). The solid was collected by filtration and dried to afford crude 4-(2-(benzyloxy)ethyl)-6-(trifluoromethyl) picolinic acid (280.0 mg, 97% yield) as a yellow solid. LCMS [M+H]⁺=326.1.

A third intermediate, 4-(2-(benzyloxy)ethyl)-N-(3-(3-((4-methyl-4H-1,2,4-triazol-3-yl)methyl)oxetan-3-yl)phenyl)-6-(trifluoromethyl)picolinamide, can be synthesized as follows. To a solution of 4-(2-(benzyloxy)ethyl)-6-(trifluoromethyl) picolinic acid (280.0 mg, 0.86 mmol) and 3-(3-((4-methyl-4H-1,2,4-triazol-3-yl)methyl)oxetan-3-yl)aniline (231.6 mg, 0.95 mmol) in acetonitrile (7 mL) was added pyridine (0.21 mL, 2.6 mmol) and 2,4,6-tripropyl-1,3,5,2,4,6-trioxatriphosphinane 2,4,6-trioxide (50% in ethyl acetate, 0.94 mL, 1.34 mmol). The mixture was stirred at 25° C. for 12 h and then concentrated under reduced pressure. The residue was purified by silica gel chromatography (mobile phase: methyl alcohol/dichloromethane, gradient 0% to 6%) to afford 4-(2-(benzyloxy)ethyl)-N-(3-(3-((4-methyl-4H-1,2,4-triazol-3-yl)methyl)oxetan-3-yl)phenyl)-6-(trifluoromethyl)picolinamide (174.4 mg, 37% yield) as yellow oil. LCMS [M+H]⁺=552.2.

Compound 506 is then formed as follows. A mixture of 4-(2-(benzyloxy)ethyl)-N-(3-(3-((4-methyl-4H-1,2,4-triazol-3-yl)methyl)oxetan-3-yl)phenyl)-6-(trifluoromethyl)-picolinamide (154.0 mg, 0.28 mmol), Palladium (10% on carbon, 234.9 mg, 0.03 mmol) and Palladium hydroxide (77.7 mg, 0.03 mmol) in methanol (10 mL) was hydrogenated (15 psi) at 25° C. for 2 h and filtered. The filtrate was concentrated under reduced pressure. The residue was purified by RP-HPLC (acetonitrile 20-50/0.05% NH₃·H₂O in water) to afford 4-(2-hydroxyethyl)-N-(3-(3-((4-methyl-4H-1,2,4-triazol-3-yl)methyl)oxetan-3-yl)phenyl)-6-(trifluoromethyl)picolinamide (13.8 mg, 10% yield) as a white solid. ¹H NMR (400 MHz, methanol-d₄) δ 8.35 (s, 1H), 8.19 (s, 1H), 7.97 (d, J=1.2 Hz, 1H), 7.75-7.69 (m, 1H), 7.39-7.30 (m, 1H), 6.67-6.61 (m, 1H), 5.10-5.01 (m, 1H), 4.62 (s, 1H), 3.95-3.86 (m, 1H), 3.65 (s, 1H), 3.07 (t, J=6.0 Hz, 1H), 2.87 (s, 1H). LCMS [M+H]⁺=462.2.

### Example 23: Compound 507

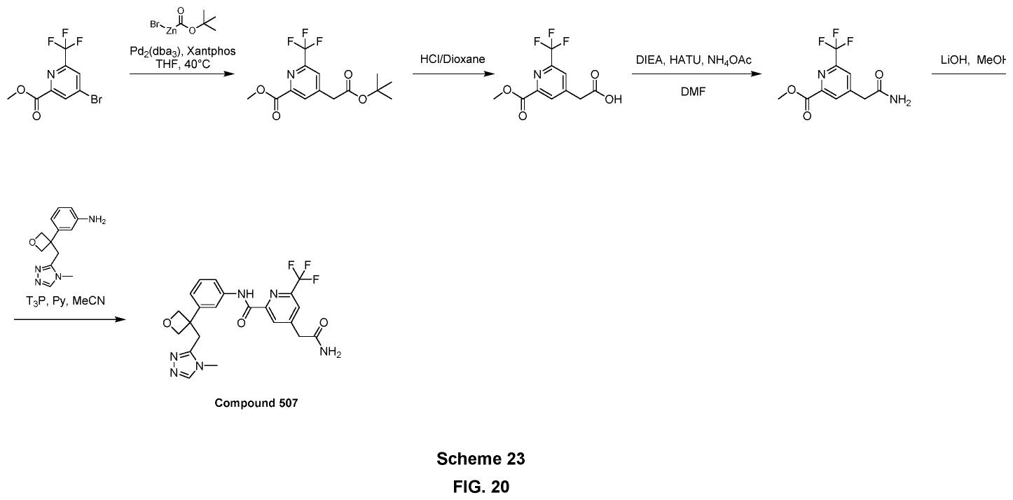

Compound 507, 4-(2-amino-2-oxoethyl)-N-(3-(3-((4-methyl-4H-1,2,4-triazol-3-yl)methyl)oxetan-3-yl)phenyl)-6-(trifluoromethyl)picolinamide, can be synthesized according to Scheme 23, FIG. 20.

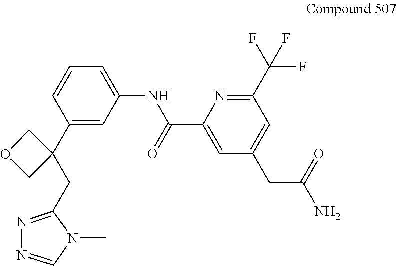 [SMILES: Cn1cnnc1CC1(c2cccc(NC(=O)c3cc(CC(N)=O)cc(C(F)(F)F)n3)c2)COC1]

A first intermediate, methyl 4-(2-(tert-butoxy)-2-oxoethyl)-6-(trifluoromethyl)-picolinate, can be synthesized as follows. A mixture of methyl 4-bromo-6-(trifluoromethyl)-picolinate (1.0 g, 3.52 mmol), (tert-butoxycarbonyl)zinc(II) bromide (1.0 M in tetrahydrofuran, 5.28 mL, 5.28 mmol), dicyclohexyl(2′,4′,6′-triisopropyl-[1,1′-biphenyl]-2-yl)phosphine (336 mg, 0.70 mmol) and tri(dibenzylideneacetone)dipalladium(0) (322 mg, 0.35 mmol) in tetrahydrofuran (25 mL) was stirred at 40° C. for 16 h under nitrogen protection. The reaction solution was quenched by addition of saturated aqueous citric acid (5 mL) and extracted with ethyl acetate (3×50 mL). The combined organic phases were washed with water (50 mL), dried over anhydrous sodium sulfate and concentrated under reduced pressure. The residue was purified by silica gel chromatography (mobile phase: ethyl acetate/petroleum ether, gradient 0% to 15%) to afford methyl 4-(2-(tert-butoxy)-2-oxoethyl)-6-(trifluoromethyl)picolinate (828.0 mg, 74% yield) as a yellow oil. LCMS [M+H]⁺=320.1.

A second intermediate, 2-(2-(methoxycarbonyl)-6-(trifluoromethyl)pyridin-4-yl)acetic acid, can be synthesized as follows. A mixture of methyl 4-(2-(tert-butoxy)-2-oxoethyl)-6-(trifluoromethyl)picolinate (820 mg, 2.57 mmol) and hydrogen chloride (4 M in ethyl acetate, 0.64 mL, 2.57 mmol) in ethyl acetate (20 mL) was stirred at 25° C. for 12 h and concentrated under reduced pressure to afford crude 2-(2-(methoxycarbonyl)-6-(trifluoromethyl)pyridin-4-yl)acetic acid (675 mg, 99% yield) as a yellow solid. LCMS [M+H]+=264.0.

A third intermediate, methyl 4-(2-amino-2-oxoethyl)-6-(trifluoromethyl)picolinate, can be synthesized as follows. To a solution of 2-(2-(methoxycarbonyl)-6-(trifluoromethyl)-pyridin-4-yl)acetic acid (300 mg, 1.14 mmol), HATU (650 mg, 1.71 mmol) and ammonium acetate (264 mg, 3.42 mmol) in N,N-Dimethylformamide (10 mL) was added N-ethyl-N-isopropylpropan-2-amine (0.6 mL, 3.42 mmol). The mixture was stirred at 25° C. for 16 h. The resulting mixture was diluted with water (30 mL) then extracted with ethyl acetate (3×30 mL). The combined organic layers were washed with brine (30 mL) and concentrated under reduced pressure. The residue was purified by preparative TLC (solvent gradient: ethyl acetate) to afford methyl 4-(2-amino-2-oxoethyl)-6-(trifluoromethyl)picolinate (75 mg, 25% yield) as a yellow oil. LCMS [M+H]⁺=263.1.

A fourth intermediate, 4-(2-amino-2-oxoethyl)-6-(trifluoromethyl)picolinic acid, can be synthesized as follows. To a mixture of methyl 4-(2-amino-2-oxoethyl)-6-(trifluoromethyl)picolinate (70 mg, 0.23 mmol) in water (1 mL) and methanol (2 mL) was added lithium hydroxide (34 mg, 0.69 mmol). The mixture was stirred 25° C. for 4 h and adjusted to pH=6 by addition of hydrochloric acid (1 M). The mixture was then concentrated under reduced pressure to afford 4-(2-amino-2-oxoethyl)-6-(trifluoromethyl)picolinic acid (65 mg, 99% yield) as a yellow solid. LCMS [M+H]⁺=249.1.

Compound 507 can be formed as follows. To a solution of 3-(3-((4-methyl-4H-1,2,4-triazol-3-yl)methyl)oxetan-3-yl)aniline (70.5 mg, 0.29 mmol) and 2,4,6-tripropyl-1,3,5,2,4,6-trioxatriphosphinane 2,4,6-trioxide (0.29 mL, 0.41 mmol, 50% in ethyl acetate) in acetonitrile (3 mL) was added 4-(2-amino-2-oxoethyl)-6-(trifluoromethyl) picolinic acid (65.0 mg, 0.26 mmol) and pyridine (0.06 mL, 0.79 mmol). The mixture was stirred at 25° C. for 12 h then concentrated under reduced pressure. The residue was purified by RP-HPLC (20% to 50% ACN/(0.05% NH₃H₂O in water)) to afford 4-(2-amino-2-oxoethyl)-N-(3-(3-((4-methyl-4H-1,2,4-triazol-3-yl)methyl)oxetan-3-yl)phenyl)-6-(trifluoromethyl)picolinamide (15.4 mg, 12% yield) as a white solid. ¹H NMR (400 MHz, methanol-d₄) δ 8.38 (s, 1H), 8.20 (s, 1H), 8.01 (s, 1H), 7.72 (d, J=8.0 Hz, 1H), 7.40-7.27 (m, 2H), 6.64 (d, J=7.6 Hz, 1H), 5.12-5.00 (m, 4H), 3.82 (s, 2H), 3.64 (s, 2H), 2.86 (s, 3H). LCMS [M+H]⁺=475.1.

### Example 24: Quantitative Data for Additional Picolinamide Compounds

Additional amide (such as picolinamide) compounds can be synthesized according to the methods herein. Quantitative data for examples of such compounds is shown in Table 2.

TABLE 2
Quantitative Analytical Data on Representative
Amide (such as Picolinamide) Compounds

| | | LCMS |
|---|---|---|
| | | [M + H]⁺ |
| Compound # | ¹H NMR | or [M − H]⁻ |
| 60 | ¹H NMR (400 MHz, CDCl₃): δ 9.74 (br s, 1H), 8.38 (s, 1H), | 537.1 |
| | 7.89 (s, 1H), 7.85 (s, 1H), 7.66 (dd, J = 1.2, 8.0 Hz, 1H), | |
| | 7.40 (s, 1H), 7.32 (t, J = 8.0 Hz, 1H), 6.64 (d, J = 8.0 Hz, | |
| | 1H), 6.50 (d, J = 46 Hz, 1H), 5.31-5.21 (m, 3H), 5.00 (dd, | |
| | J = 5.6, 6.0 Hz, 1H), 3.88 (s, 2H), 3.51-3.43 (m, 2H), 3.40- | |
| | 3.29 (m, 2H), 2.97 (d, J = 1.6 Hz, 3H), 1.66 (d, J = 22.4 Hz, | |
| | 3H). | |
| 61 | ¹H NMR (400 MHz, CDCl₃): δ 9.75 (s, 1H), 8.38 (s, 1H), 7.94- | 537.1 |
| | 7.84 (m, 2H), 7.70-7.64 (m, 1H), 7.40 (s, 1H), 7.36-7.29 | |
| | (m, 1H), 6.67-6.61 (m, 1H), 6.50 (d, J = 46 Hz, 1H), 5.33- | |
| | 5.20 (m, 3H), 5.04-4.97 (m, 1H), 3.89 (s, 2H), 3.55-3.44 | |
| | (m, 2H), 3.43-3.29 (m, 2H), 2.97 (s, 3H), 1.63 (s, 3H). | |
| 62 | ¹H NMR (400 MHz, CDCl₃): δ 9.82 (br s, 1H), 8.08 (s, 1H), | 518.3 |
| | 7.89 (s, 1H), 7.71 (dd, J = 1.2, 8.0 Hz, 1H), 7.35-7.29 (m, | |
| | 1H), 6.66 (d, J = 7.6 Hz, 1H), 6.48 (d, J = 46 Hz, 1H), 5.34- | |
| | 5.29 (m, 3H), 5.01-4.96 (m, 1H), 3.82 (s, 2H), 2.98 (d, J = | |
| | 1.2 Hz, 3H), 2.86 (t, J = 6.8 Hz, 2H), 2.62 (s, 2H), 2.43-2.33 | |
| | (m, 1H), 1.86 (t, J = 6.8 Hz, 2H), 1.25-1.12 (m, 4H), 0.57 | |
| | (s, 4H). | |
| 63 | ¹H NMR (400 MHz, CDCl₃): δ 9.83 (br s, 1H), 8.07 (s, 1H), | 518.3 |
| | 7.89 (s, 1H), 7.70 (dd, J = 1.6, 8.0 Hz, 1H), 7.35-7.28 (m, | |
| | 1H), 6.66 (d, J = 8.0 Hz, 1H), 6.48 (d, J = 46 Hz, 1H), 5.33- | |
| | 5.29 (m, 3H), 5.02-4.92 (m, 1H), 3.81 (s, 2H), 2.98 (d, | |
| | J = 1.6 Hz, 3H), 2.85 (t, J = 7.2 Hz, 2H), 2.62 (s, 2H), 2.43- | |
| | 2.31 (m, 1H), 1.86 (t, J = 6.8 Hz, 2H), 1.26-1.10 (m, 4H), | |
| | 0.57 (d, J = 1.2 Hz, 3H). | |
| 64 | ¹H NMR (400 MHz, methanol-d₄): δ 8.52 (s, 1H), 8.40 (br s, | 465.1 |
| | 1H), 8.27 (s, 1H), 8.15 (d, J = 1.2 Hz, 1H), 7.77-7.72 (m, | |
| | 1H), 7.49 (t, J = 2.0 Hz, 1H), 7.37 (t, J = 8.0 Hz, 1H), 6.85 | |
| | (d, J = 8.0 Hz, 1H), 6.30 (d, J = 45.2 Hz, 1H), 5.46 (d, J = | |
| | 6.4 Hz, 1H), 5.30 (d, J = 6.4 Hz, 1H), 5.19 (dd, J = 1.6, 6.4 | |
| | Hz, 1H), 5.00 (dd, J = 4.0, 6.0 Hz, 1H), 4.36 (s, 2H), 3.10 (s, | |
| | 3H). | |
| 65 | ¹H NMR (400 MHz, methanol-d₄): δ 8.51 (s, 1H), 8.41 (br s, | 465.1 |
| | 1H), 8.27 (s, 1H), 8.14 (s, 1H), 7.74 (dd, J = 1.2, 8.0 Hz, | |
| | 1H), 7.49 (t, J = 1.6 Hz, 1H), 7.37 (t, J = 8.0 Hz, 1H), 6.85 | |
| | (d, J = 8.0 Hz, 1H), 6.30 (d, J = 45.2 Hz, 1H), 5.46 (d, J = | |
| | 6.8 Hz, 1H), 5.30 (d, J = 6.4 Hz, 1H), 5.20 (dd, J = 1.6, 6.4 | |
| | Hz, 1H), 5.00 (dd, J = 4.0, 6.0 Hz, 1H), 4.34 (s, 2H), 3.10 (s, | |
| | 3H). | |
| 67 | ¹H NMR (400 MHz, methanol-d₄): δ 8.41 (br s, 1H), 8.18 (s, | 475.1 |
| | 1H), 8.03 (d, J = 1.2 Hz, 1H), 7.71 (ddd, J = 0.4, 2.0, 8.0 | |
| | Hz, 1H), 7.43-7.28 (m, 2H), 6.67-6.61 (m, 1H), 5.10-5.02 | |
| | (m, 4H), 3.71 (s, 2H), 3.64 (s, 2H), 2.87 (s, 3H), 2.31 (s, 6H) | |
| 98 | ¹H NMR (400 MHz, DMSO-d₆): δ 10.33 (s, 1H), 8.34 (s, 1H), | 527.1 |
| | 8.20 (s, 1H), 8.07 (s, 1H), 7.80 (d, J = 8.4 Hz, 1H), 7.46 (s, | |
| | 1H), 7.28 (t, J = 8.0 Hz, 1H), 6.67 (d, J = 8.0 Hz, 1H), 4.94 | |
| | (d, J = 6.0 Hz, 2H), 4.86 (d, J = 6.0 Hz, 2H), 3.86 (s, 2H), | |
| | 3.49 (s, 2H), 2.92 (s, 3H), 2.72 (t, J = 6.8 Hz, 2H), 1.78 (t, | |
| | J = 6.8 Hz, 2H), 0.52 (d, J = 4.4 Hz, 4H). | |
| 112 | ¹H NMR (400 MHz, methanol-d₄): δ 8.28 (s, 1H), 7.92 (d, | 523.2 |
| | J = 0.8 Hz, 1H), 7.73-7.66 (m, 1H), 7.45-7.39 (m, 2H), 7.35 | |
| | (t, J = 8.0 Hz, 1H), 6.79 (d, J = 8.4 Hz, 1H), 6.29 (d, J = 45.2 | |
| | Hz, 1H), 5.47 (d, J = 6.4 Hz, 1H), 5.30 (d, J = 6.4 Hz, 1H), | |
| | 5.19 (dd, J = 1.6, 6.8 Hz, 1H), 5.01 (dd, J = 4.0, 6.0 Hz, 1H), | |
| | 3.63-3.34 (m, 3H), 3.26-3.18 (m, 2H), 3.07 (s, 3H), 2.27- | |
| | 2.16 (m, 1H), 1.58 (d, J = 22 Hz, 3H), 1.26 (d, J = 6.4 Hz, | |
| | 3H), 1.16-1.07 (m, 4H) | |
| 113 | ¹H NMR (400 MHz, methanol-d₄): δ 8.27 (s, 1H), 7.91 (d, | 523.2 |
| | J = 1.2 Hz, 1H), 7.71-7.67 (m, 1H), 7.43-7.39 (m, 2H), 7.35 | |
| | (t, J = 8.0 Hz, 1H), 6.78 (d, J = 7.6 Hz, 1H), 6.29 (d, J = 44.8 | |
| | Hz, 1H), 5.47 (d, J = 6.4 Hz, 1H), 5.30 (d, J = 6.4 Hz, 1H), | |
| | 5.19 (dd, J = 1.6, 6.4 Hz, 1H), 5.01 (dd, J = 4.0, 6.0 Hz, 1H), | |
| | 3.65-3.34 (m, 3H), 3.26-3.14 (m, 2H), 3.07 (s, 3H), 2.25- | |
| | 2.17 (m, 1H), 1.57 (d, J = 21.6 Hz, 3H), 1.26 (d, J = 6.8 Hz, | |
| | 3H), 1.20-1.05 (m, 4H). | |
| 114 | ¹H NMR (400 MHz, CDCl₃): δ 9.90 (s, 1H), 7.96 (s, 1H), 7.92 | 523.1 |
| | (s, 1H), 7.54 (d, J = 7.6 Hz, 1H), 7.40 (br s, 1H), 7.36-7.28 | |
| | (m, 2H), 6.64 (d, J = 7.2 Hz, 1H), 6.46 (d, J = 46 Hz, 1H), | |
| | 5.28 (t, J = 6.8 Hz, 2H), 5.23 (d, J = 6.4 Hz, 1H), 4.99 (t, J = | |
| | 5.6 Hz, 1H), 3.58 (br s, 2H), 3.35 (br s, 2H), 2.99 (s, 3H), | |
| | 2.23-2.12 (m, 1H), 1.64 (d, J = 22.4 Hz, 3H), 1.35-1.22 | |
| | (m, 4H), 1.18-1.06 (m, 4H). | |
| 115 | ¹H NMR (400 MHz, CDCl₃): δ 9.87 (s, 1H), 7.97 (m, 2H), 7.53- | 523.1 |
| | 7.40 (m, 3H), 7.32 (t, J = 7.6 Hz, 1H), 6.69 (d, J = 6.8 Hz, | |
| | 1H), 6.45 (d, J = 45.6 Hz, 1H), 5.28 (t, J = 6.4 Hz, 2H), 5.22 | |
| | (d, J = 6.4 Hz, 2H), 4.99 (t, J = 5.6 Hz, 1H), 3.80 (br s, 1H), | |
| | 3.59 (br s, 2H), 3.15-3.06 (m, 1H), 3.02 (s, 3H), 2.26-2.15 | |
| | (m, 1H), 1.68 (d, J = 22 Hz, 3H), 1.42-1.36 (m, 4H), 1.19- | |
| | 1.10 (m, 4H). | |
| 121 | ¹H NMR (400 MHz, CDCl₃): δ 9.18 (br s, 1H), 7.94 (s, 1H), | 393.1 |
| | 7.56-7.48 (m, 2H), 7.36 (s, 1H), 7.28-7.22 (m, 1H), 6.92 | |
| | (d, J = 7.2 Hz, 1H), 4.14 (s, 3H), 3.37 (q, J = 7.2 Hz, 1H), | |
| | 3.23 (s, 3H), 3.10 (dd, J = 6.8, 14.8 Hz, 1H), 2.93 (dd, J = | |
| | 7.6, 14.8 Hz, 1H), 1.44 (d, J = 6.8 Hz, 3H). | |
| 122 | ¹H NMR (400 MHz, CDCl₃): δ 9.12 (br s, 1H), 7.97 (s, 1H), | 351.2 |
| | 7.55 (d, J = 8.4 Hz, 1H), 7.53 (br s, 1H), 7.24 (t, J = 8.0 Hz, | |
| | 1H), 6.90 (d, J = 7.6 Hz, 1H), 3.36 (q, J = 7.2 Hz, 1H), 3.22 | |
| | (s, 3H), 3.09 (dd, J = 6.8, 14.8 Hz, 1H), 2.93 (dd, J₁ = 7.2, | |
| | 14.8 Hz, 1H), 1.94-1.85 (m, 1H), 1.44 (d, J = 7.2 Hz, 3H), | |
| | 0.97-0.89 (m, 2H), 0.80-0.73 (m, 2H). | |
| 123 | ¹H NMR (400 MHz, DMSO) δ 13.90 (s, 1H), 10.47 (s, 1H), | 407.2 |
| | 8.19 (s, 1H), 8.03 (s, 1H), 7.78 (d, J = 9.7 Hz, 1H), 7.46 (d, | |
| | J = 2.0 Hz, 1H), 7.25 (t, J = 7.9 Hz, 1H), 6.67 (d, J = 7.7 | |
| | Hz, 1H), 4.93 (d, J = 6.0 Hz, 2H), 4.83 (d, J = 5.9 Hz, 2H), | |
| | 3.47 (s, 2H), 2.90 (s, 3H). | |
| 124 | ¹H NMR (400 MHz, DMSO) δ 13.90 (s, 1H), 10.48 (s, 1H), | 425.2 |
| | 8.36 (s, 1H), 8.01 (d, J = 1.3 Hz, 1H), 7.81 (ddd, J = 8.3, | |
| | 2.1, 1.0 Hz, 1H), 7.61 (t, J = 2.0 Hz, 1H), 7.28 (t, J = 8.0 | |
| | Hz, 1H), 6.86 (ddd, J = 7.8, 1.9, 1.0 Hz, 1H), 6.23 (d, J = | |
| | 45.8 Hz, 1H), 5.33 (dd, J = 6.8, 1.1 Hz, 1H), 5.17 (dd, J = | |
| | 6.2, 1.3 Hz, 1H), 5.05 (dd, J = 6.7, 1.9 Hz, 1H), 4.77 (dd, | |
| | J = 6.2, 4.1 Hz, 1H), 3.18 (s, 3H). | |
| 224 | ¹H NMR (400 MHz, CDCl₃) δ 9.91 (s, 1H), 8.02 (s, 1H), 7.85 | 578.4 |
| | (s, 1H), 7.47 (s, 1H), 7.55 (s, 1H), 7.46 (dd, J = 1.2, 8.0 Hz, | |
| | 1H), 7.30 (s, 1H), 7.24 (t, J = 8.0 Hz, 1H), 6.55(d, J = 8.0 | |
| | Hz, 1H), 4.19 (d, J = 14.4 Hz, 1H), 3.44-3.31 (m, 2H), 3.28 | |
| | (s, 2H), 3.15 (d, J = 14.4 Hz, 1H), 3.08-2.93 (m, 3H), 2.77- | |
| | 2.65 (m, 4H), 2.66-2.34 (m, 6H), 2.33-2.08 (m, 4H), 1.17- | |
| | 1.05 (m, 4H), 1.01 (d, J = 6.8 Hz, 3H), 0.97 (d, J = 6.8 Hz, | |
| | 3H). | |
| 223 | ¹H NMR (400 MHz, CDCl₃) δ 9.89 (s, 1H), 8.03 (s, 1H), 7.86 | 596.4 |
| | (s, 1H), 7.57 (dd, J = 1.2, 8.0 Hz, 1H), 7.47 (s, 1H), 7.29 (t, | |
| | J = 8.0 Hz, 1H), 7.25 (s, 1H), 6.70 (d, J = 7.6 Hz, 1H), 6.07 | |
| | (d, J = 44.8 Hz, 1H), 4.21 (d, J = 14.4 Hz, 1H), 3.68-3.38 | |
| | (m, 2H), 3.32-3.06 (m, 4H), 3.05-2.89 (m, 2H), 2.99-2.76 | |
| | (m, 4H), 2.76-2.62 (m, 5H), 2.60-2.42 (m, 1H), 2.39-2.26 | |
| | (m, 1H), 2.20-2.09 (m, 1H), 1.19-1.07 (m, 4H), 1.04 (d, | |
| | J = 7.2 Hz, 3H), 0.97 (d, J = 6.8 Hz, 3H). | |
| 225 | ¹H NMR (400 MHz, CDCl₃) δ 9.87 (s, 1H), 8.06 (s, 1H), 7.90 | 596.4 |
| | (s, 1H), 7.60-7.52 (m, 1H), 7.46 (s, 1H), 7.33-7.31(m, 1H), | |
| | 7.30 (t, J = 8.0 Hz, 1H), 7.25 (s, 1H), 6.73 (d, J = 8.0 Hz, | |
| | 1H), 6.08 (d, J = 44.8 Hz, 1H), 4.26 (d, J = 14.4 Hz, 1H), | |
| | 3.68-3.53 (m, 1H), 3.53-3.43 (m, 1H), 3.43-3.31 (m, 2H), | |
| | 3.28-3.09 (m, 3H), 3.08-2.90 (m, 3H), 2.88-2.71 (m, 8H), | |
| | 2.46-2.30 (m, 1H), 2.24-2.07 (m, 1H), 1.18-1.08 (m, 4H), | |
| | 1.06 (d, J = 7.2 Hz, 3H), 1.00 (d, J = 7.2 Hz, 3H). | |
| 495 | ¹H NMR (400 MHz, methanol-d₄) δ 8.18 (s, 1H), 7.88 (s, | 523.9 |
| | 1H), 7.65 (d, J = 8.0 Hz, 1H), 7.42 (s, 1H), 7.37 (s, 1H), 7.34- | |
| | 7.29 (m, 1H), 6.71 (d, J = 7.6 Hz, 1H), 3.77 (s, 2H), 3.40- | |
| | 3.25 (m, 6H), 3.15-3.12 (m, 2H), 3.04-3.00 (m, 2H), | |
| | 2.75 (s, 3H), 2.22-2.20 (m, 1H), 1.49 (s, 3H), 1.16-1.08 | |
| | (m, 4H). | |
| 496 | ¹H NMR (400 MHz, methanol-d₄) δ 8.18 (s, 1H), 7.89 (s, | 537.3 |
| | 1H), 7.65 (d, J = 8.4 Hz, 1H), 7.43 (s, 1H), 7.36 (s, 1H), 7.34- | |
| | 7.29 (m, 1H), 6.71 (d, J = 8.0 Hz, 1H), 3.77 (s, 2H), 3.53 | |
| | (s, 2H), 3.33-3.25 (m, 6H), 3.07-3.00 (m, 4H), 2.75 (s, | |
| | 3H), 2.23-2.19 (m, 1H), 1.27 (s, 3H), 1.15-1.08 (m, 4H). | |
| 497 | ¹H NMR (400 MHz, methanol-d₄) δ 8.25 (s, 1H), 7.89 (s, | 555.6 |
| | 1H), 7.69 (d, J = 8.0 Hz, 1H), 7.52 (s, 1H), 7.34 (t, J = 8.0 | |
| | Hz, 2H), 6.84 (d, J = 7.6 Hz, 1H), 6.05 (d, J = 44.4 Hz, 1H), | |
| | 3.79 (s, 2H), 3.64-3.54 (m, 1H), 3.53-3.45 (m, 4H), 3.34- | |
| | 3.31 (m, 1H), 3.20-3.14 (m, 1H), 3.14-3.06 (m, 3H), | |
| | 2.90 (s, 3H), 2.23-2.19 (m, 1H), 1.27 (s, 3H), 1.16-1.13 | |
| | (m, 2H), 1.13-1.08 (m, 2H). | |
| 498 | ¹H NMR (400 MHz, methanol-d₄) δ 8.19 (s, 1H), 7.96 (s, | 544.7 |
| | 1H), 7.65 (d, J = 8.0 Hz, 1H), 7.43 (s, 1H), 7.36-7.30 (m, | |
| | 2H), 6.62 (d, J = 8.0 Hz, 1H), 5.08-5.03 (m, 4H), 4.22 (d, | |
| | J = 14.8 Hz, 1H), 3.64 (s, 2H), 3.21 (d, J = 14.8 Hz, 1H), | |
| | 2.86 (s, 3H), 2.86-2.82 (m, 1H), 2.74-2.71 (m, 2H), 2.37- | |
| | 2.33 (m, 1H), 2.33 (s, 3H), 2.32-2.16 (m, 5H), 1.14- | |
| | 1.10 (m, 2H), 1.09-1.05 (m, 2H), 1.01 (d, J = 6.8 Hz, 3H), | |
| | 0.97 (d, J = 6.4, | |
| | Hz, 3H). | |
| 499 | ¹H NMR (400 MHz, methanol-d₄) δ 8.28 (s, 1H), 7.96 (s, | 562.4 |
| | 1H), 7.69 (d, J = 8.0 Hz, 1H), 7.43 (s, 2H), 7.35 (t, J = 8.0 | |
| | Hz, 1H), 6.78 (d, J = 8.0 Hz, 1H), 6.29 (d, J = 45.2 Hz, 1H), | |
| | 5.48-5.46 (m, 1H), 5.31-5.29 (m, 1H), 5.20-5.17 (m, | |
| | 1H), 5.02-4.88 (m, 1H), 4.22 (d, J = 14.8 Hz, 1H), 3.20 (d, | |
| | J = 14.8 Hz, 1H), 3.07 (s, 3H), 2.82-2.75 (m, 1H), 2.73- | |
| | 2.65 (m, 2H), 2.32-2.22 (m, 6H), 2.22-2.18 (m, 2H), 2.18- | |
| | 2.15 (m, 1H), 1.14-1.08 (m, 4H), 1.01 (d, J = 6.8 Hz, 3H), | |
| | 0.97 (d, J = 6.8 Hz, 3H). | |
| 500 | ¹H NMR (400 MHz, methanol-d₄) δ 8.27 (s, 1H), 7.96 (s, | 562.3 |
| | 1H), 7.68 (d, J = 8.0 Hz, 1H), 7.42 (s, 2H), 7.35 (t, J = 8.0 | |
| | Hz, 1H), 6.78 (d, J = 8.0 Hz, 1H), 6.29 (d, J = 45.2 Hz, 1H), | |
| | 5.47 (d, J = 6.4 Hz, 1H), 5.30 (d, J = 6.4 Hz, 1H), 5.20-5.17 | |
| | (m, 1H), 5.02-5.00 (m, 1H), 4.22 (d, J = 14.8 Hz, 1H), 3.20 | |
| | (d, J = 14.8 Hz, 1H), 3.07 (s, 3H), 2.85-2.80 (m, 1H), 2.74- | |
| | 2.71 (m, 2H), 2.36-2.20 (m, 9H), 1.14-1.07 (m, 4H), | |
| | 1.01 (d, J = 6.8 Hz, 3H), 0.97 (d, J = 6.8 Hz, 3H). | |
| 501 | ¹H NMR (400 MHz, methanol-d₄) δ 8.46 (d, J = 8.0 Hz, 1H), | 436.1 |
| | 8.42 (s, 1H), 8.30 (t, J = 8.0 Hz, 1H), 8.06 (d, J = 7.2 Hz, | |
| | 1H), 7.77 (d, J = 8.0 Hz, 1H), 7.73 (s, 1H), 7.40 (t, J = 8.0 | |
| | Hz, 1H), 7.03 (d, J = 8.0 Hz, 1H), 6.07 (d, J = 44.4 Hz, 1H), | |
| | 4.55-4.52 (m, 1H), 4.44-4.41 (m, 1H), 3.52-3.48 (m, | |
| | 1H), 3.39 (s, 3H), 2.92-2.89 (m, 1H). | |
| 502 | ¹H NMR (400 MHz, methanol-d₄) δ 8.45 (d, J = 8.0 Hz, 1H), | 436.1 |
| | 8.42 (s, 1H), 8.29 (t, J = 8.0 Hz, 1H), 8.05 (d, J = 8.0 Hz, | |
| | 1H), 7.77 (d, J = 8.0 Hz, 1H), 7.73 (s, 1H), 7.40 (t, J = 8.0 | |
| | Hz, 1H), 7.03 (d, J = 7.6 Hz, 1H), 6.07 (d, J = 44.4 Hz, 1H), | |
| | 4.54-4.51 (m, 1H), 4.43-4.40 (m, 1H), 3.52-3.48 (m, | |
| | 1H), 3.39 (s, 3H), 2.92-2.89 (m, 1H). | |
| 503 | ¹H NMR (400 MHz, methanol-d₄) δ 8.45 (d, J = 7.6 Hz, 1H), | 436.0 |
| | 8.35 (s, 1H), 8.29 (t, J = 7.6 Hz, 1H), 8.05 (d, J = 7.6 Hz, | |
| | 1H), 7.74 (d, J = 8.0 Hz, 1H), 7.65 (s, 1H), 7.36 (t, J = 8.0 | |
| | Hz, 1H), 7.02 (d, J = 8.0 Hz, 1H), 6.07 (d, J = 43.6 Hz, 1H), | |
| | 4.74-4.70 (m, 1H), 4.60-4.57 (m, 1H), 3.74 (s, 3H), 3.59- | |
| | 3.53 (m, 1H), 2.94-2.91 (m, 1H). | |
| 504 | ¹H NMR (400 MHz, methanol-d₄) δ 8.45 (d, J = 8.0 Hz, 1H), | 436.1 |
| | 8.35 (s, 1H), 8.29 (t, J = 8.0 Hz, 1H), 8.05 (d, J = 7.6 Hz, | |
| | 1H), 7.74 (d, J = 7.6 Hz, 1H), 7.65 (s, 1H), 7.36 (t, J = 8.0 | |
| | Hz, 1H), 7.02 (d, J = 8.0 Hz, 1H), 6.07 (d, J = 43.6 Hz, 1H), | |
| | 4.74-4.71 (m, 1H), 4.60-4.57 (m, 1H), 3.74 (s, 3H), 3.59- | |
| | 3.55 (m, 1H), 2.94-2.90 (m, 1H). | |
| 505 | ¹H NMR (400 MHz, methanol-d₄) δ 8.48 (s, 1H), 8.19 (s, | 581.2 |
| | 1H), 8.11 (s, 1H), 7.72 (d, J = 9.2 Hz, 1H), 7.34 (t, J = 8.0 | |
| | Hz, 2H), 6.65 (d, J = 7.2 Hz, 1H), 5.09-5.04 (m, 4H), 3.64 | |
| | (s, 2H), 3.60-3.55 (m, 1H), 2.87 (s, 3H), 2.86-2.81 (m, | |
| | 1H), 2.62-2.59 (m, 2H), 2.42-2.39 (m, 1H), 1.85-1.81 | |
| | (m, 4H), 0.57-0.55 (m, 4H), 0.37-0.24 (m, 2H), 0.25- | |
| | 0.22 (m, 1H), −0.02-−0.04 (m, 1H), −0.23-−0.24 (m, 1H). | |

### Example 25: Cbl-b and C-cbl LCK Ub TR-FRET Assay

Compounds were 3-fold serially diluted in DMSO in a 384-well polypropylene plate (#P-05525-BC; Labcyte) to generate a source plate with 10 concentrations of each compound, top concentration=2 mM. 80 nL of DMSO or compounds were transferred to each well of a black 384-well ProxiPlate (#6008260; PerkinElmer) using a Labcyte Echo. 1× assay buffer (50 mM HEPES pH7.0, 100 mM NaCl, 0.01% BSA, 0.01% Triton-X100, 1 mM DTT), 2× enzyme solution (16 nM Biotin-Cbl-b or 12 nM Biotin-c-Cbl in 1× assay buffer), 2× kinase mixture (120 nM His-LCK, 1 mM ATP, 10 mM MgCl₂ in assay buffer) and 2.33× detection mixture (4.66× solution 1: 163 nM Anti-HA-D2 antibody (#610HADAB; PerkinElmer), 27.96 nM Streptavidin-EU (#AD0062; PerkinElmer), 1.398 mM EDTA in 1× assay buffer+4.66× solution 2: 2.796 μM UBE2D2/Methylated-HA-Ubiquitin thioester adduct (BostonBiochem) in 1× assay buffer) were prepared. 4 μL of 2× enzyme solution was added to each well containing compound, briefly centrifuged to mix, and incubated for 60 min at room temperature. 4 μL of 2× kinase mixture was added, briefly centrifuged to mix, and incubated for 90 min. at room temperature. 6 μL of detection mixture was added to all wells and briefly centrifuged before incubating for 20 min at room temperature. Plates were read for TR-FRET using an Envision at excitation 340 nm, emission at 615 and 665 nm, 4 flashes per well. IC₅₀ was generated using no LCK as the low control and DMSO as the high control.

### Example 26: PBMC IL-2 Assay

Immune response to compounds described herein can be assessed via a PBMC IL-2 assay, conducted according to the following protocol. PBMCs (#A19K379053, A19K261022; TPCS) are thawed into complete medium: 1640 medium (#2085568; Gibco), 10% FBS (#SH30084.03; HyClone), and 1× pen/strep. Compounds are 3-fold serially diluted in DMSO in a 384-well polypropylene plate (#P-05525-BC; Labcyte) using the Tecan EVO to generate a source plate with 10 concentrations of each compound, top concentration=4 mM. Compounds are dispensed into a 96-well plate (#6005680; PerkinElmer) using a Labcyte Echo; final dispensed volume of each control and compound is 1000 nL (final DMSO=0.5%). After recovery overnight, cells are seeded at 2×10⁵ cells/well into 96-well plates containing compounds and incubated at 37° C., 5% CO₂ for 30 min. Cells are stimulated by adding 20 μL/well 1/10 TransAct (#130-111-160; Miltenyi) diluted in complete medium, placed on a shaker for 2 min at 600 rpm, and incubated for 24 h at 37° C., 5% CO₂. Plates are centrifuged at 1200 rpm for 5 min and 120 μL cell supernatant collected. Supernatants are diluted 10-fold and IL-2 concentrations of each sample are determined using the IL-2 MSD kit (#K151AHB-4; MSD) per the manufacturer's instructions.

### Example 27: Liver Microsomes Metabolic Stability Assays

Metabolic stability of test compounds was evaluated in pooled rat, mouse, dog, and cynomolgus monkey liver microsomes (BD Biosciences, San Jose, CA). The incubation conditions were as follows: 1 μM of the tested compound, 1 mM NADPH, 0.5 mg/mL microsomal protein in 0.1 M potassium phosphate buffer (pH 7.4). Following a 5-minute pre-incubation period, the enzymatic reactions were initiated by the addition of NADPH and test compound to the microsomes diluted in phosphate buffered saline. The mixtures were incubated at 37° C. for 0, 20, 40, and 60 min.

Compound concentrations were assessed by LC-MS/MS. Intrinsic clearance based upon microsomal stability data was determined using a substrate depletion method and scaled to hepatic clearance using the well-stirred model (Obach, R. S.; Baxter, J. G.; Liston, T. E.; Silber, B. M.; Jones, B. C.; MacIntyre, F.; Rance, D. J.; Wastall, P., “The Prediction of Human Pharmacokinetic Parameters from Preclinical and in vitro Metabolism Data”, J. Pharmacol. Exp. Ther., (1997), 283 (1), 46-58).

### Example 28: Hepatocyte Metabolic Stability Assays

Metabolic stability assays of test compounds were evaluated in cryopreserved pooled rat, mouse, dog, and cynomolgus monkey hepatocytes (CellzDirect; Durham, NC, USA). Membrane integrity of the cells was assessed by trypan blue exclusion. Test compounds (1.0 μM with 0.1% dimethylsulfoxide) were incubated with cells (0.5 million cells/mL) at 37° C. in a 95% air/5% CO₂ atmosphere for 0, 20, 40, or 60 minutes. Concentrations of test compounds in hepatocyte incubations were determined by LC/MS/MS. Intrinsic clearance was determined using a substrate depletion method and scaled to hepatic clearance using the well-stirred model as described above for the liver microsomes metabolic stability assays.

### Example 29: In Vitro Plasma Protein Binding

In vitro plasma protein binding (n=2) was determined in pooled mouse, rat, and human plasma (Bioreclamation, Inc., Hicksville, NY) by equilibrium dialysis using a Rapid Equilibrium Dialysis (RED) device (Pierce Biotechnology/Thermo Fisher Scientific; Rockford, IL) with a molecular weight cut-off of 8000 Daltons. Test compounds were added to plasma. Plasma samples were equilibriated with phosphate-buffered saline at 37° C. for 4 hours. Compound concentrations in post-dialysis plasma and buffer samples were measured by LC-MS/MS. The percent unbound fraction in plasma for each compound was calculated by dividing the compound concentration in the post-dialysis buffer by that measured in the post-dialysis plasma and multiplying by 100%.

### Example 30: In Vitro Permeability Assay in gMDCK (Madin-Darby Canine Kidney) Cells

The permeability of test compounds can be determined in gMDCK cells (American Type Culture Collection; Manassas, VA). Four days prior to use, MDCK cells were seeded at a density of 2.5×10⁵ cells/mL in 24 well plates. Compounds were dissolved in transport buffer consisting of Hank's Balanced Salt Solution with 10 mM HEPES (Invitrogen Corporation, Grand Island, NY) at a concentration of 10 μM, and permeability was assessed in the apical to basolateral (A-B) and basolateral to apical (B-A) directions following a 3 hour incubation. Lucifer Yellow (Sigma Aldrich, St. Louis, MO) was used as the cell monolayer integrity marker. Test compound concentrations in the donor and receiving compartments were determined by LC-MS/MS. The apparent permeability (Pₐₚₚ) of test compounds was determined as follows:

$${P}_{app} = ( dQ / dt ) * ( 1 / {AC}_{0} )$$

Where dQ/dt is the rate of compound appearance in the receiver compartment, Q is the quantity of compound), C₀ is the concentration in the donor compartment and A is the surface area of the insert. Efflux ratio was calculated as P_{app, B-A}/P_{app, A-B}.

### Example 31: Reversible CYP Inhibition

Reversible CYP inhibition by compounds described herein can be measured by protocols described by Halladay, J. S.; Delarosa, E. M.; Tran, D.; Wang, L.; Wong, S.; Khojasteh, S. C., “High-Throughput, 384-Well, LC-MS/MS CYP Inhibition Assay Using Automation, Cassette-Analysis Technique, and Streamlined Data Analysis”, Drug. Metab. Lett. 2011, 5 (3), 220-230, incorporated herein by reference.

### Example 32: CYP3A Time-Dependent Inhibition (TDI)

Time-dependent inhibition by compounds described herein can be measured by various methods. Exemplary such protocols for CYP3A automated AUC shift dilution TDI assay and definitive K_{I}/K_{inact} TDI assay are described by Kenny, J. R.; Mukadam, S.; Zhang, C.; Tay, S.; Collins, C.; Galetin, A.; Khojasteh, S. C., “Drug-Drug Interaction Potential of Marketed Oncology Drugs: in vitro Assessment of Time-Dependent Cytochrome P450 Inhibition, Reactive Metabolite Formation and Drug-Drug Interaction Prediction,” Pharm. Res. 2012, 29 (7), 1960-1976.

### Example 33: In Vivo Pharmacokinetics (PK)

The pharmacokinetics of test compounds were evaluated following a single intravenous bolus (IV) of solution at a dose of 0.2-1 mg/kg and oral administration (PO) of solution/suspension at doses of 1-5 mg/kg in male cynomolgus monkey, beagle dogs, or Sprague Dawley rats using a parallel study design. Blood samples for the IV dose group were collected prior to administration (predose) and at 0.033, 0.083, 0.25, 0.5, 1, 3, 6, 9 and 24 hours post dose. Blood samples for PO dose groups were collected prior to administration (predose) and at 0.083, 0.25, 0.5, 1, 3, 6, 9 and 24 hours post dose. For the IV group, urine was collected from each animal at predose and from 0-6 and 6-24 hours post dose. The vehicle used for IV dose groups was a combination of PEG400 with citrate buffer (pH 5.0) or PEG400/Cremphor with DMSO/H₂O, and for PO groups was MCT.

Plasma and urine concentrations were quantitated at Genentech Inc. using a non-validated LC/MS/MS method. The lower limit of quantitation (LLOQ) of the plasma and urine assays was 0.005 μM. PK parameters were determined by non-compartmental methods using WinNonlin (version 5.2, Pharsight Corporation, Mountain View, CA).

### Example 34: Pharmacodynamics (PD) Study; Enhancement of CD4 and CD8 T Cell Activation in Response to Systemic Anti-CD3 Administration in the Presence of Cbl-b Inhibitor

Female C57BL/6 or Balb/c mice are administered with anti-CD3 antibody (2 ug/mouse, clone 2C11) or an isotype control (2 μg/mouse, hamster IgG) is administered by tail vein injection. A Cbl-b inhibitor is administered PO starting at time 0 (immediately before anti-CD3 administration) and again 8 hrs later. Four hours after anti-CD3 administration, mice are bled and cytokines are quantified in serum via Luminex. Twenty-four hours after anti-CD3 administration, mice are euthanized and activation of CD4 and CD8 T cells quantified in spleens and blood. Expression of 4-1 BB, CD25, CD40L, and CD69 as well as cell surface TCR levels will be quantified by flow cytometry. Serum are collected for cytokine analysis via Luminex.

### Example 35: Tumor PD/Efficacy Study; Evaluation of Tumor Growth and Immune Cell Infiltration in Mice with Syngeneic Tumors Treated Prophylactically or Therapeutically with Cbl-b Inhibitor

Female C57Bl/6 mice age 6-12 weeks are inoculated subcutaneously in the 5th mammary fat pad with 0.1 million E0771 cells in 100 microliters of HBSS+matrigel under manual restraint. For prophylactic studies, a Cbl-b inhibitor is administered PO BIDx3 weeks starting 1 hr prior to tumor inoculation. Three weeks after tumor inoculation, mice are euthanized and tumor, spleen, blood and draining lymph node are harvested and immune cell infiltrate and phenotype are assessed by flow cytometry. Serum are obtained at various time points for cytokine analysis via Luminex. For therapeutic efficacy assessment, tumors are inoculated as described above and allowed to grow until tumors reach a median volume of 120-250 mm³. Dosing with a Cbl-b inhibitor is then be initiated as above and continued until end of study. Tumor volumes and mouse body weight and condition are recorded twice weekly or more as needed until end of study. Efficacy of a Cbl-b inhibitor can also be assessed in additional syngeneic tumor models, including CT26 and TC-1.

TABLE 3
Cbl-b and C-Cbl Selectivity of Picolinamide Compounds

| | Cbl-b Lck HTRF | C-cbl Lck HTRF | (fold- |
|---|---|---|---|
| Compound | IC₅₀ (μM) | IC₅₀ (μM) | selectivity) |
| 1 | 0.11 | 0.25 | (2.2x) |
| 2 | 0.22 | 0.37 | (1.7x) |
| 3 | 0.025 | 0.047 | (1.9x) |
| 4 | 0.067 | 0.13 | (1.9x) |
| 5 | 0.019 | 0.030 | (1.6x) |
| 6 | 0.020 | 0.033 | (1.7x) |
| 7 | 0.017 | 0.031 | (1.8x) |
| 8 | 0.030 | 0.060 | (2.0x) |
| 16 | 0.26 | 0.41 | (1.6x) |
| 18 | 0.26 | 0.28 | (1.1x) |
| 19 | 0.28 | 0.57 | (2.1x) |
| 20 | 0.27 | 0.35 | (1.3x) |
| 21 | 0.51 | 0.67 | (1.3x) |
| 22 | 0.012 | 0.010 | (0.87x) |
| 23 | 0.050 | 0.047 | (0.94x) |
| 24 | 0.063 | 0.058 | (0.92x) |
| 25 | 0.020 | 0.020 | (1.0x) |
| 26 | 1.5 | 2.2 | (1.5x) |
| 35 | 0.023 | 0.40 | (1.8x) |
| 36 | 0.14 | 0.24 | (1.8x) |
| 37 | 0.78 | 1.5 | (2.0x) |
| 38 | 0.025 | 0.042 | (1.7x) |
| 39 | 0.18 | 0.34 | (1.9x) |
| 40 | 0.88 | 2.1 | (2.3x) |
| 41 | 4.8 | 12 | (2.5x) |
| 42 | 0.19 | 0.27 | (1.4x) |
| 43 | 0.32 | 0.74 | (2.3x) |
| 44 | 2.3 | 4.9 | (2.1x) |
| 45 | 12 | 16 | (1.4x) |
| 46 | 0.42 | 0.80 | (1.9x) |
| 60 | 0.13 | 0.33 | (2.5x) |
| 61 | 0.18 | 0.42 | (2.3x) |
| 62 | 0.12 | 0.14 | (1.2x) |
| 63 | 0.17 | 0.18 | (1.1x) |
| 64 | 0.20 | 0.23 | (1.2x) |
| 65 | 0.20 | 0.23 | (1.2x) |
| 67 | 0.21 | 0.34 | (1.6x) |
| 98 | 0.044 | 0.078 | (1.8x) |
| 112 | 0.23 | 0.21 | (0.94x) |
| 113 | 0.021 | 0.027 | (1.2x) |
| 114 | 0.29 | 0.22 | (0.76x) |
| 115 | 0.050 | 0.056 | (1.1x) |
| 121 | 4.4 | 2.9 | (0.67x) |
| 122 | 2.8 | 2.2 | (0.79x) |
| 123 | 2.3 | 1.8 | (0.77x) |
| 124 | 3.7 | 3.9 | (1.1x) |
| 223 | 0.011 | 0.011 | (1.0x) |
| 224 | 0.0075 | 0.0050 | (0.66x) |
| 225 | 0.0071 | 0.0084 | (1.2x) |
| 495 | 0.017 | 0.012 | (0.68x) |
| 496 | 0.013 | 0.010 | (0.76x) |
| 497 | 0.034 | 0.033 | (0.95x) |
| 498 | 0.010 | 0.0062 | (0.62x) |
| 499 | 0.011 | 0.0070 | (0.63x) |
| 500 | 0.0099 | 0.0080 | (0.82x) |
| 501 | 15 | 17 | (1.1x) |
| 502 | 20 | 20 | (1.0x) |
| 503 | 20 | 17 | (0.85x) |
| 504 | 16 | 8.7 | (0.55x) |
| 505 | 0.026 | 0.039 | (1.5x) |
| 506 | 0.15 | 0.16 | (1.1x) |
| 507 | 0.10 | 0.089 | (0.89x) |

Cbl-b Lck HTRF data in Table 3 is measured according to Example 25 herein; C-cbl Lck HTRF data is measured according to Example 25 herein.

### Example 36: Cbl-b and c-cbl SPR Assay

Affinity of binding to Cbl-b and c-Cbl for compounds described herein can be assessed by surface plasmon resonance (SPR) according to the following protocol. All experiments were recorded on a Biacore™ 8K or Biacore™ 8K+ (Cytiva) with both surface preparation and experimental measurements performed at 20° C. in an assay buffer consisting of 50 mM HEPES, pH 7.5, 0.15 M NaCl, 0.001% (v/v) Tween® 20, 0.2 mM tris(2-carboxyethyl)phosphine, 0.025% (w/v) carboxymethylated dextran (average MW 10 kDa), 0.2% (w/v) PEG 3350, and 2% DMSO.

Human Cbl-b (residues 40-426) or c-Cbl (residues 47-435) were irreversibly captured to a Series S sensor chip SA (Cytiva 29104992) via an N-terminal avi-tag, biotinylated by co-expression in E. coli with BirA. A surface capture range of 1300-1500 RU of protein was used for both isoforms.

For SPR measurements, 6 concentrations with 2 fold serial dilution were measured with blanks flanking each series for double referencing. Initial concentrations between 20 and 0.5 μM were used depending on the anticipated affinity of the tested compound. SPR sensorgrams were recorded in multi-cycle kinetics format, with a contact time of 60 seconds and a flow rate of 40 μl/min, the dissociation time was varied between 120-1200 seconds aiming for 4-5 half-lives of the measured interaction.

Kinetic and affinity parameters were extracted from the multicycle kinetics data fitting to a 1:1 binding model using the Biacore™ Incyte evaluation software (Cytiva).

### Multi-Point Chaser SPR Assay Variation

For the purposes of this type of experiment the term “chaser compound” refers to a low affinity analogue of the compound under investigation which binds close to saturation at the used concentration and fully dissociates within 120 seconds. For the studies presented herein the chaser compound is ((S)-6-((2-isopropyl-4-methylpiperazin-1-yl)-methyl)-2-(3-(3-((5-methyl-1H-1,2,4-triazol-1-yl)methyl)oxetan-3-yl)phenyl)-4-(trifluoro-methyl)isoindolin-1-one).

Affinity of binding to Cbl-b and c-Cbl for potent compounds described herein (Kd<10 nM) were assessed by surface plasmon resonance (SPR) using a “Chaser” assay format.

A “Chaser” assay utilizes a single cycle kinetics SPR experiment with a contact time of 120 seconds, a flow rate of 50 I/min and a dissociation time of 450 seconds. Single cycle kinetics titration utilized an initial blank injection and 5 concentrations with 2 fold serial dilution with a maximum concentration of 500 nM, blanked to a preceding 6 point blank single cycle kinetics injection for double referencing.

In the case of potent compounds, the protein-compound half-life cannot be accurately measured using routine fitting of the single cycle titration data. The k_{d} is measured independently by determining the percentage unoccupied compound binding site over time by measuring the binding of a chaser compound measured by SPR.

Chaser binding was measured by a multicycle kinetics SPR experiment using a contact time of 20 seconds, a flow rate of 30 I/min, and a dissociation time of 120 seconds. 7 injections of a single chaser concentration of 15 μM with a preceding blank injection, were recorded spaced out between 674 and 30,263 seconds after the last single cycle kinetics titration injection. The % compound bound at a given time was determined by comparison to a single injection of chaser preceding the single cycle kinetics titration defined below.

$$\% Compound Bound = ( 1 - ( RUT / RUT 0 ) ) * 100$$

Where RUT is the observed SPR signal for chaser injection at time T, and RUT0 is the observed SPR signal for chaser injected prior to the titration of compound under investigation.

The % compound bound is plotted against time in seconds and fit to a single exponential, where the exponent represents the kd of the compound-protein complex. The single cycle kinetics experiment of the compound is then fit with a fixed kd determined using the chaser.

SPR and the LCK biochemical assay are orthogonal assays: SPR is a protein binding assay while the LCK assay is an enzyme activity assay. SPR measures compound binding affinity to CBL-B/C-CBL, whereas LCK assay measures compound inhibition of CBL-B/C-CBL ubiquitin transfer activity.

### Example 37: LCMS Methods

The following LCMS methods were used to obtain mass spectrometric data of compounds described elsewhere herein.

LC-MS Method A: 5-95AB_1.5min_220 and 254_Shimadzu

| | |
|---|---|
| Column | MERCK, RP-18e 25-3 mm |

| | |
|---|---|
| Mobile Phase | A: water (4 L) + TFA (1.5 mL) |
| | B: acetonitrile (4 L) + TFA (0.75 mL) |

| TIME (min) | B % |
|---|---|
| 0 | 5 |
| 0.7 | 95 |
| 1.1 | 95 |
| 1.11 | 5 |
| 1.5 | 5 |

| | |
|---|---|
| Flow Rate | 1.5 mL/min |
| wavelength | UV 220 nm, 254 nm |
| Oven Temp | 50° C. |
| Instrument | SHIMADZU LC20-MS2010 |
| MS ionization | ESI |

### Description:

Mobile Phase: 1.5 mL/4 L TFA in water (solvent A) and 0.75 mL/4 L TFA in acetonitrile (solvent B), using the elution gradient 5%-95% (solvent B) over 0.7 minutes and holding at 95% for 0.4 minutes at a flow rate of 1.5 ml/min;
Column: MERCK, RP-18e 25-3 mm;
Wavelength: UV 220 nm, 254 nm;
Column temperature: 50° C.; MS ionization: ESI
LC-MS Method B: 10-80 CD_3MIN_220 and 254_Shimadzu

| | |
|---|---|
| Column | Xbridge Shield RP-18, 5 um, 2.1*50 mm |

| | |
|---|---|
| Mobile Phase | A: water (4 L) + NH₃•H₂O (0.8 mL) |
| | B: acetonitrile |

| TIME (min) | B % |
|---|---|
| 0 | 10 |
| 2 | 80 |
| 2.48 | 80 |
| 2.49 | 10 |
| 3 | 10 |

| | |
|---|---|
| Flow Rate | 1 mL/min |
| wavelength | UV 220 nm & 254 nm |
| Oven Temp | 50° C. |
| MS ionization | ESI |
| Instrument | SHIMADZU LC20-MS2020 |

### Description:

Mobile Phase: 0.8 mL/4 L NH₃·H₂O in water (solvent A) and acetonitrile (solvent B), using the elution gradient 10%-80% (solvent B) over 2 minutes and holding at 80% for 0.48 minutes at a flow rate of 1 ml/min;
Column: XBridge C18 5 um, 2.1*50 mm;
Wavelength: UV 220 nm and 254 nm;
Column temperature: 50° C.; MS ionization: ESI
   LC-MS Method C: 5-95AB_1 min_220 and 254_Agilent

| Column | Agilent Poroshell 120 EC-C18 2.7 um 3.0*30 mm | |
|---|---|---|
| Mobile Phase | A: water (4 L) + TFA (1.5 mL) | |
| | B: acetonitrile (4 L) + TFA (0.75 mL) | |
| | TIME (min) | B % |
| | 0 | 5 |
| | 0.4 | 95 |
| | 0.7 | 95 |
| | 0.71 | 5 |
| | 1 | 5 |

| | |
|---|---|
| Flow Rate | 2.0 mL/min |
| wavelength | UV 220 nm and 254 nm |
| Oven Temp | 50° C. |
| Instrument | Agilent Prime-6125B |
| MS ionization | ESI |

### Description:

Mobile Phase: 1.5 mL/4 LTFA in water (solvent A) and 0.75 mL/4 LTFA in acetonitrile (solvent B), using the elution gradient 5%-95% (solvent B) over 0.4 minutes and holding at 95% for 0.3 minutes at a flow rate of 2.0 ml/min; Column: Agilent Poroshell 120 EC-C18 2.7 um 3.0*30 mm;
Wavelength: UV 220 nm and 254 nm;
Column temperature: 50° C.;
MS ionization: ESI

### LC-MS Method D: 5-95AB_220 and 254_Agilent

| | |
|---|---|
| Column | MERCK, RP-18e 25-2mm |

| | |
|---|---|
| Mobile Phase | A: water (4 L) + TFA (1.5 mL) |
| | B: acetonitrile (4 L) + TFA (0.75 mL) |

| TIME (min) | B % |
|---|---|
| 0 | 5 |
| 0.7 | 95 |
| 1.1 | 95 |
| 1.11 | 5 |
| 1.5 | 5 |

| | |
|---|---|
| Flow Rate | 1.5 mL/min |
| wavelength | UV 220 nm and 254 nm |
| Oven Temp | 50° C. |
| Instrument | Agilent LC1200-MS6110 |
| MS ionization | ESI |

### Description:

Mobile Phase: 1.5 mL/4 L TFA in water (solvent A) and 0.75 mL/4 LTFA in acetonitrile (solvent B), using the elution gradient 5%-95% (solvent B) over 0.7 minutes and holding at 95% for 0.4 minutes at a flow rate of 1.5 ml/min.
Column: MERCK, RP-18e 25-2 mm;
Wavelength: UV 220 nm and 254 nm;
Column temperature: 50° C.;
MS ionization: ESI.

### LC-MS Method E: 10-80AB_7min 220 and 254_Shimadzu

| | |
|---|---|
| Column | Xtimate C18 2.1*30 mm, 3 μm |

| | |
|---|---|
| Mobile Phase | A: water (4 L) + TFA (1.5 mL) |
| | B: acetonitrile (4 L) + TFA (0.75 mL) |

| TIME (min) | B % |
|---|---|
| 0 | 10 |
| 6 | 80 |
| 6.5 | 80 |
| 6.51 | 10 |
| 7 | 10 |

| | |
|---|---|
| Flow Rate | 0.8 mL/min |
| wavelength | UV 220 nm, 254 nm |
| Oven Temp | 50° C. |
| Instrument | SHIMADZU LC20-MS2010 |
| MS ionization | ESI |

### Description:

Mobile Phase: 1.5 mL/4 L, TFA in water (solvent A) and 0.75 mL/4 L TFA in acetonitrile (solvent B), using the elution gradient 10%-80% (solvent B) over 6 minutes and holding at 80% for 0.5 minutes at a flow rate of 0.8 ml/min;
Column: Xtimate C18 2.1*30 mm, 3 um;
Wavelength: UV 220 nm, 254 nm;
Column temperature: 50° C.;
MS ionization: ESI

### Example 38: Comparative Activity and Physicochemical Data

Table 4 shows comparative potency and selectivity data for various compounds herein, as compared to a compound known in the art. In one aspect, the compounds of the present disclosure have greater human liver microsome stability (LM(H)) than a compound in the art.

TABLE 4
Comparative Data

| | Example 519 | |
|---|---|---|
| | WO2019148005 | Compound 224 |
| Lck IC₅₀ (nM) (select.) | 19.0 (0.89x) | 7.5 (0.68x) |
| SPR Kd kin (nM) (select.) | 2.9 (1.7x) | 5.6 (1.3x) |
| PBMC IL-2 Cell EC₅₀ (nM) | 100 | 32 |
| IL-2 hWB EC₅₀ (nM) | 430 | 250 |
| PPB % (H/R/M) | 65/—/74 | 86/—/94 |
| gMDCK Pₐₚₚ AtoB (ratio) | 3.6 (0.9) | |
| LM (H/R/M/D/C) | 17/41/70/28/— | 18/38/60/29/40 |
| Hep (H/R/M) | 16/41/66 | |
| ePBPK (human)* | 0.43 | 0.25 |
| CYP3A4 IC₅₀ (M/T) (uM) | 0.29/2.48 | 0.64/6.7 |
| PXR induction (h) @ 10 uM | 6.2x/22x (control) | |
| Mouse PK | CLp 95 mL/min/kg | CLp 41 mL/min/kg |
| | (unbound CLp 365) | (unbound CLp 683) |
| | F % 2.3% | F % 3.4% |
| LogD (cgLogD) | 2.2 (2.0) | 2.2 (2.9) |
| cLogP | 1.5 | 3.4 |
| tPSA | 85 | 79 |
| Kinet. Solub. (uM) | 177 | 8.3 (8.5) |
| Measured pKa (calc.) | 8.2 (7.0) | |

In Table 4, the ePBPK (human) data is a predicted human unbound conc. @12 hr over PBMC cell EC₅₀ at 5 g dose.

In the CYP3A4 measurements, potency is less desirable. The compounds of the present examples are less potent than the reference compound in the art.

All references cited herein are incorporated by reference in their entireties.

The foregoing description and the associated drawings is intended to illustrate various aspects of the instant technology. It is not intended that the examples presented herein limit the scope of the appended claims. The invention now being fully described, it will be apparent to one of ordinary skill in the art that many changes and modifications can be made thereto without departing from the spirit or scope of the appended claims.

1. A compound of formula (II-A), (II-B), (II-C), (II-D), (II-E) or (II-F): 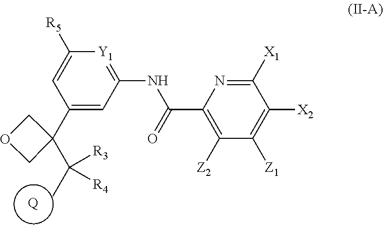
wherein:
Q is a 5-membered heteroaryl, optionally substituted by one or more alkyl, cycloalkyl, or haloalkyl groups;
Y₁ is CH, CF, or N;
R₃, R₄ are independently selected from: H, halogen, alkyl, CN, OH, alkoxy, and haloalkyl;
R₅ is selected from: H, halo, or L₁-R₁₀, wherein L₁ is —N(R₁₁)—, —C(═O)N(R₁₁)—, O, S, carbonyl, or a bond, wherein R₁₀ and Ru are independently H, alkyl, cycloalkyl, alkenyl, alkynyl, aryl, heteroaryl, or heterocyclyl, and R₁₀ and Ru are optionally substituted by one or more groups selected from: halo, CN, amino, carbonyl, OH, alkoxy, alkyl, or haloalkyl, heterocyclyl, aryl, and heteroaryl;
X₁ and X₂ are independently H, halo, haloalkyl, or cycloalkyl, wherein at least one of X₁ and X₂ is not H;
and
one of Z₁ and Z₂ is H and the other is -L₂NR₇R₈,
wherein:
L₂ is —C(H)R₆—, or —C(═O)—, or a bond;
R₆=H, alkyl, cycloalkyl, or haloalkyl; and
R₇ and R₈ are independently selected from H, alkyl, cycloalkyl, hydroxyalkyl, heterocyclyl, and haloalkyl,
wherein, if any of R₇ or R₈ is cycloalkyl or heterocyclyl, said cycloalkyl or heterocyclyl group is optionally substituted with one or more groups selected from: sulfonyl, halo, hydroxyl, alkoxy, alkyl, cycloalkyl, alkoxyalkyl, alkenyl, hydroxyalkyl, cyano, carboxylalkyl, and haloalkyl;
or
R₇ and R₈ together with the nitrogen atom to which they are both bonded form a:
3-8 membered saturated monocyclic ring,
wherein the saturated monocyclic ring is optionally substituted with one or more groups selected from: sulfonyl, halo, hydroxyl, alkoxy, alkyl, cycloalkyl, alkoxyalkyl, alkenyl, hydroxyalkyl, oxo, carboxylalkyl, and haloalkyl;
with the proviso that:
when Y₁ is CH, R₃=R₄=R₅=H, L₂ is CH₂, X₁=CF₃, X₂=H, Z₂=H, and Q is 2-methyl triazol-1-yl, the substituted saturated monocyclic ring is not 3-fluoro-pyrrolidin-1-yl or 2-methyl morpholin-4-yl;
or
R₇ and R₈ together with the nitrogen atom to which they are both bonded form a:
3-8 membered saturated spirocyclic ring,
wherein the saturated spirocyclic ring is optionally substituted with one or more groups selected from: sulfonyl, halo, hydroxyl, cyano, alkyl, alkenyl, cycloalkyl, alkoxy, alkoxyalkyl, hydroxyalkyl, oxo, carboxylalkyl, and haloalkyl;
with the proviso that:
when Y₁ is CH, R₃=R₄=R₅=H, L₂ is CH₂, X₁=CF₃, X₂=H, Z₂=H, and Q is 2-methyl triazol-1-yl, the substituted saturated spirocyclic ring is not 5-azaspiro[2.4]hept-5-yl;
or
R₇ and R₈ together with the nitrogen atom to which they are both bonded form a:
3-8 membered saturated fused bicyclic ring,
wherein the saturated fused bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxylalkyl, and haloalkyl,
with the proviso that:
when Y₁ is CH, R₃=R₄=R₅=H, L₂ is CH₂, X₁=CF₃, X₂=H, and Q is 2-methyl triazol-1-yl, the fused bicyclic ring is not 2-azabicyclo[3.1.0]hex-2-yl, 6,6-difluoro-3-azabicyclo[3.1.0]hex-3-yl, or 6-oxa-3-azabicyclo[3.1.0]hex-3-yl;
or
R₇ and R₈ together with the nitrogen atom to which they are both bonded form a:
3-8 membered saturated bridged bicyclic ring,
wherein the saturated bridged bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxylalkyl, and haloalkyl;
or one of Z₁ and Z₂ is H and the other is 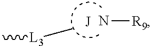
wherein:
L₃ is —C(H)R₆—, —N(R₆)—, O, S, or a bond;
J is a saturated 3-10 membered monocyclic ring, spirocyclic ring, bridged bicyclic ring, or fused bicyclic ring, a 5 or 6 member heteroaromatic ring, or a 3-10 member fused heteroaromatic ring system, and J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, or haloalkyl groups; and
R₉=H, alkyl, cycloalkyl, aminoalkyl, haloalkyl, or carboxyalkyl;
else
Z₁=Z₂=H; 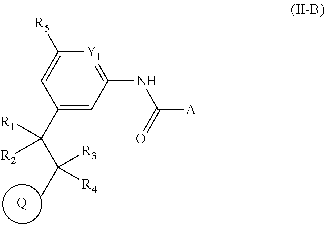
wherein: 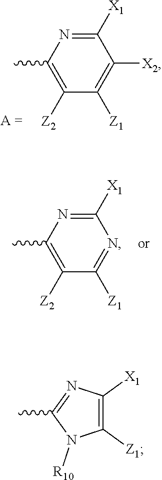
Y₁=CH, CF, or N;
R₁, R₂, R₃, R₄ are independently selected from: H, halo, alkyl, cycloalkyl, CN, OH, alkoxy, and haloalkyl; or
R₂ and R₃ together with the carbon atoms to which they are respectively bonded form a 3-6 membered cycloalkyl, heterocyclyl, or heteroaryl ring, optionally substituted with one or more groups selected from: halo, OH, alkoxy, alkyl, or fluoroalkyl:
R₅ is selected from: H, halo, or L₁-R₁₀, wherein L₁ is —N(R₁₁)—, —C(═O)N(R₁₁)—, O, S, carbonyl, or a bond, wherein R₁₀ and Rn are independently H, alkyl, cycloalkyl, alkenyl, alkynyl, aryl, heteroaryl, or heterocyclyl, and R₁₀ and R₁₁ are optionally substituted by one or more groups selected from: halo, CN, amino, carbonyl, OH, alkoxy, alkyl, or haloalkyl, heterocyclyl, aryl, and heteroaryl;
Q is a 5-membered heteroaryl, optionally substituted by one or more alkyls or fluoro alkyls;
X₁ and X₂ are independently H, halo, haloalkyl, or cycloalkyl, wherein at least one of X₁ and X₂ is not H; and
one of Z₁ and Z₂ is H and the other is —C(H)(R₆)NR₇R₈, wherein:
R₆=H, alkyl, cycloalkyl, or haloalkyl; and
R₇ and R₈ are independently selected from H, alkyl, cycloalkyl, hydroxyalkyl, heterocyclyl, and haloalkyl,
or
R₇ and R₈ together with the nitrogen atom to which they are both bonded form a:
3-8 membered saturated monocyclic ring,
wherein the saturated monocyclic ring is optionally substituted with one or more groups selected from: sulfonyl, halo, hydroxyl, alkoxy, alkyl, cycloalkyl, alkoxyalkyl, alkenyl, hydroxyalkyl, oxo, carboxylalkyl, and haloalkyl;
with the proviso that:
when Y₁ is CH, R₃=R₄=R₅=H, L₂ is CH₂, X₁=CF₃, X₂=H, Z₂=H, A is a substituted pyrimidinyl ring, and Q is 2-methyl triazol-1-yl, the substituted saturated monocyclic ring is not 3-fluoro-pyrrolidin-1-yl;
or
R₇ and R₈ together with the nitrogen atom to which they are both bonded form a:
3-8 membered saturated spirocyclic ring,
wherein the saturated spirocyclic ring is optionally substituted with one or more groups selected from: sulfonyl, halo, hydroxyl, cyano, alkyl, alkenyl, cycloalkyl, alkoxy, alkoxyalkyl, hydroxyalkyl, oxo, carboxylalkyl, and haloalkyl;
or
R₇ and R₈ together with the nitrogen atom to which they are both bonded form a:
3-8 membered saturated fused bicyclic ring,
wherein the saturated fused bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxylalkyl, and haloalkyl;
or
R₇ and R₈ together with the nitrogen atom to which they are both bonded form a:
3-8 membered saturated bridged bicyclic ring,
wherein the saturated bridged bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxylalkyl, and haloalkyl;
or one of Z₁ and Z₂ is H and the other is 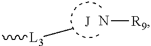
wherein:
L₃ is —C(H)R₆—, —N(R₆)—, O, S, or a bond:
J is a saturated 3-10 membered monocyclic ring, spirocyclic ring, bridged bicyclic ring, or fused bicyclic ring, a 5 or 6 member heteroaromatic ring, or a 3-10 member fused heteroaromatic ring system, and J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, or haloalkyl groups; and
R₉=H, alkyl, or carboxyalkyl;
else Z₁=Z₂=H: 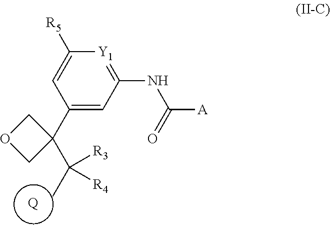
wherein:
Q is a 5-membered heteroaryl ring, optionally substituted by one or more alkyl, cycloalkyl, or haloalkyl groups; 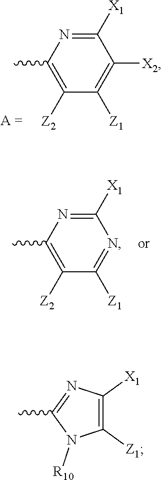
Y₁ is CH, CF, or N;
R₃, R₄ are independently selected from: H, halogen, alkyl, CN, OH, alkoxy, and haloalkyl, and at least one of R₃ and R₄ is halogen;
R₅ is selected from: H, halo, or L₁-R₁₀, wherein L₁ is —N(R₁₁)—, —C(═O)N(R₁₁)—, O, S, carbonyl, or a bond, wherein R₁₀ and Rn are independently H, alkyl, cycloalkyl, alkenyl, alkynyl, aryl, heteroaryl, or heterocyclyl, and R₁₀ and Rn are optionally substituted by one or more groups selected from: halo, CN, amino, carbonyl, OH, alkoxy, alkyl, or haloalkyl, heterocyclyl, aryl, and heteroaryl;
X₁ and X₂ are independently H, halo, haloalkyl, or cycloalkyl, wherein at least one of X₁ and X₂ is not H:
and
one of Z₁ and Z₂ is H and the other is -L₂NR₇R₈, -L₂C(═O)NR₇R₈, or -L₂CR₇R₈(OH);
wherein:
L₂ is —C(H)R₆ₐ—, —C(H)CH₂(R_{6b}), or —C(═O)—, or a bond;
R₆ₐ=H, alkyl, cycloalkyl, or haloalkyl;
R_{6b}=cycloalkyl, or halo; and
R₇ and R₈ are independently selected from H, alkyl, cycloalkyl, hydroxyalkyl, heterocyclyl, and haloalkyl,
wherein, if any of R₇ or R₈ is cycloalkyl or heterocyclyl, said cycloalkyl or heterocyclyl group is optionally substituted with one or more groups selected from: sulfonyl, halo, hydroxyl, alkoxy, alkyl, cycloalkyl, alkoxyalkyl, alkenyl, hydroxyalkyl, cyano, carboxylalkyl, and haloalkyl;
or
R₇ and R₈ together with the nitrogen atom to which they are both bonded form a:
3-8 membered saturated monocyclic ring,
wherein the saturated monocyclic ring is optionally substituted with one or more groups selected from: sulfonyl, halo, hydroxyl, alkoxy, alkyl, cycloalkyl, alkoxyalkyl, alkenyl, hydroxyalkyl, oxo, carboxylalkyl, and haloalkyl;
or
R₇ and R₈ together with the nitrogen atom to which they are both bonded form a:
3-8 membered saturated spirocyclic ring,
wherein the saturated spirocyclic ring is optionally substituted with one or more groups selected from: sulfonyl, halo, hydroxyl, cyano, alkyl, alkenyl, cycloalkyl, alkoxy, alkoxyalkyl, hydroxyalkyl, oxo, carboxylalkyl, and haloalkyl;
or
R₇ and R₈ together with the nitrogen atom to which they are both bonded form a:
3-8 membered saturated fused bicyclic ring,
wherein the saturated fused bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxylalkyl, and haloalkyl;
or
R₇ and R₈ together with the nitrogen atom to which they are both bonded form a:
3-8 membered saturated bridged bicyclic ring,
wherein the saturated bridged bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxylalkyl, and haloalkyl;
or one of Z₁ and Z₂ is H and the other is 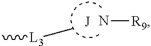
wherein:
L₃ is —C(H)R₆—, —N(R₆)—, O, S, or a bond:
J is a saturated 3-10 membered monocyclic ring, spirocyclic ring, bridged bicyclic ring, or fused bicyclic ring, a 5 or 6 member heteroaromatic ring, or a 3-10 member fused heteroaromatic ring system, and J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, or haloalkyl groups; and
R₉=H, alkyl, cycloalkyl, haloalkyl, aminoalkyl, or carboxyalkyl:
else
Z₁=Z₂=H; 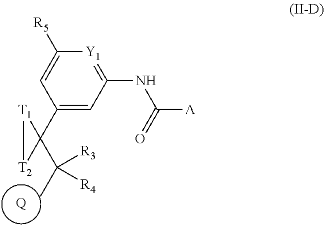
wherein: 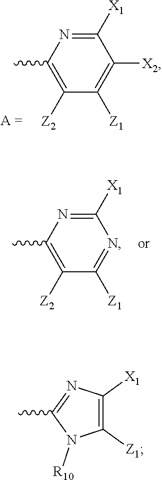
Q is a 5-membered heteroaryl, optionally substituted by one or more alkyl, cycloalkyl, or haloalkyl groups;
Y₁ is CH, CF, or N;
T₁ and T₂ are each independently a group selected from: [—C(R₁)(R₂)—]ₙ, —O—, —C(R₁)(R₂)—O—, >C═O, —C(R₁)(R₂)—C(═O)—, —C(R₁)(R₂)—S(═O)₂—, and >S(═O)₂, with the proviso that T₁ and T₂ together are not —CH₂—O—, —O—O—, or —O—C(═O)—O—;
wherein n=0, 1 or 2, and each of R₁ and R₂ is independently selected from: H, halo, alkyl, alkenyl, cyano, hydroxyl, alkoxy, cycloalkyl, hydroxyalkyl, and haloalkyl, with the proviso that, when T₁ and T₂ together are —CH₂C(R₁)(R₂)CH₂—, neither of R₁ and R₂ is cyano;
or R₁ and R₂ together with the carbon atom to which they are bonded form a cycloalkyl or heterocyclyl ring;
R₃, and R₄ are independently selected from: H, and halo, alkyl, CN, OH, alkoxy, and haloalkyl;
R₅ is selected from: H, halo, or L₁-R₁₀, wherein L₁ is —N(R₁₁)—, —C(═O)N(R₁₁)—, O, S, carbonyl, or a bond, wherein R₁₀ and Ru are independently H, alkyl, cycloalkyl, alkenyl, alkynyl, aryl, heteroaryl, or heterocyclyl, and R₁₀ and Ru are optionally substituted by one or more groups selected from: halo, CN, amino, carbonyl, OH, alkoxy, alkyl, or haloalkyl, heterocyclyl, aryl, and heteroaryl;
X₁ and X₂ are independently H, halo, haloalkyl, or cycloalkyl, wherein at least one of X₁ and X₂ is not H; and
one of Z₁ and Z₂ is H and the other is -L₂NR₇R₈,
wherein:
L₂ is —C(H)R₆—, —C(O)—, or a bond:
R₆=H, alkyl, cycloalkyl, or haloalkyl; and
R₇ and R₈ are independently selected from H, alkyl, cycloalkyl, hydroxyalkyl, heterocyclyl, and haloalkyl,
wherein, if any of R₇ or R₈ is cycloalkyl or heterocyclyl, said cycloalkyl or heterocyclyl group is optionally substituted with one or more groups selected from: sulfonyl, halo, hydroxyl, alkoxy, alkyl, cycloalkyl, alkoxyalkyl, alkenyl, hydroxyalkyl, cyano, carboxylalkyl, and haloalkyl;
or
R₇ and R₈ together with the nitrogen atom to which they are both bonded form a:
3-8 membered saturated monocyclic ring,
wherein the saturated monocyclic ring is optionally substituted with one or more groups selected from: sulfonyl, halo, hydroxyl, alkoxy, alkyl, cycloalkyl, alkoxyalkyl, alkenyl, hydroxyalkyl, oxo, carboxylalkyl, and haloalkyl;
or
R₇ and R₈ together with the nitrogen atom to which they are both bonded form a:
3-8 membered saturated spirocyclic ring,
wherein the saturated spirocyclic ring is optionally substituted with one or more groups selected from: sulfonyl, halo, hydroxyl, cyano, alkyl, alkenyl, cycloalkyl, alkoxy, alkoxyalkyl, hydroxyalkyl, oxo, carboxylalkyl, and haloalkyl;
or
R₇ and R₈ together with the nitrogen atom to which they are both bonded form a:
3-8 membered saturated fused bicyclic ring,
wherein the saturated fused bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxylalkyl, and haloalkyl;
or
R₇ and R₈ together with the nitrogen atom to which they are both bonded form a:
3-8 membered saturated bridged bicyclic ring,
wherein the saturated bridged bicyclic ring is optionally substituted with one or more groups selected from: sulfonyl, halo, hydroxyl, cyano, alkoxy, alkyl, cycloalkyl, alkoxyalkyl, alkenyl, hydroxyalkyl, oxo, carboxylalkyl, and haloalkyl;
or one of Z₁ and Z₂ is H and the other is 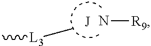
wherein:
L₃ is —C(H)R₆—, —N(R₆)—, O, S, or a bond:
J is a saturated 3-10 membered monocyclic ring, spirocyclic ring, bridged bicyclic ring, or fused bicyclic ring, a 5 or 6 member heteroaromatic ring, or a 3-10 member fused heteroaromatic ring system, and J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, or haloalkyl groups; and
R₉=H, alkyl, cycloalkyl, aminoalkyl, haloalkyl, or carboxyalkyl;
else
Z₁=Z₂=H: 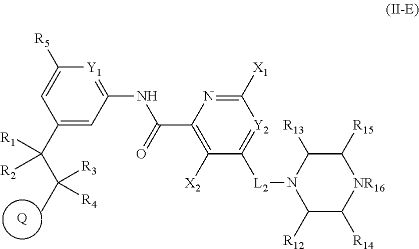
wherein:
Q is a 5-membered heteroaryl group, optionally substituted by one or more alkyl, cycloalkyl, or haloalkyl groups:
Y₁ and Y₂ are independently CH, CF, or N:
R₁, R₂, R₃, R₄ are independently selected from: H, halogen, alkyl, cycloalkyl, CN, OH, alkoxy, and haloalkyl; or
R₁ and R₂, together with the carbon atom to which they are both bonded form a 3-5 membered cycloalkyl or heterocyclyl, optionally substituted with one or more groups independently selected from: halogen, CN, OH, sulfonyl, alkoxy, alkyl, cycloalkyl, hydroxyalkyl, or haloalkyl; or
R₂ and R₃ together with the two carbon atoms to which they are respectively bonded form a 3-6 membered cycloalkyl, cycloalkenyl, heterocyclyl, heteroaryl ring, optionally substituted with one or more groups independently selected from: halogen, OH, alkoxy, cyano, sulfonyl, haloalkyl, hydroxyalkyl, alkyl, cycloalkyl, or alkoxyalkyl;
R₅ is selected from: H, halo, or L₁-R₁₀, wherein L₁ is —N(R₁₁)—, —C(═O)N(R₁₁)—, O, S, carbonyl, or a bond, wherein R₁₀ and Rn are independently H, alkyl, cycloalkyl, alkenyl, alkynyl, aryl, heteroaryl, or heterocyclyl, and R₁₀ and Rn are optionally substituted by one or more groups selected from: halo, CN, amino, carbonyl, OH, alkoxy, alkyl, or haloalkyl, heterocyclyl, aryl, and heteroaryl;
X₁ and X₂ are independently H, halo, haloalkyl, or cycloalkyl, but at least one of X₁ and X₂ is not H; and
L₂ is —C(H)R₁₇—, —C(O)—, or a bond, wherein R₁₇=H, alkyl, cycloalkyl, or haloalkyl;
R₁₂, R₁₃, R₁₄, R₁₅, and R₁₆ are each independently selected from: H, sulfonyl, halo, hydroxyl, alkoxy, alkyl, cycloalkyl, heterocycloakyl, alkoxyalkyl, alkenyl, hydroxyalkyl, oxo, carboxylalkyl, and haloalkyl, wherein, if either of R₁₂, R₁₃, R₁₄, R₁₅, or R₁₆ is alkyl, alkenyl, or haloalkyl, then said alkyl, alkenyl, or haloalkyl may optionally be substituted by one or more cycloalkyl or heterocyclyl groups:
with the proviso that, when Y₁ and Y₂ are both CH, R₁=F, R₂=methyl, R₃=R₄=F, R₅=H, R₁₃=R₁₄=R₁₅=H, X₁=CF₃, X₂=H, Q is 2-methyl triazol-1-yl, and L₁ is a bond, then R₁₂ is not methyl, and R₁₆ is not H or methyl; 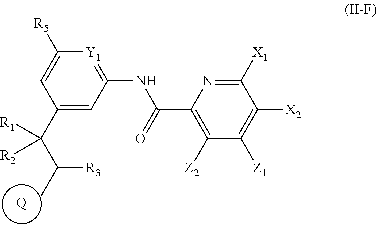
wherein:
Q is a 5-membered heteroaryl, optionally substituted by one or more alkyl, cycloalkyl, or haloalkyl groups;
Y₁ is CH, CF, or N:
R₁ is alkyl and R₂ is H, or R₁ and R₂ together are —CH₂OCH₂—;
R₃ is H or alkyl;
R₅ is selected from: H, halo, or L₁-R₁₀, wherein L₁ is —N(R₁₁)—, —C(═O)N(R₁₁)—, O, S, carbonyl, or a bond, wherein R₁₀ and Ru are independently H, alkyl, cycloalkyl, alkenyl, alkynyl, aryl, heteroaryl, or heterocyclyl, and R₁₀ and Ru are optionally substituted by one or more groups selected from: halo, CN, amino, carbonyl, OH, alkoxy, alkyl, or haloalkyl, heterocyclyl, aryl, and heteroaryl;
X₁ and X₂ are independently H, halo, haloalkyl, or cycloalkyl, wherein at least one of X₁ and X₂ is not H;
and
one of Z₁ and Z₂ is H and the other is -L₂NR₇R₈,
wherein:
L₂ is —C(H)R₆—, —C(O)—, or a bond:
R₆=H, alkyl, cycloalkyl, or haloalkyl; and
R₇ and R₈ are independently selected from H, alkyl, cycloalkyl, hydroxyalkyl, aminoalkyl, heterocyclyl, and haloalkyl,
wherein, if any of R₇ or R₈ is cycloalkyl or heterocyclyl, said cycloalkyl or heterocyclyl group is optionally substituted with one or more groups selected from: sulfonyl, halo, hydroxyl, alkoxy, alkyl, cycloalkyl, alkoxyalkyl, alkenyl, hydroxyalkyl, cyano, carboxylalkyl, and haloalkyl;
or
R₇ and R₈ together with the nitrogen atom to which they are both bonded form a 3-8-membered saturated monocyclic ring, optionally substituted with one or more groups selected from: sulfonyl, halo, alkoxy, alkyl, cycloalkyl, alkoxyalkyl, alkenyl, aminoalkyl, hydroxyalkyl, carboxylalkyl, and haloalkyl;
or
or one of Z₁ and Z₂ is H and the other is 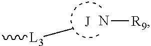
wherein:
L₃ is —C(H)R₆—, —N(R₆)—, O, S, or a bond:
J is a saturated 3-10 membered monocyclic ring, spirocyclic ring, bridged bicyclic ring, or fused bicyclic ring, a 5 or 6 member heteroaromatic ring, or a 3-10 member fused heteroaromatic ring system, and J is optionally substituted by one or more sulfonyl, halo, hydroxyl, cyano, alkoxy, alkyl, cycloalkyl, alkoxyalkyl, alkenyl, hydroxyalkyl, oxo, carboxyalkyl, or haloalkyl groups; and
R₉=H, alkyl, aminoalkyl, haloalkyl, or carboxyalkyl;
or an enantiomer, diastereomer or a pharmaceutically acceptable salt or solvate thereof.

2.-6. (canceled)

7. A compound selected from the group consisting of:
(S)-4-(1-(3-fluoroazetidin-1-yl)ethyl)-N-(3-(3-((4-methyl-4H-1,2,4-triazol-3-yl)methyl)oxetan-3-yl)phenyl)-6-(trifluoromethyl)picolinamide
(R)-4-(1-(3-fluoroazetidin-1-yl)ethyl)-N-(3-(3-((4-methyl-4H-1,2,4-triazol-3-yl)methyl)oxetan-3-yl)phenyl)-6-(trifluoromethyl)picolinamide
(S)-4-(1-(5-azaspiro[2.4]heptan-5-yl)ethyl)-N-(3-(3-((4-methyl-4H-1,2,4-triazol-3-yl)methyl)oxetan-3-yl)phenyl)-6-(trifluoromethyl)picolinamide
(R)-4-(1-(5-azaspiro[2.4]heptan-5-yl)ethyl)-N-(3-(3-((4-methyl-4H-1,2,4-triazol-3-yl)methyl)oxetan-3-yl)phenyl)-6-(trifluoromethyl)picolinamide
(R)-4-(5-azaspiro[2.4]heptan-5-ylmethyl)-6-cyclopropyl-N-(3-(3-(fluoro(4-methyl-4H-1,2,4-triazol-3-yl)methyl)oxetan-3-yl)phenyl)picolinamide
(S)-4-(5-azaspiro[2.4]heptan-5-ylmethyl)-6-cyclopropyl-N-(3-(3-(fluoro(4-methyl-4H-1,2,4-triazol-3-yl)methyl)oxetan-3-yl)phenyl)picolinamide
(R)-6-cyclopropyl-N-(3-(3-(fluoro(4-methyl-4H-1,2,4-triazol-3-yl)methyl)oxetan-3-yl)phenyl)-4-((3-fluoro-3-methylazetidin-1-yl)methyl)picolinamide
(S)-6-cyclopropyl-N-(3-(3-(fluoro(4-methyl-4H-1,2,4-triazol-3-yl)methyl)oxetan-3-yl)phenyl)-4-((3-fluoro-3-methylazetidin-1-yl)methyl)picolinamide
N-(3-(3,3-difluoro-1-((4-methyl-4H-1,2,4-triazol-3-yl)methyl)cyclobutyl)phenyl)-6-(trifluoromethyl)picolinamide
N-(3-((1R,3S)-3-fluoro-1-((4-methyl-4H-1,2,4-triazol-3-yl)methyl)cyclobutyl)phenyl)-6-(trifluoromethyl)picolinamide
N-(3-((1S,3R)-3-fluoro-1-((4-methyl-4H-1,2,4-triazol-3-yl)methyl)cyclobutyl)phenyl)-6-(trifluoromethyl)picolinamide
(R)-2-cyclopropyl-N-(3-(3-(fluoro(4-methyl-4H-1,2,4-triazol-3-yl)methyl)oxetan-3-yl)phenyl)-6-((3-fluoro-3-methylazetidin-1-yl)methyl)pyrimidine-4-carboxamide
2-cyclopropyl-6-[(3-fluoro-3-methyl-azetidin-1-yl)methyl]-N-[3-[3-[(S)-fluoro-(4-methyl-1,2,4-triazol-3-yl)methyl]oxetan-3-yl]phenyl]pyrimidine-4-carboxamide
4-((S)-1-(5-azaspiro[2.4]heptan-5-yl)ethyl)-6-cyclopropyl-N-(3-(3-((R)-fluoro(4-methyl-4H-1,2,4-triazol-3-yl)methyl)oxetan-3-yl)phenyl)-picolinamide
4-((R)-1-(5-azaspiro[2.4]heptan-5-yl)ethyl)-6-cyclopropyl-N-(3-(3-((R)-fluoro(4-methyl-4H-1,2,4-triazol-3-yl)methyl)oxetan-3-yl)phenyl)-picolinamide
4-((S)-1-(5-azaspiro[2.4]heptan-5-yl)ethyl)-6-cyclopropyl-N-(3-(3-((S)-fluoro(4-methyl-4H-1,2,4-triazol-3-yl)methyl)oxetan-3-yl)phenyl)-picolinamide
4-((R)-1-(5-azaspiro[2.4]heptan-5-yl)ethyl)-6-cyclopropyl-N-(3-(3-((S)-fluoro(4-methyl-4H-1,2,4-triazol-3-yl)methyl)oxetan-3-yl)phenyl)picolinamide
(R)-5-(5-azaspiro[2.4]heptan-5-ylmethyl)-N-(3-(1-(4-methyl-4H-1,2,4-triazol-3-yl)propan-2-yl)phenyl)-4-(trifluoromethyl)-1H-imidazole-2-carboxamide
4-(5-azaspiro[2.4]heptan-5-ylmethyl)-6-cyclopropyl-N-(3-((1S,2R)-1,2-difluoro-1-(4-methyl-4H-1,2,4-triazol-3-yl)propan-2-yl)phenyl)picolinamide
4-(5-azaspiro[2.4]heptan-5-ylmethyl)-6-cyclopropyl-N-(3-((1S,2S)-1,2-difluoro-1-(4-methyl-4H-1,2,4-triazol-3-yl)propan-2-yl)phenyl)picolinamide
4-(5-azaspiro[2.4]heptan-5-ylmethyl)-6-cyclopropyl-N-(3-((1R,2S)-1,2-difluoro-1-(4-methyl-4H-1,2,4-triazol-3-yl)propan-2-yl)phenyl)picolinamide
4-(5-azaspiro[2.4]heptan-5-ylmethyl)-6-cyclopropyl-N-(3-((1R,2R)-1,2-difluoro-1-(4-methyl-4H-1,2,4-triazol-3-yl)propan-2-yl)phenyl)picolinamide
6-(5-azaspiro[2.4]heptan-5-ylmethyl)-2-cyclopropyl-N-(3-((1S,2R)-1,2-difluoro-1-(4-methyl-4H-1,2,4-triazol-3-yl)propan-2-yl)phenyl)pyrimidine-4-carboxamide
6-(5-azaspiro[2.4]heptan-5-ylmethyl)-2-cyclopropyl-N-(3-((1S,2S)-1,2-difluoro-1-(4-methyl-4H-1,2,4-triazol-3-yl)propan-2-yl)phenyl)pyrimidine-4-carboxamide
6-(5-azaspiro[2.4]heptan-5-ylmethyl)-2-cyclopropyl-N-(3-((1R,2S)-1,2-difluoro-1-(4-methyl-4H-1,2,4-triazol-3-yl)propan-2-yl)phenyl)pyrimidine-4-carboxamide
6-(5-azaspiro[2.4]heptan-5-ylmethyl)-2-cyclopropyl-N-(3-((1R,2R)-1,2-difluoro-1-(4-methyl-4H-1,2,4-triazol-3-yl)propan-2-yl)phenyl)pyrimidine-4-carboxamide
2-cyclopropyl-N-(3-((1S,2R)-1,2-difluoro-1-(4-methyl-4H-1,2,4-triazol-3-yl)propan-2-yl)phenyl)-6-((3-fluoro-3-methylazetidin-1-yl)methyl)pyrimidine-4-carboxamide
2-cyclopropyl-N-(3-((1S,2S)-1,2-difluoro-1-(4-methyl-4H-1,2,4-triazol-3-yl)propan-2-yl)phenyl)-6-((3-fluoro-3-methylazetidin-1-yl)methyl)pyrimidine-4-carboxamid
2-cyclopropyl-N-(3-((1R,2S)-1,2-difluoro-1-(4-methyl-4H-1,2,4-triazol-3-yl)propan-2-yl)phenyl)-6-((3-fluoro-3-methylazetidin-1-yl)methyl)pyrimidine-4-carboxamide
2-cyclopropyl-N-(3-((1R,2R)-1,2-difluoro-1-(4-methyl-4H-1,2,4-triazol-3-yl)propan-2-yl)phenyl)-6-((3-fluoro-3-methylazetidin-1-yl)methyl)pyrimidine-4-carboxamide
(R)-N-(3-(3-(fluoro(4-methyl-4H-1,2,4-triazol-3-yl)methyl)oxetan-3-yl)phenyl)-4-((3-fluoro-3-methylazetidin-1-yl)methyl)-6-(trifluoromethyl)picolinamide
(S)-N-(3-(3-(fluoro(4-methyl-4H-1,2,4-triazol-3-yl)methyl)oxetan-3-yl)phenyl)-4-((3-fluoro-3-methylazetidin-1-yl)methyl)-6-(trifluoromethyl)picolinamide
(R)-6-((5-azaspiro[2.4]heptan-5-yl)methyl)-2-cyclopropyl-N-(3-(3-(fluoro(4-methyl-4H-1,2,4-triazol-3-yl)methyl)oxetan-3-yl)phenyl)pyrimidine-4-carboxamide
(S)-6-((5-azaspiro[2.4]heptan-5-yl)methyl)-2-cyclopropyl-N-(3-(3-(fluoro(4-methyl-4H-1,2,4-triazol-3-yl)methyl)oxetan-3-yl)phenyl)pyrimidine-4-carboxamide
(R)-4-(aminomethyl)-N-(3-(3-(fluoro(4-methyl-4H-1,2,4-triazol-3-yl)methyl)oxetan-3-yl)phenyl)-6-(trifluoromethyl)picolinamide formate
(S)-4-(aminomethyl)-N-(3-(3-(fluoro(4-methyl-4H-1,2,4-triazol-3-yl)methyl)oxetan-3-yl)phenyl)-6-(trifluoromethyl)picolinamide formate
4-((dimethylamino)methyl)-N-(3-(3-((4-methyl-4H-1,2,4-triazol-3-yl)methyl)oxetan-3-yl)phenyl)-6-(trifluoromethyl)picolinamide
4-((5-azaspiro[2.4]heptan-5-yl)methyl)-N-(3-(3-((4-methyl-4H-1,2,4-triazol-3-yl)methyl)oxetan-3-yl)phenyl)-6-(trifluoromethyl)picolinamide
6-cyclopropyl-N-(3-(3-((R)-fluoro(4-methyl-4H-1,2,4-triazol-3-yl)methyl)oxetan-3-yl)phenyl)-4-((S)-1-(3-fluoro-3-methylazetidin-1-yl)ethyl)picolinamide
6-cyclopropyl-N-(3-(3-((R)-fluoro(4-methyl-4H-1,2,4-triazol-3-yl)methyl)oxetan-3-yl)phenyl)-4-((R)-1-(3-fluoro-3-methylazetidin-1-yl)ethyl)picolinamide
6-cyclopropyl-N-(3-(3-((S)-fluoro(4-methyl-4H-1,2,4-triazol-3-yl)methyl)oxetan-3-yl)phenyl)-4-((S)-1-(3-fluoro-3-methylazetidin-1-yl)ethyl)picolinamide
6-cyclopropyl-N-(3-(3-((S)-fluoro(4-methyl-4H-1,2,4-triazol-3-yl)methyl)oxetan-3-yl)phenyl)-4-((R)-1-(3-fluoro-3-methylazetidin-1-yl)ethyl)picolinamide
(R)-1-methyl-N-(3-(1-(4-methyl-4H-1,2,4-triazol-3-yl)propan-2-yl)phenyl)-4-(trifluoromethyl)-1H-imidazole-2-carboxamide
(R)-4-cyclopropyl-N-(3-(1-(4-methyl-4H-1,2,4-triazol-3-yl)propan-2-yl)phenyl)-1H-imidazole-2-carboxamide
N-(3-(3-((4-methyl-4H-1,2,4-triazol-3-yl)methyl)oxetan-3-yl)phenyl)-4-(trifluoromethyl)-1H-imidazole-2-carboxamide
N-(3-(3-(fluoro(4-methyl-4H-1,2,4-triazol-3-yl)methyl)oxetan-3-yl)phenyl)-4-(trifluoromethyl)-1H-imidazole-2-carboxamide
6-cyclopropyl-N-(3-(3,3-difluoro-1-((S)-fluoro(4-methyl-4H-1,2,4-triazol-3-yl)methyl)cyclobutyl)phenyl)-4-(((S)-2-isopropyl-4-methylpiperazin-1-yl)methyl)picolinamide
(S)-6-cyclopropyl-N-(3-(3,3-difluoro-1-((4-methyl-4H-1,2,4-triazol-3-yl)methyl)cyclobutyl)phenyl)-4-((2-isopropyl-4-methylpiperazin-1-yl)methyl)picolinamide
6-cyclopropyl-N-(3-(3,3-difluoro-1-((R)-fluoro(4-methyl-4H-1,2,4-triazol-3-yl)methyl)cyclobutyl)phenyl)-4-(((S)-2-isopropyl-4-methylpiperazin-1-yl)methyl)picolinamide
6-cyclopropyl-N-(3-(3,3-difluoro-1-((4-methyl-4H-1,2,4-triazol-3-yl)methyl)cyclobutyl)phenyl)-4-((3-hydroxy-3-methylazetidin-1-yl)methyl)picolinamide
6-cyclopropyl-N-(3-(3,3-difluoro-1-((4-methyl-4H-1,2,4-triazol-3-yl)methyl)cyclobutyl)phenyl)-4-((3-(hydroxymethyl)-3-methylazetidin-1-yl)methyl)picolinamide
(R)-6-cyclopropyl-N-(3-(3,3-difluoro-1-(fluoro(4-methyl-4H-1,2,4-triazol-3-yl)methyl)cyclobutyl)phenyl)-4-((3-(hydroxymethyl)-3-methylazetidin-1-yl)methyl)picolinamide
(S)-6-cyclopropyl-4-((2-isopropyl-4-methylpiperazin-1-yl)methyl)-N-(3-(3-((4-methyl-4H-1,2,4-triazol-3-yl)methyl)oxetan-3-yl)phenyl)picolinamide
6-cyclopropyl-N-(3-(3-((R)-fluoro(4-methyl-4H-1,2,4-triazol-3-yl)methyl)oxetan-3-yl)phenyl)-4-(((S)-2-isopropyl-4-methylpiperazin-1-yl)methyl)picolinamide
6-cyclopropyl-N-(3-(3-((S)-fluoro(4-methyl-4H-1,2,4-triazol-3-yl)methyl)oxetan-3-yl)phenyl)-4-(((S)-2-isopropyl-4-methylpiperazin-1-yl)methyl)picolinamide
N-(3-((S)-2-((S)-fluoro(4-methyl-4H-1,2,4-triazol-3-yl)methyl)oxetan-2-yl)phenyl)-6-(trifluoromethyl)picolinamide
N-(3-((S)-2-((R)-fluoro(4-methyl-4H-1,2,4-triazol-3-yl)methyl)oxetan-2-yl)phenyl)-6-(trifluoromethyl)picolinamide
N-(3-((R)-2-((S)-fluoro(4-methyl-4H-1,2,4-triazol-3-yl)methyl)oxetan-2-yl)phenyl)-6-(trifluoromethyl)picolinamide
N-(3-((R)-2-((R)-fluoro(4-methyl-4H-1,2,4-triazol-3-yl)methyl)oxetan-2-yl)phenyl)-6-(trifluoromethyl)picolinamide
4-(2-cyclopropyl-1-(5-azaspiro[2.4]heptan-5-yl)ethyl)-N-(3-(3-((4-methyl-4H-1,2,4-triazol-3-yl)methyl)oxetan-3-yl)phenyl)-6-(trifluoromethyl)picolinamide
4-(2-hydroxyethyl)-N-(3-(3-((4-methyl-4H-1,2,4-triazol-3-yl)methyl)oxetan-3-yl)phenyl)-6-(trifluoromethyl)-picolinamide; and
4-(2-amino-2-oxoethyl)-N-(3-(3-((4-methyl-4H-1,2,4-triazol-3-yl)methyl)oxetan-3-yl)phenyl)-6-(trifluoromethyl)picolinamide;
or a pharmaceutically acceptable salt or solvate thereof.

8. A pharmaceutical composition, comprising a compound according to claim 1 in combination with one or more pharmaceutically acceptable excipients.

9. A method of treating a cancer, the method comprising administering to a subject in need thereof, a therapeutically effective amount of a compound according to claim 1.

10. A method of treating a cancer, the method comprising administering to a subject in need thereof, a therapeutically effective amount of a composition according to claim 8.

11. A pharmaceutical composition, comprising a compound according to claim 7 in combination with one or more pharmaceutically acceptable excipients.

12. A method of treating a cancer, the method comprising administering to a subject in need thereof, a therapeutically effective amount of a compound according to claim 7.

13. A method of treating a cancer, the method comprising administering to a subject in need thereof, a therapeutically effective amount of a composition according to claim 11.
